(19) United States
(12) Patent Application Publication
(10) Pub. No.: US 2022/0306734 A1
(43) Pub. Date: Sep. 29, 2022

(54) ANTI-MGLUR5 ANTIBODIES AND USES THEREOF

(71) Applicant: H. LUNDBECK A/S, Valby (DK)

(72) Inventors: Carol J. RAPORT, Bothell, WA (US); Leon F. GARCIA-MARTINEZ, Woodinville, WA (US); Benjamin H. DUTZAR, Seattle, WA (US); Daniel S. ALLISON, Forest Park, WA (US); Jens BILLGREN, Seattle, WA (US); Michelle SCALLEY-KIM, Seattle, WA (US); Ethan Wayne OJALA, Snohomish, WA (US); Jenny MULLIGAN, Lake Forest Park, WA (US); Pei FAN, Bothell, WA (US); Maria-Cristina LOOMIS, Bothell, WA (US); Jeffrey T.L. SMITH, Dublin (IE); John A. LATHAM, Seattle, WA (US); Vanessa Lisbeth RUBIN, Seattle, WA (US)

(21) Appl. No.: 17/629,213

(22) PCT Filed: Jul. 22, 2020
(86) PCT No.: PCT/IB2020/056921

§ 371 Date: Jan. 21, 2022

Related U.S. Application Data
(60) Provisional application No. 62/877,889, filed on Jul. 24, 2019.

(51) Int. Cl. C07K 16/28; A61P 1/04; A61P 13/10; A61P 25/06

(52) U.S. Cl. CPC C07K 16/28; A61P 1/04; A61P 13/10; A61P 25/06; C07K 2317/22; C07K 2317/24; C07K 2317/31; C07K 2317/51; C07K 2317/52; C07K 2317/54; C07K 2317/55; C07K 2317/565; C07K 2317/622

(57) ABSTRACT

This invention generally pertains to antibodies and antigen-binding antibody Fragments, preferably humanized, chimeric, and human antibodies and antigen-binding antibody fragments. compositions containing such antibodies and antigen-binding antibody fragments or cells, e.g., immune cells such as T, Treg, or NK cells which express same, wherein such antibodies and antigen-binding antibody Fragments specifically bind to mGluR5. The invention also relates to therapeutic and diagnostic uses for the antibodies, antigen-binding antibody fragments, and compositions thereof.

### RELATED APPLICATION

The instant application claims priority to U.S. Provisional Application No. 62/877,889 filed Jul. 24, 2019 (Attorney Docket No. 1143257.008201), the entirety of which is incorporated by reference herein.

### FIELD OF THE INVENTION

### Sequence Listing Disclosure

The instant application contains a Sequence Listing which has been submitted in ASCII format via EFS-Web and is hereby incorporated by reference in its entirety. Said ASCII copy, created on Jul. 6, 2020, is named “1143257o008213.txt” and is 216,148 bytes in size.

This invention generally pertains to antibodies and antigen-binding fragments thereof, preferably humanized, chimeric, and human antibodies and antigen-binding fragments thereof, and compositions containing such antibodies and antigen-binding fragments thereof, wherein such antibodies and antigen-binding fragments thereof specifically bind to mGluR5 and preferably those which antagonize the effects of mGluR5. The invention further relates to therapeutic and diagnostic uses for the antibodies, antigen-binding fragments, and compositions thereof. The invention additionally relates to the specific use of these antibodies and antigen-binding fragments thereof as prophylactics or therapeutics for the treatment of migraine, gastro-esophageal reflux disease, irritable bowel syndrome, and other diseases involving mGluR5 activity in the peripheral or central nervous system.

### Background of the Invention

Metabotropic glutamate receptor 5 (mGluR5) is a glutamate-binding GPCR that is expressed on nerves of both the central nervous system (CNS) and the periphery, including at peripheral terminals of primary nociceptive afferent neurons. It binds to glutamate, which is a nonessential amino acid, a bioenergetic substrate for proliferating cells, and an excitatory neurotransmitter that is actively involved in biosynthetic, bioenergetic, and metabolic signaling pathways. Activation of mGluR5 and coupled Gq G-proteins initiates signaling cascades involving Phospholipase C, Inositol, and 1,4,5-triphosphate/diacylglycerol, and also results in the production of phospho-Extracellular signal-Related Kinase 1/2 (p-ERK). The architecture of mGluR5 involves a large N-terminal bi-lobed extracellular domain (ECD), followed by a cysteine-rich domain involved in dimerization and activation, seven alpha-helical transmembrane domains, and an intracellular cytoplasmic tail domain (Willard S S, Koochekpour S. Int J Biol Sci. 2013; 9(9):948-59.). mGluR5 is implicated in a number of pathologic processes including pain transmission, migraine, gastro-esophageal reflux disease (GERD), irritable bowel syndrome (IBS), overactive bladder (OAB)/incontinence, Fragile X syndrome (FXS), anxiety, drug addiction, and Parkinson's disease, among others.

Small molecule inhibitors of mGluR5 have been developed to treat many of these pathological conditions with mixed success. One in particular, 2-methyl-6-phenylethynyl pyridine hydrochloride (MPEP), has been used in numerous preclinical models to demonstrate the therapeutic potential of inhibiting mGluR5. Therapeutic small molecule inhibitors of mGluR5 have also been developed and tested successfully in early stage human clinical trials but have not advanced to successful drug approval. While small molecule inhibitors of mGluR5 have not been successfully approved, often due to unacceptable side effects and toxicities, they have nevertheless provided evidence that inhibition of mGluR5 may be effective in the treatment of a variety of diseases.

For migraine, the small molecule mGluR5 inhibitor ADX10059 (Addex) had successful outcomes in rodent models, showing that mGluR5 blockade attenuates neurogenic dural vasodilation and trigeminocervical complex neuronal activity. In phase IIa clinical trials, a statistically significant percentage of patients were pain free 2 h and 24 h after treatment. However, the drug led to adverse effects, primarily dizziness, in 79% of patients and hepatotoxicity was detected in subsequent studies. See Waung M W et al. Annals of Clinical and Translational Neurology. 2016; 3(8):560-71.

As for its relevance to GERD, mGluR5 is expressed in the peripheral gastric vagal afferent terminals, and antagonists are potent inhibitors of transient lower esophageal sphincter relaxation (TLESR) (Frisby C L et al. Gastroenterology. 2005; 129(3):995-1004). ADX10059 attained successful outcomes in phase II clinical trials: a high dose reduced esophageal acid exposure over a 24 hour period and decreased the number and duration of reflux episodes (Keywood C et al. Gut. 2009; 58(9):1192-9). Successful phase 1/2 clinical trial results were also observed with Mavoglurant/AFQ056 (Novartis), another mGluR5 small molecule inhibitor. Clinical studies on AZD2066 (AstraZeneca) demonstrated significant reduction in TLESRs and reflux episodes, but with a high percentage of adverse events (e.g. dizziness) (Rohof W O et al. Aliment Pharmacol Ther. 2012; 35:1231-42). All three drugs have since been discontinued in GERD.

For IBS, OAB, incontinence, and bladder visceral pain, successful preclinical data has been obtained with small molecule inhibitors administered in the periphery. mGluR5 inhibitors, including MPEP, have been shown to reduce responses to colorectal distension in rats. Mavoglurant was shown to be effective in colorectal distension models and bladder distension models, where it also reduced visceromotor responses and reduced bladder contractions. See, e.g., U.S. Patent Application Nos. US20070203163 and US20080207749 and U.S. Pat. No. 7,531,529.

Some successful clinical and preclinical results have also been obtained using small molecules administered peripherally for the treatment of neuropathic pain/peripheral neuropathy, inflammatory pain, and postoperative pain. See Clinical trial NCT00939094; Dogrul A et al. Neurosci Lett. 2000; 292:115-8; Bhave G et al. Nat Neurosci. 2001; 4:417-23; Walker K et al. Neuropharm. 2001; 40:1-9; and Zhu C Z et al. Pain. 2005; 114:195-202.

Therefore, in spite of their positive results in treating myriad diseases of the central and peripheral nervous system, many small molecule inhibitors of mGluR5 have demonstrated high levels of unwanted side effects (dizziness, psychosis) and have since been discontinued.

### BRIEF SUMMARY OF THE INVENTION

Many of the pathologic functions of mGluR5 may be attributed to over-activity of the peripheral mGluR5 receptors, which potentially are accessible to a blocking antibody. Diseases such as migraine, GERD, IBS and OAB, which involve inappropriate peripheral nerve signaling, may therefore be treated with an mGluR5 blocking antibody. Utilizing an antibody as a therapeutic may also avoid the unwanted side effects of small molecule inhibitors, which, without being limited to a particular theory, may be due to off-target effects and/or inhibition of mGluR5 in the CNS. Moreover, antibody therapy avoids the potential of liver toxicity due to small molecule inhibitor chemical structures. The present invention is directed to potent blocking monoclonal antibodies (mAbs) to mGluR5, with verified activity both in vitro and in vivo, and their use in the treatment of myriad diseases.

The invention provides an isolated antibody, or antigen-binding antibody fragment, that binds to human mGluR5 and antagonizes, inhibits, neutralizes or blocks at least one biological effect associated with human mGluR5.

In some embodiments, the isolated antibody or antigen-binding antibody fragment is a human, humanized, bispecific, multispecific or chimeric antibody or antigen-binding antibody fragment.

In some embodiments, the isolated antibody or antigen-binding antibody fragment is an antigen binding fragment selected from an Fab; an F(ab′)2; an Fab′ fragment; an Fv fragment; a single-chain Fv (scFv) fragment; an Fd fragment; a dAb fragment; a diabody; a nanobody; a bivalent nanobody; a shark variable IgNAR domain; a V_{H}H antibody; a camelid antibody; a chimeric antigen receptor (CAR), a BiTE (Bispecific T cell Engager) and a minibody.

In some embodiments, the antibody or antigen-binding antibody fragment is not N-glycosylated.

In some embodiments, the isolated antibody or antigen-binding antibody fragment allosterically inhibits human mGluR5.

In some embodiments, the isolated antibody or antigen-binding antibody fragment does not compete with quisqualate for binding to human mGluR5, optionally as measured via a radioligand binding inhibition assay.

In some embodiments, the isolated antibody or antigen-binding antibody fragment does not bind to human mGluR1.

In some embodiments, the isolated antibody or antigen-binding antibody fragment cross-reacts with rat and/or cynomolgus monkey mGluR5.

In some embodiments, the isolated antibody or antigen-binding antibody fragment inhibits the production of cytosolic phospho-ERK (pERK) in a pERK signaling assay, optionally wherein the IC50 of the antibody or antigen-binding antibody fragment in the pERK signaling assay is less than 100 nM, less than 50 nM, or less than 10 nM.

In some embodiments, the isolated antibody or antigen-binding antibody fragment inhibits migraine associated symptoms.

In some embodiments, the isolated antibody or antigen-binding antibody fragment inhibits umbellulone-induced lacrimation and/or umbellulone-induced facial temperature increase when administered to a subject.

In some embodiments, the isolated antibody or antigen-binding antibody fragment does not elicit the adverse side-effects associated with small molecule mGluR5 antagonists, e.g., liver toxicity and/or impaired motor coordination, optionally when peripherally administered. In some embodiments, the impaired motor coordination side-effect is dizziness.

In some embodiments, the isolated antibody or antigen-binding antibody fragment comprises:

(i) a V_{H} chain comprising a CDR1 sequence consisting of SEQ ID NO: 9; a CDR2 sequence consisting of SEQ ID NO: 11; and a CDR3 sequence consisting of SEQ ID NO: 13; and/or a V_{L} chain comprising a CDR1 sequence consisting of SEQ ID NO: 17; a CDR2 sequence consisting of SEQ ID NO: 19; and a CDR3 sequence consisting of SEQ ID NO: 21;

(ii) a V_{H} chain comprising a CDR1 sequence consisting of SEQ ID NO: 25; a CDR2 sequence consisting of SEQ ID NO: 27; and a CDR3 sequence consisting of SEQ ID NO: 29; and/or a V_{L} chain comprising a CDR1 sequence consisting of SEQ ID NO: 33; a CDR2 sequence consisting of SEQ ID NO: 35; and a CDR3 sequence consisting of SEQ ID NO: 37;

(iii) a V_{H} chain comprising a CDR1 sequence consisting of SEQ ID NO: 41; a CDR2 sequence consisting of SEQ ID NO: 43; and a CDR3 sequence consisting of SEQ ID NO: 45; and/or a V_{L} chain comprising a CDR1 sequence consisting of SEQ ID NO: 49; a CDR2 sequence consisting of SEQ ID NO: 51; and a CDR3 sequence consisting of SEQ ID NO: 53;

(iv) a V_{H} chain comprising a CDR1 sequence consisting of SEQ ID NO: 57; a CDR2 sequence consisting of SEQ ID NO: 59; and a CDR3 sequence consisting of SEQ ID NO: 61; and/or a V_{L} chain comprising a CDR1 sequence consisting of SEQ ID NO: 65; a CDR2 sequence consisting of SEQ ID NO: 67; and a CDR3 sequence consisting of SEQ ID NO: 69;

(v) a V_{H} chain comprising a CDR1 sequence consisting of SEQ ID NO: 73; a CDR2 sequence consisting of SEQ ID NO: 75; and a CDR3 sequence consisting of SEQ ID NO: 77; and/or a V_{L} chain comprising a CDR1 sequence consisting of SEQ ID NO: 81; a CDR2 sequence consisting of SEQ ID NO: 83; and a CDR3 sequence consisting of SEQ ID NO: 85;

(vi) a V_{H} chain comprising a CDR1 sequence consisting of SEQ ID NO: 89; a CDR2 sequence consisting of SEQ ID NO: 91; and a CDR3 sequence consisting of SEQ ID NO: 93; and/or a V_{L} chain comprising a CDR1 sequence consisting of SEQ ID NO: 97; a CDR2 sequence consisting of SEQ ID NO: 99; and a CDR3 sequence consisting of SEQ ID NO: 101;

(vii) a V_{H} chain comprising a CDR1 sequence consisting of SEQ ID NO: 105; a CDR2 sequence consisting of SEQ ID NO: 107; and a CDR3 sequence consisting of SEQ ID NO: 109; and/or a V_{L} chain comprising a CDR1 sequence consisting of SEQ ID NO: 113; a CDR2 sequence consisting of SEQ ID NO: 115; and a CDR3 sequence consisting of SEQ ID NO: 117;

(viii) a V_{H} chain comprising a CDR1 sequence consisting of SEQ ID NO: 121; a CDR2 sequence consisting of SEQ ID NO: 123; and a CDR3 sequence consisting of SEQ ID NO: 125; and/or a V_{L} chain comprising a CDR1 sequence consisting of SEQ ID NO: 129; a CDR2 sequence consisting of SEQ ID NO: 131; and a CDR3 sequence consisting of SEQ ID NO: 133;

(ix) a V_{H} chain comprising a CDR1 sequence consisting of SEQ ID NO: 137; a CDR2 sequence consisting of SEQ ID NO: 139; and a CDR3 sequence consisting of SEQ ID NO: 141; and/or a V_{L} chain comprising a CDR1 sequence consisting of SEQ ID NO: 145; a CDR2 sequence consisting of SEQ ID NO: 147; and a CDR3 sequence consisting of SEQ ID NO: 149;

(x) a V_{H} chain comprising a CDR1 sequence consisting of SEQ ID NO: 153; a CDR2 sequence consisting of SEQ ID NO: 155; and a CDR3 sequence consisting of SEQ ID NO: 157; and/or a V_{L} chain comprising a CDR1 sequence consisting of SEQ ID NO: 161; a CDR2 sequence consisting of SEQ ID NO: 163; and a CDR3 sequence consisting of SEQ ID NO: 165;

(xi) a V_{H} chain comprising a CDR1 sequence consisting of SEQ ID NO: 169; a CDR2 sequence consisting of SEQ ID NO: 171; and a CDR3 sequence consisting of SEQ ID NO: 173; and/or a V_{L} chain comprising a CDR1 sequence consisting of SEQ ID NO: 177; a CDR2 sequence consisting of SEQ ID NO: 179; and a CDR3 sequence consisting of SEQ ID NO: 181;

(xii) a V_{H} chain comprising a CDR1 sequence consisting of SEQ ID NO: 185; a CDR2 sequence consisting of SEQ ID NO: 187; and a CDR3 sequence consisting of SEQ ID NO: 189; and/or a V_{L} chain comprising a CDR1 sequence consisting of SEQ ID NO: 193; a CDR2 sequence consisting of SEQ ID NO: 195; and a CDR3 sequence consisting of SEQ ID NO: 197;

(xiii) a V_{H} chain comprising a CDR1 sequence consisting of SEQ ID NO: 201; a CDR2 sequence consisting of SEQ ID NO: 203; and a CDR3 sequence consisting of SEQ ID NO: 205; and/or a V_{L} chain comprising a CDR1 sequence consisting of SEQ ID NO: 209; a CDR2 sequence consisting of SEQ ID NO: 211; and a CDR3 sequence consisting of SEQ ID NO: 213;

(xiv) a V_{H} chain comprising a CDR1 sequence consisting of SEQ ID NO: 217; a CDR2 sequence consisting of SEQ ID NO: 219; and a CDR3 sequence consisting of SEQ ID NO: 221; and/or a V_{L} chain comprising a CDR1 sequence consisting of SEQ ID NO: 225; a CDR2 sequence consisting of SEQ ID NO: 227; and a CDR3 sequence consisting of SEQ ID NO: 229;

(xv) a V_{H} chain comprising a CDR1 sequence consisting of SEQ ID NO: 233; a CDR2 sequence consisting of SEQ ID NO: 235; and a CDR3 sequence consisting of SEQ ID NO: 237; and/or a V_{L} chain comprising a CDR1 sequence consisting of SEQ ID NO: 241; a CDR2 sequence consisting of SEQ ID NO: 243; and a CDR3 sequence consisting of SEQ ID NO: 245;

(xvi) a V_{H} chain comprising a CDR1 sequence consisting of SEQ ID NO: 249; a CDR2 sequence consisting of SEQ ID NO: 251; and a CDR3 sequence consisting of SEQ ID NO: 253; and/or a V_{L} chain comprising a CDR1 sequence consisting of SEQ ID NO: 257; a CDR2 sequence consisting of SEQ ID NO: 259; and a CDR3 sequence consisting of SEQ ID NO: 261;

(xvii) a V_{H} chain comprising a CDR1 sequence consisting of SEQ ID NO: 265; a CDR2 sequence consisting of SEQ ID NO: 267; and a CDR3 sequence consisting of SEQ ID NO: 269; and/or a V_{L} chain comprising a CDR1 sequence consisting of SEQ ID NO: 273; a CDR2 sequence consisting of SEQ ID NO: 275; and a CDR3 sequence consisting of SEQ ID NO: 277;

(xviii) a V_{H} chain comprising a CDR1 sequence consisting of SEQ ID NO: 281; a CDR2 sequence consisting of SEQ ID NO: 283; and a CDR3 sequence consisting of SEQ ID NO: 285; and/or a V_{L} chain comprising a CDR1 sequence consisting of SEQ ID NO: 289; a CDR2 sequence consisting of SEQ ID NO: 291; and a CDR3 sequence consisting of SEQ ID NO: 293;

(xix) a V_{H} chain comprising a CDR1 sequence consisting of SEQ ID NO: 297; a CDR2 sequence consisting of SEQ ID NO: 299; and a CDR3 sequence consisting of SEQ ID NO: 301; and/or a V_{L} chain comprising a CDR1 sequence consisting of SEQ ID NO: 305; a CDR2 sequence consisting of SEQ ID NO: 307; and a CDR3 sequence consisting of SEQ ID NO: 309;

(xx) a V_{H} chain comprising a CDR1 sequence consisting of SEQ ID NO: 313; a CDR2 sequence consisting of SEQ ID NO: 315; and a CDR3 sequence consisting of SEQ ID NO: 317; and/or a V_{L} chain comprising a CDR1 sequence consisting of SEQ ID NO: 321; a CDR2 sequence consisting of SEQ ID NO: 323; and a CDR3 sequence consisting of SEQ ID NO: 325;

(xxi) a V_{H} chain comprising a CDR1 sequence consisting of SEQ ID NO: 329; a CDR2 sequence consisting of SEQ ID NO: 331; and a CDR3 sequence consisting of SEQ ID NO: 333; and/or a V_{L} chain comprising a CDR1 sequence consisting of SEQ ID NO: 337; a CDR2 sequence consisting of SEQ ID NO: 339; and a CDR3 sequence consisting of SEQ ID NO: 341;

(xxii) a V_{H} chain comprising a CDR1 sequence consisting of SEQ ID NO: 345; a CDR2 sequence consisting of SEQ ID NO: 347; and a CDR3 sequence consisting of SEQ ID NO: 349; and/or a V_{L} chain comprising a CDR1 sequence consisting of SEQ ID NO: 353; a CDR2 sequence consisting of SEQ ID NO: 355; and a CDR3 sequence consisting of SEQ ID NO: 357;

(xxiii) a V_{H} chain comprising a CDR1 sequence consisting of SEQ ID NO: 361; a CDR2 sequence consisting of SEQ ID NO: 363; and a CDR3 sequence consisting of SEQ ID NO: 365; and/or a V_{L} chain comprising a CDR1 sequence consisting of SEQ ID NO: 369; a CDR2 sequence consisting of SEQ ID NO: 371; and a CDR3 sequence consisting of SEQ ID NO: 373;

(xxiv) a V_{H} chain comprising a CDR1 sequence consisting of SEQ ID NO: 377; a CDR2 sequence consisting of SEQ ID NO: 379; and a CDR3 sequence consisting of SEQ ID NO: 381; and/or a V_{L} chain comprising a CDR1 sequence consisting of SEQ ID NO: 385; a CDR2 sequence consisting of SEQ ID NO: 387; and a CDR3 sequence consisting of SEQ ID NO: 389;

(xxv) a V_{H} chain comprising a CDR1 sequence consisting of SEQ ID NO: 393; a CDR2 sequence consisting of SEQ ID NO: 395; and a CDR3 sequence consisting of SEQ ID NO: 397; and/or a V_{L} chain comprising a CDR1 sequence consisting of SEQ ID NO: 401; a CDR2 sequence consisting of SEQ ID NO: 403; and a CDR3 sequence consisting of SEQ ID NO: 405;

(xxvi) a V_{H} chain comprising a CDR1 sequence consisting of SEQ ID NO: 409; a CDR2 sequence consisting of SEQ ID NO: 411; and a CDR3 sequence consisting of SEQ ID NO: 413; and/or a V_{L} chain comprising a CDR1 sequence consisting of SEQ ID NO: 417; a CDR2 sequence consisting of SEQ ID NO: 419; and a CDR3 sequence consisting of SEQ ID NO: 421;

(xxvii) a V_{H} chain comprising a CDR1 sequence consisting of SEQ ID NO: 425; a CDR2 sequence consisting of SEQ ID NO: 427; and a CDR3 sequence consisting of SEQ ID NO: 429; and/or a V_{L} chain comprising a CDR1 sequence consisting of SEQ ID NO: 433; a CDR2 sequence consisting of SEQ ID NO: 435; and a CDR3 sequence consisting of SEQ ID NO: 437;

(xxviii) a V_{H} chain comprising a CDR1 sequence consisting of SEQ ID NO: 441; a CDR2 sequence consisting of SEQ ID NO: 443; and a CDR3 sequence consisting of SEQ ID NO: 445; and/or a V_{L} chain comprising a CDR1 sequence consisting of SEQ ID NO: 449; a CDR2 sequence consisting of SEQ ID NO: 451; and a CDR3 sequence consisting of SEQ ID NO: 453; or

(xxix) a V_{H} chain comprising a CDR1 sequence consisting of SEQ ID NO: 457; a CDR2 sequence consisting of SEQ ID NO: 459; and a CDR3 sequence consisting of SEQ ID NO: 461; and/or a V_{L} chain comprising a CDR1 sequence consisting of SEQ ID NO: 465; a CDR2 sequence consisting of SEQ ID NO: 467; and a CDR3 sequence consisting of SEQ ID NO: 469.

In some embodiments, the isolated antibody or antigen-binding antibody fragment comprises:

(i) a V_{H} chain comprising an amino acid sequence with at least 80, 85, 90, 95, 96, 97, 98, 99, or 100% sequence identity to SEQ ID NO: 7, and/or a V_{L} chain comprising an amino acid sequence with at least 80, 85, 90, 95, 96, 97, 98, 99, or 100% sequence identity to SEQ ID NO: 15;

(ii) a V_{H} chain comprising an amino acid sequence with at least 80, 85, 90, 95, 96, 97, 98, 99, or 100% sequence identity to SEQ ID NO: 23, and/or a V_{L} chain comprising an amino acid sequence with at least 80, 85, 90, 95, 96, 97, 98, 99, or 100% sequence identity to SEQ ID NO: 31;

(iii) a V_{H} chain comprising an amino acid sequence with at least 80, 85, 90, 95, 96, 97, 98, 99, or 100% sequence identity to SEQ ID NO: 39, and/or a V_{L} chain comprising an amino acid sequence with at least 80, 85, 90, 95, 96, 97, 98, 99, or 100% sequence identity to SEQ ID NO: 47;

(iv) a V_{H} chain comprising an amino acid sequence with at least 80, 85, 90, 95, 96, 97, 98, 99, or 100% sequence identity to SEQ ID NO: 55, and/or a V_{L} chain comprising an amino acid sequence with at least 80, 85, 90, 95, 96, 97, 98, 99, or 100% sequence identity to SEQ ID NO: 63;

(v) a V_{H} chain comprising an amino acid sequence with at least 80, 85, 90, 95, 96, 97, 98, 99, or 100% sequence identity to SEQ ID NO: 71, and/or a V_{L} chain comprising an amino acid sequence with at least 80, 85, 90, 95, 96, 97, 98, 99, or 100% sequence identity to SEQ ID NO: 79;

(vi) a V_{H} chain comprising an amino acid sequence with at least 80, 85, 90, 95, 96, 97, 98, 99, or 100% sequence identity to SEQ ID NO: 87, and/or a V_{L} chain comprising an amino acid sequence with at least 80, 85, 90, 95, 96, 97, 98, 99, or 100% sequence identity to SEQ ID NO: 95;

(vii) a V_{H} chain comprising an amino acid sequence with at least 80, 85, 90, 95, 96, 97, 98, 99, or 100% sequence identity to SEQ ID NO: 103, and/or a V_{L} chain comprising an amino acid sequence with at least 80, 85, 90, 95, 96, 97, 98, 99, or 100% sequence identity to SEQ ID NO: 111;

(viii) a V_{H} chain comprising an amino acid sequence with at least 80, 85, 90, 95, 96, 97, 98, 99, or 100% sequence identity to SEQ ID NO: 119, and/or a V_{L} chain comprising an amino acid sequence with at least 80, 85, 90, 95, 96, 97, 98, 99, or 100% sequence identity to SEQ ID NO: 127;

(ix) a V_{H} chain comprising an amino acid sequence with at least 80, 85, 90, 95, 96, 97, 98, 99, or 100% sequence identity to SEQ ID NO: 135, and/or a V_{L} chain comprising an amino acid sequence with at least 80, 85, 90, 95, 96, 97, 98, 99, or 100% sequence identity to SEQ ID NO: 143;

(x) a V_{H} chain comprising an amino acid sequence with at least 80, 85, 90, 95, 96, 97, 98, 99, or 100% sequence identity to SEQ ID NO: 151, and/or a V_{L} chain comprising an amino acid sequence with at least 80, 85, 90, 95, 96, 97, 98, 99, or 100% sequence identity to SEQ ID NO: 159;

(xi) a V_{H} chain comprising an amino acid sequence with at least 80, 85, 90, 95, 96, 97, 98, 99, or 100% sequence identity to SEQ ID NO: 167, and/or a V_{L} chain comprising an amino acid sequence with at least 80, 85, 90, 95, 96, 97, 98, 99, or 100% sequence identity to SEQ ID NO: 175;

(xii) a V_{H} chain comprising an amino acid sequence with at least 80, 85, 90, 95, 96, 97, 98, 99, or 100% sequence identity to SEQ ID NO: 183, and/or a V_{L} chain comprising an amino acid sequence with at least 80, 85, 90, 95, 96, 97, 98, 99, or 100% sequence identity to SEQ ID NO: 191;

(xiii) a V_{H} chain comprising an amino acid sequence with at least 80, 85, 90, 95, 96, 97, 98, 99, or 100% sequence identity to SEQ ID NO: 199, and/or a V_{L} chain comprising an amino acid sequence with at least 80, 85, 90, 95, 96, 97, 98, 99, or 100% sequence identity to SEQ ID NO: 207;

(xiv) a V_{H} chain comprising an amino acid sequence with at least 80, 85, 90, 95, 96, 97, 98, 99, or 100% sequence identity to SEQ ID NO: 215, and/or a V_{L} chain comprising an amino acid sequence with at least 80, 85, 90, 95, 96, 97, 98, 99, or 100% sequence identity to SEQ ID NO: 223;

(xv) a V_{H} chain comprising an amino acid sequence with at least 80, 85, 90, 95, 96, 97, 98, 99, or 100% sequence identity to SEQ ID NO: 231, and/or a V_{L} chain comprising an amino acid sequence with at least 80, 85, 90, 95, 96, 97, 98, 99, or 100% sequence identity to SEQ ID NO: 239;

(xvi) a V_{H} chain comprising an amino acid sequence with at least 80, 85, 90, 95, 96, 97, 98, 99, or 100% sequence identity to SEQ ID NO: 247, and/or a V_{L} chain comprising an amino acid sequence with at least 80, 85, 90, 95, 96, 97, 98, 99, or 100% sequence identity to SEQ ID NO: 255;

(xvii) a V_{H} chain comprising an amino acid sequence with at least 80, 85, 90, 95, 96, 97, 98, 99, or 100% sequence identity to SEQ ID NO: 263, and/or a V_{L} chain comprising an amino acid sequence with at least 80, 85, 90, 95, 96, 97, 98, 99, or 100% sequence identity to SEQ ID NO: 271;

(xviii) a V_{H} chain comprising an amino acid sequence with at least 80, 85, 90, 95, 96, 97, 98, 99, or 100% sequence identity to SEQ ID NO: 279, and/or a V_{L} chain comprising an amino acid sequence with at least 80, 85, 90, 95, 96, 97, 98, 99, or 100% sequence identity to SEQ ID NO: 287;

(xix) a V_{H} chain comprising an amino acid sequence with at least 80, 85, 90, 95, 96, 97, 98, 99, or 100% sequence identity to SEQ ID NO: 295, and/or a V_{L} chain comprising an amino acid sequence with at least 80, 85, 90, 95, 96, 97, 98, 99, or 100% sequence identity to SEQ ID NO: 303;

(xx) a V_{H} chain comprising an amino acid sequence with at least 80, 85, 90, 95, 96, 97, 98, 99, or 100% sequence identity to SEQ ID NO: 311, and/or a V_{L} chain comprising an amino acid sequence with at least 80, 85, 90, 95, 96, 97, 98, 99, or 100% sequence identity to SEQ ID NO: 319;

(xxi) a V_{H} chain comprising an amino acid sequence with at least 80, 85, 90, 95, 96, 97, 98, 99, or 100% sequence identity to SEQ ID NO: 327, and/or a V_{L} chain comprising an amino acid sequence with at least 80, 85, 90, 95, 96, 97, 98, 99, or 100% sequence identity to SEQ ID NO: 335;

(xxii) a V_{H} chain comprising an amino acid sequence with at least 80, 85, 90, 95, 96, 97, 98, 99, or 100% sequence identity to SEQ ID NO: 343, and/or a V_{L} chain comprising an amino acid sequence with at least 80, 85, 90, 95, 96, 97, 98, 99, or 100% sequence identity to SEQ ID NO: 351;

(xxiii) a V_{H} chain comprising an amino acid sequence with at least 80, 85, 90, 95, 96, 97, 98, 99, or 100% sequence identity to SEQ ID NO: 359, and/or a V_{L} chain comprising an amino acid sequence with at least 80, 85, 90, 95, 96, 97, 98, 99, or 100% sequence identity to SEQ ID NO: 367;

(xxiv) a V_{H} chain comprising an amino acid sequence with at least 80, 85, 90, 95, 96, 97, 98, 99, or 100% sequence identity to SEQ ID NO: 375, and/or a V_{L} chain comprising an amino acid sequence with at least 80, 85, 90, 95, 96, 97, 98, 99, or 100% sequence identity to SEQ ID NO: 383;

(xxv) a V_{H} chain comprising an amino acid sequence with at least 80, 85, 90, 95, 96, 97, 98, 99, or 100% sequence identity to SEQ ID NO: 391, and/or a V_{L} chain comprising an amino acid sequence with at least 80, 85, 90, 95, 96, 97, 98, 99, or 100% sequence identity to SEQ ID NO: 399;

(xxvi) a V_{H} chain comprising an amino acid sequence with at least 80, 85, 90, 95, 96, 97, 98, 99, or 100% sequence identity to SEQ ID NO: 407, and/or a V_{L} chain comprising an amino acid sequence with at least 80, 85, 90, 95, 96, 97, 98, 99, or 100% sequence identity to SEQ ID NO: 415;

(xxvii) a V_{H} chain comprising an amino acid sequence with at least 80, 85, 90, 95, 96, 97, 98, 99, or 100% sequence identity to SEQ ID NO: 423, and/or a V_{L} chain comprising an amino acid sequence with at least 80, 85, 90, 95, 96, 97, 98, 99, or 100% sequence identity to SEQ ID NO: 431;

(xxviii) a V_{H} chain comprising an amino acid sequence with at least 80, 85, 90, 95, 96, 97, 98, 99, or 100% sequence identity to SEQ ID NO: 439, and/or a V_{L} chain comprising an amino acid sequence with at least 80, 85, 90, 95, 96, 97, 98, 99, or 100% sequence identity to SEQ ID NO: 447; or

(xxix) a V_{H} chain comprising an amino acid sequence with at least 80, 85, 90, 95, 96, 97, 98, 99, or 100% sequence identity to SEQ ID NO: 455, and/or a V_{L} chain comprising an amino acid sequence with at least 80, 85, 90, 95, 96, 97, 98, 99, or 100% sequence identity to SEQ ID NO: 463.

The invention is further directed to an isolated antibody or antigen-binding antibody fragment that competes with and/or which binds to the same or overlapping epitope as an antibody according to the foregoing sequence-specific embodiments.

In some embodiments, the isolated antibody or antigen-binding antibody fragment is selected from the group consisting of: a monoclonal antibody; a monospecific antibody; a polyspecific antibody; a humanized antibody; a tetrameric antibody; a tetravalent antibody; a multispecific antibody; a single chain antibody; a domain-specific antibody; a single domain antibody; a domain-deleted antibody; an scFc fusion protein; a chimeric antibody; a synthetic antibody; a recombinant antibody; a hybrid antibody; a mutated antibody; CDR-grafted antibodies; an antibody fragment; an Fab; an F(ab′)2; an Fab′ fragment; an Fv fragment; a single-chain Fv (scFv) fragment; an Fd fragment; a dAb fragment; a diabody; a nanobody; a bivalent nanobody; a shark variable IgNAR domain; a V_{H}H antibody; a camelid antibody; a BiTE (Bispecific T cell Engager), a chimeric antigen receptor (CAR) and a minibody.

In some embodiments, the isolated antibody or antigen-binding antibody fragment comprises a human IgG1, IgG2, IgG3, or IgG4 Fc region.

In some embodiments, the isolated antibody or antigen-binding antibody fragment comprises an Fc region that has been modified to alter at least one of effector function, half-life, proteolysis, or glycosylation.

In some embodiments, the isolated antibody or antigen-binding antibody fragment may be modified to decrease effector function, eliminate N-glycosylation, and/or decrease Fc receptor binding.

In some embodiments, the isolated antibody or antigen-binding antibody fragment binds to the extracellular domain of human mGluR5 with an EC50 of less than 10 nM, less than 5 nM, or less than 2 nM, as measured via an HTRF or AlphaLISA fluorescence assay.

In some embodiments, the isolated antibody or antigen-binding antibody fragment additionally has one or more of the following modifications:

(i) is conjugated to a cytotoxic agent;
(ii) is comprised in a bispecific antibody;
(iii) is comprised in a multispecific antigen-binding protein;
(iv) is conjugated to a label; and
(v) is conjugated to another therapeutic agent.

In some embodiments, the label is a chemiluminescent label, a paramagnetic label, an MRI contrast agent, a fluorescent label, a bioluminescent label, or a radioactive label.

In some embodiments, the isolated antibody or antigen-binding antibody fragment is suitable for treating a subject having migraine, gastro-esophageal reflux disease (GERD), irritable bowel syndrome (IBS), pain, overactive bladder (OAB)/incontinence, symptoms of an autism spectrum disorder, a psychiatric disorder, or a neurological disorder.

The invention additionally provides an anti-idiotypic antibody or antigen-binding antibody fragment produced against an anti-mGluR5 antibody or antigen-binding antibody fragment according to any of the preceding embodiments, which optionally neutralizes one or more biological effects of the anti-mGluR5 antibody or antigen-binding antibody fragment to which it binds.

The invention also provides a method of using said anti-idiotypic antibody or antibody fragment to monitor the in vivo levels of said anti-mGluR5 antibody or antigen-binding antibody fragment in a subject or to neutralize the in vivo effects of said anti-mGluR5 antibody or antigen-binding antibody fragment in a subject.

In another aspect, the invention provides an anti-anti-idiotypic antibody or antigen-binding antibody fragment produced against an anti-idiotypic antibody or antigen-binding antibody fragment according to the foregoing, optionally wherein the anti-anti-idiotypic antibody or antigen-binding antibody fragment neutralizes the anti-idiotypic antibody or antigen-binding antibody fragment to which it binds.

The invention also provides a pharmaceutical composition comprising a pharmaceutically effective amount of an isolated antibody or antigen-binding antibody fragment according to any of the foregoing embodiments, or a cell which expresses an antibody or antigen-binding antibody fragment according to any of the foregoing embodiments, e.g., an immune cell such as a T, Treg or NK cell, which further comprising a pharmaceutical diluent, carrier, or excipient.

In some embodiments, said anti-idiotypic antibody or antibody fragment is peripherally administered.

In some embodiments, said anti-idiotypic antibody or antibody fragment is centrally administered.

In some embodiments, said anti-idiotypic antibody or antibody fragment is peripherally and centrally administered.

The invention also provides an isolated polynucleotide encoding the antibody or antigen-binding antibody fragment according to any of the foregoing embodiments. The invention also provides an expression vector comprising the polynucleotide. The invention also provides a host cell comprising the expression vector.

The invention also provides a method of producing an isolated anti-mGluR5 antibody or antigen-binding antibody fragment comprising culturing the host cell under conditions that allow expression of the antibody or antigen-binding antibody fragment; and recovering the antibody or antigen-binding antibody fragment from the culture medium or host cell.

The invention also provides a method for treating or preventing a disorder associated with the peripheral or central nervous system comprising administering to a subject in need thereof a therapeutically or prophylactically effective amount of an antibody, antigen-binding antibody fragment, or pharmaceutical composition according to any of the foregoing embodiments.

In some embodiments, said antibody, antigen-binding antibody fragment, or pharmaceutical composition is peripherally administered.

In some embodiments, said antibody, antigen-binding antibody fragment, or pharmaceutical composition is centrally administered.

In some embodiments, said antibody, antigen-binding antibody fragment, or pharmaceutical composition is peripherally and centrally administered.

In some embodiments, administration of the antibody, antigen-binding antibody fragment, or pharmaceutical composition has one or more of the following effects:

(i) inhibits mGluR5 signaling;
(ii) does not inhibit mGluR1;
(iii) inhibits the production of cytosolic pERK;
(iv) does not result in a dizziness and/or other motor function side effect.

In some embodiments, the method is used to treat or prevent a peripheral nervous system disorder.

In some embodiments, the method is used to treat or prevent a central nervous system disorder.

In some embodiments, the method is used to treat or prevent migraine.

In some embodiments, the method is used to treat or prevent pain.

In some embodiments, the method is used to treat or prevent GERD.

In some embodiments, the method is used to treat or prevent IBS.

In some embodiments, the method is used to treat or prevent overactive bladder (OAB) or incontinence.

In some embodiments, the method is used to treat or prevent a neurological or psychiatric disorder.

The invention also provides a method for treating or preventing a condition, disease, or disorder associated with mGluR5 activity, comprising administering to a subject in need thereof a therapeutically or prophylactically effective amount of an antibody, antigen-binding antibody fragment, or pharmaceutical composition according to any of the foregoing embodiments.

The invention also provides a method for treating or preventing migraine, comprising administering to a subject in need thereof a therapeutically or prophylactically effective amount of an antibody, antigen-binding antibody fragment, or pharmaceutical composition according to any of the foregoing embodiments.

In some embodiments, the method treats or prevents one or more symptoms of migraine, or reduces the frequency and/or severity of such one or more symptoms, optionally vasomotor symptoms (e.g. hot flashes, facial flushing, sweating, and night sweats), photophobia, phonophobia, sensitivity to smells, tearing/lacrimation, vertigo, dizziness, nausea, vomiting, headache pain, and aura.

In some embodiments, the monthly incidence of migraine is reduced following administration of the antibody, antigen-binding antibody fragment, or pharmaceutical composition.

In some embodiments, the subject has or is diagnosed with episodic migraine or chronic migraine.

In some embodiments, the subject has or is diagnosed with cluster headache.

In some embodiments, the patient has not previously received prophylactic therapy for migraine headaches.

In some embodiments, the patient has failed or is intolerant to at least one other migraine headache prophylactic therapy, optionally an antiepileptic, a tricyclic antidepressant, or a beta-blocker.

The invention also provides a method for treating or preventing GERD, comprising administering to a subject in need thereof a therapeutically or prophylactically effective amount of an antibody, antigen-binding antibody fragment, or pharmaceutical composition according to any of the foregoing embodiments.

In some embodiments, the gastroesophageal reflux disease is nonerosive reflux disease.

In some embodiments, the gastroesophageal reflux disease is erosive esophagitis.

The invention also provides a method for treating or preventing irritable bowel syndrome (IBS) comprising administering to a subject in need thereof a therapeutically or prophylactically effective amount of an antibody, antigen-binding antibody fragment, or pharmaceutical composition according to any of the foregoing embodiments.

In some embodiments, the IBS is diarrhea predominant IBS, constipation predominant IBS, or alternating bowel movement predominant IBS.

The invention also provides a method for treating or preventing overactive bladder (OAB) comprising administering to a subject in need thereof a therapeutically or prophylactically effective amount of an antibody, antigen-binding antibody fragment, or pharmaceutical composition according to any of the foregoing embodiments.

In some embodiments, the method treats or prevents one or more of urgency, urinary frequency, nocturia, or urge incontinence.

The invention also provides a method for treating or preventing urinary incontinence comprising administering to a subject in need thereof a therapeutically or prophylactically effective amount of an antibody, antigen-binding antibody fragment, or pharmaceutical composition according to any of the foregoing embodiments.

In some embodiments, the method is used to treat incontinence selected from stress incontinence, urge incontinence, overflow incontinence, mixed incontinence, structural incontinence, functional incontinence, nocturnal incontinence, transient incontinence, giggle incontinence, double incontinence, post-void dribbling, and coital incontinence.

The invention also provides a method for treating, or alleviating or preventing one or more symptoms associated with, an autism spectrum disorder, comprising administering to a subject in need thereof a therapeutically or prophylactically effective amount of an antibody, antigen-binding antibody fragment, or pharmaceutical composition according to any of the foregoing embodiments.

In some embodiments, the one or more symptoms are selected from impaired social and functional communication, anxiety, inattention, hyperactivity, altered sensory reactivity, self-injury, aggression, impaired cognitive function, and compromised daily living skills.

The invention also provides a method for treating or preventing a neurological or psychiatric disorder comprising administering to a subject in need thereof a therapeutically or prophylactically effective amount of an antibody, antigen-binding antibody fragment, or pharmaceutical composition according to any of the foregoing embodiments.

In some embodiments, the neurological or psychiatric disorder is selected from schizophrenia, schizophreniform disorder, schizoaffective disorder, delusional disorder, brief psychotic disorder, shared psychotic disorder, psychotic disorder due to a general medical condition, substance-induced psychotic disorder, psychotic disorder not otherwise specified, psychosis associated with dementia, major depressive disorder, dysthymic disorder, premenstrual dysphoric disorder, depressive disorder not otherwise specified, bipolar I disorder, bipolar II disorder, cyclothymic disorder, bipolar disorder not otherwise specified, mood disorder due to a general medical condition, substance-induced mood disorder, mood disorder not otherwise specified, generalized anxiety disorder, obsessive-compulsive disorder, panic disorder, acute stress disorder, post-traumatic stress disorder, mental retardation, pervasive developmental disorders, attention deficit disorders, attention-deficit/hyperactivity disorder, disruptive behavior disorders, personality disorder of the paranoid type, personality disorder of the schizoid type, personality disorder of the schizotypical type, tic disorders, Tourette's syndrome, substance dependence, substance abuse, substance withdrawal, trichotillomania, and conditions wherein cognition is impaired, Alzheimer's disease, Parkinson's disease, levodopa-induced dyskinesia in Parkinson's disease patients, Huntingdon's disease, Lewy Body Dementia, dementia due to HIV disease, dementia due to Creutzfeldt-Jakob disease, amnestic disorders, mild cognitive impairment, age-related cognitive decline, feeding disorders such as anorexia and bulimia, and obesity.

In some embodiments, the method is used for treating or preventing behavior and dependence disorders, including alcohol, nicotine, cocaine, amphetamine, benzodiazepine, analgesics, opiate or other substance tolerance or dependence, bulimia nervosa, anorexia nervosa, gambling dependence, sex dependence, or obsessive compulsive disorders.

In some embodiments, the method is used for treating or preventing a neurological disorder selected from the group of neurodegeneration, neurotoxicity, ischemia, Parkinson's disease, memory impairment, Alzheimer's disease, dementia, and delirium tremens.

The invention also provides a method for treating or preventing pain comprising administering to a subject in need thereof a therapeutically or prophylactically effective amount of an antibody, antigen-binding antibody fragment, or pharmaceutical composition according to any of the foregoing embodiments.

In some embodiments, the pain is selected from neuropathic pain, central pain syndromes, postoperative pain, bone and joint pain, repetitive motion pain, dental pain, cancer pain, myofascial pain, perioperative pain, chronic pain, acute pain, dysmenorrhea, pain associated with angina, inflammatory pain, headache, migraine and cluster headache, primary hyperalgesia, secondary hyperalgesia, primary allodynia, secondary allodynia, or other pain.

In some embodiments, the subject is a mammal.

In some embodiments, the mammal is a human.

In some embodiments, the mammal is a non-human primate.

In some embodiments, the mammal is a rodent.

In some embodiments, the antibody or antigen-binding antibody fragment is not N-glycosylated.

In some embodiments, the antibody, antigen-binding antibody fragment, or pharmaceutical composition is administered as a monotherapy.

In some embodiments, the antibody, antigen-binding antibody fragment, or pharmaceutical composition is administered in combination with a second therapeutic agent.

In some embodiments, the antibody, antigen-binding antibody fragment, or pharmaceutical composition is administered peripherally.

In some embodiments, the antibody, antigen-binding antibody fragment, or pharmaceutical composition is administered centrally.

In some embodiments, the antibody, antigen-binding antibody fragment, or pharmaceutical composition is administered peripherally and centrally.

In some embodiments, the antibody, antigen-binding antibody fragment, or pharmaceutical composition is administered enterally, parenterally, or topically, preferably wherein the administration is parenteral.

In some embodiments, the parenteral administration is intravenous.

In some embodiments, the administration is intrathecal.

In some embodiments, administration of the antibody, antigen-binding antibody fragment, or pharmaceutical composition does not substantially cause an adverse central nervous system side effect in the patient.

In some embodiments, the method does not elicit side-effects associated with small molecule mGluR5 antagonists.

In some embodiments, the method does not cause dizziness.

The invention also provides a method for diagnosing a condition associated with upregulation of mGluR5 expression, said method comprising:

(i) isolating the cells or tissues responsible for mediating the condition;
(ii) contacting said cells with an anti-mGluR5 antibody or antigen-binding antibody fragment according to any of the foregoing embodiments; and
(iii) detecting the level of anti-mGluR5 antibody or antigen-binding antibody fragment bound to said cells.

### DETAILED DESCRIPTION OF THE SEVERAL VIEWS OF THE DRAWINGS

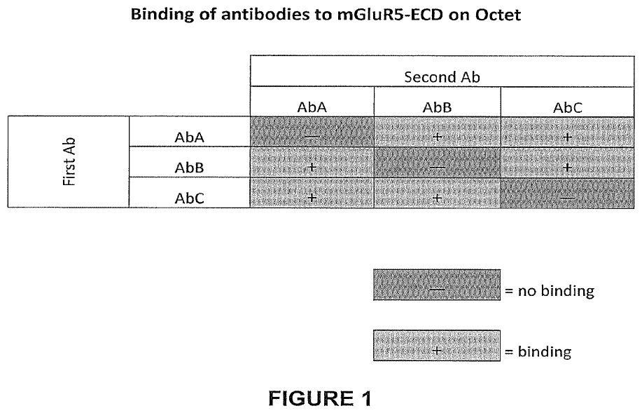

FIG. 1 contains a table demonstrating the binding analysis (bin determination) for exemplary mGluR5 antibodies AbA, AbB, and AbC. Biotinylated mGluR5-extracellular domain (ECD) was immobilized onto the surface of an Octet™ streptavidin sensor. “First Ab” indicates the antibody that was first bound to saturation to the immobilized mGluR5-ECD protein. The “Second Ab” was then allowed to interact with the “First Ab”: biotinylated mGluR5-ECD complex. A minus sign indicates no binding observed for the second antibody. A plus sign indicates that binding was observed for the second antibody, indicating that “First Ab” did not preclude binding of “Second Ab” to biotinylated mGluR5-ECD.

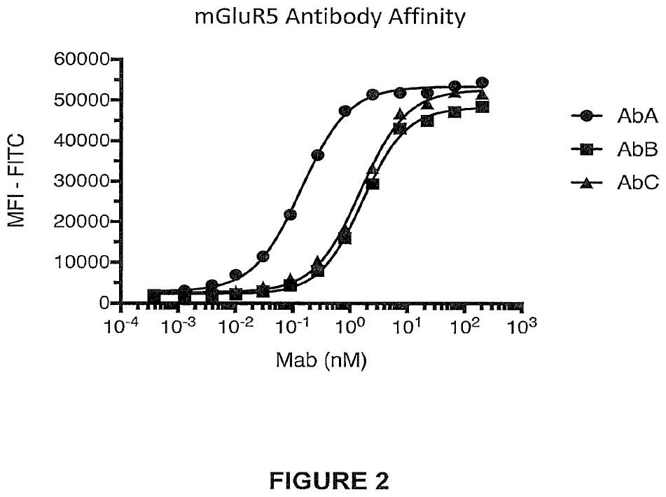

FIG. 2 shows the binding of increasing concentrations of antibody onto BA/F3 cells expressing mGluR5 detected by flow cytometry used to determine the binding affinities for exemplary antibodies AbA, AbB, and AbC. Mean fluorescence intensity of the secondary antibody (y-axis) was recorded as a function of primary antibody concentration (x-axis).

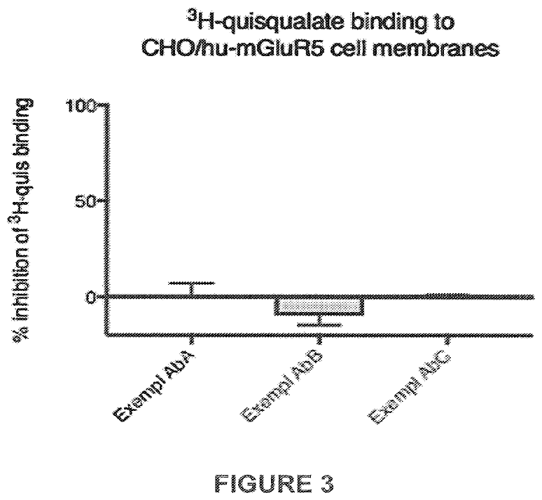

FIG. 3 contains the results of binding competition of ³H-labelled quisqualate ligand by exemplary antibodies AbA, AbB, and AbC. For each of the three exemplary antibodies representative of the three different binding epitope bins, the graph shows the percent inhibition of ³H-quisqualate binding to mGluR5 in cell membrane homogenates from CHO cells transfected with human mGluR5. Error bars represent the standard error of two replicate samples. The graph is representative of two separate experiments.

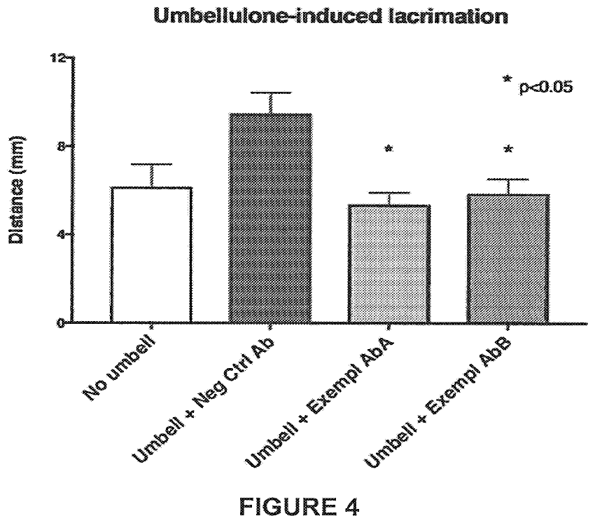

FIG. 4 contains the results of the in vivo inhibition of intranasal umbellulone-induced lacrimation in Sprague-Dawley rats. From left to right, the four bars represent the results of the experiment for the following conditions: the no umbellulone control, corresponding to normal lacrimation (white bar); umbellulone administered 24 hours after a negative control antibody (dark gray bar); umbellulone administered 24 hours after exemplary antibody AbA (light gray bar); umbellulone administered 24 hours after exemplary antibody AbB (medium gray bar). Error bars represent the standard error of results from 12 animals.*—statistically significant results, p<0.05, when compared to negative control antibody treatment.

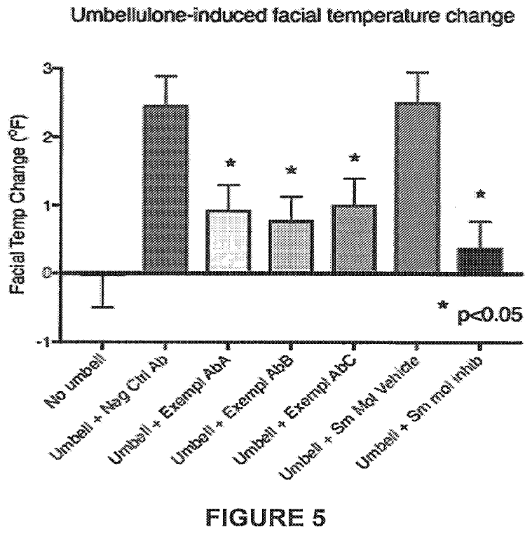

FIG. 5 contains the results of the in vivo inhibition of intranasal umbellulone-induced facial temperature increase. From left to right, the seven bars represent the results of the study for the following conditions: no umbellulone control; and umbellulone administered after negative control antibody; exemplary antibody AbA; exemplary antibody AbB; exemplary antibody AbC; small molecule vehicle; or small molecule mGluR5 inhibitor. Error bars represent the standard error of results from 12 animals. *—statistically significant results, p<0.05, when compared to negative control antibody or to small molecule vehicle treatments.

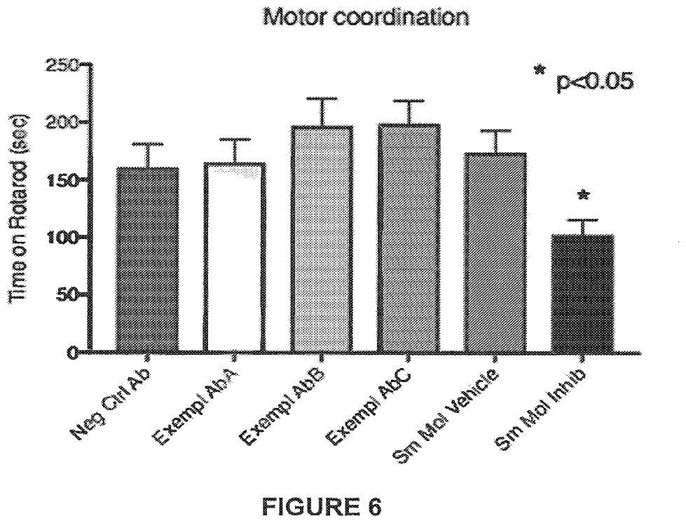

FIG. 6 contains the results of the analysis of motor coordination post administration of, from left to right: negative control antibody; exemplary antibody AbA; exemplary antibody AbB; exemplary antibody AbC; small molecule vehicle; and small molecule mGluR5 inhibitor. Error bars represent the standard error of results from 12 animals. *—statistically significant results, p<0.05, when compared to small molecule vehicle treatment.

### DETAILED DESCRIPTION OF THE INVENTION

The present invention relates to antibodies and antigen-binding fragments thereof that bind to metabotropic glutamate receptor 5 (“mGluR5”), nucleic acids encoding said antibodies and antigen-binding fragments thereof, compositions comprising said antibodies and antigen-binding fragments thereof, and methods of using said antibodies, antibody fragments, and compositions in diagnostics and therapy. The invention also provides particular anti-mGluR5 antibodies (Ab1-Ab29) and sequences thereof, as disclosed in SEQ ID NOS:6-469. The invention is further directed to any antibody having the CDR sequences of Ab1-Ab29.

### Antibody Target: mGluR5

The metabotropic glutamate receptors (mGluR) are G-protein coupled receptors that are involved in the regulation and activity of many synapses in the nerves of the central and peripheral nervous systems. Eight metabotropic glutamate receptor subtypes have been identified and are subdivided into three groups based on sequence similarity. Group I consists of mGluR1 and mGluR5. These receptors stimulate phospholipase C and/or p-ERK generation, and increase neuronal excitability. Group II, consisting of mGluR2 and mGluR3 as well as group III, consisting of mGluR4, mGluR6, mGluR7 and mGluR8 are capable of inhibiting adenylyl cyclase activity and reduce synaptic transmission. Several of the receptors also exist in various isoforms, occurring by alternative splicing (Chen, C-Y et al., Journal of Physiology (2002), 538.3, pp. 773-786; Pin, J-P et al., European Journal of Pharmacology (1999), 375, pp. 277-294; Brauner-Osborne, H et al. Journal of Medicinal Chemistry (2000), 43, pp. 2609-2645; Schoepp, D. D, Jane D. E. Monn J. A. Neuropharmacology (1999), 38, pp. 1431-1476).

The subject application provides novel anti-mGluR5, particularly anti-human mGluR5, antibodies including those comprising the same CDRS as mGluR5 Ab1-Ab29. To the best of the inventors' knowledge, prior to the present invention, no monoclonal anti-mGluR5 antibodies or antibody fragments that block the function of mGluR5 have been reported.

The binding of an anti-mGluR5 antibody or antibody fragment to mGluR5 according to the invention will reduce, suppress, diminish, or otherwise inhibit at least one of the functions of mGluR5. Inhibition may be partial or complete. Also as demonstrated in the Examples, the anti-mGluR5 antibodies of the invention may inhibit mGluR5 allosterically rather than competitively.

Antibodies that block or inhibit one or more of the functions of mGluR5 may be used to treat or alleviate symptoms associated with diseases of the central or peripheral nervous system. In particular, these antibodies may be used to treat ailments including but not limited to migraine, pain, GERD, IBS, OAB, incontinence, autism, addiction, and neurologic and psychiatric disorders, including but not limited to Alzheimer's, fragile X-syndrome, Tourette's syndrome, dementia, anxiety, and Parkinson's disease.

mGluR5 has different isoforms produced by alternative splicing. mGluR5a and mGluR5b differ in the absence or presence of amino acids 877-908, which are contained within the intracellular C-terminal domain. While expression levels of the isoforms vary, both isoforms activate phospholipase C and have similar pharmacological profiles. See Minakami R et al. Biochemical and Biophysical Research Communications. 1994; 199(3):1136-43. The present invention encompasses anti-mGluR5 antibodies which bind to and inhibit the activity of either or both of these isoforms.

Human mGluR5 (longer isoform) has the following amino acid sequence
(SEQ ID NO: 1); NCBI accession number NP_001137303.1.
SEQ ID NO: 1
MVLLLILSVLLLKEDVRGSAQSSERRVVAHMPGDIIIGALFSVHHQPTVDKVH
ERKCGAVREQYGIQRVEAMLHTLERINSDPTLLPNITLGCEIRDSCWHSAVALEQSIE
FIRDSLISSEEEEGLVRCVDGSSSSFRSKKPIVGVIGPGSSSVAIQVQNLLQLFNIPQIAY
SATSMDLSDKTLFKYFMRVVPSDAQQARAMVDIVKRYNWTYVSAVHTEGNYGESG
MEAFKDMSAKEGICIAHSYKIYSNAGEQSFDKLLKKLTSHLPKARVVACFCEGMTV
RGLLMAMRRLGLAGEFLLLGSDGWADRYDVTDGYQREAVGGITIKLQSPDVKWFD
DYYLKLRPETNHRNPWFQEFWQHRFQCRLEGFPQENSKYNKTCNSSLTLKTHHVQD
SKMGFVINAIYSMAYGLHNMQMSLCPGYAGLCDAMKPIDGRKLLESLMKTNFTGV
SGDTILFDENGDSPGRYEIMNFKEMGKDYFDYINVGSWDNGELKMDDDEVWSKKS
NIIRSVCSEPCEKGQIKVIRKGEVSCCWTCTPCKENEYVFDEYTCKACQLGSWPTDDL
TGCDLIPVQYLRWGDPEPIAAVVFACLGLLATLFVTVVFIIYRDTPVVKSSSRELCYII
LAGICLGYLCTFCLIAKPKQIYCYLQRIGIGLSPAMSYSALVTKTNRIARILAGSKKKIC
TKKPRFMSACAQLVIAFILICIQLGIIVALFIMEPPDIMHDYPSIREVYLICNTTNLGVVT
PLGYNGLLILSCTFYAFKTRNVPANFNEAKYIAFTMYTTCIIWLAFVPIYFGSNYKIIT
MCFSVSLSATVALGCMFVPKVYIILAKPERNVRSAFTTSTVVRMHVGDGKSSSAASR
SSSLVNLWKRRGSSGETLRYKDRRLAQHKSEIECFTPKGSMGNGGRATMSSSNGKS
VTWAQNEKSSRGQHLWQRLSIHINKKENPNQTAVIKPFPKSTESRGLGAGAGAGGS
AGGVGATGGAGCAGAGPGGPESPDAGPKALYDVAEAEEHFPAPARPRSPSPISTLSH
RAGSASRTDDDVPSLHSEPVARSSSSQGSLMEQISSVVTRFTANISELNSMMLSTAAP
SPGVGAPLCSSYLIPKEIQLPTTMTTFAEIQPLPAIEVTGGAQPAAGAQAAGDAARESP
AAGPEAAAAKPDLEELVALTPPSPFRDSVDSGSTTPNSPVSESALCIPSSPKYDTLIIRD
YTQSSSSL
Human mGluR5 (shorter isoform) has the following amino acid sequence
(SEQ ID NO: 2); NCBI accession number NP_000833.1.
SEQ ID NO: 2
MVLLLILSVLLLKEDVRGSAQSSERRVVAHMPGDIIIGALFSVHHQPTVDKVH
ERKCGAVREQYGIQRVEAMLHTLERINSDPTLLPNITLGCEIRDSCWHSAVALEQSIE
FIRDSUSSEEEEGLVRCVDGSSSSFRSKKPIVGVIGPGSSSVAIQVQNLLQLFNIPQIAY
SATSMDLSDKTLFKYFMRVVPSDAQQARAMVDIVKRYNWTYVSAVHTEGNYGESG
MEAFKDMSAKEGICIAHSYKIYSNAGEQSFDKLLKKLTSHLPKARVVACFCEGMTV
RGLLMAMRRLGLAGEFLLLGSDGWADRYDVTDGYQREAVGGITIKLQSPDVKWFD
DYYLKLRPETNHRNPWFQEFWQHRFQCRLEGFPQENSKYNKTCNSSLTLKTHHVQD
SKMGFVINAIYSMAYGLHNMQMSLCPGYAGLCDAMKPIDGRKLLESLMKTNFTGV
SGDTILFDENGDSPGRYEIMNFKEMGKDYFDYINVGSWDNGELKMDDDEVWSKKS
NIIRSVCSEPCEKGQIKVIRKGEVSCCWTCTPCKENEYVFDEYTCKACQLGSWPTDDL
TGCDLIPVQYLRWGDPEPIAAVVFACLGLLATLFVTVVFIIYRDTPVVKSSSRELCYII
LAGICLGYLCTFCLIAKPKQIYCYLQRIGIGLSPAMSYSALVTKTNRIARILAGSKKKIC
TKKPRFMSACAQLVIAFILICIQLGIIVALFIMEPPDIMHDYPSIREVYLICNTTNLGVVT
PLGYNGLLILSCTFYAFKTRNVPANFNEAKYIAFTMYTTCIIWLAFVPIYFGSNYKIIT
MCFSVSLSATVALGCMFVPKVYIILAKPERNVRSAFTTSTVVRMHVGDGKSSSAASR
SSSINNLWKRRGSSGETLSSNGKSVTWAQNEKSSRGQHLWQRLSIHINKKENPNQT
AVIKPFPKSTESRGLGAGAGAGGSAGGVGATGGAGCAGAGPGGPESPDAGPKALYD
VAEAEEHFPAPARPRSPSPISTLSHRAGSASRTDDDVPSLHSEPVARSSSSQGSLMEQI
SSVVTRFTANISELNSMMLSTAAPSPGVGAPLCSSYLIPKEIQLPTTMTTFAEIQPLPAI
EVTGGAQPAAGAQAAGDAARESPAAGPEAAAAKPDLEELVALTPPSPFRDSVDSGS
TTPNSPVSESALCIPSSPKYDTLIIRDYTQSSSSL
Rat mGluR5 has the following sequence (SEQ ID NO: 3); NCBI Accession
No. NP_058708.1.
SEQ ID NO: 3
MVLLLILSVLLLKEDVRGSAQSSERRVVAHMPGDIIIGALFSVHHQPTVDKVH
ERKCGAVREQYGIQRVEAMLHTLERINSDPTLLPNITLGCEIRDSCWHSAVALEQSIE
FIRDSLISSEEEEGLVRCVDGSSSFRSKKPIVGVIGPGSSSVAIQVQNLLQLFNIPQIAYS
ATSMDLSDKTLFKYFMRVVPSDAQQARAMVDIVKRYNWTYVSAVHTEGNYGESG
MEAFKDMSAKEGICIAHSYKIYSNAGEQSFDKLLKKLRSHLPKARVVACFCEGMTV
RGLLMAMRRLGLAGEFLLLGSDGWADRYDVTDGYQREAVGGITIKLQSPDVKWFD
DYYLKLRPETNLRNPWFQEFWQHRFQCRLEGFAQENSKYNKTCNSSLTLRTHHVQD
SKMGFVINAIYSMAYGLHNMQMSLCPGYAGLCDAMKPIDGRKLLDSLMKTNFTGV
SGDMILFDENGDSPGRYEIMNFKEMGKDYFDYINVGSWDNGELKMDDDEVWSKKN
NIIRSVCSEPCEKGQIKVIRKGEVSCCWTCTPCKFNEYVFDEYTCKACQLGSWPTDDL
TGCDLIPVQYLRWGDPEPIAAVVFACLGLLATLFVTVIFIIYRDTPVVKSSSRELCYIIL
AGICLGYLCTFCLIAKPKQIYCYLQRIGIGLSPAMSYSALVTKTNRIARILAGSKKKICT
KKPRFMSACAQLVIAFILICIQLGIIVALFIMEPPDIMHDYPSIREVYLICNTTNLGVVTP
LGYNGLLILSCTFYAFKTRNVPANFNEAKYIAFTMYTTCIIWLAFVPIYFGSNYKIITM
CFSVSLSATVALGCMFVPKVYIILAKPERNVRSAFTTSTVVRMHVGDGKSSSAASRSS
SLVNLWKRRGSSGETLSSNGKSVTWAQNEKSTRGQHLWQRLSVHINKKENPNQTA
VIKPFPKSTENRGPGAAAGGGSGPGVAGAGNAGCTATGGPEPPDAGPKALYDVAEA
EESFPAAARPRSPSPISTLSHLAGSAGRTDDDAPSLHSETAARSSSSQGSLMEQISSVV
TRFTANISELNSMMLSTAATPGPPGTPICSSYLIPKEIQLPTTMTTFAEIQPLPAIEVTGG
AQGATGVSPAQETPTGAESAPGKPDLEELVALTPPSPFRDSVDSGSTTPNSPVSESAL
CIPSSPKYDTLIIRDYTQSSSSL
Cynomolgus monkey mGluR5 has the following sequence (SEQ ID NO: 4);
NCBI Accession No. XP_005579366.1.
SEQ ID NO: 4
MVLLLILSVLLLKEDVRGSAQSSERRVVAHMPGDIIIGALFSVHHQPTVDKVH
ERKCGAVREQYGIQRVEAMLHTLERINSDPTLLPNITLGCEIRDSCWHSAVALEQSIE
FIRDSLISSEEEEGLVRCVDGSSSSFRSKKPIVGVIGPGSSSVAIQVQNLLQLFNIPQIAY
SATSMDLSDKTLFKYFMRVVPSDAQQARAMVDIVKRYNWTYVSAVHTEGNYGESG
MEAFKDMSAKEGICIAHSYKIYSNAGEQSFDKLLKKLTSHLPKARVVACFCEGMTV
RGLLMAMRRLGLAGEFLLLGSDGWADRYDVTDGYQREAVGGITIKLQSPDVKWFD
DYYLKLRPETNHRNPWFQEFWQHRFQCRLEGFPQENSKYNKTCDSSLTLKTHHVQD
SKMGFVINAIYSMAYGLHNMQMSLCPGYAGLCDAMKPIDGRKLLESLMKTNFTGV
SGDMILFDENGDSPGRYEIMNFKEMGKDYFDYINVGSWDNGELKMDDDEVWSKKS
NIIRSVCSEPCEKGQIKVIRKGEVSCCWTCTPCKENEYVFDEYTCKACQLGSWPTDDL
TGCDLIPVQYLRWGDPEPIAAVVFACLGLLATLFVTVVFIIYRDTPVVKSSSRELCYII
LAGICLGYLCTFCLIAKPKQIYCYLQRIGIGLSPAMSYSALVTKTNRIARILAGSKKKIC
TKKPRFMSACAQLVIAFILICIQLGIIVALFIMEPPDIMHDYPSIREVYLICNTTNLGVVT
PLGYNGLLILSCTFYAFKTRNVPANFNEAKYIAFTMYTTCIIWLAFVPIYFGSNYKIIT
MCFSVSLSATVALGCMFVPKVYIILAKPERNVRSAFTTSTVVRMHVGDGKSSSAASR
SSSLVNLWKRRGSSGETLRYKDRRLAQHKSEIECFTPKGSMGNGGRATMSSSNGKS
VTWAQNEKSSRGQHLWQRLSIHINKKENPNQTAVIKPFPKSTESRGLGAGAGAGGS
AGGMGATGGAGCAGAGPGGPEPPDASPKGLYDVAETEEHFPAPTRPRSPSPISTLSH
RAGSASRTDDDVPSLHSEPAARSSSSQGSLMEQISSVVTRFTANISELNSMMLSTAAP
SPGVGAPLCSSYLIPKEIQLPTTMTTFAEIQPLPAIEVTGGAQPAAGAQAAGDAARESP
AAGPKAAAAKPDLEELVALTPPSPFRDSVDSGSTTPNSPVSESALCIPSSPKYDTLIIRD
YTQSSSSL
The related metabotropic glutamate receptor 1 (mGluR1) in humans has the
following sequence (SEQ ID NO: 5); NCBI Accession No. NP_001264993.1.
SEQ ID NO: 5
MVGLLLFFFPAIFLEVSLLPRSPGRKVLLAGASSQRSVARMDGDVIIGALFSVH
HQPPAEKVPERKCGEIREQYGIQRVEAMFHTLDKINADPVLLPNITLGSEIRDSCWHS
SVALEQSIEFIRDSLISIRDEKDGINRCLPDGQSLPPGRTKKPIAGVIGPGSSSVAIQVQN
LLQLFDIPQIAYSATSIDLSDKTLYKYFLRVVPSDTLQARAMLDIVKRYNWTYVSAV
HTEGNYGESGMDAFKELAAQEGLCIAHSDKIYSNAGEKSFDRLLRKLRERLPKARV
VVCFCEGMTVRGLLSAMRRLGVVGEFSLIGSDGWADRDEVIEGYEVEANGGITIKLQ
SPEVRSFDDYFLKLRLDTNTRNPWFPEFWQHRFQCRLPGHLLENPNFKRICTGNESLE
ENYVQDSKMGFVINAIYAMAHGLQNMHHALCPGHVGLCDAMKPIDGSKLLDFLIKS
SFIGVSGEEVWFDEKGDAPGRYDIMNLQYTEANRYDYVHVGTWHEGVLNIDDYKIQ
MNKSGVVRSVCSEPCLKGQIKVIRKGEVSCCWICTACKENEYVQDEFTCKACDLGW
WPNADLTGCEPIPVRYLEWSNIESIIAIAFSCLGILVTLFVTLIFVLYRDTPVVKSSSREL
CYIILAGIFLGYVCPFTLIAKPTTTSCYLQRLLVGLSSAMCYSALVTKTNRIARILAGS
KKKICTRKPRFMSAWAQVIIASILISVQLTLVVTLIIMEPPMPILSYPSIKEVYLICNTSN
LGVVAPLGYNGLLIMSCTYYAFKTRNVPANFNEAKYIAFTMYTTCIIWLAFVPIYFGS
NYKIITTCFAVSLSVTVALGCMFTPKMYIIIAKPERNVRSAFTTSDVVRMHVGDGKLP
CRSNTFLNIFRRKKAGAGNANSNGKSVSWSEPGGGQVPKGQHMWHRLSVHVKTNE
TACNQTAVIKPLTKSYQGSGKSLTFSDTSTKTLYNVEEEEDAQPIRFSPPGSPSMVVH
RRVPSAATTPPLPSHLTAEETPLFLAEPALPKGLPPPLQQQQQPPPQQKSLMDQLQGV
VSNFSTAIPDFHAVLAGPGGPGNGLRSLYPPPPPPQHLQMLPLQLSTFGEELVSPPAD
DDDDSERFKLLQEYVYEHEREGNTEEDELEEEEEDLQAASKLTPDDSPALTPPSPFRD
SVASGSSVPSSPVSESVLCTPPNVSYASVILRDYKQSSSTL

In some embodiments, the antibodies or antigen-binding antibody fragments of the invention may bind specifically to mGluR5, but not to mGluR1. In some embodiments, the antibodies or antigen-binding antibody fragments of the invention may cross-react with human, rat, and/or cynomolgus monkey mGluR5.

### Binding to mGluR5 and Inhibition of mGluR5 Function

An anti-mGluR5 antibody of the invention can have any suitable affinity and/or avidity for mGluR5. Affinity refers to the strength of binding of an anti-mGluR5 antibody or other antigen-binding protein to an epitope or antigenic determinant. Typically, affinity is measured in terms of an equilibrium dissociation constant K_{D} defined as [Ab]×[Ag]/[Ab−Ag] where [Ab−Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Suitable methods for determining binding polypeptide specificity and affinity by competitive inhibition, equilibrium dialysis, and the like can be found in, e.g., Harlow, et al., Antibodies: A Laboratory Manual, Cold Spring Harbor Laboratory Press, Cold Spring Harbor, N.Y., 1988); Colligan et al., eds., Current Protocols in Immunology, Greene Publishing Assoc. and Wiley Interscience, N.Y., (1992, 1993), and Muller, Meth. Enzymol. 92:589-601 (1983).

Affinity can be determined by any of the methods described elsewhere herein or their known equivalents in the art. An example of one method that can be used to determine affinity is provided in Scatchard analysis of Munson & Pollard, Anal. Biochem. 107:220 (1980). Binding affinity also may be determined by KINEXA®, equilibrium methods, enzyme-linked immunoabsorbent assay (ELISA), radioimmunoassay (RIA), kinetics analysis, Biacore™ analysis, AlphaLISA® analysis, or Octet® RED96 analysis.

In yet another embodiment of the invention, anti-mGluR5 antibodies and antigen binding fragments, preferably human, humanized or chimerized anti-mGluR5 antibodies or antibody fragments, may bind to mGluR5 with a binding affinity (K_{D}) of less than or equal to 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰, 10⁻¹⁰, 5×10⁻¹¹, 10⁻¹¹ M, 5×10⁻¹², 10⁻¹² M, 5×10⁻¹³ M, or 10⁻¹³ M, as determined e.g. by ELISA, bio-layer interferometry (“BLI”) (e.g., using an Octet® RED96 system), KINEXA, surface plasmon resonance (e.g., using a Biacore™ or ProteOn system), or flow cytometry, optionally at 25° C. or 37° C. Typically, an anti-mGluR5 antibody provided by the invention has an affinity for mGluR5 in the range of about 10⁻⁴ to about 10⁻¹² M (e.g., about 10⁻⁷ to about 10⁻¹⁰ M). The term immunoreact herein typically refers to binding of an anti-mGluR5 antibody to mGluR5 with an affinity lower than about 10⁻⁴ M, as determined by, e.g., a flow cytometry saturation assay.

Additionally, the anti-mGluR5 antibodies and antigen binding fragments, preferably human, humanized or chimerized anti-mGluR5 antibodies or antibody fragments, of the invention may include anti-mGluR5 antibodies or antibody fragments which bind to mGluR5 with an off-rate (k_{off}) of less than or equal to 5×10⁻⁴ s⁻¹, 10⁻⁴ s⁻¹, 5×10⁻⁵ s⁻¹, or 10⁻⁵ s⁻¹.

Avidity refers to the overall strength of the total interactions between a binding protein and antigen (e.g., the total strength of interactions between an anti-mGluR5 antibody and a mGluR5). Affinity is the strength of the total noncovalent interactions between a single antigen-binding site on an antibody or other binding peptide and a single epitope or antigenic determinant. Avidity typically is governed by three major factors: the intrinsic affinity of the binding protein for the epitope(s) or antigenic determinant(s) to which it binds, the valence of the antibody or binding protein and antigen (e.g., an anti-mGluR5 antibody with a valency of three, four, or more will typically exhibit higher levels of avidity for an antigen than a bivalent antibody and a bivalent antibody can will have a higher avidity for an antigen than a univalent antibody, especially where there are repeated epitopes in the antigen), and/or the geometric arrangement of the interacting components. Avidity typically is measured by the same type of techniques used to assess affinity.

Anti-mGluR5 antibodies can be characterized on the basis of their ability to bind to mGluR5 and thereby inhibit one or more functions of mGluR5. Such anti-mGluR5 antibodies may be used directly as therapeutic agents in a native form. Inhibitory anti-mGluR5 antibodies may partially or fully inhibit the various functions of mGluR5, such as the downstream effects of ligand binding, e.g., agonists, antagonists or inverse agonists, e.g., glutamate or quisqualate; the stimulation of phospholipase C and/or p-ERK generation; and the increase of neuronal excitability. In a particular embodiment, the antibodies of the invention inhibit the mGluR5-mediated stimulation of p-ERK generation. Inhibition can be measured by any suitable method. In one embodiment, the method is AlphaLISA. In one aspect, inhibition is reflected in that the inhibiting anti-mGluR5 antibody inhibits p-ERK generation and optionally comprises an IC₅₀ of less than 10 nM. In another aspect, mGluR5 monoclonal antibodies can also be characterized by their lack of inhibition of ligand binding. In one aspect, inhibition of quisqualate binding can be measured via a radio-ligand binding assay. The effect of anti-mGluR5 antibodies on ligand binding can be determined by comparison with controls, e.g., in comparison with the results of ligand binding to cells that are treated with vehicle alone (negative control) or treated with excess unlabeled ligand (positive control).

### Production of Anti-mGluR5 Antibodies

Anti-mGluR5 monoclonal antibodies (mAbs) and antigen-binding fragments according to the present invention can be produced by a variety of techniques, including conventional monoclonal antibody methodology e.g., the standard somatic cell hybridization technique of Kohler and Milstein (1975) Nature 256:495. Although somatic cell hybridization procedures are preferred, in principle, other techniques for producing monoclonal antibody can be employed e.g., viral or oncogenic transformation of B lymphocytes.

A preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known. Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter and U.S. Pat. Nos. 5,530,101; 5,585,089; 5,693,762 and 6,180,370 to Queen et al.).

According to at least some embodiments of the invention, the antibodies are human monoclonal antibodies. Such human monoclonal antibodies directed against mGluR5 can be generated using transgenic or transchromosomic mice carrying parts of the human immune system rather than the mouse system, e.g., HuMAb Mouse™, KM Mouse™ (see e.g., Lonberg, et al. (1994) Nature 368(6474): 856-859). Accordingly, the mice exhibit reduced expression of mouse IgM or K and in response to immunization, the introduced human heavy and light chain transgenes undergo class switching and somatic mutation to generate high affinity human IgG K monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N. Y Acad. Sci. 764:536-546). In another embodiment, human antibodies according to at least some embodiments of the invention can be raised using a mouse that carries human immunoglobulin sequences on transgenes and transchomosomes, such as a mouse that carries a human heavy chain transgene and a human light chain transchromosome. Such mice, referred to herein as “KM Mice™”, are described in detail in PCT Publication WO 02/43478 to Ishida et al.

Still further, alternative transgenic animal systems expressing human immunoglobulin genes are available in the art and can be used to raise anti-mGluR5 antibodies according to at least some embodiments of the invention. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-mGluR5 antibodies according to at least some embodiments of the invention. For example, mice carrying both a human heavy chain transchromosome and a human light chain transchromosome, referred to as “TC mice” can be used; such mice are described in Tomizuka et al. (2000) Proc. Natl. Acad Sci. USA 97:722-727’. Furthermore, cows carrying human heavy and light chain transchromosomes have been described in the art (Kuroiwa et al. (2002) Nature Biotechnology 20:889-894) and can be used to raise anti-mGluR5 antibodies according to at least some embodiments of the invention.

Human monoclonal antibodies according to at least some embodiments of the invention can also be prepared using phage display methods for screening libraries of human immunoglobulin genes. Such phage display methods for isolating human antibodies are established in the art. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; and 6,555,313; 6,582,915 and 6,593,081 to Griffiths et al.

Human monoclonal antibodies according to at least some embodiments of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In some embodiments human Ig mice are used to raise human anti-mGluR5 antibodies according to the invention, e.g., by immunizing such mice with a purified or enriched preparation of mGluR5 antigen and/or recombinant mGluR5, or mGluR5 fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (dose ranging from 0.5-500 μg) of mGluR5 antigen can be used to immunize the human Ig mice intraperitoneally.

In general, transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA, and mice with sufficient titers of anti-mGluR5 human immunoglobulin can be used for fusions. Mice can be boosted intravenously with antigen 3 days before sacrifice and removal of the spleen. It is expected that 2-3 fusions for each immunization may need to be performed. Between 6 and 24 mice are typically immunized for each antigen.

In certain embodiments, hybridomas producing a human monoclonal anti-mGluR5 antibody according to the invention may be generated using splenocytes and/or lymph node cells from immunized mice which are isolated and fused to an appropriate immortalized cell line, such as a mouse myeloma cell line. The resulting hybridomas can be screened for the production of antigen-specific antibodies.

Exemplary teachings related to methods for obtaining clonal populations of antigen-specific B-cells from immunized rabbit hosts are disclosed in U.S. Patent Publication No. US2013/0316353, the disclosure of which is herein incorporated by reference in its entirety.

In certain embodiments, an anti-mGluR5 antibody according to the invention can be produced in a host cell transfectoma using, for example, a combination of recombinant DNA techniques and gene transfection methods as is well known in the art (e.g., Morrison, S. (1985) Science 229: 1202). For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segments within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

### Expression of Anti-mGluR5 Antibodies

A suitable host cell generally includes any cell wherein the subject anti-mGluR5 antibodies and antigen-binding fragments thereof can be produced recombinantly using techniques and materials readily available. For example, the anti-mGluR5 antibodies and antigen binding fragments thereof of the present invention can be produced in genetically engineered host cells according to conventional techniques. Suitable host cells are those cell types that can be transformed or transfected with exogenous DNA and grown in culture, and include bacteria, fungal cells (e.g., yeast), and cultured higher eukaryotic cells (including cultured cells of multicellular organisms), particularly cultured mammalian cells, e.g., human or non-human mammalian cells. In an exemplary embodiment these antibodies may be expressed in CHO cells or HEK-293 cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor, N.Y.: Cold Spring Harbor Laboratory Press (1989), and Current Protocols in Molecular Biology, Ausubel et al, editors, New York, N.Y.: Green and Wiley and Sons (1993).

In some exemplary embodiments the antibodies may be expressed in mating competent yeast, e.g., any haploid, diploid, or tetraploid yeast that can be grown in culture. Yeast useful in fermentation expression methods may exist in a haploid, diploid, or other polyploid form. The cells of a given ploidy may, under appropriate conditions, proliferate for an indefinite number of generations in that form. Diploid cells can also sporulate to form haploid cells. Sequential mating can result in tetraploid strains through further mating or fusion of diploid strains. The present invention contemplates the use of haploid yeast, as well as diploid or other polyploid yeast cells produced, for example, by mating or spheroplast fusion. By way of example, such yeast may include members of the Saccharomycetaceae family, which includes the genera Arxiozyma; Ascobotryozyma; Citeromyces; Debaryomyces; Dekkera; Eremothecium; Issatchenkia; Kazachstania; Kluyveromyces; Kodamaea; Lodderomyces; Pachysolen; Pichia; Saccharomyces; Saturnispora; Tetrapisispora; Torulaspora; Williopsis; and Zygosaccharomyces. Other types of yeast potentially useful in the invention include Yarrowia; Rhodosporidium; Candida; Hansenula; Filobasium; Sporidiobolus; Bullera; Leucosporidium and Filobasidella.

In a preferred exemplary embodiment of the invention, the mating competent yeast used for antibody expression may comprise a member of the genus Pichia. In a further preferred exemplary embodiment of the invention, the mating competent yeast of the genus Pichia is one of the following species: Pichia pastoris, Pichia methanolica, and Hansenula polymorpha (Pichia angusta).

The polypeptide coding sequence of interest is operably linked to transcriptional and translational regulatory sequences that provide for expression of the polypeptide in the desired host cells, e.g., yeast or mammalian cells. These vector components may include, but are not limited to, one or more of the following: an enhancer element, a promoter, and a transcription termination sequence. Sequences for the secretion of the polypeptide may also be included, e.g. a signal sequence, and the like. An origin of replication, e.g., a yeast origin of replication, is optional, as expression vectors are often integrated into the host cell genome. In one embodiment of the invention, the polypeptide of interest is operably linked, or fused, to sequences providing for optimized secretion of the polypeptide from yeast diploid cells.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (that increase levels of transcription in response to absence of a repressor). Inducible promoters may initiate increased levels of transcription from DNA under their control in response to some change in culture conditions, e.g., the presence or absence of a nutrient or a change in temperature. The promoter fragment may also serve as the site for homologous recombination and integration of the expression vector into the same site in the host cell, e.g., yeast cell, genome; alternatively, a selectable marker may be used as the site for homologous recombination. Pichia transformation is described in Cregg et al, Mol. Cell. Biol, 5: 3376-3385 (1985). Suitable promoters for use in different eukaryotic and prokaryotic cells are well known and commercially available.

The polypeptides of interest may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the polypeptide coding sequence that is inserted into the vector. The heterologous signal sequence selected preferably is one that is recognized and processed through one of the standard pathways available within the host cell, e.g., a mammalian cell, an insect cell, or a yeast cell. Additionally, these signal peptide sequences may be engineered to provide for enhanced secretion in expression systems. Secretion signals of interest also include mammalian and yeast signal sequences, which may be heterologous to the protein being secreted, or may be a native sequence for the protein being secreted. Signal sequences include pre-peptide sequences, and in some instances may include propeptide sequences. Many such signal sequences are known in the art, including the signal sequences found on immunoglobulin chains, e.g., K28 preprotoxin sequence, PHA-E, FACE, human MCP-1, human serum albumin signal sequences, human Ig heavy chain, human Ig light chain, and the like. For example, see Hashimoto et. al, Protein Eng., 11 (2):75 (1998); and Kobayashi et. al., Therapeutic Apheresis, 2(4):257 (1998).

Transcription may be increased by inserting a transcriptional activator sequence into the vector. These activators are cis-acting elements of DNA, usually about from 10 to 300 bp, which act on a promoter to increase its transcription. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter.

Expression vectors used in eukaryotic host cells may also contain sequences necessary for the termination of transcription and for stabilizing the mRNA. Such sequences are commonly available from 3′ to the translation termination codon, in untranslated regions of eukaryotic or viral DNAs or cDNAs. These regions contain nucleotide segments transcribed as polyadenylated fragments in the untranslated portion of the mRNA.

Construction of suitable vectors containing one or more of the above-listed components employs standard ligation techniques or PCR/recombination methods. Isolated plasmids or DNA fragments are cleaved, tailored, and re-ligated in the form desired to generate the plasmids required or via recombination methods. For analysis to confirm correct sequences in plasmids constructed, the ligation mixtures are used to transform host cells, and successful transformants selected by antibiotic resistance (e.g. ampicillin or Zeocin) where appropriate. Plasmids from the transformants are prepared, analyzed by restriction endonuclease digestion, and/or sequenced.

As an alternative to restriction and ligation of fragments, recombination methods based on specific attachment (“att”) sites and recombination enzymes may be used to insert DNA sequences into a vector. Such methods are described, for example, by Landy, Ann. Rev. Biochem., 58: 913-949 (1989); and are known to those of skill in the art. Such methods utilize intermolecular DNA recombination that is mediated by a mixture of lambda and E. coli-encoded recombination proteins. Recombination occurs between att sites on the interacting DNA molecules. For a description of att sites see Weisberg and Landy, Site-Specific Recombination in Phage Lambda, in Lambda II, p. 21 1-250, Cold Spring Harbor, N.Y.: Cold Spring Harbor Press (1983). The DNA segments flanking the recombination sites are switched, such that after recombination, the att sites are hybrid sequences comprised of sequences donated by each parental vector. The recombination can occur between DNAs of any topology.

Att sites may be introduced into a sequence of interest by ligating the sequence of interest into an appropriate vector; generating a PCR product containing att B sites through the use of specific primers; generating a cDNA library cloned into an appropriate vector containing att sites; and the like.

Folding, as used herein, refers to the three-dimensional structure of polypeptides and proteins, where interactions between amino acid residues act to stabilize the structure. While non-covalent interactions are important in determining structure, usually the proteins of interest will have intra- and/or intermolecular covalent disulfide bonds formed by two cysteine residues. For naturally occurring proteins and polypeptides or derivatives and variants thereof, the proper folding is typically the arrangement that results in optimal biological activity, and can conveniently be monitored by assays for activity, e.g. ligand binding, enzymatic activity, etc.

In some instances, for example where the desired product is of synthetic origin, assays based on biological activity will be less meaningful. The proper folding of such molecules may be determined on the basis of physical properties, energetic considerations, modeling studies, and the like.

The expression host may be further modified by the introduction of sequences encoding one or more enzymes that enhance folding and disulfide bond formation, i.e. foldases, chaperonins, etc. Such sequences may be constitutively or inducibly expressed in the yeast host cell, using vectors, markers, etc. as known in the art. Preferably the sequences, including transcriptional regulatory elements sufficient for the desired partem of expression, are stably integrated in the yeast genome through a targeted methodology.

For example, the eukaryotic protein disulfide isomerase (“PDI”) is not only an efficient catalyst of protein cysteine oxidation and disulfide bond isomerization, but also exhibits chaperone activity. Co-expression of PDI can facilitate the production of active proteins having multiple disulfide bonds. Also of interest is the expression of immunoglobulin heavy chain binding protein (“BIP”); cyclophilin; and the like. In one embodiment of the invention, each of the haploid parental strains expresses a distinct folding enzyme, e.g. one strain may express BIP, and the other strain may express PDI or combinations thereof.

Cultured mammalian cells are also preferred exemplary hosts for production of the disclosed anti-mGluR5 antibodies and antigen binding fragments thereof. As mentioned, CHO cells are particularly suitable for expression of antibodies. Many procedures are known in the art for manufacturing monoclonal antibodies in mammalian cells. (See, Galfre, G. and Milstein, C, Methods Enzym., 73:3-46, 1981; Basalp et al., Turk J. Biol, 24: 189-196, 2000; Wurm, F. M., Nat. Biotechnol, 22: 1393-1398, 2004; and Li et al., mAbs, 2(5):466-477, 2010). As mentioned in further detail infra, common host cell lines employed in mammalian monoclonal antibody manufacturing schemes include, but are not limited to, human embryonic retinoblast cell line PER.C6® (Crucell N. V., Leiden, The Netherlands), NSO murine myeloma cells (Medical Research Council, London, UK), CV1 monkey kidney cell line, 293 human embryonic kidney cell line, BHK baby hamster kidney cell line, VERO African green monkey kidney cell line, human cervical carcinoma cell line HELA, MDCK canine kidney cells, BRL buffalo rat liver cells, W138 human lung cells, HepG2 human liver cells, MMT mouse mammary tumor cells, TRI cells, MRC5 cells, Fs4 cells, myeloma or lymphoma cells, or Chinese Hamster (Cricetulus griseus) Ovary (CHO) cells, and the like. Many different subclones or sub-cell lines of CHO cells known in the art that are useful and optimized for production of recombinant monoclonal antibodies, such as the DP12 (CHO K1 dhfr⁻) cell line. NSO cells are a non-Ig secreting, non-light chain-synthesizing subclone of NS-1 cells that are resistant to azaguanine. Other Chinese Hamster and CHO cells are commercially available (from ATCC, etc.), including CHO-DXB11 (CHO-DUKX), CHO-pro3, CHO-DG44, CHO 1-15, CHO DP-12, Lec2, M1WT3, Lec8, pgsA-745, and the like, all of which are genetically altered to optimize the cell line for various parameters. Monoclonal antibodies are commonly manufactured using a batch fed method whereby the monoclonal antibody chains are expressed in a mammalian cell line and secreted into the tissue culture medium in a bioreactor. Medium (or feed) is continuously supplied to the bioreactor to maximize recombinant protein expression. Recombinant monoclonal antibody is then purified from the collected media. In some circumstances, additional steps are needed to reassemble the antibodies through reduction of disulfide bonds, etc. Such production methods can be scaled to be as large as 10,000 L in a single batch or more. It is now routine to obtain as much as 20 pg/cell/day through the use of such cell lines and methodologies, providing titers as high as 10 g/L or more, amounting to 15 to 100 kg from bioreactors of 10 kL to 25 kL. (Li et al, 2010). Various details of this production methodology, including cloning of the polynucleotides encoding the antibodies into expression vectors, transfecting cells with these expression vectors, selecting for transfected cells, and expressing and purifying the recombinant monoclonal antibodies from these cells are provided below.

For recombinant production of an anti-mGluR5 antibody or antigen binding fragment in mammalian cells, nucleic acids encoding the antibody or fragment thereof are generally inserted into a replicable vector for further cloning (amplification of the DNA) or for expression. DNA encoding the antibody is readily isolated or synthesized using conventional procedures (e.g., by using oligonucleotide probes that are capable of binding specifically to DNAs encoding the heavy and light chains of the antibody). The vector components generally include, but are not limited to, one or more of the following: a signal sequence, an origin of replication, one or more marker genes, an enhancer element, a promoter, and a transcription termination sequence. Selection of promoters, terminators, selectable markers, vectors, and other elements is a matter of routine design within the level of ordinary skill in the art. Many such elements are known in the art and are available through commercial suppliers.

The antibodies of this invention may be produced recombinantly not only directly, but also as a fusion polypeptide with a heterologous polypeptide, which is preferably a signal sequence or other polypeptide having a specific cleavage site at the N-terminus of the mature protein or polypeptide. The homologous or heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

Such expression vectors and cloning vectors will generally contain a nucleic acid sequence that enables the vector to replicate in one or more selected host cells. Typically, in cloning vectors this sequence is one that enables the vector to replicate independently of the host chromosomal DNA, and includes origins of replication or autonomously replicating sequences. Such sequences are well known for a variety of bacteria, yeast, and viruses, e.g., the origin of replication from the plasmid pBR322 is suitable for most Gram-negative bacteria, the 2mu plasmid origin is suitable for yeast, and various viral origins (Simian Virus 40 (“SV40”), polyoma, adenovirus, vesicular stomatitis virus (“VSV”), or bovine papillomavirus (“BPV”) are useful for cloning vectors in mammalian cells. Generally, the origin of replication component is not needed for mammalian expression vectors (the SV40 origin may typically be used only because it contains the early promoter).

These vectors will also typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” Examples of such dominant selection use the drugs neomycin, mycophenolic acid, and hygromycin. An exemplary selectable marker is a gene encoding resistance to the antibiotic neomycin. Selection is carried out in the presence of a neomycin-type drug, such as G-418 or the like. Those cells that are successfully transformed with a heterologous gene produce a protein conferring drug resistance and thus survive the selection regimen.

Selection systems can also be used to increase the expression level of the gene of interest, a process referred to as “amplification.” Amplification of transfectants typically occurs by culturing the cells in the presence of a low level of the selective agent and then increasing the amount of selective agent to select for cells that produce high levels of the products of the introduced genes. Exemplary suitable selectable markers for mammalian cells are those that enable the identification of cells competent to take up the antibody nucleic acid, such as dihydrofolate reductase (“DHFR”), thymidine kinase, metallothionein-I and -II, preferably primate metallothionein genes, adenosine deaminase, ornithine decarboxylase, etc.

For example, an amplifiable selectable marker for mammalian cells is dihydrofolate reductase, which confers resistance to methotrexate. Other drug resistance genes (e.g. hygromycin resistance, multi-drug resistance, puromycin acetyltransferase) can also be used. Cells transformed with the DHFR selection gene are first identified by culturing all of the transformants in a culture medium that contains methotrexate (“MTX”), a competitive antagonist of DHFR. An appropriate host cell when wild-type DHFR is employed is the Chinese hamster ovary (“CHO”) cell line deficient in DHFR activity.

Alternatively, host cells (particularly wild-type hosts that contain endogenous DHFR) transformed or co-transformed with DNA sequences encoding antibody, wild-type DHFR protein, and another selectable marker such as aminoglycoside 3′-phosphotransferase (“APH”) can be selected by cell growth in medium containing a selection agent for the selectable marker such as an aminoglycosidic antibiotic, e.g., kanamycin, neomycin, or G-418. See U.S. Pat. No. 4,965,199.

These vectors may comprise an enhancer sequence that facilitates transcription of a DNA encoding the antibody. Many enhancer sequences are known from mammalian genes (for example, globin, elastase, albumin, alpha-fetoprotein, and insulin). A frequently used enhancer is one derived from a eukaryotic cell virus. Examples thereof include the SV40 enhancer on the late side of the replication origin (bp 100-270), the cytomegalovirus early promoter enhancer, the polyoma enhancer on the late side of the replication origin, and adenovirus enhancers (See, also Yaniv, Nature, 297: 17-18, 1982, on enhancing elements for activation of eukaryotic promoters). The enhancer may be spliced into the vector at a position 5′ or 3′ to the antibody-encoding sequence, but is preferably located at a site 5′ from the promoter.

Expression and cloning vectors will also generally comprise a promoter that is recognized by the host organism and is operably linked to the antibody nucleic acid. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody transcription from vectors in mammalian host cells is controlled, for example, by promoters obtained from the genomes of viruses such as polyoma virus, fowlpox virus, adenovirus (such as Adenovirus 2), BPV, avian sarcoma virus, cytomegalovirus, a retrovirus, hepatitis-B virus, and most preferably SV40, from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the BPV as a vector is disclosed in U.S. Pat. No. 4,419,446. A modification of this system is described in U.S. Pat. No. 4,601,978. See also Reyes et al., Nature, 297:598-601 (1982) on expression of human beta-interferon cDNA in mouse cells under the control of a thymidine kinase promoter from herpes simplex virus. Alternatively, the rous sarcoma virus long terminal repeat can be used as the promoter.

Strong transcription promoters can be used, such as promoters from SV40, cytomegalovirus, or myeloproliferative sarcoma virus. See, e.g., U.S. Pat. No. 4,956,288 and U.S. Patent Publication No. 20030103986. Other suitable promoters include those from metallothionein genes (U.S. Pat. Nos. 4,579,821 and 4,601,978) and the adenovirus major late promoter. Expression vectors for use in mammalian cells include pZP-1, pZP-9, and pZMP21, which have been deposited with the American Type Culture Collection, 10801 University Blvd., Manassas, Va. USA under accession numbers 98669, 98668, and PTA-5266, respectively, and derivatives of these vectors.

Expression vectors used in eukaryotic host cells (yeast, fungus, insect, plant, animal, human, or a nucleated cell from other multicellular organism) will also generally contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the antibody. One useful transcription termination component is the bovine growth hormone polyadenylation region. See WO 94/11026 and the expression vector disclosed therein.

Suitable host cells for cloning or expressing the subject antibodies include prokaryote, yeast, or higher eukaryote cells described above. However, interest has been greatest in vertebrate cells, and propagation of vertebrate cells in culture has become a routine procedure. Examples of useful mammalian host cell lines are monkey kidney CV1 line transformed by SV40 (COS-1 (ATCC No. CRL 1650); and COS-7, ATCC CRL 1651); human embryonic kidney line (293 or 293 cells subcloned for growth in suspension culture, (ATCC No. CRL 1573; Graham et al, J Gen. Virol, 36:59-72 (1977)); baby hamster kidney cells (BHK, ATCC CCL 10, ATCC No. CRL 1632; BHK 570, ATCC No. CRL 10314); CHO cells (CHO-KI, ATCC No. CCL 61; CHO-DG44, Urlaub et al, Proc. Natl. Acad Sci. USA, 77:4216-4220 (1980)); mouse Sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL-34); buffalo rat liver cells (BRL 3A, ATCC CRL 1442); human lung cells (W138, ATCC CCL-75); human liver cells (Hep G2, HB 8065); mouse mammary tumor (MMT 060562, ATCC CCL-51); TRI cells (Mather et al., Annals N. Y. Acad. Sci., 383:44-68 (1982)); MRC 5 cells; FS4 cells; and a human hepatoma line (Hep G2). Additional suitable cell lines are known in the art and available from public depositories such as the American Type Culture Collection, Manassas, Va.

Host cells are transformed with the above-described expression or cloning vectors for antibody production and cultured in conventional nutrient media modified as appropriate for inducing promoters, selecting transformants, or amplifying the genes encoding the desired sequences as discussed supra.

The mammalian host cells used to produce the antibody of this invention may be cultured in a variety of media. Commercially available media such as Ham's F10 (Sigma-Aldrich Corporation, St. Louis, Mo.), Minimal Essential Medium ((“MEM”) (Sigma-Aldrich Corporation, St. Louis, Mo.), Roswell Park Memorial Institute-1640 medium (“RPMI-1640”, Sigma-Aldrich Corporation, St. Louis, Mo.), and Dulbecco's Modified Eagle's Medium ((“DMEM”) Sigma-Aldrich Corporation, St. Louis, Mo.) are suitable for culturing the host cells. In addition, any of the media described in Ham et al., Meth. Era., 58:44 (1979); Barnes et al., Anal. Biochem., 102:255 (1980); U.S. Pat. Nos. 4,767,704; 4,657,866; 4,927,762; 4,560,655; or 5,122,469; WO 90/03430; WO 87/00195; or U.S. Patent Reexam No. 30,985, can be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan. Methods of development and optimization of media and culture conditions are known in the art. (See, Gronemeyer et al, Bioengineering, 1(4): 188-212, 2014).

After culture conditions are optimized and a preferred cell line clone is selected, these cells are cultured (either adherent cells or suspension cultures) most typically in a batch-fed process in a bioreactor (many models are commercially available) that involves continuously feeding the cell culture with medium and feed, optimized for the particular cell line chosen and selected for this purpose. (See, Butler, M., Appl. Microbiol. Biotechnol, 68:283-291, 2005; and Kelley, B., mAbs, 1(5):443-452, 2009). Perfusion systems are also available in which media and feed are continuously supplied to the culture while the same volume of media is being withdrawn from the bioreactor. (Wurm F M. Nature Biotechnology. 2004; 22(11):1393). Synthetic media, also commercially available, are available for growing cells in a batch-fed culture, avoiding the possibility of contamination from outside sources, such as with the use of animal components, such as bovine serum albumin, etc. However, animal-component-free hydrolysates are commercially available to help boost cell density, culture viability and productivity. (Li H, Aluko R E. Journal of Agricultural and Food Chemistry. 2010; 58(21):11471-6.). Many studies have been performed in an effort to optimize cell culture media, including careful attention to head space available in roller bottles, redox potentials during growth and expression phases, presence of reducing agents to maintain disulfide bonds during production, etc. (See, for instance, Hutterer et al., mAbs, 5(4):608-613, 2013; and Mullan et al, BMC Proceed., 5(Suppl 8):P110, 2011). Various methodologies have been developed to address the possibility of harmful oxidation during recombinant monoclonal antibody production. (See, for example, U.S. Pat. No. 8,574,869). Cultured cells may be grown by feeding nutrients continuously or as separately administered amounts. Often various process parameters such as cell concentration, pH, temperature, C02, d02, osmolality, amount of metabolites such as glucose, lactate, glutamine and glutamate, and the like, are monitored by the use of probes during the cell growth either on-line by direct connection to calibrated analyzers or off-line by intervention of operators. The culturing step also typically involves ensuring that the cells growing in culture maintain the transfected recombinant genes by any means known in the art for cell selection.

Following fermentation, i.e., upon reaching maximum cell growth and recombinant protein expression, the culturing step is typically followed by a harvesting step, whereby the cells are separated from the medium and a harvested cell culture media is thereby obtained. (See, Liu et al, mAbs, 2(5):480-499, 2010). Typically various purification steps, involving column chromatography and the like, follow culturing to separate the recombinant monoclonal antibody from cell components and cell culture media components. The exact purification steps needed for this phase of the production of recombinant monoclonal antibodies depends on the site of expression of the proteins, i.e., in the cytosol of the cells themselves, or the more commonly preferred route of protein excreted into the cell culture medium. Various cell components may be separated using techniques known in the art such as differential centrifugation techniques, gravity-based cell settling, and/or size exclusion chromatograph/filtration techniques that can include tangential flow micro-filtration or depth filtration. (See, Pollock et al, Biotechnol. Bioeng., 110:206-219, 2013). Centrifugation of cell components may be achieved on a large scale by use of continuous disk stack centrifuges followed by clarification using depth and membrane filters. (See, Kelley B, Blank G, Lee A. Process scale purification of antibodies. 2009:1-23). Most often, after clarification, the recombinant protein is further purified by Protein A chromatography due to the high affinity of Protein A for the Fc domain of antibodies, and typically occurs using a low pH/acidification elution step (typically the acidification step is combined with a precautionary virus inactivation step). Flocculation and/or precipitation steps using acidic or cationic polyelectrolytes may also be employed to separate animal cells in suspension cultures from soluble proteins. Lastly, anion- and cation-exchange chromatography, hydrophobic interaction chromatography (“HIC”), hydrophobic charge induction chromatography (HCIC), hydroxyapatite chromatography using ceramic hydroxyapatite (Ca₅(PO₄)₃OH)₂, and combinations of these techniques are typically used to polish the solution of recombinant monoclonal antibody. Final formulation and concentration of the desired monoclonal antibody may be achieved by use of ultracentrifugation techniques. Purification yields are typically 70 to 80%.

### Anti-Idiotypic Antibodies

Another aspect of the invention is directed to anti-idiotypic antibodies and anti-anti-idiotypic antibodies. An anti-idiotypic antibody is an antibody that recognizes determinants of another antibody (a target antibody). Generally, the anti-idiotypic antibody recognizes determinants of the antigen-binding site of the target antibody. Typically, the target antibody is a monoclonal antibody. An anti-idiotypic antibody is generally prepared by immunizing an animal (particularly, mice) of the same species and genetic type as the source of the target monoclonal antibody, with the target monoclonal antibody. The immunized animal mounts an immune response to the idiotypic determinants of the target monoclonal antibody and produces antibodies against the idiotypic determinants of the target monoclonal antibody. Antibody-producing cells, such as splenic cells, of the immunized animal may be used to generate anti-idiotypic monoclonal antibodies. Furthermore, an anti-idiotypic antibody may also be used to immunize animals to produce anti-anti-idiotypic antibodies. These immunized animals may be used to generate anti-anti-idiotypic monoclonal antibodies using standard techniques. The anti-anti-idiotypic antibodies may bind to the same epitope as the original, target monoclonal antibody used to prepare the anti-idiotypic antibody. The anti-anti-idiotypic antibodies represent other monoclonal antibodies with the same antigen specificity as the original target monoclonal antibody.

If the binding of the anti-idiotypic antibody with the target antibody is inhibited by the relevant antigen of the target antibody, and if the anti-idiotypic antibody induces an antibody response with the same specificity as the target antibody, it mimics the antigen of the target antibody. Such an anti-idiotypic antibody is an “internal image anti-idiotypic” and is capable of inducing an antibody response as if it were the original antigen. (Bona and Kohler, Anti-Idiotypic Antibodies And Internal Image, In Monoclonal And Anti-Idiotypic Antibodies: Probes For Receptor Structure And Function, Venter J. C., Frasser, C. M., Lindstrom, J. (Eds.), Alan R. Liss, N. Y., 1984. pp 141-149). Vaccines incorporating internal image anti-idiotype antibodies have been shown to induce protective responses against viruses, bacteria, and parasites (Kennedy et al. (1986) 232:220-223; McNamara et al. (1985) Science 226:1325-1326). Internal image anti-idiotypic antibodies have also been shown to induce immunity to tumor related antigens (Raychauhuri el al. (1986) J. Immunol. 137:1743-1749; Raychauhuri et al. (1987) J Immunol. 139:3902-3910; Bhattacharya-Chatterjee et al. (1987) J. Immunol. 139:1354-1360; Bhattacharya-Chatterjee et al. (1988) J. Immunol. 141:1398-1403; Herlyn, D. et al. (1989) Intern. Rev. Immunol. 4:347-357; Chen, Z.-J et al. (1990) Cell Imm. Immunother. Cancer 351-359; Herlyn, D. et al. (1991) In Vivo 5:615-624; Furuya et al. (1992) Anticancer Res. 12:27-32; Mittelman A. et al. (1992) Proc. Natl. Acad. Sci., USA 89:466-470; Durrant, L. G. et al. (1994) Cancer Res. 54:4837-4840; Mittelman, A. et al. (1994) Cancer Res 54:415-421; Schmitt, H. et al. (1994) Hybridoma 13:389-396; Chakrobarty, M. et al. (1995) J. Immunother. 18:95-103; Chakrobarty, M. et al. (1995) Cancer Res. 55:1525-1530; Foon, K. A. et al. (1995) Clin. Cancer Res. 1:1205-1294; Herlyn, D, et al. (1995) Hybridoma 14:159-166; Sclebusch, H. et al. (1995) Hybridoma 14:167-174; and Herlyn, D. et al. (1996) Cancer Immunol Immunother. 43:65-76).

Anti-idiotypic antibodies for mGluR5 may be prepared, for example, by immunizing an animal, such as a mouse, with an immunogenic amount of a composition comprising mGluR5 or immunogenic portions thereof, containing at least one antigenic epitope of mGluR5. The composition may also contain a suitable adjuvant, and any carrier necessary to provide immunogenicity. Monoclonal antibodies recognizing mGluR5 may be prepared from the cells of the immunized animal as described above. A monoclonal antibody recognizing an epitope of mGluR5 is then selected and used to prepare a composition comprising an immunogenic amount of the anti-mGluR5 monoclonal antibody. Typically, a 25 to 200 sg dose of purified mGluR5 monoclonal would be sufficient in a suitable adjuvant.

Animals may be immunized 2-6 times at 14 to 30 day intervals between doses. Typically, animals are immunized by any suitable route of administration, such as intraperitoneal, subcutaneous, intravenous, or a combination of these. Anti-idiotypic antibody production may be monitored during the immunization period using standard immunoassay methods. Animals with suitable titers of antibodies reactive with the target monoclonal antibodies may be re-immunized with the monoclonal antibody used as the immunogen three days before harvesting the antibody producing cells. Preferably, spleen cells are used, although other antibody producing cells may be selected. Antibody-producing cells are harvested and fused with myeloma cells to produce hybridomas, as described above, and suitable anti-idiotypic antibody-producing cells are selected.

Anti-anti-idiotypic antibodies are produced by another round of immunization and hybridoma production by using the anti-idiotypic monoclonal antibody as the immunogen. Exemplary teachings related to methods for obtaining clonal populations of antigen-specific B-cells from immunized rabbit hosts are disclosed in U.S. Patent Publication No. US2013/0316353, the disclosure of which is herein incorporated by reference in its entirety.

### Competition, Epitope Mapping, and Structural Similarity

The identification of one or more antibodies that bind(s) to substantially or essentially the same epitope as the monoclonal antibodies described herein can be readily determined using alanine scanning. Additionally, any one of a variety of immunological screening assays in which antibody competition can be assessed. A number of such assays are routinely practiced and well known in the art (see, e.g., U.S. Pat. No. 5,660,827, issued Aug. 26, 1997, which is specifically incorporated herein by reference). It will be understood that actually determining the epitope to which an antibody described herein binds is not in any way required to identify an antibody that binds to the same or substantially the same or overlapping epitope as the monoclonal antibodies described herein.

For example, a simple competition assay may be employed in which the control antibody (mGluR5 Ab1-Ab29 or a fragment or variant thereof, for example) is mixed with the test antibody and then applied to a sample containing mGluR5, which is known to be bound by mGluR5 Ab1-Ab29. Alternatively, the antibodies may be added sequentially to the mGluR5 sample. Protocols based upon ELISAs, AlphaLISAs, radioimmunoassays, Western blotting, Biacore™, Octet®, and ProteOn™ analysis are suitable for use in such simple competition studies.

In certain embodiments, the method comprises pre-mixing the control antibody with varying amounts of the test antibody (e.g., in ratios of about 1:1, 1:2, 1:10, or about 1:100) for a period of time prior to applying to the mGluR5 antigen sample. In other embodiments, the control and varying amounts of test antibody can be added separately and admixed during exposure to the mGluR5 antigen sample. Bound antibodies can be distinguished from free antibodies (e.g., by using separation or washing techniques to eliminate unbound antibodies) and control antibody may be distinguished from the test antibody (e.g., by using species specific or isotype specific secondary antibodies or by specifically labelling the control antibody with a detectable label). Thereby these methods can be used to determine whether a test antibody reduces the binding of the control antibody to the mGluR5 antigen, indicating that the test antibody recognizes substantially the same or overlapping epitope as the control antibody (e.g., mGluR5 Ab1-Ab29). The binding of the (labeled) control antibody in the presence of a completely irrelevant antibody (that does not bind mGluR5) can serve as the control high value. The control low value can be obtained by incubating the labeled control antibody with the same but unlabeled control antibody, where competition would occur and reduce binding of the labeled antibody. In a test assay, a significant reduction in labeled antibody reactivity in the presence of a test antibody is indicative of a test antibody that recognizes substantially the same epitope, i.e., one that competes with the labeled control antibody. For example, any test antibody that reduces the binding of mGluR5 Ab1-Ab29 to mGluR5 by at least about 50%, such as at least about 60%, or more preferably at least about 70% (e.g., about 65-100%), at any ratio of control mGluR5 antibody:test antibody between about 1:1 or 1:10 and about 1:100 is considered to be an antibody that binds to substantially the same epitope or determinant as mGluR5 Ab1-Ab29. Preferably, such test antibody will reduce the binding of mGluR5 Ab1-Ab29 to mGluR5 by at least about 50%, at least about 60%, at least about 80% or at least about 90% (e.g., about 95%) of the binding of mGluR5 Ab1-Ab29 observed in the absence of the test antibody. These methods can be adapted to identify and/or evaluate antibodies that compete with other control antibodies.

Preferably, such test antibody will reduce the binding of the control antibody to mGluR5 antigen preferably at least about 50%, at least about 60%, at least about 80%, or at least about 90% (e.g., about 95%) of the binding of the control antibody observed in the absence of the test antibody.

A simple competition assay in which a test antibody is applied at saturating concentration to a surface onto which mGluR5 is immobilized also may be advantageously employed. The surface in the simple competition assay is preferably of a media suitable for OCTET® and/or PROTEON®. The binding of a control antibody (e.g., mGluR5 Ab1-Ab29) to the mGluR5-coated surface is measured. This binding to the mGluR5-containing surface of the control antibody alone is compared with the binding of the control antibody in the presence of a test antibody. A significant reduction in binding to the mGluR5-containing surface by the control antibody in the presence of a test antibody indicates that the test antibody recognizes substantially the same epitope as the control antibody such that the test antibody “competes” with the control antibody. Any test antibody that reduces the binding of control antibody (such as mGluR5 Ab1-Ab29) to mGluR5 by at least about 20% or more, at least about 40%, at least about 50%, at least about 70%, or more, can be considered to be an antibody that binds to substantially the same epitope or determinant as the control antibody (e.g., mGluR5 Ab1-Ab29). Preferably, such test antibody will reduce the binding of the control antibody (e.g., mGluR5 Ab1-Ab29) to the mGluR5 antigen by at least about 50% (e.g., at least about 60%, at least about 70%, or more). It will be appreciated that the order of control and test antibodies can be reversed; i.e. the control antibody can be first bound to the surface and then the test antibody is brought into contact with the surface thereafter in a competition assay. Preferably, the antibody having higher affinity for mGluR5 is bound to the mGluR5-containing surface first, as it will be expected that the decrease in binding seen for the second antibody (assuming the antibodies are competing) will be of greater magnitude. Further examples of such assays are provided in, e.g., Saunal and Regenmortel, J. Immunol. Methods, 183:33-41 (1989), the disclosure of which is incorporated herein by reference.

In addition, whether an antibody binds the same or overlapping epitope(s) on mGluR5 as another antibody or the epitope bound by a test antibody may in particular be determined using a Western-blot based assay. In this assay a library of peptides corresponding to the antigen bound by the antibody, the mGluR5 protein, is made, that comprise overlapping portions of the protein, typically 10-25, 10-20, or 10-15 amino acids long. These different overlapping amino acid peptides encompassing the mGluR5 sequence are synthesized and covalently bound to a PEPSPOTS™ nitrocellulose membrane (JPT Peptide Technologies, Berlin, Germany). Blots are then prepared and probed according to the manufacturer's recommendations.

Essentially, the immunoblot assay then detects by fluorometric means what peptides in the library bind to the test antibody and thereby can identify what residues on the antigen, i.e., mGluR5, interact with the test antibody. (See U.S. Pat. No. 7,935,340, incorporated by reference herein).

Various epitope mapping techniques are known in the art. By way of example, X-ray co-crystallography of the antigen and antibody; NMR; SPR (e.g., at 25° or 37° C.); array-based oligo-peptide scanning (or “pepscan analysis”); site-directed mutagenesis (e.g., alanine scanning); mutagenesis mapping; hydrogen-deuterium exchange; phage display; and limited proteolysis are all epitope mapping techniques that are well known in the art (See, e.g., Epitope Mapping Protocols: Second Edition, Methods in Molecular Biology, editors Mike Schutkowski and Ulrich Reineke, 2nd Ed., New York, N.Y.: Humana Press (2009), and Epitope Mapping Protocols, Methods in Molecular Biology, editor Glenn Morris, 1st Ed., New York, N.Y.: Humana Press (1996), both of which are herein incorporated by referenced in their entirety).

The identification of one or more antibodies that bind(s) to substantially or essentially the same epitope as the monoclonal antibodies described herein, e.g., mGluR5 Ab1-Ab29 or a variant thereof, can be readily determined using any one of variety of immunological screening assays in which antibody competition can be assessed. A number of such assays are routinely practiced and well known in the art (see, e.g., U.S. Pat. No. 5,660,827, issued Aug. 26, 1997, which is incorporated herein by reference). It will be understood that determining the epitope to which an antibody described herein binds is not in any way required to identify an antibody that binds to the same or substantially the same epitope as the monoclonal antibody described herein.

Determination of whether an antibody, antigen binding fragment thereof, or antibody derivative binds within one of the epitope regions defined above can be carried out in ways known to the person skilled in the art. In another example of such mapping/characterization methods, an epitope region for an anti-mGluR5 antibody may be determined by epitope “footprinting” using chemical modification of the exposed amines/carboxyls in the mGluR5 protein. One specific example of such a foot-printing technique is the use of hydrogen-deuterium exchange detected by mass spectrometry (“HXMS”), wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry (See, e.g., Ehring H., Analytical Biochemistry, 267(2):252-259 (1999) and Engen, J. R. & Smith, D. L., Anal. Chem., 73:256A-265A (2001)). Another example of a suitable epitope identification technique is nuclear magnetic resonance epitope mapping (“NMR”), where typically the position of the signals in two-dimensional NMR spectres of the free antigen and the antigen complexed with the antigen binding peptide, such as an antibody, are compared. The antigen typically is selectively isotopically labeled with ¹⁵N so that only signals corresponding to the antigen and no signals from the antigen binding peptide are seen in the NMR-spectrum. Antigen signals originating from amino acids involved in the interaction with the antigen binding peptide typically will shift position in the spectres of the complex compared to the spectres of the free antigen, and the amino acids involved in the binding can be identified that way. See, e.g., Ernst Schering Res. Found. Workshop, (44): 149-67 (2004); Huang et al, J. Mol. Biol, 281(1):61-67 (1998); and Saito and Patterson, Methods, 9(3):516-24 (1996). Epitope mapping/characterization also can be performed using mass spectrometry (“MS”) methods (See, e.g., Downard, J. Mass Spectrum., 35(4):493-503 (2000) and Kiselar and Downard, Anal. Chem., 71(9): 1792-801 (1999)).

Protease digestion techniques also can be useful in the context of epitope mapping and identification. Antigenic determinant-relevant regions/sequences can be determined by protease digestion, e.g. by using trypsin in a ratio of about 1:50 to mGluR5 overnight (“o/n”) digestion at 37° C. and pH 7-8, followed by mass spectrometry (“MS”) analysis for peptide identification. The peptides protected from trypsin cleavage by the anti-mGluR5 antibody can subsequently be identified by comparison of samples subjected to trypsin digestion and samples incubated with antibody and then subjected to digestion by e.g. trypsin (thereby revealing a footprint for the antibody). Other enzymes like chymotrypsin or pepsin can be used in similar epitope characterization methods. Moreover, enzymatic digestion can provide a quick method for analyzing whether a potential antigenic determinant sequence is within a region of mGluR5 in the context of a mGluR5-binding polypeptide. If the polypeptide is not surface exposed, it is most likely not relevant in terms of immunogenicity/antigenicity (See, e.g., Manca, Ann. 1st. Super. Sanita., 27(1): 15-9 (1991) for a discussion of similar techniques).

Site-directed mutagenesis is another technique useful for characterization of a binding epitope. For example, in “alanine-scanning” site-directed mutagenesis (also known as alanine scanning, alanine scanning mutagenesis, alanine scanning mutations, combinatorial alanine scanning, or creation of alanine point mutations, for example), each residue within a protein segment is replaced with an alanine residue (or another residue such as valine where alanine is present in the wild-type sequence) through such methodologies as direct peptide or protein synthesis, site-directed mutagenesis, the GENEART™ Mutagenesis Service (Thermo Fisher Scientific, Waltham, Mass. U.S.A.) or shotgun mutagenesis, for example. A series of single point mutants of the molecule is thereby generated using this technique; the number of mutants generated is equivalent to the number of residues in the molecule, each residue being replaced, one at a time, by a single alanine residue. Alanine is generally used to replace native (wild-type) residues because of its non-bulky, chemically inert, methyl functional group that can mimic the secondary structure preferences that many other amino acids may possess. Subsequently, the effects replacing a native residue with an alanine has on binding affinity of an alanine scanning mutant and its binding partner can be measured using such methods as, but not limited to, SPR binding experiments. If a mutation leads to a significant reduction in binding affinity, it is most likely that the mutated residue is involved in binding. Monoclonal antibodies specific for structural epitopes (i.e., antibodies that do not bind the unfolded protein) can be used as a positive control for binding affinity experiments to verify that the alanine-replacement does not influence the overall tertiary structure of the protein (as changes to the overall fold of the protein may indirectly affect binding and thereby produce a false positive result). See, e.g., Clackson and Wells, Science, 267:383-386 (1995); Weiss et al, Proc. Natl. Acad. Sci. USA, 97(16):8950-8954 (2000); and Wells, Proc. Natl. Acad. Sci. USA, 93: 1-6 (1996).

Electron microscopy can also be used for epitope “footprinting”. For example, Wang et al., Nature, 355:275-278 (1992) used coordinated application of cryoelectron microscopy, three-dimensional image reconstruction, and X-ray crystallography to determine the physical footprint of a Fab-fragment on the capsid surface of native cowpea mosaic virus.

Other forms of “label-free” assay for epitope evaluation include SPR (sold commercially as the BIiacore™ system, GE Healthcare Life Sciences, Marlborough, Mass.) and reflectometric interference spectroscopy (“RifS”) (See, e.g., Fagerstam et al, Journal of Molecular Recognition, 3:208-14 (1990); Nice et al, J. Chromatogr., 646: 159-168 (1993); Leipert et al, Angew. Chem. Int. Ed., 37:3308-3311 (1998); Kroger et al, Biosensors and Bioelectronics, 17:937-944 (2002)). In a particular embodiment, epitope evaluation may be conducted via an Octet® RED96 system, using immobilization of the mGluR5 extracellular domain, as demonstrated in the Examples herein.

In some embodiments, an anti-mGluR5 antibody of the invention may have the same or similar structure to another anti-mGluR5 antibody. Structural similarity may be assessed via a structural alignment of three dimensional protein structures attained through x-ray crystallography, NMR, or other known methods. A similar structure may be determined through an analysis of the difference in positions between the C alpha carbons in the CDRs of the two proteins being compared. Generally, an average RMSD of less than 5 Å, less than 4 Å, less than 3 Å, less than 2 Å, less than 1 Å, or less than 0.5 Å in one or more of the CDRs is indicative of a similar protein structure.

### Sequences of Anti-mGluR5 Antibodies

The following are particular, non-limiting embodiments of the invention. In each case, the invention also comprises antibodies and antibody fragments binding to similar epitopes and having similar sequences, as described in further detail as follows.

In one embodiment, an antibody or antibody fragment of the invention is mGluR5 Ab1. In one embodiment, an antibody or antibody fragment comprises the V_{H} chain CDRs (SEQ ID NOS:9,11,13) of mGluR5 Ab1. In one embodiment, an antibody or antibody fragment comprises the V_{L} chain CDRs (SEQ ID NOS:17,19,21) of mGluR5 Ab1. In one embodiment, an antibody or antibody fragment comprises the V_{H} chain CDRs (SEQ ID NOS:9,11,13) and the V_{L} chain CDRs (SEQ ID NOS:17,19,21) of mGluR5 Ab1. In a further embodiment, an antibody or antibody fragment of the invention binds to the same epitope as mGluR5 Ab1. In another embodiment, an antibody or antibody fragment of the invention comprises the V_{H} chain (SEQ ID NO:7) and the V_{L} chain (SEQ ID NO:15) of mGluR5 Ab1 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:7. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:15. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:7 and a V_{L} chain having at least 80%, at least 85%, at least 90%, at least 95%, at least 98%, at least 99%, or 100% identity to the amino acid sequence of SEQ ID NO:15.

In one embodiment, an antibody or antibody fragment of the invention is mGluR5 Ab2. In one embodiment, an antibody or antibody fragment comprises the V_{H} chain CDRs (SEQ ID NOS:25,27,29) of mGluR5 Ab2. In one embodiment, an antibody or antibody fragment comprises the V_{L} chain CDRs (SEQ ID NOS:33,35,37) of mGluR5 Ab2. In one embodiment, an antibody or antibody fragment comprises the V_{H} chain CDRs (SEQ ID NOS:25,27,29) and the V_{L} chain CDRs (SEQ ID NOS:33,35,37) of mGluR5 Ab2. In a further embodiment, an antibody or antibody fragment of the invention binds to the same epitope as mGluR5 Ab2. In another embodiment, an antibody or antibody fragment of the invention comprises the V_{H} chain (SEQ ID NO:23) and the V_{L} chain (SEQ ID NO:31) of mGluR5 Ab2 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:23. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:31. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:23 and a V_{L} chain having at least 80%, at least 85%, at least 90%, at least 95%, at least 98%, at least 99%, or 100% identity to the amino acid sequence of SEQ ID NO:31.

In one embodiment, an antibody or antibody fragment of the invention is mGluR5 Ab3. In one embodiment, an antibody or antibody fragment comprises the V_{H} chain CDRs (SEQ ID NOS:41,43,45) of mGluR5 Ab3. In one embodiment, an antibody or antibody fragment comprises the V_{L} chain CDRs (SEQ ID NOS:49,51,53) of mGluR5 Ab3. In one embodiment, an antibody or antibody fragment comprises the V_{H} chain CDRs (SEQ ID NOS:41,43,45) and the V_{L} chain CDRs (SEQ ID NOS:49,51,53) of mGluR5 Ab3. In a further embodiment, an antibody or antibody fragment of the invention binds to the same epitope as mGluR5 Ab3. In another embodiment, an antibody or antibody fragment of the invention comprises the V_{H} chain (SEQ ID NO:39) and the V_{L} chain (SEQ ID NO:47) of mGluR5 Ab3 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:39. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:47. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:39 and a V_{L} chain having at least 80%, at least 85%, at least 90%, at least 95%, at least 98%, at least 99%, or 100% identity to the amino acid sequence of SEQ ID NO:47.

In one embodiment, an antibody or antibody fragment of the invention is mGluR5 AM. In one embodiment, an antibody or antibody fragment comprises the V_{H} chain CDRs (SEQ ID NOS:57,59,61) of mGluR5 AM. In one embodiment, an antibody or antibody fragment comprises the V_{L} chain CDRs (SEQ ID NOS:65,67,69) of mGluR5 AM. In one embodiment, an antibody or antibody fragment comprises the V_{H} chain CDRs (SEQ ID NOS:57,59,61) and the V_{L} chain CDRs (SEQ ID NOS:65,67,69) of mGluR5 AM. In a further embodiment, an antibody or antibody fragment of the invention binds to the same epitope as mGluR5 AM. In another embodiment, an antibody or antibody fragment of the invention comprises the V_{H} chain (SEQ ID NO:55) and the V_{L} chain (SEQ ID NO:63) of mGluR5 AM or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:55. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:63. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:55 and a V_{L} chain having at least 80%, at least 85%, at least 90%, at least 95%, at least 98%, at least 99%, or 100% identity to the amino acid sequence of SEQ ID NO:63.

In one embodiment, an antibody or antibody fragment of the invention is mGluR5 Ab5. In one embodiment, an antibody or antibody fragment comprises the V_{H} chain CDRs (SEQ ID NOS:73,75,77) of mGluR5 Ab5. In one embodiment, an antibody or antibody fragment comprises the V_{L} chain CDRs (SEQ ID NOS:81,83,85) of mGluR5 Ab5. In one embodiment, an antibody or antibody fragment comprises the V_{H} chain CDRs (SEQ ID NOS:73,75,77) and the V_{L} chain CDRs (SEQ ID NOS:81,83,85) of mGluR5 Ab5. In a further embodiment, an antibody or antibody fragment of the invention binds to the same epitope as mGluR5 Ab5. In another embodiment, an antibody or antibody fragment of the invention comprises the V_{H} chain (SEQ ID NO:71) and the V_{L} chain (SEQ ID NO:79) of mGluR5 Ab5 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:71. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:79. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:71 and a V_{L} chain having at least 80%, at least 85%, at least 90%, at least 95%, at least 98%, at least 99%, or 100% identity to the amino acid sequence of SEQ ID NO:79.

In one embodiment, an antibody or antibody fragment of the invention is mGluR5 Ab6. In one embodiment, an antibody or antibody fragment comprises the V_{H} chain CDRs (SEQ ID NOS:89,91,93) of mGluR5 Ab6. In one embodiment, an antibody or antibody fragment comprises the V_{L} chain CDRs (SEQ ID NOS:97,99,101) of mGluR5 Ab6. In one embodiment, an antibody or antibody fragment comprises the V_{H} chain CDRs (SEQ ID NOS:89,91,93) and the V_{L} chain CDRs (SEQ ID NOS:97,99,101) of mGluR5 Ab6. In a further embodiment, an antibody or antibody fragment of the invention binds to the same epitope as mGluR5 Ab6. In another embodiment, an antibody or antibody fragment of the invention comprises the V_{H} chain (SEQ ID NO:87) and the V_{L} chain (SEQ ID NO:95) of mGluR5 Ab6 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:87. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:95. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:87 and a V_{L} chain having at least 80%, at least 85%, at least 90%, at least 95%, at least 98%, at least 99%, or 100% identity to the amino acid sequence of SEQ ID NO:95.

In one embodiment, an antibody or antibody fragment of the invention is mGluR5 Ab7. In one embodiment, an antibody or antibody fragment comprises the V_{H} chain CDRs (SEQ ID NOS:105,107,109) of mGluR5 Ab7. In one embodiment, an antibody or antibody fragment comprises the V_{L} chain CDRs (SEQ ID NOS:113,115,117) of mGluR5 Ab7. In one embodiment, an antibody or antibody fragment comprises the V_{H} chain CDRs (SEQ ID NOS:105,107,109) and the V_{L} chain CDRs (SEQ ID NOS:113,115,117) of mGluR5 Ab7. In a further embodiment, an antibody or antibody fragment of the invention binds to the same epitope as mGluR5 Ab7. In another embodiment, an antibody or antibody fragment of the invention comprises the V_{H} chain (SEQ ID NO:103) and the V_{L} chain (SEQ ID NO:111) of mGluR5 Ab7 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:103. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:111. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:103 and a V_{L} chain having at least 80%, at least 85%, at least 90%, at least 95%, at least 98%, at least 99%, or 100% identity to the amino acid sequence of SEQ ID NO:111.

In one embodiment, an antibody or antibody fragment of the invention is mGluR5 Ab8. In one embodiment, an antibody or antibody fragment comprises the V_{H} chain CDRs (SEQ ID NOS:121,123,125) of mGluR5 Ab8. In one embodiment, an antibody or antibody fragment comprises the V_{L} chain CDRs (SEQ ID NOS:129,131,133) of mGluR5 Ab8. In one embodiment, an antibody or antibody fragment comprises the V_{H} chain CDRs (SEQ ID NOS:121,123,125) and the V_{L} chain CDRs (SEQ ID NOS:129,131,133) of mGluR5 Ab8. In a further embodiment, an antibody or antibody fragment of the invention binds to the same epitope as mGluR5 Ab8. In another embodiment, an antibody or antibody fragment of the invention comprises the V_{H} chain (SEQ ID NO: 119) and the V_{L} chain (SEQ ID NO:127) of mGluR5 Ab8 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:119. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:127. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:119 and a V_{L} chain having at least 80%, at least 85%, at least 90%, at least 95%, at least 98%, at least 99%, or 100% identity to the amino acid sequence of SEQ ID NO:127.

In one embodiment, an antibody or antibody fragment of the invention is mGluR5 Ab9. In one embodiment, an antibody or antibody fragment comprises the V_{H} chain CDRs (SEQ ID NOS:137,139,141) of mGluR5 Ab9. In one embodiment, an antibody or antibody fragment comprises the V_{L} chain CDRs (SEQ ID NOS:145,147,149) of mGluR5 Ab9. In one embodiment, an antibody or antibody fragment comprises the V_{H} chain CDRs (SEQ ID NOS:137,139,141) and the V_{L} chain CDRs (SEQ ID NOS:145,147,149) of mGluR5 Ab9. In a further embodiment, an antibody or antibody fragment of the invention binds to the same epitope as mGluR5 Ab9. In another embodiment, an antibody or antibody fragment of the invention comprises the V_{H} chain (SEQ ID NO:135) and the V_{L} chain (SEQ ID NO:143) of mGluR5 Ab9 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:135. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:143. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:135 and a V_{L} chain having at least 80%, at least 85%, at least 90%, at least 95%, at least 98%, at least 99%, or 100% identity to the amino acid sequence of SEQ ID NO:143.

In one embodiment, an antibody or antibody fragment of the invention is mGluR5 Ab10. In one embodiment, an antibody or antibody fragment comprises the V_{H} chain CDRs (SEQ ID NOS:153,155,157) of mGluR5 Ab10. In one embodiment, an antibody or antibody fragment comprises the V_{L} chain CDRs (SEQ ID NOS:161,163,165) of mGluR5 Ab10. In one embodiment, an antibody or antibody fragment comprises the V_{H} chain CDRs (SEQ ID NOS:153,155,157) and the V_{L} chain CDRs (SEQ ID NOS:161,163,165) of mGluR5 Ab10. In a further embodiment, an antibody or antibody fragment of the invention binds to the same epitope as mGluR5 Ab10. In another embodiment, an antibody or antibody fragment of the invention comprises the V_{H} chain (SEQ ID NO:151) and the V_{L} chain (SEQ ID NO:159) of mGluR5 Ab10 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:151. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:159. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:151 and a V_{L} chain having at least 80%, at least 85%, at least 90%, at least 95%, at least 98%, at least 99%, or 100% identity to the amino acid sequence of SEQ ID NO:159.

In one embodiment, an antibody or antibody fragment of the invention is mGluR5 Ab11. In one embodiment, an antibody or antibody fragment comprises the V_{H} chain CDRs (SEQ ID NOS:169,171,173) of mGluR5 Ab11. In one embodiment, an antibody or antibody fragment comprises the V_{L} chain CDRs (SEQ ID NOS:177,179,181) of mGluR5 Ab11. In one embodiment, an antibody or antibody fragment comprises the V_{H} chain CDRs (SEQ ID NOS:169,171,173) and the V_{L} chain CDRs (SEQ ID NOS:177,179,181) of mGluR5 Ab11. In a further embodiment, an antibody or antibody fragment of the invention binds to the same epitope as mGluR5 Ab11. In another embodiment, an antibody or antibody fragment of the invention comprises the V_{H} chain (SEQ ID NO:167) and the V_{L} chain (SEQ ID NO:175) of mGluR5 Ab11 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:167. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:175. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:167 and a V_{L} chain having at least 80%, at least 85%, at least 90%, at least 95%, at least 98%, at least 99%, or 100% identity to the amino acid sequence of SEQ ID NO:175.

In one embodiment, an antibody or antibody fragment of the invention is mGluR5 Ab12. In one embodiment, an antibody or antibody fragment comprises the V_{H} chain CDRs (SEQ ID NOS:185,187,189) of mGluR5 Ab12. In one embodiment, an antibody or antibody fragment comprises the V_{L} chain CDRs (SEQ ID NOS:193,195,197) of mGluR5 Ab12. In one embodiment, an antibody or antibody fragment comprises the V_{H} chain CDRs (SEQ ID NOS:185,187,189) and the V_{L} chain CDRs (SEQ ID NOS:193,195,197) of mGluR5 Ab12. In a further embodiment, an antibody or antibody fragment of the invention binds to the same epitope as mGluR5 Ab12. In another embodiment, an antibody or antibody fragment of the invention comprises the V_{H} chain (SEQ ID NO:183) and the V_{L} chain (SEQ ID NO:191) of mGluR5 Ab12 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:183. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:191. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:183 and a V_{L} chain having at least 80%, at least 85%, at least 90%, at least 95%, at least 98%, at least 99%, or 100% identity to the amino acid sequence of SEQ ID NO:191.

In one embodiment, an antibody or antibody fragment of the invention is mGluR5 Ab13. In one embodiment, an antibody or antibody fragment comprises the V_{H} chain CDRs (SEQ ID NOS:201,203,205) of mGluR5 Ab13. In one embodiment, an antibody or antibody fragment comprises the V_{L} chain CDRs (SEQ ID NOS:209,211,213) of mGluR5 Ab13. In one embodiment, an antibody or antibody fragment comprises the V_{H} chain CDRs (SEQ ID NOS:201,203,205) and the V_{L} chain CDRs (SEQ ID NOS:209,211,213) of mGluR5 Ab13. In a further embodiment, an antibody or antibody fragment of the invention binds to the same epitope as mGluR5 Ab13. In another embodiment, an antibody or antibody fragment of the invention comprises the V_{H} chain (SEQ ID NO:199) and the V_{L} chain (SEQ ID NO:207) of mGluR5 Ab13 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:199. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:207. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:199 and a V_{L} chain having at least 80%, at least 85%, at least 90%, at least 95%, at least 98%, at least 99%, or 100% identity to the amino acid sequence of SEQ ID NO:207.

In one embodiment, an antibody or antibody fragment of the invention is mGluR5 Ab14. In one embodiment, an antibody or antibody fragment comprises the V_{H} chain CDRs (SEQ ID NOS:217,219,221) of mGluR5 Ab14. In one embodiment, an antibody or antibody fragment comprises the V_{L} chain CDRs (SEQ ID NOS:225,227,229) of mGluR5 Ab14. In one embodiment, an antibody or antibody fragment comprises the V_{H} chain CDRs (SEQ ID NOS:217,219,221) and the V_{L} chain CDRs (SEQ ID NOS:225,227,229) of mGluR5 Ab14. In a further embodiment, an antibody or antibody fragment of the invention binds to the same epitope as mGluR5 Ab14. In another embodiment, an antibody or antibody fragment of the invention comprises the V_{H} chain (SEQ ID NO:215) and the V_{L} chain (SEQ ID NO:223) of mGluR5 Ab14 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:215. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:223. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:215 and a V_{L} chain having at least 80%, at least 85%, at least 90%, at least 95%, at least 98%, at least 99%, or 100% identity to the amino acid sequence of SEQ ID NO:223.

In one embodiment, an antibody or antibody fragment of the invention is mGluR5 Ab15. In one embodiment, an antibody or antibody fragment comprises the V_{H} chain CDRs (SEQ ID NOS:233,235,237) of mGluR5 Ab15. In one embodiment, an antibody or antibody fragment comprises the V_{L} chain CDRs (SEQ ID NOS:241,243,245) of mGluR5 Ab15. In one embodiment, an antibody or antibody fragment comprises the V_{H} chain CDRs (SEQ ID NOS:233,235,237) and the V_{L} chain CDRs (SEQ ID NOS:241,243,245) of mGluR5 Ab15. In a further embodiment, an antibody or antibody fragment of the invention binds to the same epitope as mGluR5 Ab15. In another embodiment, an antibody or antibody fragment of the invention comprises the V_{H} chain (SEQ ID NO:231) and the V_{L} chain (SEQ ID NO:239) of mGluR5 Ab15 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:231. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:239. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:231 and a V_{L} chain having at least 80%, at least 85%, at least 90%, at least 95%, at least 98%, at least 99%, or 100% identity to the amino acid sequence of SEQ ID NO:239.

In one embodiment, an antibody or antibody fragment of the invention is mGluR5 Ab16. In one embodiment, an antibody or antibody fragment comprises the V_{H} chain CDRs (SEQ ID NOS:249,251,253) of mGluR5 Ab16. In one embodiment, an antibody or antibody fragment comprises the V_{L} chain CDRs (SEQ ID NOS:257,259,261) of mGluR5 Ab16. In one embodiment, an antibody or antibody fragment comprises the V_{H} chain CDRs (SEQ ID NOS:249,251,253) and the V_{L} chain CDRs (SEQ ID NOS:257,259,261) of mGluR5 Ab16. In a further embodiment, an antibody or antibody fragment of the invention binds to the same epitope as mGluR5 Ab16. In another embodiment, an antibody or antibody fragment of the invention comprises the V_{H} chain (SEQ ID NO:247) and the V_{L} chain (SEQ ID NO:255) of mGluR5 Ab16 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:247. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:255. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:247 and a V_{L} chain having at least 80%, at least 85%, at least 90%, at least 95%, at least 98%, at least 99%, or 100% identity to the amino acid sequence of SEQ ID NO:255.

In one embodiment, an antibody or antibody fragment of the invention is mGluR5 Ab17. In one embodiment, an antibody or antibody fragment comprises the V_{H} chain CDRs (SEQ ID NOS:265,267,269) of mGluR5 Ab17. In one embodiment, an antibody or antibody fragment comprises the V_{L} chain CDRs (SEQ ID NOS:273,275,277) of mGluR5 Ab17. In one embodiment, an antibody or antibody fragment comprises the V_{H} chain CDRs (SEQ ID NOS:265,267,269) and the V_{L} chain CDRs (SEQ ID NOS:273,275,277) of mGluR5 Ab17. In a further embodiment, an antibody or antibody fragment of the invention binds to the same epitope as mGluR5 Ab17. In another embodiment, an antibody or antibody fragment of the invention comprises the V_{H} chain (SEQ ID NO:263) and the V_{L} chain (SEQ ID NO:271) of mGluR5 Ab17 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:263. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:271. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:263 and a V_{L} chain having at least 80%, at least 85%, at least 90%, at least 95%, at least 98%, at least 99%, or 100% identity to the amino acid sequence of SEQ ID NO:271.

In one embodiment, an antibody or antibody fragment of the invention is mGluR5 Ab18. In one embodiment, an antibody or antibody fragment comprises the V_{H} chain CDRs (SEQ ID NOS:281,283,285) of mGluR5 Ab18. In one embodiment, an antibody or antibody fragment comprises the V_{L} chain CDRs (SEQ ID NOS:289,291,293) of mGluR5 Ab18. In one embodiment, an antibody or antibody fragment comprises the V_{H} chain CDRs (SEQ ID NOS:281,283,285) and the V_{L} chain CDRs (SEQ ID NOS:289,291,293) of mGluR5 Ab18. In a further embodiment, an antibody or antibody fragment of the invention binds to the same epitope as mGluR5 Ab18. In another embodiment, an antibody or antibody fragment of the invention comprises the V_{H} chain (SEQ ID NO:279) and the V_{L} chain (SEQ ID NO:287) of mGluR5 Ab18 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:279. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:287. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:279 and a V_{L} chain having at least 80%, at least 85%, at least 90%, at least 95%, at least 98%, at least 99%, or 100% identity to the amino acid sequence of SEQ ID NO:287.

In one embodiment, an antibody or antibody fragment of the invention is mGluR5 Ab19. In one embodiment, an antibody or antibody fragment comprises the V_{H} chain CDRs (SEQ ID NOS:297,299,301) of mGluR5 Ab19. In one embodiment, an antibody or antibody fragment comprises the V_{L} chain CDRs (SEQ ID NOS:305,307,309) of mGluR5 Ab19. In one embodiment, an antibody or antibody fragment comprises the V_{H} chain CDRs (SEQ ID NOS:297,299,301) and the V_{L} chain CDRs (SEQ ID NOS:305,307,309) of mGluR5 Ab19. In a further embodiment, an antibody or antibody fragment of the invention binds to the same epitope as mGluR5 Ab19. In another embodiment, an antibody or antibody fragment of the invention comprises the V_{H} chain (SEQ ID NO:295) and the V_{L} chain (SEQ ID NO:303) of mGluR5 Ab19 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:295. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:303. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:295 and a V_{L} chain having at least 80%, at least 85%, at least 90%, at least 95%, at least 98%, at least 99%, or 100% identity to the amino acid sequence of SEQ ID NO:303.

In one embodiment, an antibody or antibody fragment of the invention is mGluR5 Ab20. In one embodiment, an antibody or antibody fragment comprises the V_{H} chain CDRs (SEQ ID NOS:313,315,317) of mGluR5 Ab20. In one embodiment, an antibody or antibody fragment comprises the V_{L} chain CDRs (SEQ ID NOS:321,323,325) of mGluR5 Ab20. In one embodiment, an antibody or antibody fragment comprises the V_{H} chain CDRs (SEQ ID NOS:313,315,317) and the V_{L} chain CDRs (SEQ ID NOS:321,323,325) of mGluR5 Ab20. In a further embodiment, an antibody or antibody fragment of the invention binds to the same epitope as mGluR5 Ab20. In another embodiment, an antibody or antibody fragment of the invention comprises the V_{H} chain (SEQ ID NO:311) and the V_{L} chain (SEQ ID NO:319) of mGluR5 Ab20 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:311. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:319. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:311 and a V_{L} chain having at least 80%, at least 85%, at least 90%, at least 95%, at least 98%, at least 99%, or 100% identity to the amino acid sequence of SEQ ID NO:319.

In one embodiment, an antibody or antibody fragment of the invention is mGluR5 Ab21. In one embodiment, an antibody or antibody fragment comprises the V_{H} chain CDRs (SEQ ID NOS:329,331,333) of mGluR5 Ab21. In one embodiment, an antibody or antibody fragment comprises the V_{L} chain CDRs (SEQ ID NOS:337,339,341) of mGluR5 Ab21. In one embodiment, an antibody or antibody fragment comprises the V_{H} chain CDRs (SEQ ID NOS:329,331,333) and the V_{L} chain CDRs (SEQ ID NOS:337,339,341) of mGluR5 Ab21. In a further embodiment, an antibody or antibody fragment of the invention binds to the same epitope as mGluR5 Ab21. In another embodiment, an antibody or antibody fragment of the invention comprises the V_{H} chain (SEQ ID NO:327) and the V_{L} chain (SEQ ID NO:335) of mGluR5 Ab21 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:327. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:335. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:327 and a V_{L} chain having at least 80%, at least 85%, at least 90%, at least 95%, at least 98%, at least 99%, or 100% identity to the amino acid sequence of SEQ ID NO:335.

In one embodiment, an antibody or antibody fragment of the invention is mGluR5 Ab22. In one embodiment, an antibody or antibody fragment comprises the V_{H} chain CDRs (SEQ ID NOS:345,347,349) of mGluR5 Ab22. In one embodiment, an antibody or antibody fragment comprises the V_{L} chain CDRs (SEQ ID NOS:353,355,357) of mGluR5 Ab22. In one embodiment, an antibody or antibody fragment comprises the V_{H} chain CDRs (SEQ ID NOS:345,347,349) and the V_{L} chain CDRs (SEQ ID NOS:353,355,357) of mGluR5 Ab22. In a further embodiment, an antibody or antibody fragment of the invention binds to the same epitope as mGluR5 Ab22. In another embodiment, an antibody or antibody fragment of the invention comprises the V_{H} chain (SEQ ID NO:343) and the V_{L} chain (SEQ ID NO:351) of mGluR5 Ab22 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:343. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:351. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:343 and a V_{L} chain having at least 80%, at least 85%, at least 90%, at least 95%, at least 98%, at least 99%, or 100% identity to the amino acid sequence of SEQ ID NO:351.

In one embodiment, an antibody or antibody fragment of the invention is mGluR5 Ab23. In one embodiment, an antibody or antibody fragment comprises the V_{H} chain CDRs (SEQ ID NOS:361,363,365) of mGluR5 Ab23. In one embodiment, an antibody or antibody fragment comprises the V_{L} chain CDRs (SEQ ID NOS:369,371,373) of mGluR5 Ab23. In one embodiment, an antibody or antibody fragment comprises the V_{H} chain CDRs (SEQ ID NOS:361,363,365) and the V_{L} chain CDRs (SEQ ID NOS:369,371,373) of mGluR5 Ab23. In a further embodiment, an antibody or antibody fragment of the invention binds to the same epitope as mGluR5 Ab23. In another embodiment, an antibody or antibody fragment of the invention comprises the V_{H} chain (SEQ ID NO:359) and the V_{L} chain (SEQ ID NO:367) of mGluR5 Ab23 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:359. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:367. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:359 and a V_{L} chain having at least 80%, at least 85%, at least 90%, at least 95%, at least 98%, at least 99%, or 100% identity to the amino acid sequence of SEQ ID NO:367.

In one embodiment, an antibody or antibody fragment of the invention is mGluR5 Ab24. In one embodiment, an antibody or antibody fragment comprises the V_{H} chain CDRs (SEQ ID NOS:377,379,381) of mGluR5 Ab24. In one embodiment, an antibody or antibody fragment comprises the V_{L} chain CDRs (SEQ ID NOS:385,387,389) of mGluR5 Ab24. In one embodiment, an antibody or antibody fragment comprises the V_{H} chain CDRs (SEQ ID NOS:377,379,381) and the V_{L} chain CDRs (SEQ ID NOS:385,387,389) of mGluR5 Ab24. In a further embodiment, an antibody or antibody fragment of the invention binds to the same epitope as mGluR5 Ab24. In another embodiment, an antibody or antibody fragment of the invention comprises the V_{H} chain (SEQ ID NO:375) and the V_{L} chain (SEQ ID NO:383) of mGluR5 Ab24 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:375. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:383. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:375 and a V_{L} chain having at least 80%, at least 85%, at least 90%, at least 95%, at least 98%, at least 99%, or 100% identity to the amino acid sequence of SEQ ID NO:383.

In one embodiment, an antibody or antibody fragment of the invention is mGluR5 Ab25. In one embodiment, an antibody or antibody fragment comprises the V_{H} chain CDRs (SEQ ID NOS:393,395,397) of mGluR5 Ab25. In one embodiment, an antibody or antibody fragment comprises the V_{L} chain CDRs (SEQ ID NOS:401,403,405) of mGluR5 Ab25. In one embodiment, an antibody or antibody fragment comprises the V_{H} chain CDRs (SEQ ID NOS:393,395,397) and the V_{L} chain CDRs (SEQ ID NOS:401,403,405) of mGluR5 Ab25. In a further embodiment, an antibody or antibody fragment of the invention binds to the same epitope as mGluR5 Ab25. In another embodiment, an antibody or antibody fragment of the invention comprises the V_{H} chain (SEQ ID NO:391) and the V_{L} chain (SEQ ID NO:399) of mGluR5 Ab25 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:391. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:399. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:391 and a V_{L} chain having at least 80%, at least 85%, at least 90%, at least 95%, at least 98%, at least 99%, or 100% identity to the amino acid sequence of SEQ ID NO:399.

In one embodiment, an antibody or antibody fragment of the invention is mGluR5 Ab26. In one embodiment, an antibody or antibody fragment comprises the V_{H} chain CDRs (SEQ ID NOS:409,411,413) of mGluR5 Ab26. In one embodiment, an antibody or antibody fragment comprises the V_{L} chain CDRs (SEQ ID NOS:417,419,421) of mGluR5 Ab26. In one embodiment, an antibody or antibody fragment comprises the V_{H} chain CDRs (SEQ ID NOS:409,411,413) and the V_{L} chain CDRs (SEQ ID NOS:417,419,421) of mGluR5 Ab26. In a further embodiment, an antibody or antibody fragment of the invention binds to the same epitope as mGluR5 Ab26. In another embodiment, an antibody or antibody fragment of the invention comprises the V_{H} chain (SEQ ID NO:407) and the V_{L} chain (SEQ ID NO:415) of mGluR5 Ab26 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:407. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:415. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:407 and a V_{L} chain having at least 80%, at least 85%, at least 90%, at least 95%, at least 98%, at least 99%, or 100% identity to the amino acid sequence of SEQ ID NO:415.

In one embodiment, an antibody or antibody fragment of the invention is mGluR5 Ab27. In one embodiment, an antibody or antibody fragment comprises the V_{H} chain CDRs (SEQ ID NOS:425,427,429) of mGluR5 Ab27. In one embodiment, an antibody or antibody fragment comprises the V_{L} chain CDRs (SEQ ID NOS:433,435,437) of mGluR5 Ab27. In one embodiment, an antibody or antibody fragment comprises the V_{H} chain CDRs (SEQ ID NOS:425,427,429) and the V_{L} chain CDRs (SEQ ID NOS:433,435,437) of mGluR5 Ab27. In a further embodiment, an antibody or antibody fragment of the invention binds to the same epitope as mGluR5 Ab27. In another embodiment, an antibody or antibody fragment of the invention comprises the V_{H} chain (SEQ ID NO:423) and the V_{L} chain (SEQ ID NO:431) of mGluR5 Ab27 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:423. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:431. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:423 and a V_{L} chain having at least 80%, at least 85%, at least 90%, at least 95%, at least 98%, at least 99%, or 100% identity to the amino acid sequence of SEQ ID NO:431.

In one embodiment, an antibody or antibody fragment of the invention is mGluR5 Ab28. In one embodiment, an antibody or antibody fragment comprises the V_{H} chain CDRs (SEQ ID NOS:441,443,445) of mGluR5 Ab28. In one embodiment, an antibody or antibody fragment comprises the V_{L} chain CDRs (SEQ ID NOS:449,451,453) of mGluR5 Ab28. In one embodiment, an antibody or antibody fragment comprises the V_{H} chain CDRs (SEQ ID NOS:441,443,445) and the V_{L} chain CDRs (SEQ ID NOS:449,451,453) of mGluR5 Ab28. In a further embodiment, an antibody or antibody fragment of the invention binds to the same epitope as mGluR5 Ab28. In another embodiment, an antibody or antibody fragment of the invention comprises the V_{H} chain (SEQ ID NO:439) and the V_{L} chain (SEQ ID NO:447) of mGluR5 Ab28 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:439. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:447. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:439 and a V_{L} chain having at least 80%, at least 85%, at least 90%, at least 95%, at least 98%, at least 99%, or 100% identity to the amino acid sequence of SEQ ID NO:447.

In one embodiment, an antibody or antibody fragment of the invention is mGluR5 Ab29. In one embodiment, an antibody or antibody fragment comprises the V_{H} chain CDRs (SEQ ID NOS:457,459,461) of mGluR5 Ab29. In one embodiment, an antibody or antibody fragment comprises the V_{L} chain CDRs (SEQ ID NOS:465,467,469) of mGluR5 Ab29. In one embodiment, an antibody or antibody fragment comprises the V_{H} chain CDRs (SEQ ID NOS:457,459,461) and the V_{L} chain CDRs (SEQ ID NOS:465,467,469) of mGluR5 Ab29. In a further embodiment, an antibody or antibody fragment of the invention binds to the same epitope as mGluR5 Ab29. In another embodiment, an antibody or antibody fragment of the invention comprises the V_{H} chain (SEQ ID NO:455) and the V_{L} chain (SEQ ID NO:463) of mGluR5 Ab29 or a V_{L} chain and a V_{H} chain possessing at least 90 or 95% sequence identity therewith. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:455. In one embodiment, an antibody or antibody fragment of the invention comprises a V_{L} chain having at least 80%, at least 85%, at least 90%, at least 95%, at least 98%, at least 99%, or 100% identity to the amino acid sequence of SEQ ID NO:463. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:455 and a V_{L} chain having at least 80%, at least 85%, at least 90%, at least 95%, at least 98%, at least 99%, or 100% identity to the amino acid sequence of SEQ ID NO:463.

### Polynucleotides Encoding Anti-mGluR5 Antibodies

The invention further encompasses polynucleotides encoding antibodies and antibody fragments.

In one embodiment, a polynucleotide of the invention encodes mGluR5 Ab1. In one embodiment, a polynucleotide of the invention encodes an antibody or antibody fragment comprising the V_{H} chain CDRs (SEQ ID NOS:8,10,12) of mGluR5 Ab1. In one embodiment, a polynucleotide of the invention encodes an antibody or antibody fragment comprising the V_{L} chain CDRs (SEQ ID NOS:16,18,20) of mGluR5 Ab1. In one embodiment, a polynucleotide of the invention encodes an antibody or antibody fragment comprising the V_{H} chain CDRs (SEQ ID NOS:8,10,12) and the V_{L} chain CDRs (SEQ ID NOS:16,18,20) of mGluR5 Ab1. In a further embodiment, a polynucleotide of the invention encodes an antibody or antibody fragment binding to the same epitope as mGluR5 Ab1. In one embodiment, a polynucleotide of the invention encodes an antibody or antibody fragment comprising the V_{H} chain (SEQ ID NO:6) and the V_{L} chain (SEQ ID NO:14) of mGluR5 Ab1. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:6. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:14. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:6 and a V_{L} chain having at least 80%, at least 85%, at least 90%, at least 95%, at least 98%, at least 99%, or 100% identity to the amino acid sequence of SEQ ID NO:14.

In one embodiment, a polynucleotide of the invention encodes mGluR5 Ab2. In one embodiment, a polynucleotide of the invention encodes an antibody or antibody fragment comprising the V_{H} chain CDRs (SEQ ID NOS:24,26,28) of mGluR5 Ab2. In one embodiment, a polynucleotide of the invention encodes an antibody or antibody fragment comprising the V_{L} chain CDRs (SEQ ID NOS:32,34,36) of mGluR5 Ab2. In one embodiment, a polynucleotide of the invention encodes an antibody or antibody fragment comprising the V_{H} chain CDRs (SEQ ID NOS:24,26,28) and the V_{L} chain CDRs (SEQ ID NOS:32,34,36) of mGluR5 Ab2. In a further embodiment, a polynucleotide of the invention encodes an antibody or antibody fragment binding to the same epitope as mGluR5 Ab2. In one embodiment, a polynucleotide of the invention encodes an antibody or antibody fragment comprising the V_{H} chain (SEQ ID NO:22) and the V_{L} chain (SEQ ID NO:30) of mGluR5 Ab2. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:22. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:30. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:22 and a V_{L} chain having at least 80%, at least 85%, at least 90%, at least 95%, at least 98%, at least 99%, or 100% identity to the amino acid sequence of SEQ ID NO:30.

In one embodiment, a polynucleotide of the invention encodes mGluR5 Ab3. In one embodiment, a polynucleotide of the invention encodes an antibody or antibody fragment comprising the V_{H} chain CDRs (SEQ ID NOS:40,42,44) of mGluR5 Ab3. In one embodiment, a polynucleotide of the invention encodes an antibody or antibody fragment comprising the V_{L} chain CDRs (SEQ ID NOS:48,50,52) of mGluR5 Ab3. In one embodiment, a polynucleotide of the invention encodes an antibody or antibody fragment comprising the V_{H} chain CDRs (SEQ ID NOS:40,42,44) and the V_{L} chain CDRs (SEQ ID NOS:48,50,52) of mGluR5 Ab3. In a further embodiment, a polynucleotide of the invention encodes an antibody or antibody fragment binding to the same epitope as mGluR5 Ab3. In one embodiment, a polynucleotide of the invention encodes an antibody or antibody fragment comprising the V_{H} chain (SEQ ID NO:38) and the V_{L} chain (SEQ ID NO:46) of mGluR5 Ab3. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:38. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:46. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:38 and a V_{L} chain having at least 80%, at least 85%, at least 90%, at least 95%, at least 98%, at least 99%, or 100% identity to the amino acid sequence of SEQ ID NO:46.

In one embodiment, a polynucleotide of the invention encodes mGluR5 Ab4. In one embodiment, a polynucleotide of the invention encodes an antibody or antibody fragment comprising the V_{H} chain CDRs (SEQ ID NOS:56,58,60) of mGluR5 AM. In one embodiment, a polynucleotide of the invention encodes an antibody or antibody fragment comprising the V_{L} chain CDRs (SEQ ID NOS:64,66,68) of mGluR5 AM. In one embodiment, a polynucleotide of the invention encodes an antibody or antibody fragment comprising the V_{H} chain CDRs (SEQ ID NOS:56,58,60) and the V_{L} chain CDRs (SEQ ID NOS:64,66,68) of mGluR5 AM. In a further embodiment, a polynucleotide of the invention encodes an antibody or antibody fragment binding to the same epitope as mGluR5 AM. In one embodiment, a polynucleotide of the invention encodes an antibody or antibody fragment comprising the V_{H} chain (SEQ ID NO:54) and the V_{L} chain (SEQ ID NO:62) of mGluR5 AM. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:54. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:62. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:54 and a V_{L} chain having at least 80%, at least 85%, at least 90%, at least 95%, at least 98%, at least 99%, or 100% identity to the amino acid sequence of SEQ ID NO:62.

In one embodiment, a polynucleotide of the invention encodes mGluR5 Ab5. In one embodiment, a polynucleotide of the invention encodes an antibody or antibody fragment comprising the V_{H} chain CDRs (SEQ ID NOS:72,74,76) of mGluR5 Ab5. In one embodiment, a polynucleotide of the invention encodes an antibody or antibody fragment comprising the V_{L} chain CDRs (SEQ ID NOS:80,82,84) of mGluR5 Ab5. In one embodiment, a polynucleotide of the invention encodes an antibody or antibody fragment comprising the V_{H} chain CDRs (SEQ ID NOS:72,74,76) and the V_{L} chain CDRs (SEQ ID NOS:80,82,84) of mGluR5 Ab5. In a further embodiment, a polynucleotide of the invention encodes an antibody or antibody fragment binding to the same epitope as mGluR5 AbS. In one embodiment, a polynucleotide of the invention encodes an antibody or antibody fragment comprising the V_{H} chain (SEQ ID NO:70) and the V_{L} chain (SEQ ID NO:78) of mGluR5 Ab5. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:70. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:78. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:70 and a V_{L} chain having at least 80%, at least 85%, at least 90%, at least 95%, at least 98%, at least 99%, or 100% identity to the amino acid sequence of SEQ ID NO:78.

In one embodiment, a polynucleotide of the invention encodes mGluR5 Ab6. In one embodiment, a polynucleotide of the invention encodes an antibody or antibody fragment comprising the V_{H} chain CDRs (SEQ ID NOS:88,90,92) of mGluR5 Ab6. In one embodiment, a polynucleotide of the invention encodes an antibody or antibody fragment comprising the V_{L} chain CDRs (SEQ ID NOS:96,98,100) of mGluR5 Ab6. In one embodiment, a polynucleotide of the invention encodes an antibody or antibody fragment comprising the V_{H} chain CDRs (SEQ ID NOS:88,90,92) and the V_{L} chain CDRs (SEQ ID NOS:96,98,100) of mGluR5 Ab6. In a further embodiment, a polynucleotide of the invention encodes an antibody or antibody fragment binding to the same epitope as mGluR5 Ab6. In one embodiment, a polynucleotide of the invention encodes an antibody or antibody fragment comprising the V_{H} chain (SEQ ID NO:86) and the V_{L} chain (SEQ ID NO:94) of mGluR5 Ab6. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:86. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:94. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:86 and a V_{L} chain having at least 80%, at least 85%, at least 90%, at least 95%, at least 98%, at least 99%, or 100% identity to the amino acid sequence of SEQ ID NO:94.

In one embodiment, a polynucleotide of the invention encodes mGluR5 Ab7. In one embodiment, a polynucleotide of the invention encodes an antibody or antibody fragment comprising the V_{H} chain CDRs (SEQ ID NOS:104,106,108) of mGluR5 Ab7. In one embodiment, a polynucleotide of the invention encodes an antibody or antibody fragment comprising the V_{L} chain CDRs (SEQ ID NOS: 112,114,116) of mGluR5 Ab7. In one embodiment, a polynucleotide of the invention encodes an antibody or antibody fragment comprising the V_{H} chain CDRs (SEQ ID NOS:104,106,108) and the V_{L} chain CDRs (SEQ ID NOS: 112,114,116) of mGluR5 Ab7. In a further embodiment, a polynucleotide of the invention encodes an antibody or antibody fragment binding to the same epitope as mGluR5 Ab7. In one embodiment, a polynucleotide of the invention encodes an antibody or antibody fragment comprising the V_{H} chain (SEQ ID NO:102) and the V_{L} chain (SEQ ID NO:110) of mGluR5 Ab7. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:102. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:110. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:102 and a V_{L} chain having at least 80%, at least 85%, at least 90%, at least 95%, at least 98%, at least 99%, or 100% identity to the amino acid sequence of SEQ ID NO:110.

In one embodiment, a polynucleotide of the invention encodes mGluR5 Ab8. In one embodiment, a polynucleotide of the invention encodes an antibody or antibody fragment comprising the V_{H} chain CDRs (SEQ ID NOS:120,122,124) of mGluR5 Ab8. In one embodiment, a polynucleotide of the invention encodes an antibody or antibody fragment comprising the V_{L} chain CDRs (SEQ ID NOS:128,130,132) of mGluR5 Ab8. In one embodiment, a polynucleotide of the invention encodes an antibody or antibody fragment comprising the V_{H} chain CDRs (SEQ ID NOS:120,122,124) and the V_{L} chain CDRs (SEQ ID NOS:128,130,132) of mGluR5 Ab8. In a further embodiment, a polynucleotide of the invention encodes an antibody or antibody fragment binding to the same epitope as mGluR5 Ab8. In one embodiment, a polynucleotide of the invention encodes an antibody or antibody fragment comprising the V_{H} chain (SEQ ID NO: 118) and the V_{L} chain (SEQ ID NO:126) of mGluR5 Ab8. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:118. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:126. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:118 and a V_{L} chain having at least 80%, at least 85%, at least 90%, at least 95%, at least 98%, at least 99%, or 100% identity to the amino acid sequence of SEQ ID NO:126.

In one embodiment, a polynucleotide of the invention encodes mGluR5 Ab9. In one embodiment, a polynucleotide of the invention encodes an antibody or antibody fragment comprising the V_{H} chain CDRs (SEQ ID NOS:136,138,140) of mGluR5 Ab9. In one embodiment, a polynucleotide of the invention encodes an antibody or antibody fragment comprising the V_{L} chain CDRs (SEQ ID NOS:144,146,148) of mGluR5 Ab9. In one embodiment, a polynucleotide of the invention encodes an antibody or antibody fragment comprising the V_{H} chain CDRs (SEQ ID NOS:136,138,140) and the V_{L} chain CDRs (SEQ ID NOS:144,146,148) of mGluR5 Ab9. In a further embodiment, a polynucleotide of the invention encodes an antibody or antibody fragment binding to the same epitope as mGluR5 Ab9. In one embodiment, a polynucleotide of the invention encodes an antibody or antibody fragment comprising the V_{H} chain (SEQ ID NO:134) and the V_{L} chain (SEQ ID NO:142) of mGluR5 Ab9. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:134. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:142. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:134 and a V_{L} chain having at least 80%, at least 85%, at least 90%, at least 95%, at least 98%, at least 99%, or 100% identity to the amino acid sequence of SEQ ID NO:142.

In one embodiment, a polynucleotide of the invention encodes mGluR5 Ab10. In one embodiment, a polynucleotide of the invention encodes an antibody or antibody fragment comprising the V_{H} chain CDRs (SEQ ID NOS:152,154,156) of mGluR5 Ab10. In one embodiment, a polynucleotide of the invention encodes an antibody or antibody fragment comprising the V_{L} chain CDRs (SEQ ID NOS:160,162,164) of mGluR5 Ab10. In one embodiment, a polynucleotide of the invention encodes an antibody or antibody fragment comprising the V_{H} chain CDRs (SEQ ID NOS:152,154,156) and the V_{L} chain CDRs (SEQ ID NOS:160,162,164) of mGluR5 Ab10. In a further embodiment, a polynucleotide of the invention encodes an antibody or antibody fragment binding to the same epitope as mGluR5 Ab10. In one embodiment, a polynucleotide of the invention encodes an antibody or antibody fragment comprising the V_{H} chain (SEQ ID NO:150) and the V_{L} chain (SEQ ID NO:158) of mGluR5 Ab10. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:150. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:158. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:150 and a V_{L} chain having at least 80%, at least 85%, at least 90%, at least 95%, at least 98%, at least 99%, or 100% identity to the amino acid sequence of SEQ ID NO:158.

In one embodiment, a polynucleotide of the invention encodes mGluR5 Ab11. In one embodiment, a polynucleotide of the invention encodes an antibody or antibody fragment comprising the V_{H} chain CDRs (SEQ ID NOS:168,170,172) of mGluR5 Ab11. In one embodiment, a polynucleotide of the invention encodes an antibody or antibody fragment comprising the V_{L} chain CDRs (SEQ ID NOS:176,178,180) of mGluR5 Ab11. In one embodiment, a polynucleotide of the invention encodes an antibody or antibody fragment comprising the V_{H} chain CDRs (SEQ ID NOS:168,170,172) and the V_{L} chain CDRs (SEQ ID NOS:176,178,180) of mGluR5 Ab11. In a further embodiment, a polynucleotide of the invention encodes an antibody or antibody fragment binding to the same epitope as mGluR5 Ab11. In one embodiment, a polynucleotide of the invention encodes an antibody or antibody fragment comprising the V_{H} chain (SEQ ID NO:166) and the V_{L} chain (SEQ ID NO:174) of mGluR5 Ab11. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:166. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:174. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:166 and a V_{L} chain having at least 80%, at least 85%, at least 90%, at least 95%, at least 98%, at least 99%, or 100% identity to the amino acid sequence of SEQ ID NO:174.

In one embodiment, a polynucleotide of the invention encodes mGluR5 Ab12. In one embodiment, a polynucleotide of the invention encodes an antibody or antibody fragment comprising the V_{H} chain CDRs (SEQ ID NOS:184,186,188) of mGluR5 Ab12. In one embodiment, a polynucleotide of the invention encodes an antibody or antibody fragment comprising the V_{L} chain CDRs (SEQ ID NOS:192,194,196) of mGluR5 Ab12. In one embodiment, a polynucleotide of the invention encodes an antibody or antibody fragment comprising the V_{H} chain CDRs (SEQ ID NOS:184,186,188) and the V_{L} chain CDRs (SEQ ID NOS:192,194,196) of mGluR5 Ab12. In a further embodiment, a polynucleotide of the invention encodes an antibody or antibody fragment binding to the same epitope as mGluR5 Ab12. In one embodiment, a polynucleotide of the invention encodes an antibody or antibody fragment comprising the V_{H} chain (SEQ ID NO:182) and the V_{L} chain (SEQ ID NO:190) of mGluR5 Ab12. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:182. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:190. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:182 and a V_{L} chain having at least 80%, at least 85%, at least 90%, at least 95%, at least 98%, at least 99%, or 100% identity to the amino acid sequence of SEQ ID NO:190.

In one embodiment, a polynucleotide of the invention encodes mGluR5 Ab13. In one embodiment, a polynucleotide of the invention encodes an antibody or antibody fragment comprising the V_{H} chain CDRs (SEQ ID NOS:200,202,204) of mGluR5 Ab13. In one embodiment, a polynucleotide of the invention encodes an antibody or antibody fragment comprising the V_{L} chain CDRs (SEQ ID NOS:208,210,212) of mGluR5 Ab13. In one embodiment, a polynucleotide of the invention encodes an antibody or antibody fragment comprising the V_{H} chain CDRs (SEQ ID NOS:200,202,204) and the V_{L} chain CDRs (SEQ ID NOS:208,210,212) of mGluR5 Ab13. In a further embodiment, a polynucleotide of the invention encodes an antibody or antibody fragment binding to the same epitope as mGluR5 Ab13. In one embodiment, a polynucleotide of the invention encodes an antibody or antibody fragment comprising the V_{H} chain (SEQ ID NO:198) and the V_{L} chain (SEQ ID NO:206) of mGluR5 Ab13. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:198. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:206. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:198 and a V_{L} chain having at least 80%, at least 85%, at least 90%, at least 95%, at least 98%, at least 99%, or 100% identity to the amino acid sequence of SEQ ID NO:206.

In one embodiment, a polynucleotide of the invention encodes mGluR5 Ab14. In one embodiment, a polynucleotide of the invention encodes an antibody or antibody fragment comprising the V_{H} chain CDRs (SEQ ID NOS:216,218,220) of mGluR5 Ab14. In one embodiment, a polynucleotide of the invention encodes an antibody or antibody fragment comprising the V_{L} chain CDRs (SEQ ID NOS:224,226,228) of mGluR5 Ab14. In one embodiment, a polynucleotide of the invention encodes an antibody or antibody fragment comprising the V_{H} chain CDRs (SEQ ID NOS:216,218,220) and the V_{L} chain CDRs (SEQ ID NOS:224,226,228) of mGluR5 Ab14. In a further embodiment, a polynucleotide of the invention encodes an antibody or antibody fragment binding to the same epitope as mGluR5 Ab14. In one embodiment, a polynucleotide of the invention encodes an antibody or antibody fragment comprising the V_{H} chain (SEQ ID NO:214) and the V_{L} chain (SEQ ID NO:222) of mGluR5 Ab14. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:214. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:222. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:214 and a V_{L} chain having at least 80%, at least 85%, at least 90%, at least 95%, at least 98%, at least 99%, or 100% identity to the amino acid sequence of SEQ ID NO:222.

In one embodiment, a polynucleotide of the invention encodes mGluR5 Ab15. In one embodiment, a polynucleotide of the invention encodes an antibody or antibody fragment comprising the VHchain CDRs (SEQ ID NOS:232,234,236) of mGluR5 Ab15. In one embodiment, a polynucleotide of the invention encodes an antibody or antibody fragment comprising the V_{L} chain CDRs (SEQ ID NOS:240,242,244) of mGluR5 Ab15. In one embodiment, a polynucleotide of the invention encodes an antibody or antibody fragment comprising the V_{H} chain CDRs (SEQ ID NOS:232,234,236) and the V_{L} chain CDRs (SEQ ID NOS:240,242,244) of mGluR5 Ab15. In a further embodiment, a polynucleotide of the invention encodes an antibody or antibody fragment binding to the same epitope as mGluR5 Ab15. In one embodiment, a polynucleotide of the invention encodes an antibody or antibody fragment comprising the V_{H} chain (SEQ ID NO:230) and the V_{L} chain (SEQ ID NO:238) of mGluR5 Ab15. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:230. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:238. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:230 and a V_{L} chain having at least 80%, at least 85%, at least 90%, at least 95%, at least 98%, at least 99%, or 100% identity to the amino acid sequence of SEQ ID NO:238.

In one embodiment, a polynucleotide of the invention encodes mGluR5 Ab16. In one embodiment, a polynucleotide of the invention encodes an antibody or antibody fragment comprising the V_{H} chain CDRs (SEQ ID NOS:248,250,252) of mGluR5 Ab16. In one embodiment, a polynucleotide of the invention encodes an antibody or antibody fragment comprising the V_{L} chain CDRs (SEQ ID NOS:256,258,260) of mGluR5 Ab16. In one embodiment, a polynucleotide of the invention encodes an antibody or antibody fragment comprising the V_{H} chain CDRs (SEQ ID NOS:248,250,252) and the V_{L} chain CDRs (SEQ ID NOS:256,258,260) of mGluR5 Ab16. In a further embodiment, a polynucleotide of the invention encodes an antibody or antibody fragment binding to the same epitope as mGluR5 Ab16. In one embodiment, a polynucleotide of the invention encodes an antibody or antibody fragment comprising the V_{H} chain (SEQ ID NO:246) and the V_{L} chain (SEQ ID NO:254) of mGluR5 Ab16. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:246. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:254. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:246 and a V_{L} chain having at least 80%, at least 85%, at least 90%, at least 95%, at least 98%, at least 99%, or 100% identity to the amino acid sequence of SEQ ID NO:254.

In one embodiment, a polynucleotide of the invention encodes mGluR5 Ab17. In one embodiment, a polynucleotide of the invention encodes an antibody or antibody fragment comprising the V_{H} chain CDRs (SEQ ID NOS:264,266,268) of mGluR5 Ab17. In one embodiment, a polynucleotide of the invention encodes an antibody or antibody fragment comprising the V_{L} chain CDRs (SEQ ID NOS:272,274,276) of mGluR5 Ab17. In one embodiment, a polynucleotide of the invention encodes an antibody or antibody fragment comprising the V_{H} chain CDRs (SEQ ID NOS:264,266,268) and the V_{L} chain CDRs (SEQ ID NOS:272,274,276) of mGluR5 Ab17. In a further embodiment, a polynucleotide of the invention encodes an antibody or antibody fragment binding to the same epitope as mGluR5 Ab17. In one embodiment, a polynucleotide of the invention encodes an antibody or antibody fragment comprising the V_{H} chain (SEQ ID NO:262) and the V_{L} chain (SEQ ID NO:270) of mGluR5 Ab17. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:262. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:270. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:262 and a V_{L} chain having at least 80%, at least 85%, at least 90%, at least 95%, at least 98%, at least 99%, or 100% identity to the amino acid sequence of SEQ ID NO:270.

In one embodiment, a polynucleotide of the invention encodes mGluR5 Ab18. In one embodiment, a polynucleotide of the invention encodes an antibody or antibody fragment comprising the V_{H} chain CDRs (SEQ ID NOS:280,282,284) of mGluR5 Ab18. In one embodiment, a polynucleotide of the invention encodes an antibody or antibody fragment comprising the V_{L} chain CDRs (SEQ ID NOS:288,290,292) of mGluR5 Ab18. In one embodiment, a polynucleotide of the invention encodes an antibody or antibody fragment comprising the V_{H} chain CDRs (SEQ ID NOS:280,282,284) and the V_{L} chain CDRs (SEQ ID NOS:288,290,292) of mGluR5 Ab18. In a further embodiment, a polynucleotide of the invention encodes an antibody or antibody fragment binding to the same epitope as mGluR5 Ab18. In one embodiment, a polynucleotide of the invention encodes an antibody or antibody fragment comprising the V_{H} chain (SEQ ID NO:278) and the V_{L} chain (SEQ ID NO:286) of mGluR5 Ab18. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:278. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:286. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:278 and a V_{L} chain having at least 80%, at least 85%, at least 90%, at least 95%, at least 98%, at least 99%, or 100% identity to the amino acid sequence of SEQ ID NO:286.

In one embodiment, a polynucleotide of the invention encodes mGluR5 Ab19. In one embodiment, a polynucleotide of the invention encodes an antibody or antibody fragment comprising the V_{H} chain CDRs (SEQ ID NOS:296,298,300) of mGluR5 Ab19. In one embodiment, a polynucleotide of the invention encodes an antibody or antibody fragment comprising the V_{L} chain CDRs (SEQ ID NOS:304,306,308) of mGluR5 Ab19. In one embodiment, a polynucleotide of the invention encodes an antibody or antibody fragment comprising the V_{H} chain CDRs (SEQ ID NOS:296,298,300) and the V_{L} chain CDRs (SEQ ID NOS:304,306,308) of mGluR5 Ab19. In a further embodiment, a polynucleotide of the invention encodes an antibody or antibody fragment binding to the same epitope as mGluR5 Ab19. In one embodiment, a polynucleotide of the invention encodes an antibody or antibody fragment comprising the V_{H} chain (SEQ ID NO:294) and the V_{L} chain (SEQ ID NO:302) of mGluR5 Ab19. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:294. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:302. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:294 and a V_{L} chain having at least 80%, at least 85%, at least 90%, at least 95%, at least 98%, at least 99%, or 100% identity to the amino acid sequence of SEQ ID NO:302.

In one embodiment, a polynucleotide of the invention encodes mGluR5 Ab20. In one embodiment, a polynucleotide of the invention encodes an antibody or antibody fragment comprising the V_{H} chain CDRs (SEQ ID NOS:312,314,316) of mGluR5 Ab20. In one embodiment, a polynucleotide of the invention encodes an antibody or antibody fragment comprising the V_{L} chain CDRs (SEQ ID NOS:320,322,324) of mGluR5 Ab20. In one embodiment, a polynucleotide of the invention encodes an antibody or antibody fragment comprising the V_{H} chain CDRs (SEQ ID NOS:312,314,316) and the V_{L} chain CDRs (SEQ ID NOS:320,322,324) of mGluR5 Ab20. In a further embodiment, a polynucleotide of the invention encodes an antibody or antibody fragment binding to the same epitope as mGluR5 Ab20. In one embodiment, a polynucleotide of the invention encodes an antibody or antibody fragment comprising the V_{H} chain (SEQ ID NO:310) and the V_{L} chain (SEQ ID NO:318) of mGluR5 Ab20. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:310. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:318. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:310 and a V_{L} chain having at least 80%, at least 85%, at least 90%, at least 95%, at least 98%, at least 99%, or 100% identity to the amino acid sequence of SEQ ID NO:318.

In one embodiment, a polynucleotide of the invention encodes mGluR5 Ab21. In one embodiment, a polynucleotide of the invention encodes an antibody or antibody fragment comprising the V_{H} chain CDRs (SEQ ID NOS:328,330,332) of mGluR5 Ab21. In one embodiment, a polynucleotide of the invention encodes an antibody or antibody fragment comprising the V_{L} chain CDRs (SEQ ID NOS:336,338,340) of mGluR5 Ab21. In one embodiment, a polynucleotide of the invention encodes an antibody or antibody fragment comprising the V_{H} chain CDRs (SEQ ID NOS:328,330,332) and the V_{L} chain CDRs (SEQ ID NOS:336,338,340) of mGluR5 Ab21. In a further embodiment, a polynucleotide of the invention encodes an antibody or antibody fragment binding to the same epitope as mGluR5 Ab21. In one embodiment, a polynucleotide of the invention encodes an antibody or antibody fragment comprising the V_{H} chain (SEQ ID NO:326) and the V_{L} chain (SEQ ID NO:334) of mGluR5 Ab21. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:326. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:334. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:326 and a V_{L} chain having at least 80%, at least 85%, at least 90%, at least 95%, at least 98%, at least 99%, or 100% identity to the amino acid sequence of SEQ ID NO:334.

In one embodiment, a polynucleotide of the invention encodes mGluR5 Ab22. In one embodiment, a polynucleotide of the invention encodes an antibody or antibody fragment comprising the V_{H} chain CDRs (SEQ ID NOS:344,346,348) of mGluR5 Ab22. In one embodiment, a polynucleotide of the invention encodes an antibody or antibody fragment comprising the V_{L} chain CDRs (SEQ ID NOS:352,354,356) of mGluR5 Ab22. In one embodiment, a polynucleotide of the invention encodes an antibody or antibody fragment comprising the V_{H} chain CDRs (SEQ ID NOS:344,346,348) and the V_{L} chain CDRs (SEQ ID NOS:352,354,356) of mGluR5 Ab22. In a further embodiment, a polynucleotide of the invention encodes an antibody or antibody fragment binding to the same epitope as mGluR5 Ab22. In one embodiment, a polynucleotide of the invention encodes an antibody or antibody fragment comprising the V_{H} chain (SEQ ID NO:342) and the V_{L} chain (SEQ ID NO:350) of mGluR5 Ab22. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:342. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:350. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:342 and a V_{L} chain having at least 80%, at least 85%, at least 90%, at least 95%, at least 98%, at least 99%, or 100% identity to the amino acid sequence of SEQ ID NO:350.

In one embodiment, a polynucleotide of the invention encodes mGluR5 Ab23. In one embodiment, a polynucleotide of the invention encodes an antibody or antibody fragment comprising the V_{H} chain CDRs (SEQ ID NOS:360,362,364) of mGluR5 Ab23. In one embodiment, a polynucleotide of the invention encodes an antibody or antibody fragment comprising the V_{L} chain CDRs (SEQ ID NOS:368,370,372) of mGluR5 Ab23. In one embodiment, a polynucleotide of the invention encodes an antibody or antibody fragment comprising the V_{H} chain CDRs (SEQ ID NOS:360,362,364) and the V_{L} chain CDRs (SEQ ID NOS:368,370,372) of mGluR5 Ab23. In a further embodiment, a polynucleotide of the invention encodes an antibody or antibody fragment binding to the same epitope as mGluR5 Ab23. In one embodiment, a polynucleotide of the invention encodes an antibody or antibody fragment comprising the V_{H} chain (SEQ ID NO:358) and the V_{L} chain (SEQ ID NO:366) of mGluR5 Ab23. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:358. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:366. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:358 and a V_{L} chain having at least 80%, at least 85%, at least 90%, at least 95%, at least 98%, at least 99%, or 100% identity to the amino acid sequence of SEQ ID NO:366.

In one embodiment, a polynucleotide of the invention encodes mGluR5 Ab24. In one embodiment, a polynucleotide of the invention encodes an antibody or antibody fragment comprising the V_{H} chain CDRs (SEQ ID NOS:376,378,380) of mGluR5 Ab24. In one embodiment, a polynucleotide of the invention encodes an antibody or antibody fragment comprising the V_{L} chain CDRs (SEQ ID NOS:384,386,388) of mGluR5 Ab24. In one embodiment, a polynucleotide of the invention encodes an antibody or antibody fragment comprising the V_{H} chain CDRs (SEQ ID NOS:376,378,380) and the V_{L} chain CDRs (SEQ ID NOS:384,386,388) of mGluR5 Ab24. In a further embodiment, a polynucleotide of the invention encodes an antibody or antibody fragment binding to the same epitope as mGluR5 Ab24. In one embodiment, a polynucleotide of the invention encodes an antibody or antibody fragment comprising the V_{H} chain (SEQ ID NO:374) and the V_{L} chain (SEQ ID NO:382) of mGluR5 Ab24. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:374. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:382. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:374 and a V_{L} chain having at least 80%, at least 85%, at least 90%, at least 95%, at least 98%, at least 99%, or 100% identity to the amino acid sequence of SEQ ID NO:382.

In one embodiment, a polynucleotide of the invention encodes mGluR5 Ab25. In one embodiment, a polynucleotide of the invention encodes an antibody or antibody fragment comprising the V_{H} chain CDRs (SEQ ID NOS:392,394,396) of mGluR5 Ab25. In one embodiment, a polynucleotide of the invention encodes an antibody or antibody fragment comprising the V_{L} chain CDRs (SEQ ID NOS:400,402,404) of mGluR5 Ab25. In one embodiment, a polynucleotide of the invention encodes an antibody or antibody fragment comprising the V_{H} chain CDRs (SEQ ID NOS:392,394,396) and the V_{L} chain CDRs (SEQ ID NOS:400,402,404) of mGluR5 Ab25. In a further embodiment, a polynucleotide of the invention encodes an antibody or antibody fragment binding to the same epitope as mGluR5 Ab25. In one embodiment, a polynucleotide of the invention encodes an antibody or antibody fragment comprising the V_{H} chain (SEQ ID NO:390) and the V_{L} chain (SEQ ID NO:398) of mGluR5 Ab25. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:390. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:398. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:390 and a V_{L} chain having at least 80%, at least 85%, at least 90%, at least 95%, at least 98%, at least 99%, or 100% identity to the amino acid sequence of SEQ ID NO:398.

In one embodiment, a polynucleotide of the invention encodes mGluR5 Ab26. In one embodiment, a polynucleotide of the invention encodes an antibody or antibody fragment comprising the V_{H} chain CDRs (SEQ ID NOS:408,410,412) of mGluR5 Ab26. In one embodiment, a polynucleotide of the invention encodes an antibody or antibody fragment comprising the V_{L} chain CDRs (SEQ ID NOS:416,418,420) of mGluR5 Ab26. In one embodiment, a polynucleotide of the invention encodes an antibody or antibody fragment comprising the V_{H} chain CDRs (SEQ ID NOS:408,410,412) and the V_{L} chain CDRs (SEQ ID NOS:416,418,420) of mGluR5 Ab26. In a further embodiment, a polynucleotide of the invention encodes an antibody or antibody fragment binding to the same epitope as mGluR5 Ab26. In one embodiment, a polynucleotide of the invention encodes an antibody or antibody fragment comprising the V_{H} chain (SEQ ID NO:406) and the V_{L} chain (SEQ ID NO:414) of mGluR5 Ab26. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:406. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:414. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:406 and a V_{L} chain having at least 80%, at least 85%, at least 90%, at least 95%, at least 98%, at least 99%, or 100% identity to the amino acid sequence of SEQ ID NO:414.

In one embodiment, a polynucleotide of the invention encodes mGluR5 Ab27. In one embodiment, a polynucleotide of the invention encodes an antibody or antibody fragment comprising the V_{H} chain CDRs (SEQ ID NOS:424,426,428) of mGluR5 Ab27. In one embodiment, a polynucleotide of the invention encodes an antibody or antibody fragment comprising the V_{L} chain CDRs (SEQ ID NOS:432,434,436) of mGluR5 Ab27. In one embodiment, a polynucleotide of the invention encodes an antibody or antibody fragment comprising the V_{H} chain CDRs (SEQ ID NOS:424,426,428) and the V_{L} chain CDRs (SEQ ID NOS:432,434,436) of mGluR5 Ab27. In a further embodiment, a polynucleotide of the invention encodes an antibody or antibody fragment binding to the same epitope as mGluR5 Ab27. In one embodiment, a polynucleotide of the invention encodes an antibody or antibody fragment comprising the V_{H} chain (SEQ ID NO:422) and the V_{L} chain (SEQ ID NO:430) of mGluR5 Ab27. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:422. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:430. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:422 and a V_{L} chain having at least 80%, at least 85%, at least 90%, at least 95%, at least 98%, at least 99%, or 100% identity to the amino acid sequence of SEQ ID NO:430.

In one embodiment, a polynucleotide of the invention encodes mGluR5 Ab28. In one embodiment, a polynucleotide of the invention encodes an antibody or antibody fragment comprising the V_{H} chain CDRs (SEQ ID NOS:440,442,444) of mGluR5 Ab28. In one embodiment, a polynucleotide of the invention encodes an antibody or antibody fragment comprising the V_{L} chain CDRs (SEQ ID NOS:448,450,452) of mGluR5 Ab28. In one embodiment, a polynucleotide of the invention encodes an antibody or antibody fragment comprising the V_{H} chain CDRs (SEQ ID NOS:440,442,444) and the V_{L} chain CDRs (SEQ ID NOS:448,450,452) of mGluR5 Ab28. In a further embodiment, a polynucleotide of the invention encodes an antibody or antibody fragment binding to the same epitope as mGluR5 Ab28. In one embodiment, a polynucleotide of the invention encodes an antibody or antibody fragment comprising the V_{H} chain (SEQ ID NO:438) and the V_{L} chain (SEQ ID NO:446) of mGluR5 Ab28. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:438. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:446. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:438 and a V_{L} chain having at least 80%, at least 85%, at least 90%, at least 95%, at least 98%, at least 99%, or 100% identity to the amino acid sequence of SEQ ID NO:446.

In one embodiment, a polynucleotide of the invention encodes mGluR5 Ab29. In one embodiment, a polynucleotide of the invention encodes an antibody or antibody fragment comprising the V_{H} chain CDRs (SEQ ID NOS:456,458,460) of mGluR5 Ab29. In one embodiment, a polynucleotide of the invention encodes an antibody or antibody fragment comprising the V_{L} chain CDRs (SEQ ID NOS:464,466,468) of mGluR5 Ab29. In one embodiment, a polynucleotide of the invention encodes an antibody or antibody fragment comprising the V_{H} chain CDRs (SEQ ID NOS:456,458,460) and the V_{L} chain CDRs (SEQ ID NOS:464,466,468) of mGluR5 Ab29. In a further embodiment, a polynucleotide of the invention encodes an antibody or antibody fragment binding to the same epitope as mGluR5 Ab29. In one embodiment, a polynucleotide of the invention encodes an antibody or antibody fragment comprising the V_{H} chain (SEQ ID NO:454) and the V_{L} chain (SEQ ID NO:462) of mGluR5 Ab29. In another embodiment, a polynucleotide of the invention encodes an antibody or antibody fragment comprising a V_{H} chain having at least 80%, at least 85%, at least 90%, at least 95%, at least 98%, at least 99%, or 100% identity to the amino acid sequence of SEQ ID NO:454. In one embodiment, a polynucleotide of the invention encodes an antibody or antibody fragment comprising a V_{L} chain having at least 80%, at least 85%, at least 90%, at least 95%, at least 98%, at least 99%, or 100% identity to the amino acid sequence of SEQ ID NO:462. In one embodiment, an antibody or antibody fragment of the invention comprises a V_{H} chain having at least 80%, at least 85%, at least 90%, at least 95%, at least 98%, at least 99%, or 100% identity to the amino acid sequence of SEQ ID NO:454 and a V_{L} chain having at least 80%, at least 85%, at least 90%, at least 95%, at least 98%, at least 99%, or 100% identity to the amino acid sequence of SEQ ID NO:462.

### Further Modifications

Antibody Conjugates

In some embodiments, the present invention features antibody-drug conjugates (ADCs), consisting of an antibody (or antibody fragment such as a single-chain variable fragment (scFv) linked to a payload drug (often cytotoxic). The antibody causes the ADC to bind to the target cells. Often the ADC is then internalized by the cell and the drug is released into the cell. Because of the targeting, the side effects are lower and give a wider therapeutic window. Hydrophilic linkers (e.g., PEG4Mal) help prevent the drug being pumped out of resistant cells through MDR (multiple drug resistance) transporters.

In another aspect, the present invention features immunoconjugates comprising an anti-mGluR5 antibody, or a fragment thereof, conjugated to a therapeutic agent, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include Taxol, cytochalasin B, gramicidin D, ethidium bromide, emetine, mitomycin, etoposide, teniposide, vincristine, vinblastine, colchicine, doxorubicin, daunorubicin, dihydroxy anthracin dione, mitoxantrone, mithramycin, actinomycin D, 1-dehydrotestosterone, glucocorticoids, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other examples of therapeutic cytotoxins that can be conjugated to an antibody according to at least some embodiments of the invention include duocarmycins, calicheamicin, maytansines and auristatins, and derivatives thereof. An example of a calicheamicin antibody conjugate is commercially available (Mylotarg™ Wyeth).

Cytotoxins can be conjugated to antibodies according to at least some embodiments of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55: 199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3: 1089-1091; Senter, P. D. and Springer, C. J. (2001) Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine 131, indium 111, yttrium 90 and lutetium 177. Methods for preparing radioimmunoconjugates are established in the art. Radioimmunoconjugates are commercially available, including Zevalin® (Spectrum), and similar methods can be used to prepare radioimmunoconjugates using the antibodies according to at least some embodiments of the invention.

The anti-human mGluR5 antibodies and conjugates containing according to at least some embodiments of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62: 119-58 (1982).

Modifications to the Constant Regions, Fc Domain, and Post-Translational Modifications

In addition or as an alternative to modifications made within the framework or CDR regions, antibodies according to at least some embodiments of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, and T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In some embodiments, one or more amino acids selected from amino acid residues 329, 331 and 322 can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al.

In another embodiment, one or more amino acid residues within amino acid positions 231 and 239 are altered to thereby alter the ability of the antibody to fix complement. This approach is described further in PCT Publication WO 94/29351 by Bodmer et al.

In yet another embodiment, the Fc region is modified to decrease or increase the affinity of the antibody for an Fγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A. Furthermore, mutations such as M252Y/S254T/f256E or M428L/N434S improve binding to FcRn and increase antibody circulation half-life (see Chan C A and Carter P J (2010) Nature Rev Immunol 10:301-316).

In still another embodiment, the antibody can be modified to abrogate in vivo Fab arm exchange. Specifically, this process involves the exchange of IgG4 half-molecules (one heavy chain plus one light chain) between other IgG4 antibodies that effectively results in bispecific antibodies which are functionally monovalent. Mutations to the hinge region and constant domains of the heavy chain can abrogate this exchange (see Aalberse, R C, Schuurman J., 2002, Immunology 105:9-19).

In still another embodiment, the glycosylation of an antibody is modified. For example, an aglycosylated antibody can be made (i.e., the antibody lacks glycosylation). Glycosylation can be altered to, for example, increase the affinity of the antibody for antigen, and/or to decrease ADCC and CDC activity. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglyclosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Altered glycosylation patterns have been demonstrated to increase or decrease the ADCC ability of antibodies. In a preferred embodiment of the invention, the antibody is modified to decrease effector function, by modifying the antibody to diminish or abolish the conserved Fc N-linked glycosylation at Asn297 (standard nomenclature). In other embodiments, carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (a (1,6) fucosyltransferase), such that antibodies expressed in the Ms704, Ms705, and Ms709 cell lines lack fucose on their carbohydrates. The Ms704, Ms705, and Ms709 FUT8 cell lines are created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the a 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) Biol. Chem. 277:26733-26740). PCT Publication WO 99/54342 by Umana et al. describes cell lines engineered to express glycoprotein-modifying glycosyl transferases (e.g., P(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180). Alternatively, the fucose residues of the antibody may be cleaved off using a fucosidase enzyme. For example, the fucosidase-L-fucosidase removes fucosyl residues from antibodies (Tarentino, A. L. et al. (1975) Biochem. 14:5516-23).

Another modification of the antibodies or fragments herein that is contemplated by the invention is pegylation or the addition of other water soluble moieties, typically polymers, e.g., in order to enhance half-life. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (Ci-Cio) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies according to at least some embodiments of the invention. See for example, EP 0 154 316 by Nishimura et al. and EP 0 401 384 by Ishikawa et al.

### Nucleic Acid Molecules

The invention further provides nucleic acids which encode an anti-mGluR5 antibody according to the invention, or a fragment or conjugate thereof. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See Ausubel, et al. (2011) Current Protocols in Molecular Biology, John Wiley & Sons, Inc. A nucleic acid according to at least some embodiments of the invention can be, for example, DNA or RNA and may or may not contain intronic sequences. In a preferred embodiment, the nucleic acid is a cDNA molecule.

Nucleic acids according to at least some embodiments of the invention can be obtained using standard molecular biology techniques. For antibodies expressed by hybridomas (e.g., hybridomas prepared from transgenic mice carrying human immunoglobulin genes as described further below) or B cells, cDNAs encoding the light and heavy chains of the antibody made by the cells can be obtained by standard PCR amplification or cDNA cloning techniques. For antibodies obtained from an immunoglobulin gene library (e.g., using phage display techniques), nucleic acid encoding the antibody can be recovered from the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to an scFv gene. In these manipulations, a V_{L}- or V_{H}-encoding DNA fragment is operatively linked to another DNA fragment encoding another protein, such as an antibody constant region or a flexible linker. As previously defined, “operatively linked” means that that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1, IgG2 or IgG4 constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, CL—The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa (κ) or lambda (λ) constant region, but most preferably is a u constant region.

To create an scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al. (1988) Science 242:423-426; Huston et al. (1988) Proc. Natl. Acad. Sci., USA 85:5879-5883; and McCafferty et al., (1990) Nature 348:552-554).

### Vectors

The present invention also provides vectors in which a DNA of the present invention is inserted. Vectors derived from retroviruses are suitable tools to achieve long-term gene transfer since they allow for genetic stability and high expression, in addition to having a flexible genome. Furthermore, clinical experience with retroviral vectors provides guidance for optimizing efficacy and safety in their use.

In brief summary, the expression of natural or synthetic nucleic acids encoding antibodies or antigen-binding fragments thereof is typically achieved by operably linking a nucleic acid encoding the antibody or antigen-binding fragment thereof, or portions thereof, to a promoter, and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, gammaretroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, retrovirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

Various promoter sequences may be used, including, but not limited to the immediate early cytomegalovirus (CMV) promoter, Elongation Growth Factor-1α (EF-1α), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but arc not limited to a metallothionine promoter, a glucocorticoid promoter, a progesterone promoter, and a tetracycline promoter.

In order to assess the expression of an antibody, antigen-binding fragment of an antibody, or a portion thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Transfection

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20 degrees Celsius. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Therapeutic Applications of mGluR5 Antibodies

Isolated anti-mGluR5 antibodies or antigen-binding fragments thereof obtained through the above methods, or compositions containing the same, can be used as a medicament in the treatment or prevention of a disease, disorder, or condition in a subject.

### Subject

The subject referred to herein may be any living subject. In a preferred embodiment, the subject is a mammal. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes)

In some embodiments, the subject, to whom the antibodies, antibody fragments, or compositions are administered is a primate, such as a human. In some embodiments, the primate is a monkey or an ape. The subject can be male or female and can be any suitable age, including infant, juvenile, adolescent, adult, and geriatric subjects. In some examples, the patient or subject is a validated animal model for disease, antibody therapy, and/or for assessing toxic outcomes.

In some embodiments, the methods include administration of anti-mGluR5 antibodies, antibody fragments, or compositions containing to a subject, tissue, or cell. The subject to be treated, or from whom the tissue or cell is derived, may be one having, at risk for, or suspected of having a disease, condition or disorder associated with the expression of mGluR5. In some embodiments, the antibodies, antibody fragments, or compositions are administered to a subject having the particular disease or condition to be treated. In some embodiments, antibodies, antibody fragments, or compositions are administered to the subject, such as a subject having or at risk for the disease or condition. In some aspects, the methods thereby treat, e.g., ameliorate one or more symptom of the disease or condition, such as by lessening the proportion of activated T cells or B cells mediating an autoimmune disorder.

### Modes of Administration

The compositions of the present invention may be administered in a number of ways depending upon whether local or systemic treatment is desired.

In general, administration may be topical or parenteral. In some preferred embodiments the mode of administration will be peripheral and the administered antibody will block the peripheral effects of mGlu5. In some other preferred embodiments the mode of administration will be central, e.g., intrathecal administration and will permit the antibody to block the effects of mGlu5 centrally. In some other preferred embodiments the mode of administration will provide for the antibody to block the effects of mGlu5 both centrally and peripherally, e.g., the antibodies may be administered intrathecally and intravenously.

The compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, intrasynovial injection or infusions; and kidney dialytic infusion techniques. In a preferred embodiment, parenteral administration of the compositions of the present invention comprises intravenous administration. In a preferred embodiment, parenteral administration of the compositions of the present invention comprises intrathecal administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

Preferably, the formulated composition comprising isolated anti-mGluR5 antibodies or antibody fragments is suitable for administration via injection.

Pharmaceutical compositions and formulations for topical administration may include transdermal patches, ointments, lotions, creams, gels, drops, suppositories, sprays, liquids, semi-solids, monophasic compositions, multiphasic compositions (e.g., oil-in-water, water-in-oil), foams, microsponges, liposomes, nanoemulsions, aerosol foams, polymers, fullerenes, and powders. Conventional pharmaceutical carriers, aqueous, powder or oily bases, thickeners and the like may be necessary or desirable.

Compositions and formulations for parenteral, intrathecal, or intraventricular administration may include sterile aqueous solutions that may also contain buffers, diluents and other suitable additives such as, but not limited to, penetration enhancers, carder antibodies and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical compositions of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, aerosols, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention, the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Formulations comprising anti-mGluR5 antibodies or antigen-binding fragments thereof may include pharmaceutically acceptable excipient(s). Excipients included in the formulations will have different purposes depending, for example, on the antibody and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents. The formulations comprising anti-mGluR5 antibodies will typically have been prepared and cultured in the absence of any non-human components, such as animal serum (e.g., bovine serum albumin).

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The antibodies may be combined with other therapeutics which may be administered in the same or different compositions, at the same or different time and in either order. For example, the inventive antibodies may be administered in a therapeutic regimen that includes the administration of another receptor agonist or antagonist, e.g., another mGluR5 agonist or antagonist.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

### Dosing

The pharmaceutical composition in some embodiments contains the anti-mGluR5 antibodies or antibody fragments in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The antibodies or antibody fragments can be administrated in one or more doses. In some embodiments, said effective amount of antibodies can be administrated as a single dose. In some embodiments, said effective amount of antibodies can be administrated as more than one dose over a period time. Timing of administration is within the judgment of managing physician and depends on the clinical condition of the patient. While individual needs vary, determination of optimal ranges of effective amounts of a given antibody for a particular disease or conditions is within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of antibodies or composition comprising those antibodies are administrated parenterally. In some embodiments, administration can be an intravenous administration. In some embodiments, administration can be directly done by injection within a disease site.

For purposes of the invention, the amount or dose of the inventive antibodies administered should be sufficient to effect a therapeutic or prophylactic response in the subject or animal over a reasonable time frame. For example, the dose of the inventive antibody should be sufficient to bind to antigen, or detect, treat or prevent disease in a period of from about 2 hours or longer, e.g., about 12 to about 24 or more hours, from the time of administration. In certain embodiments, the time period could be even longer. The dose will be determined by the efficacy of the particular antibody and the condition of the animal (e.g., human), as well as the body weight of the animal (e.g., human) to be treated.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the present invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the anti-mGluR5 antibody, or antigen-binding fragment thereof, disclosed herein, the dosage ranges from about 0.1 to 1000 mg and more usually 100 to 300 mg. For example dosages can be 0.1 mg, 0.5 mg, 1 mg, 2 mg, 5 mg, 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or within the range of 0.1-500 mg. An exemplary treatment regime entails administration twice per day, once per day, twice per week, once per week, once every two weeks, once every three weeks, once every four weeks, once a month, once every 3 months or once every three to 6 months.

For administration of the anti-mGluR5 antibody, or antigen-binding fragment thereof, disclosed herein, the dosage ranges from about 0.0001 to 100 mg/kg, and more usually 0.01 to 50 mg/kg, of the host body weight. For example dosages can be 0.3 mg/kg body weight, 1 mg/kg body weight, 3 mg/kg body weight, 5 mg/kg body weight, 10 mg/kg body weight, 15 mg/kg body weight, 20 mg/kg body weight, 25 mg/kg body weight, 30 mg/kg body weight, 35 mg/kg body weight, 40 mg/kg body weight, 45 mg/kg body weight, 50 mg/kg body weight, or within the range of 1-50 mg/kg. An exemplary treatment regime entails administration twice per day, once per day, twice per week, once per week, once every two weeks, once every three weeks, once every four weeks, once a month, once every 3 months or once every three to 6 months. Preferred dosage regimens for an antibody disclosed herein according to at least some embodiments of the present invention include 1-5 mg/kg body weight via intravenous administration.

In some methods, two or more monoclonal antibodies with different binding specificities are administered simultaneously in which case the dosage of each antibody disclosed herein administered falls within the ranges indicated. Antibody disclosed herein is usually administered on multiple occasions. Intervals between single dosages can be, for example, daily, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Alternatively, a therapeutic agent can be administered as a sustained release formulation, in which case less frequent administration is required. Dosage and frequency vary depending on the half-life of the therapeutic agent in the patient. In general, human antibodies show the longest half-life, followed by humanized antibodies, chimeric antibodies, and nonhuman antibodies. The half-life for fusion proteins may vary widely. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regimen.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, antibodies and/or materials used in combination with the particular compositions employed, the age, sex, weight, condition, general health and prior medical history of the patient being treated, and like factors well known in the medical arts.

In some embodiments, the antibodies are administered as part of a combination treatment, such as simultaneously with or sequentially with, in any order, another therapeutic intervention, such as another antibody or engineered cell or receptor or agent, such as a cytotoxic or therapeutic agent. The antibodies in some embodiments are co-administered with one or more additional therapeutic agents or in connection with another therapeutic intervention, either simultaneously or sequentially in any order. In some contexts, the antibodies are co-administered with another therapy sufficiently close in time such that the antibodies enhance the effect of one or more additional therapeutic agents, or vice versa. In some embodiments, the antibodies are administered prior to the one or more additional therapeutic agents. In some embodiments, the antibodies are administered after to the one or more additional therapeutic agents.

### Disease Indications

mGluR5 has been implicated in myriad diseases. Some indications have been shown to be related to mGluR5 in the periphery (migraine, GERD, IBS). Other indications are presumed to be due to the effect of mGluR5 in the CNS (Fragile X syndrome, anxiety, Parkinson's disease, addiction, etc.) and would therefore be considered as appropriate indications for treatment with an mGluR5 antibody administered intrathecally.

mGluR5 antibodies can therefore be used in the preparation of a medicament to treat numerous conditions, diseases, and disorders. In some embodiments, such a medicament can be used for treating migraine. In some embodiments, the medicament can be used for the treatment of GERD. In some embodiments, the medicament can be used for the treatment of IBS. In some embodiments, the medicament can be used for the treatment of overactive bladder (OAB)/incontinence. In some embodiments, the medicament can be used for the treatment or prevention of pain; e.g., acute pain, neuropathic pain/peripheral neuropathy, inflammatory pain, postoperative pain, chronic pain, or bladder visceral pain. In some embodiments, the medicament can be used for the treatment of Parkinson's disease and/or levodopa-induced dyskinesias in Parkinson's disease patients. In some embodiments, the medicament can be used for the treatment of addiction. In some embodiments, the medicament can be used for the treatment of dystonia.

In some embodiments, the invention relates to methods of using the compounds described herein to treat subjects diagnosed with or suffering from a disease, disorder, or condition mediated by mGluR5 activity, such as: bipolar disorder I depressed, hypomanic, manic and mixed form; bipolar disorder II; depressive disorders, such as single depressive episode or recurrent major depressive disorder, minor depressive disorder, treatment-resistant depression, depressive disorder with postpartum onset, disruptive mood dysregulation disorder, depressive disorders with psychotic symptoms; persistent mood disorders, such as cyclothymia, dysthymia, euthymia; and premenstrual dysphoric disorder; anxiety disorders, general anxiety disorder, panic disorder with or without agoraphobia, specific phobia, social anxiety disorder, chronic anxiety disorders; obsessive compulsive disorder; reaction to sever stress and adjustment disorders, such as post-traumatic stress disorder (PTSD); other neurotic disorders such as depersonalisation-derealisation syndrome; pervasive developmental disorders, including but not limited to Asperger's syndrome and Rett's syndrome, autistic disorders, childhood autism and overactive disorder associated with mental retardation and stereotyped movements, specific developmental disorder of motor function, specific developmental disorders of scholastic skills; postnatal (postpartum) and prenatal depression; eating disorders, including but not limited to anorexia nervosa, bulimia nervosa, pica and binge eating disorder; Parkinson's disease; second Parkinsonism, such as postencephalitic Parkinsonism; Parkinsonism comprised in other disorders; Lewy body disease; degenerative diseases of the basal ganglia; other extrapyramidal and movement disorders including but not limited to tremor, essential tremor and drug-induced tremor, myoclonus, chorea and drug-induced chorea, drug-induced tics and tics of organic origin, drug-induced acute dystonia, drug-induced tardive dyskinesia, L-dopa-induced dyskinesia; neuroleptic-induced movement disorders including but not limited to neuroleptic malignant syndrome (NMS), neuroleptic induced parkinsonism, neuroleptic-induced early onset or acute dyskinesia, neuroleptic-induced acute dystonia, neuroleptic-induced acute akathisia, neuroleptic-induced tardive dyskinesia, neuroleptic-induced tremor; restless leg syndrome, Stiff-man syndrome; dystonia including but not limited to focal dystonia, multiple-focal or segmental dystonia, torsion dystonia, hemispheric, generalized and tardive dystonia (induced by psychopharmacological drugs); focal dystonia include cervical dystonia (torticollis), blepharospasm (cramp of the eyelid), appendicular dystonia (cramp in the extremities, like the writer's cramp), oromandibular dystonia and spasmodic dysphonia (cramp of the vocal cord); epilepsy, including localization-related (focal)(partial) idiopathic epilepsy and epileptic syndromes with seizures of localized onset, localization-related (focal)(partial) symptomatic epilepsy and epileptic syndromes with simple partial seizures, localization-related (focal)(partial) symptomatic epilepsy and epileptic syndromes with complex partial seizures, generalized idiopathic epilepsy and epileptic syndromes including but not limited to myoclonic epilepsy in infancy, neonatal convulsions (familial), childhood absence epilepsy (pyknolepsy), epilepsy with grand mal seizures on awakening, absence epilepsy, myoclonic epilepsy (impulsive petit mal) and nonspecific atonic, clonic, myoclonic, tonic, tonic-clonic epileptic seizures; epilepsy with myoclonic absences, myoclonic-astatic seizures, infantile spasms, Lennox-Gastaut syndrome, Salaam attacks, symptomatic early myoclonic encephalopathy, West's syndrome, petit and grand mal seizures; status epilepticus; persistent somatoform disorders; acute, chronic and chronic intractable pain, headache; acute and chronic pain related to physiological processes and physical disorders including but not limited to back pain, tooth pain, abdominal pain, low back pain, pain in joints; acute and chronic pain that is related to diseases of the musculoskeletal system and connective tissue including, but not limited to rheumatism, myalgia, neuralgia and fibromyalgia; acute and chronic pain that is related to nerve, nerve root and plexus disorders, such as trigeminal pain, postzoster neuralgia, phantom limb syndrome with pain, carpal tunnel syndrome, lesion of sciatic nerve, diabetic mononeuropathy; acute and chronic pain that is related to polyneuropathies and other disorders of the peripheral nervous system, such as hereditary and idiopathic neuropathy, inflammatory polyneuropathy, polyneuropathy induced by drugs, alcohol or toxic agents, polyneuropathy in neoplastic disease, diabetic polyneuropathy; and acute neurodegeneration, such as intracranial brain injuries, such as stroke, diffuse and local brain injuries, epidural, subdural and subarachnoid hemorrhage, and chronic neurodegeneration, such as Alzheimer's disease, Huntington's disease, multiple sclerosis, and ALS; subarachnoid hemorrhage, intracerebral hemorrhage and other nontraumatic intracranial hemorrhage, cerebral infarction, stroke, occlusion and stenosis or precerebral and cerebral arteries, not resulting in cerebral infarction, dissection of cerebral arteries, cerebral aneurysm, cerebral atherosclerosis, progressive vascular leukoencephalopathy, hypertensive encephalopathy, nonpyogenic thrombosis of intracranial venous system, cerebral arteritis, cerebral amyloid angiopathy and sequelae of cerebrovascular diseases; glaucoma and other neuropathies; dementias, vascular dementia, Lewy body dementia, frontotemporal dementia, and HIV-dementia; vertigo and nystagmus; tinnitus; neuropsychiatric systemic lupus erythematosus; disruptive mood dysregulation disorder; schizophrenia spectrum disorder; and sleep/wake disorders. In specific embodiments, subjects that can be treated according to the present invention are diagnosed with or suffering from major depressive disorder, treatment-resistant depression and bipolar disorder.

Migraine

Migraine is a complex, common neurological condition that is characterized by severe, episodic attacks of headache and associated features, which may include nausea, vomiting, sensitivity to light, sound or movement. In some patients, the headache is preceded or accompanied by sensory warning signs or symptoms (i.e. auras). The headache pain may be severe and may also be unilateral in certain patients. Migraine attacks are disruptive to daily life and cost billions of dollars each year in missed work days and impaired performance (Modi and Lowder, Am. Fam. Physician, Vol. 73:72-78, 2006).

Migraine is a highly prevalent disease worldwide with approximately 15% of the European population and 12% of the United States population suffering from migraine attacks (Lipton et al, Neurology, Vol. 68:343-349, 2007). Additionally, migraines have been found to be associated with a number of psychiatric and medical comorbidities such as depression and vascular disorders (Buse et al., Neurol. Neurosurg. Psychiatry, Vol. 81:428-432, 2010; and Bigal et al, Neurology, Vol. 72: 1864-1871, 2009).

Migraine headache is commonly treated acutely, primarily with analgesics and a class of drugs called triptans (Humphrey et al. Ann NY Acad Sci., Vol. 600:587-598, 1990; and Houston and Vanhoutte, Drugs, Vol. 31:149-163 1986). The triptans, which are selective serotonin 5-HT1B/1D agonists, are effective drugs for acute migraine and are generally well tolerated, but are contraindicated in the presence of cardiovascular disease due to their potential for coronary vasoconstriction. In addition, many migraine patients do not respond favorably to triptans. In a meta-analysis of 53 trials, up to a third of all people with migraine and 40% of all migraine attacks did not respond to triptans (Ferrari et al., Lancet, Vol. 358: 1668-1675, 2001).

Migraine prophylaxis is an area of large unmet medical need. Approximately 40% of the migraine patient population would benefit from preventive therapy (Lipton et al., Neurology, Vol. 68:343-349, 2007). However, only approximately 12% of patients receive any preventive therapy due in part to limited efficacy and significant tolerability and safety issues with available preventive therapies. Topiramate, an anticonvulsant that blocks voltage-dependent sodium channels and certain glutamate receptors (AMPA-kainate), is the medication most often used for migraine prophylaxis in the United States. Topiramate is the only migraine prophylactic agent with demonstrated efficacy in both episodic and chronic migraine patients through randomized placebo-controlled trials (Diener et al., Cephalalgia, Vol. 27:814-823, 2007; Silberstein et al., Headache, Vol. 47: 170-180, 2007). However, approximately 50% of patients fail to respond to topiramate and it is poorly tolerated. Common adverse events associated with topiramate treatment include paresthesia, anorexia, and cognitive adverse events, including psychomotor slowing, somnolence, language difficulties, and difficulties with memory and concentration (Brandes et al, JAMA, Vol. 291:965-973, 2004; Adelman et al, Pain Med., Vol. 9: 175-185 2008; Silberstein et al., Arch Neurol, Vol. 61:490-495, 2004). In an open-label, flexible-dose study, 20% of patients withdrew from topiramate because of adverse effects (Nelles et al., Headache, Vol. 49: 1454-1465, 2009). In addition to existing migraine therapies, four pharmaceutical companies are now developing monoclonal antibodies to calcitonin gene-related peptide (CGRP) or its receptor. Four anti-CGRP or CGRP receptor monoclonal antibodies are currently in development: galcanezumab (LY2951742), eptinezumab (ALD403), fremanezumab (TEV-48215), and erenumab (AMG-334). Results from phase II trials have been published for all four compounds, and phase III trials are underway or completed. No safety issues have arisen to date, including hepatic or cardiovascular effects, and the antibodies appear to be well tolerated. See Tso A R and Goadsby P J. Curr Treat Options Neurol. 2017; 19(8):27. On May 17, 2018, erenumab (commercially known as “Aimovig”) received FDA approval as a preventative treatment for migraine in adults.

Many currently available therapies for treating migraine headache in human patients have a poor risk-benefit profile due to adverse side effects, which many patients are unable or refuse to tolerate. The present invention addresses this problem, in part, by providing a novel regimen of anti-mGluR5 antibodies that provides effective migraine prophylaxis with no or minimal side effects. The methods of the invention described herein can effectively reduce the frequency, severity, and/or duration of migraine headache in patients suffering from episodic migraine as well as chronic migraine.

Migraine headaches are recurrent headaches lasting about 4 to about 72 hours that are characterized by unilateral, pulsating, and/or moderate to severe pain and/or pain that is exacerbated by physical activity. Migraine headaches are often accompanied by nausea, vomiting, and/or sensitivity to light (photophobia), sound (phonophobia), or smell. In some patients, an aura precedes the onset of the migraine headache. The aura is typically a visual, sensory, language, or motor disturbance that signals the headache will soon occur. The methods described herein prevent, treat, or ameliorate one or more symptoms of migraine headaches with and without aura in human patients.

In one embodiment, the present invention provides a method for preventing or reducing the occurrence of migraine headache in a patient in need thereof comprising administering to the patient a pharmaceutical composition comprising a therapeutically effective amount of an anti-mGluR5 antibody or antigen-binding antibody fragment.

As used herein, “preventing or reducing the occurrence of migraine headache” refers to a reduction in the frequency, duration, or severity of the migraine headache as compared to the frequency, duration, or severity of the migraine headache prior to administration of the composition or as compared to the frequency, duration, or severity of the migraine headache in a patient not administered the composition (i.e. a control subject). Thus, in certain embodiments, the present invention provides a method for prophylactically treating a patient for migraine headache comprising administering to the patient a pharmaceutical composition comprising a therapeutically effective amount of an anti-mGluR5 antibody or antigen-binding fragment thereof. “Prophylactic treatment” refers to treatment designed to be taken before a migraine attack to reduce the frequency, severity, and/or length of migraine headaches in the patient. In some embodiments, a prophylactic treatment may increase the effectiveness of or a patient's response to acute migraine-specific medications.

In some embodiments of the methods of the invention, administration of the anti-mGluR5 antibody or binding fragment thereof reduces the number of migraine headache days experienced by the patient over the course of a month compared to the number prior to administration of the anti-mGluR5 antibody or binding fragment (i.e. pre-treatment baseline) and/or compared to the number experienced by a patient not receiving the anti-mGluR5 antibody or binding fragment. A “migraine headache day” includes any calendar day during which a patient experiences the onset, continuation, or recurrence of a “migraine headache” with or without aura lasting greater than 30 minutes. A “migraine headache” is a headache associated with nausea or vomiting or sensitivity to light or sound and/or a headache characterized by at least two of the following pain features: unilateral pain, throbbing pain, moderate to severe pain intensity, or pain exacerbated by physical activity. The pre-treatment baseline can be established by determining the relevant parameter (e.g. number of migraine headache days) in one, two, three, four, five, or six or more months prior to administration of the anti-mGluR5 antibody or binding fragment. In some embodiments, the pre-treatment baseline is established based on the measurement of the particular parameter in the three months prior to administration of the anti-mGluR5 antibody or binding fragment.

In certain embodiments, the number of monthly migraine headache days experienced by the patient is reduced by about 10%, about 15%, about 20%, about 25%, about 30%, about 35%, about 40%), about 45%, about 50%, about 55%, or about 60% following administration of the anti-mGluR5 antibody or binding fragment as compared to a pre-treatment baseline and/or a control subject (i.e. a patient not receiving the antibody or binding fragment). In some embodiments, the number of monthly migraine headache days experienced by the patient is reduced by 65% or more, for example, by at least about 70%, at least about 75%, or at least about 80% following administration of the anti-mGluR5 antibody or binding fragment as compared to a pre-treatment baseline and/or a control subject. In one embodiment, the number of monthly migraine headache days experienced by the patient is reduced by at least 50% following administration of the anti-mGluR5 antibody or binding fragment. In another embodiment, the number of monthly migraine headache days experienced by the patient is reduced by at least 75% following administration of the anti-mGluR5 antibody or binding fragment.

A reduction in the occurrence of migraine headache can also be assessed as a reduction in the number of migraine headache hours experienced by the patient over the course of a month compared to a pre-treatment baseline and/or the number experienced by a patient not receiving the anti-mGluR5 antibody or binding fragment. A “migraine headache hour” is any hour during which a patient experiences the onset, continuation, or recurrence of a “migraine headache” with or without aura. In certain embodiments, administration of the anti-mGluR5 antibody or binding fragment thereof reduces the number of monthly migraine headache hours experienced by the patient by at least about 20%, at least about 30%, at least about 40%, at least about 50%, at least about 60%, or at least about 70% as compared to a pre-treatment baseline and/or the number in a control subject not receiving the anti-mGluR5 antibody or binding fragment.

Efficacy of the therapeutic regimens described herein can also be assessed in terms of the number of days a patient requires acute treatment with migraine-specific medication, the number of days the patient is physically or functionally impaired due to migraine, or the number of migraine attacks experienced by the patient. For instance, in some embodiments, the administration of the anti-mGluR5 antibody or binding fragment thereof reduces the number of days a patient requires the use of acute migraine treatments over the course of a month compared to a pre-treatment baseline and/or the number experienced by a patient not receiving the anti-mGluR5 antibody or binding fragment. As used herein, the term “acute migraine-specific medication treatment day” or “acute migraine-specific medication use day” refers to any calendar day during which the patient took a medication that is specific for migraine. Acute migraine-specific medications include, but are not limited to, triptans (e.g., almotriptan, frovatriptan, rizatriptan, sumatriptan, naratriptan, eletriptan, and zolmitriptan), ergotamines (e.g., dihydroergotamine and ergotamine with caffeine), non-steroidal antiinflammatory drugs (e.g., acetylsalicylic acid, ibuprofen, naproxen, indomethacin, and diclofenac), and opioids (e.g., codeine, morphine, hydrocodone, fentanyl, meperidine, and oxycodone). The number of monthly acute migraine-specific medication treatment days can be reduced by at least about 25%, at least about 30%, at least about 40%, at least about 50%, at least about 60%), at least about 70%, at least about 75%, at least about 80%, or at least about 85% following administration of the anti-mGluR5 antibody or binding fragment thereof. In certain embodiments, administration of the anti-mGluR5 antibody or binding fragment thereof completely eliminates the need for the use of acute migraine-specific medications.

In some embodiments, administration of an anti-mGluR5 antibody or binding fragment thereof according to the methods described herein can reduce the physical impairment or quality-of-life impact scores reported by patients as compared to a pre-treatment baseline and/or a patient not receiving the anti-mGluR5 antibody. Migraine headaches often impact the quality of life of patients and prevent them from engaging in leisure and everyday activities as well as cause a loss of productivity in a patient's job. These effects can be assessed using validated questionnaires and surveys, such as the modified Migraine Disability Assessment Questionnaire (MIDAS), the Headache Impact Test-6 (HIT-6), the Migraine-Specific Quality of Life Questionnaire (MSQ), the Migraine Functional Impact Questionnaire (MFIQ), and the Migraine Physical Function Impact Diary (MPFID). Thus, the methods of the invention improve one or more aspects of a patient's quality of life and/or reduce the impact of migraines on one or more aspects of a patient's physical, social, or emotional function as assessed by one or more of these questionnaires.

In certain embodiments of the methods of the invention, the number of migraine attacks experienced by the patient is reduced following administration of an anti-mGluR5 antibody or binding fragment thereof as compared to the number of migraine attacks experienced by the patient prior to treatment or the number of migraine attacks experienced by a control subject. As used herein, the term “migraine attack” refers to an episode of any migraine headache as defined herein. A migraine attack that is interrupted by sleep or temporarily remits and then recurs within 48 hours is generally considered to be a single attack. Similarly, a migraine attack that is successfully treated with acute migraine-specific medication but relapses within 48 hours is also considered to be a single attack. In some embodiments, the number of migraine attacks is reduced in the patient by at least about 25%, at least about 30%, at least about 40%), at least about 50%>, at least about 60%>, at least about 70%>, or at least about 75% following administration of an anti-mGluR5 antibody or binding fragment thereof as compared to the number of attacks prior to treatment or the number of attacks in a control subject.

In some embodiments, the therapeutic regimens of the invention ameliorate one or more symptoms associated with migraine, e.g., inhibit the severity and/or the incidence of such symptoms in a patient in need thereof. For instance, administration of an anti-mGluR5 antibody or binding fragment thereof to the patient according to the methods described herein may reduce the occurrence of or reduce the severity of one or more symptoms in the patients as compared to a control subject (i.e. a subject not receiving the anti-mGluR5 or binding fragment). In some instances the subject antibodies may block or prevent such symptoms for prolonged duration, e.g., weeks or months after antibody administration. Symptoms that can be ameliorated or treated with the methods of the invention include, but are not limited to, vasomotor symptoms (e.g. hot flashes, facial flushing, sweating, and night sweats), photophobia (sensitivity to light), phonophobia (sensitivity to sound), sensitivity to smells, tearing/lacrimation, vertigo, dizziness, nausea, vomiting, aura, and headache pain.

Administration of an anti-mGluR5 antibody or binding fragment thereof according to the methods of the invention preferably causes few or no adverse side effects in the patient. As used herein, the term “adverse side effect” refers to any abnormality, defect, mutation, lesion, degeneration, harmful or undesirable reaction, symptom, or injury, which may be caused by taking the drug. In some embodiments, administration of the anti-mGluR5 antibody or binding fragment thereof does not substantially cause one or more adverse side effects associated with other migraine prophylactic treatments (e.g. amitriptyline, divalproex, valproic acid, propranolol, timolol, topiramate, and botulinum toxin A). Side effects associated with other migraine prophylactic treatments include, but are not limited to, fatigue, nausea, dizziness, insomnia, depression, reduced exercise tolerance, tremor, paresthesia, teratogenicity, and cognitive difficulty. In other embodiments, administration of the anti-mGluR5 antibody or binding fragment thereof is associated with a lower rate or number of adverse side effects as compared to the rate or number of adverse side effects associated with other migraine prophylactic treatments. In yet other embodiments, administration of the anti-mGluR5 antibody or binding fragment thereof is associated with a lower rate of discontinuation due to adverse side effects as compared to the rate of discontinuation due to adverse side effects associated with other migraine prophylactic treatments. In certain embodiments, the number and type of adverse side effects associated with administration of the anti-mGluR5 antibody or binding fragment is not statistically different than the number and type of adverse side effects associated with administration of placebo. In some embodiments, administration of an anti-mGluR5 antibody or binding fragment thereof is not associated with an adverse event higher than grade 2 as assessed by the Common Terminology Criteria for Adverse Events v4.0 (CTCAE). In other embodiments, administration of an anti-mGluR5 antibody or binding fragment thereof is not associated with an adverse event higher than grade 1 as assessed by the CTCAE.

In certain embodiments, the patients to be treated according to the methods of the invention have, suffer from, or are diagnosed with episodic migraine. Episodic migraine is diagnosed when patients with a history of migraine (e.g. at least five lifetime attacks of migraine headache) have 14 or fewer migraine headache days as defined herein per month. In some embodiments, patients having, suffering from, or diagnosed with episodic migraine have at least four, but less than 15 migraine headache days per month on average. In related embodiments, patients having, suffering from, or diagnosed with episodic migraine have fewer than 15 headache days per month on average. As used herein, a “headache day” is any calendar day in which the patient experiences a migraine headache as defined herein or any headache that lasts greater than 30 minutes or requires acute headache treatment. In some embodiments, the patient may be classified as having or suffering from high-frequency episodic migraine. High-frequency episodic migraine can be characterized by 8 to 14 migraine headache days per month. In other embodiments, the patient may be classified as having or suffering from low-frequency episodic migraine. Low-frequency episodic migraine can be characterized by fewer than 8 migraine headache days per month.

In some embodiments, the patients to be treated according to the methods of the invention have, suffer from, or are diagnosed with chronic migraine. Chronic migraine is diagnosed when migraine patients (i.e. patients with at least five lifetime attacks of migraine headache) have 15 or more headache days per month and at least 8 of the headache days are migraine headache days. In some embodiments, patients having, suffering from, or diagnosed with chronic migraine have 15 or more migraine headache days per month on average. In certain embodiments of the methods described herein, administration of an anti-mGluR5 antibody or binding fragment thereof prevents, reduces, or delays the progression of episodic migraine in the patient to chronic migraine.

In some embodiments, the mGluR5 antibodies of the invention may be used in the treatment or prevention of cluster headache. Cluster headache is a neurological disorder characterized by recurrent, severe headaches on one side of the head, typically around the eye. In some embodiments, cluster headaches are characterized by accompanying eye watering, nasal congestion, or swelling around the eye, on the affected side. These symptoms may last 15 minutes to 3 hours. Attacks often occur in clusters which typically last for weeks or months and occasionally more than a year. In some embodiments, the therapeutic regimens of the invention ameliorate one or more symptoms associated with cluster headache in a patient in need thereof. For instance, administration of an anti-mGluR5 antibody or binding fragment thereof to the patient according to the methods described herein may reduce the occurrence of or treat one or more symptoms in the patients as compared to a control subject (i.e. a subject not receiving the anti-mGluR5 or binding fragment). Symptoms that can be ameliorated or treated with the methods of the invention include, e.g., tearing/lacrimation, headache pain, swelling, etc. In certain aspects of the invention, the inventive mGluR5 antibodies may be used to reduce or prevent the incidence of cluster headaches.

In certain embodiments of the methods described herein, the patient is treatment-naïve. In one embodiment, a patient is treatment-naïve if the patient has not previously received treatment for migraine headaches. In another embodiment, the patient is treatment-naïve if the patient was not administered a therapeutic agent for the treatment of migraine headaches. In some embodiments, a patient is treatment-naïve if the patient has not previously received prophylactic therapy for migraine headaches. For instance, in certain embodiments, a treatment-naïve patient has not received prior therapy or has not been administered a therapeutic agent for the prophylactic treatment of episodic migraine. In certain other embodiments, a treatment-naïve patient has not received prior therapy or has not been administered a therapeutic agent for the prophylactic treatment of chronic migraine.

In some embodiments of the methods described herein, the patient has failed or is intolerant to at least one other migraine headache prophylactic therapy. For example, in one particular embodiment, the patient has failed to respond to prior therapy with at least one migraine headache prophylactic agent. As used herein, “failure to respond” or “treatment failure” refers to the lack of efficacy of the prophylactic agent in reducing the frequency, duration, and/or severity of migraine headache in the patient following a standard therapeutic regimen of the agent. For instance, in one embodiment, a patient who has failed prior treatment with a migraine prophylactic agent is a patient who experienced the same or a greater number of monthly migraine headache days following administration of the migraine prophylactic agent as compared to the number of monthly migraine headache days prior to treatment with the agent.

In another embodiment, a patient who has failed prior treatment with a migraine prophylactic agent is a patient who experienced the same or a greater number of monthly acute migraine-specific medication treatment days following administration of the migraine prophylactic agent as compared to the number of monthly acute migraine-specific medication treatment days prior to treatment with the agent. In yet another embodiment, a patient who has failed prior treatment with a migraine prophylactic agent is a patient who experienced the same or a greater number of migraine attacks following administration of the migraine prophylactic agent as compared to the number of migraine attacks prior to treatment with the agent. In still another embodiment, a patient who has failed prior treatment with a migraine prophylactic agent is a patient who experienced the same level or a greater level of physical impairment (e.g. mean monthly days with physical impairment) as measured by the MPFID following administration of the migraine prophylactic agent as compared to the level of physical impairment prior to treatment with the agent.

Failure to respond to prior treatment with a migraine prophylactic agent can also include inability to tolerate the migraine prophylactic agent. For example, in some embodiments, a patient who has failed prior treatment with a migraine prophylactic agent is a patient who cannot tolerate the side effects associated with the agent. In such embodiments, the side effects associated with the agent may exacerbate or may be incompatible with another medical condition which the patient has. By way of illustration, migraine prophylactic agents having a side effect of teratogenicity would be contraindicated in a pregnant patient. In certain embodiments, a patient who has failed prior treatment with a migraine prophylactic agent is a patient who discontinues treatment with the migraine prophylactic agent due to associated side effects. In these and other embodiments, a patient who has failed prior treatment with a migraine prophylactic agent is a patient who elects to stop treatment, alter the treatment regimen, or switch to a different prophylactic agent because the impact of the side effects is greater than the therapeutic benefit of the migraine prophylactic agent.

Migraine prophylactic agents include, but are not limited to, beta-blockers (e.g., propranolol, timolol, atenolol, metoprolol, and nadolol), antiepileptics (e.g. divalproex, sodium valproate, valproic acid, topiramate, and gabapentin), tricyclic antidepressants (e.g., amitriptyline, nortriptyline, doxepin, and fluoxetine), and botulinum toxin type A. Thus, in certain embodiments, the patients treated according to the methods of the invention have failed or are intolerant to one or more of these migraine prophylactic agents. In some embodiments, the patient has failed or is intolerant to treatment with at least two migraine prophylactic agents. In other embodiments, the patient has failed or is intolerant to treatment with at least three migraine prophylactic agents. In certain embodiments, the patient has failed or is intolerant to treatment with one or more agents selected from propranolol, timolol, divalproex, valproic acid, topiramate, amitriptyline, or botulinum toxin type A. In one particular embodiment, the patient has failed or is intolerant to treatment with topiramate. In another particular embodiment, the patient has failed or is intolerant to treatment with propranolol. In yet another particular embodiment, the patient has failed or is intolerant to treatment with amitriptyline.

In some embodiments, the patient has failed or is intolerant to treatment with two different classes of migraine prophylactic agents. For instance, in one embodiment, the patient may have failed or is intolerant to treatment with an antiepileptic (e.g. topiramate) and a beta-blocker (e.g. propranolol). In another embodiment, the patient may have failed or is intolerant to treatment with an antiepileptic (e.g. topiramate) and an antidepressant (e.g. amitriptyline). In still another embodiment, the patient may have failed or is intolerant to treatment with a beta-blocker (e.g. propranolol) and an antidepressant (e.g. amitriptyline). In certain embodiments, the patient has failed or is intolerant to treatment with three different classes of migraine prophylactic agents. In such embodiments, the patient has failed or is intolerant to treatment with an antiepileptic (e.g. topiramate), a beta-blocker (e.g. propranolol), and an antidepressant (e.g. amitriptyline).

The methods described herein are also applicable to other types of headache disorders such as tension-type headaches, cluster headaches, hemiplegic migraine, and retinal migraine. Accordingly, the present invention also provides methods for treating, including prophylactically treating, or preventing any of the aforementioned headache disorders by administering an anti-mGluR5 antibody or binding fragment thereof to a patient in need thereof with any of the dosage regimens described herein.

GERD

The lower esophageal sphincter (LES) is prone to relaxing intermittently. As a consequence, fluid from the stomach can pass into the esophagus since the mechanical barrier is temporarily lost at such times, an event herein referred to as “reflux”.

Gastro-esophageal reflux disease (GERD) is the most prevalent upper gastrointestinal tract disease. GERD, also known as acid reflux, is a long-term condition where stomach contents come back up into the esophagus resulting in either symptoms or complications. Symptoms include the taste of acid in the back of the mouth, heartburn, bad breath, chest pain, vomiting, breathing problems, and wearing away of the teeth. Complications include esophagitis, esophageal strictures, Barrett's esophagus, and esophageal cancer. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

In one aspect, the antibodies of the invention may be used to treat gastro-esophageal reflux disease (GERD). In one aspect, the antibodies of the invention may be used to treat regurgitation. In one aspect, the antibodies of the invention may be used to inhibit transient lower esophageal sphincter relaxations (TLESRs), thereby treating reflux.

The effects of mGluR5 antagonists in GERD are thought to be peripheral rather than central. Young R L et al. (American Journal of Physiology-Gastrointestinal and Liver Physiology. 2007; 292(2):G501-11.) shows that mGluR5 plays a prominent role at gastroesophageal vagal afferent endings but a minor role in central gastric vagal pathways. Peripheral mGluR5 is therefore a suitable target for reducing mechanosensory input from the periphery, for therapeutic benefit.

Therefore, in some embodiments, the antibody or antigen-binding antibody fragment of the invention is used to treat GERD. In some embodiments, the antibody treats GERD by targeting mGluR5 in the periphery, rather than the CNS. In some embodiments, treatment efficacy is monitored by a reduction of one or more symptoms or complications of GERD. Heartburn is a common symptom that is indicative of GERD. Other symptoms associated with GERD include, by way of non-limiting example, odynophagia, bitter taste in the mouth, belching, nausea, dysphagia, regurgitation, laryngitis, cough, hoarseness and asthma. Accordingly, certain embodiments of the present invention provide for a method of treating or alleviating the symptoms of, or inflammation associated with, gastroesophageal reflux disease (GERD). Specifically, some embodiments of the present invention provide for a method of treating or alleviating the symptoms of, or inflammation associated with, gastroesophageal reflux disease (GERD) in an individual by administering to an individual a therapeutically effective amount of an anti-mGluR5 antibody or antigen-binding antibody fragment. In some embodiments, the invention provides a method of normalizing the function of the lower esophageal sphincter. In specific embodiments, the gastroesophageal reflux disease treated is nonerosive reflux disease (NERD). In other specific embodiments, the gastroesophageal reflux disease is erosive esophagitis (EE). In some embodiments, the invention provides a method of treating one or more of grades A, B, C, or D of EE. In some embodiments, the invention provides a method of treating one or both of grades C and/or D of EE. In some embodiments, the invention provides a method of treating Barrett's Esophagus.

IBS

IBS is herein defined as a chronic functional disorder with specific symptoms that include continuous or recurrent abdominal pain and discomfort accompanied by altered bowel function, often with abdominal bloating and abdominal distension. It is generally divided into 3 subgroups according to the predominant bowel pattern: 1) diarrhea predominant; 2) constipation predominant; 3) alternating bowel movements. Abdominal pain or discomfort is the hallmark of IBS and is present in the three subgroups. IBS symptoms have been categorized according to the Rome criteria and subsequently modified to the Rome II criteria. This conformity in describing the symptoms of IBS has helped to achieve consensus in designing and evaluating IDS clinical studies.

The Rome II diagnostic criteria are:

1) Presence of abdominal pain or discomfort for at least 12 weeks (not necessarily consecutively) out of the preceding year;

2) Two or more of the following symptoms: a) Relief with defecation; b) Onset associated with change in stool frequency; c) Onset associated with change in stool consistency

Irritable bowel syndrome (IBS) can also be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders: Diagnosis, Pathophysiology and Treatment. 2 ed. McLean, V A: Degnon Associates, Inc.; 2000:351-432 and Drossman D A, Corazziari E, Talley N J, Thompson W G and Whitehead W E. Rome II: A multinational consensus document on Functional Gastrointestinal Disorders. Gut 45(Suppl. 2), II1-II81.9-1-1999.

In some embodiments, antibodies of the invention may be used to treat IBS. In some embodiments, antibodies of the invention may be used to treat IBS of any of the three subgroups: 1) diarrhea predominant; 2) constipation predominant; 3) alternating bowel movements.

OAB/Incontinence

Overactive bladder (OAB) is a condition characterized by a frequent feeling of needing to urinate to a degree that negatively affects a person's life. The frequent need to urinate may occur during the day, at night, or both. Overactive bladder is characterized by a group of four symptoms: urgency, urinary frequency, nocturia, and urge incontinence. Existing medications for OAB, typically of the anti-muscarinic type, are associated with negative side effects, particularly in the elderly.

Urinary incontinence (also known as involuntary urination, or simply incontinence) is defined as a loss of bladder control. More than 40% of people with overactive bladder have incontinence. About 40% to 70% of urinary incontinence is due to overactive bladder. A number of medications exist to treat incontinence including: fesoterodine, tolterodine and oxybutynin. While a number appear to have a small benefit, the risk of side effects are a concern. Urinary incontinence can be of several different varieties: stress incontinence, urge incontinence, overflow incontinence, mixed incontinence, structural incontinence, functional incontinence, nocturnal incontinence, transient incontinence, giggle incontinence, double incontinence, post-void dribbling, and coital incontinence.

In some embodiments, antibodies of the invention may also be used to treat overactive bladder (OAB). In some embodiments, the inventive methods of treatment will prevent, reduce, or otherwise ameliorate one or more symptoms associated with OAB. In one aspect, treatment will help to alleviate one or more of urgency, urinary frequency, nocturia, and urge incontinence. In some embodiments, antibodies of the invention may be used to treat incontinence. The incontinence may be selected from any one or more of stress incontinence, urge incontinence, overflow incontinence, mixed incontinence, structural incontinence, functional incontinence, nocturnal incontinence, transient incontinence, giggle incontinence, double incontinence, post-void dribbling, and coital incontinence.

Pain

Antibodies of the invention may also be used to treat or prevent pain. Examples of pain include, but are not limited to pain disorders related to psychological factors, such as persistent somatoform disorders; acute, chronic and chronic intractable pain, headache; acute and chronic pain related to physiological processes and physical disorders including but not limited to back pain, tooth pain, abdominal pain, low back pain, pain in joints; acute and chronic pain that is related to diseases of the musculoskeletal system and connective tissue including, but not limited to rheumatism, myalgia, neuralgia and fibromyalgia; acute and chronic pain that is related to nerve, nerve root and plexus disorders, such as trigeminal pain, postzoster neuralgia, phantom limb syndrome with pain, carpal tunnel syndrome, lesion of sciatic nerve, diabetic mononeuropathy; acute and chronic pain that is related to polyneuropathies and other disorders of the peripheral nervous system, such as hereditary and idiopathic neuropathy, inflammatory polyneuropathy, polyneuropathy induced by drugs, alcohol or toxic agents, polyneuropathy in neoplastic disease, diabetic polyneuropathy. Pain disorders for which the antibodies of the invention may be useful include neuropathic pain (such as postherpetic neuralgia, nerve injury, the “dynias”, e.g., vulvodynia, phantom limb pain, root avulsions, painful diabetic neuropathy, painful traumatic mononeuropathy, painful polyneuropathy); central pain syndromes (potentially caused by virtually any lesion at any level of the nervous system); postsurgical pain syndromes (e.g, postmastectomy syndrome, postthoracotomy syndrome, stump pain); bone and joint pain (osteoarthritis), repetitive motion pain, dental pain, cancer pain, myofascial pain (muscular injury, fibromyalgia); perioperative pain (general surgery, gynecological), chronic pain, dysmenorrhea, as well as pain associated with angina, and inflammatory pain of varied origins (e.g. osteoarthritis, rheumatoid arthritis, rheumatic disease, teno-synovitis and gout), headache, migraine and cluster headache, headache, primary hyperalgesia, secondary hyperalgesia, primary allodynia, secondary allodynia, or other pain caused by central sensitization.

Furthermore, antibodies of the invention may be used to decrease tolerance and/or dependence to opioid treatment of pain, and for treatment of withdrawal syndrome of e.g., alcohol, opioids, and cocaine.

Autism Spectrum Disorders

Autism spectrum disorders (ASD) are highly disabling developmental disorders with a population prevalence of 1-3%. Studies suggest that dysfunction of glutamatergic signaling, in particular through mGluR5 receptors, contributes to phenotypic deficits and is an appropriate target for pharmacologic intervention. In a VPA-induced murine model of autism, MPEP significantly reduced repetitive behaviors in VPA-treated mice, but had no effect on locomotor activity (Mehta M V et al. PloS One. 2011; 6(10):e26077.). These results are consistent with preclinical data showing that mGluR5-antagonists have therapeutic efficacy for core symptoms of autism. In addition, repetitive self-grooming behavior in the BTBR mouse model of autism is blocked by the mGluR5 antagonist MPEP (Silverman J L et al. Neuropsychopharmacology. 2010; 35(4):976.). Further evidence shows that negative allosteric modulation of the mGluR5 receptor reduces repetitive behaviors and rescues social deficits in mouse models of autism (Silverman J L et al. Science Translational Medicine. 2012; 4(131):131ra51.).

Exaggerated signaling through mGluR5 can account for multiple cognitive and syndromic features of fragile X syndrome (FXS), the most common inherited form of mental retardation and autism. FXS results from an expanded CGG triplet repeat expansion resulting in methylation and transcriptional silencing of the Fragile X Mental Retardation 1 gene and transcriptional silencing of the Fragile X Mental Retardation Protein (FMRP). According to the mGluR theory of FXS, excessive protein synthesis downstream of mGluR5 activation causes the synaptic pathophysiology that underlies multiple aspects of FXS. Indeed, hypersensitivity to mGluR5 and ERK1/2 leads to excessive protein synthesis in the hippocampus of a mouse model of FXS (Osterweil E K et al. Journal of Neuroscience. 2010; 30(46):15616-27.). Since a reduction of mGluR5 signalling can reverse FXS phenotypes, numerous studies provide a rationale for the use of mGluR5 antagonists for the treatment of FXS and related disorders (Dölen G, Bear M F. The Journal of Physiology. 2008; 586(6):1503-8.). In murine models of FXS, the small molecule mGluR5 antagonist AFQ056 rescues various aspects of the disease phenotype (Levenga J et al. Neurobiology of Disease. 2011; 42(3):311-7.).

In some embodiments, the antibodies of the invention are used to treat an autism spectrum disorder. In a still further aspect, the autism spectrum disorder is selected from autism, classical autism, Asperger syndrome, Pervasive Developmental Disorder Not Otherwise Specified (PDD-NOS; also known as atypical autism), Fragile X syndrome, Rett syndrome, and Childhood Disintegrative Disorder.

Neurologic and Psychiatric Disorders

The mGluR5 antibodies of the invention may be used in the treatment of a wide array of neurologic and psychiatric disorders.

In preferred embodiments of the inventive method, the disease, disorder, or medical condition is selected from: neurologic and psychiatric disorders including, but not limited to: (1) mood disorders and mood affective disorders; (2) neurotic, stress-related and somatoform disorders including anxiety disorders; (3) disorders of psychological development; (4) behavioral syndromes associated with physiological disturbances and physical factors; (5) extrapyramidal and movement disorders; (6) episodic and paroxysmal disorders, epilepsy; (7) pain; (8) forms of neurodegeneration; (9) cerebrovascular diseases, acute and chronic; and any sequelae of cerebrovascular diseases.

Examples of mood disorders and mood affective disorders that can be treated according to the present invention include, but are not limited to, bipolar disorder I depressed, hypomanic, manic and mixed form; bipolar disorder II; depressive disorders, such as single depressive episode or recurrent major depressive disorder, minor depressive disorder, treatment-resistant depression, depressive disorder with postpartum onset, depressive disorders with psychotic symptoms; persistent mood disorders, such as cyclothymia, dysthymia, euthymia; and premenstrual dysphoric disorder. In specific embodiments, the mood disorders and mood affective disorders that can be treated according to the present invention are major depressive disorder, treatment-resistant depression and bipolar disorder.

Examples of disorders belonging to the neurotic, stress-related and somatoform disorders that can be treated according to the present invention include, but are not limited to, anxiety disorders, general anxiety disorder, panic disorder with or without agoraphobia, specific phobia, social anxiety disorder, chronic anxiety disorders; obsessive compulsive disorder; reaction to sever stress and adjustment disorders, such as post-traumatic stress disorder (PTSD); other neurotic disorders such as depersonalisation-derealisation syndrome.

Examples of disorders of psychological development that can be treated according to the present invention include, but are not limited to pervasive developmental disorders, including but not limited to Asperger's syndrome and Rett's syndrome, autistic disorders, childhood autism and overactive disorder associated with mental retardation and stereotyped movements, specific developmental disorder of motor function, specific developmental disorders of scholastic skills.

Examples of behavioral syndromes associated with physiological disturbances and physical factors according to the present invention include, but are not limited to mental and behavioral disorders associated with childbirth, including but not limited to postnatal (postpartum) and prenatal depression; eating disorders, including but not limited to anorexia nervosa, bulimia nervosa, pica and binge eating disorder.

Examples of extrapyramidal and movement disorders that can be treated according to the present invention include, but are not limited to Parkinson's disease; second Parkinsonism, such as postencephalitic Parkinsonism; Parkinsonism comprised in other disorders; Lewy body disease; degenerative diseases of the basal ganglia; other extrapyramidal and movement disorders including but not limited to tremor, essential tremor and drug-induced tremor, myoclonus, chorea and drug-induced chorea, drug-induced tics and tics of organic origin, drug-induced acute dystonia, drug-induced tardive dyskinesia, L-dopa-induced dyskinesia; neuroleptic-induced movement disorders including but not limited to neuroleptic malignant syndrome (NMS), neuroleptic induced parkinsonism, neuroleptic-induced early onset or acute dyskinesia, neuroleptic-induced acute dystonia, neuroleptic-induced acute akathisia, neuroleptic-induced tardive dyskinesia, neuroleptic-induced tremor; restless leg syndrome, Stiff-man syndrome.

Further examples of movement disorders with malfunction and/or degeneration of basal ganglia that can be treated according to the present invention include, but are not limited to dystonia including but not limited to focal dystonia, multiple-focal or segmental dystonia, torsion dystonia, hemispheric, generalized and tardive dystonia (induced by psychopharmacological drugs). Focal dystonia include cervical dystonia (torticolli), blepharospasm (cramp of the eyelid), appendicular dystonia (cramp in the extremities, like the writer's cramp), oromandibular dystonia and spasmodic dysphonia (cramp of the vocal cord);

Examples for episodic and paroxysmal disorders that can be treated according to the present invention include, but are not limited to epilepsy, including localization-related (focal)(partial) idiopathic epilepsy and epileptic syndromes with seizures of localized onset, localization-related (focal)(partial) symptomatic epilepsy and epileptic syndromes with simple partial seizures, localization-related (focal)(partial) symptomatic epilepsy and epileptic syndromes with complex partial seizures, generalized idiopathic epilepsy and epileptic syndromes including but not limited to myoclonic epilepsy in infancy, neonatal convulsions (familial), childhood absence epilepsy (pyknolepsy), epilepsy with grand mal seizures on awakening, absence epilepsy, myoclonic epilepsy (impulsive petit mal) and nonspecific atonic, clonic, myoclonic, tonic, tonic-clonic epileptic seizures.

Further examples of epilepsy that can be treated according to the present invention include, but are not limited to epilepsy with myoclonic absences, myoclonic-astatic seizures, infantile spasms, Lennox-Gastaut syndrome, Salaam attacks, symptomatic early myoclonic encephalopathy, West's syndrome, petit and grand mal seizures; status epilepticus.

Examples of diseases that include forms of neurodegeneration include, but are not limited to, acute neurodegeneration, such as intracranial brain injuries, such as stroke, diffuse and local brain injuries, epidural, subdural and subarachnoid hemorrhage, and chronic neurodegeneration, such as Alzheimer's disease, Huntington's disease, multiple sclerosis and ALS.

Examples of cerebrovascular diseases include, but are not limited to, subarachnoid hemorrhage, intracerebral hemorrhage and other nontraumatic intracranial hemorrhage, cerebral infarction, stroke, occlusion and stenosis or precerebral and cerebral arteries, not resulting in cerebral infarction, dissection of cerebral arteries, cerebral aneurysm, cerebral atherosclerosis, progressive vascular leukoencephalopathy, hypertensive encephalopathy, nonpyogenic thrombosis of intracranial venous system, cerebral arteritis, cerebral amyloid angiopathy and sequelae of cerebrovascular diseases.

In one aspect, the disorder is a neurological and/or psychiatric disorder associated with glutamate dysfunction. In a further aspect, the disorder is selected from addiction, affective disorder, age-related cognitive decline, Alzheimer's disease, amnestic disorders, amyotrophic lateral sclerosis, anxiety, anxiety disorders, Angelman syndrome, Asperger's syndrome, attention deficit hyperactivity disorder, bipolar disorder, brain edema, chronic pain, delirium, dementia, depression, diabetes, Down Syndrome, dystonia, eating disorders, epilepsy, fibromyalgia, fragile x syndrome, Huntington's-related chorea, gastroesophageal reflux disease (GERD), levadopa-induced dyskinesia, manic-depressive illness, migraine, movement disorders, multiple sclerosis, narcolepsy, neurofibromatosis type 1, neuropathic pain, obesity, pain, paranoia, Parkinson's disease, post-herpetic neuropathic pain, psychotic disorders, PTEN hamartoma syndrome, schizophrenia, senile dementia, sleep disorder, substance-related disorder, or unipolar depression.

As it pertains to Parkinson's disease, L-DOPA, an existing treatment for Parkinson's, causes motor fluctuations and dyskinesia. Metabotropic glutamate receptor type 5 (mGluR5) is a proposed target for antidyskinetic therapies. Evidence shows that enhanced mGluR5 specific binding in the posterior putamen and pallidum contributes to the pathogenesis of L-DOPA-induced dyskinesia in Parkinson's disease (Samadi P et al. Neurobiology of Aging. 2008; 29(7):1040-51.). Fenobam, a noncompetitive mGluR5 antagonist already tested in humans, improves L-DOPA-induced dyskinesia in parkinsonian rats and monkeys (Rylander D et al. Neurobiology of disease. 2010; 39(3):352-61.). In a rodent model of Parkinson's, MPEP administration virtually abolished abnormal involuntary movements and dramatically reduced the abnormal striatal expression of FosB/Delta FosB associated with chronic L-DOPA administration (Levandis G et al. Neurobiology of Disease. 2008; 29(1):161-8.).

As it pertains to addiction, a significant body of evidence suggests that metabotropic glutamate receptors (mGluRs) are involved in both addiction reinforcement and the reinstatement of addictive substance-seeking behavior. Taking cocaine as an example, research shows that mGluR5 antagonists attenuate cocaine priming- and cue-induced reinstatement of cocaine seeking behavior (Kumaresan V et al. Behavioral Brain Research. 2009; 202(2):238-44.). In addition, mGluR5 signaling has been shown to be upregulated in the nucleus accumbens of binge drinkers; MPEP administration increases the sedative effect of alcohol, while reducing alcohol-induced withdrawal (Cozzoli D K et al. Journal of Neuroscience. 2009; 29(27):8655-68; Blednov Y A, Adron Harris R. International Journal of Neuropsychopharmacology. 2008; 11(6):775-93.). Further, cue-induced reinstatement of alcohol-seeking behavior is associated with increased ERK1/2 phosphorylation in specific limbic brain regions and is blocked by MPEP administration (Schroeder J P et al. Neuropharmacology. 2008; 55(4):546-54.). For methamphetamine addiction, mGluR5 antagonism with 3-((2-methyl-1,3-thiazol-4-yl)ethynyl)pyridine (MTEP) attenuates methamphetamine reinforcement and prevents reinstatement of methamphetamine-seeking behavior in rats (Gass J T et al. Neuropsychopharmacology. 2009; 34(4):820.).

As it pertains to Alzheimer's disease, clustering of mGluR5 elevates intracellular calcium and causes synapse deterioration, responses prevented by an mGluR5 antagonist (Renner M et al. Neuron. 2010; 66(5):739-54.). In addition, experiments have demonstrated that glutamate receptor antagonists, such as MPEP, are able to prevent Aβ oligomer-induced synaptic toxicity and further support the glutamatergic system as a target for the development of improved symptomatic/neuroprotective treatments for Alzheimer's (Rammes G et al. Neuropharmacology. 2011; 60(6):982-90).

### Variations

Included in the scope of the invention are functional portions of the inventive antibodies described herein. The term “functional portion” when used in reference to an antibody refers to any part or fragment of the antibody of the invention, which part or fragment retains the biological activity of the antibody of which it is a part (the parent antibody). Functional portions encompass, for example, those parts of an antibody that retain the ability to recognize target cells, or detect, treat, or prevent a disease, to a similar extent, the same extent, or to a higher extent, as the parent antibody. In reference to the parent antibody, the functional portion can comprise, for instance, about 10%, 25%, 30%, 50%, 68%, 80%, 90%, 95%, or more, of the parent antibody.

The functional portion can comprise additional amino acids at the amino or carboxy terminus of the portion, or at both termini, which additional amino acids are not found in the amino acid sequence of the parent antibody. Desirably, the additional amino acids do not interfere with the biological function of the functional portion, e.g., recognize target cells, inhibit mGluR5 activity, etc. More desirably, the additional amino acids enhance the biological activity, as compared to the biological activity of the parent antibody.

Included in the scope of the invention are functional variants of the inventive antibodies described herein. The term “functional variant” as used herein refers to an antibody, polypeptide, or protein having substantial or significant sequence identity or similarity to a parent antibody, which functional variant retains the biological activity of the antibody of which it is a variant. Functional variants encompass, for example, those variants of the antibody described herein (the parent antibody) that retain the ability to recognize target cells to a similar extent, the same extent, or to a higher extent, as the parent antibody. In reference to the parent antibody, the functional variant can, for instance, be at least about 30%, 50%, 75%, 80%, 90%, 98% or more identical in amino acid sequence to the parent antibody.

A functional variant can, for example, comprise the amino acid sequence of the parent antibody with at least one conservative amino acid substitution. Alternatively or additionally, the functional variants can comprise the amino acid sequence of the parent antibody with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody.

Amino acid substitutions of the inventive antibodies are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

Also, amino acids may be added or removed from the sequence based on vector design.

The antibody can consist essentially of the specified amino acid sequence or sequences described herein, such that other components, e.g., other amino acids, do not materially change the biological activity of the functional variant.

The antibodies of embodiments of the invention (including functional portions and functional variants) can be of any length, i.e., can comprise any number of amino acids, provided that the antibodies (or functional portions or functional variants thereof) retain their biological activity, e.g., the ability to specifically bind to antigen, detect diseased cells in a mammal, or treat or prevent disease in a mammal, etc. For example, the antibody can be about 50 to about 5000 amino acids long, such as 50, 70, 75, 100, 125, 150, 175, 200, 300, 400, 500, 600, 700, 800, 900, 1000 or more amino acids in length.

The antibodies of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The antibodies of embodiments of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The antibodies of embodiments of the invention (including functional portions and functional variants thereof) can be obtained by methods known in the art. The antibodies may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the antibodies of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the antibodies described herein (including functional portions and functional variants thereof) can be commercially synthesized by companies. In this respect, the inventive antibodies can be synthetic, recombinant, isolated, and/or purified.

Antibodies having V_{H} and V_{L} sequences disclosed herein may be used to create new variant antibodies by modifying the V_{H} and/or V_{L} sequences, or the constant region(s) attached thereto. Thus, the structural features of a variant antibody of the invention are used to create structurally related variant antibodies that retain at least one functional property of the antibodies of the invention, such as binding to mGluR5. For example, one or more CDR regions of one anti-mGluR5 variant antibody or mutations thereof, may be combined recombinantly with known framework regions and/or other CDRs to create additional, recombinantly-engineered, anti-mGluR5 antibodies (e.g., antibodies which bind to mGluR5) of the invention, as discussed herein. The starting material for the engineering method may be one or more of the V_{H} and/or V_{L} sequences provided herein, or one or more CDR regions thereof. To create the engineered antibody, it is not necessary to actually prepare (i.e., express as a protein) an antibody having one or more of the V_{H} and/or V_{L} sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein. Standard molecular biology techniques may be used to prepare and express altered antibody sequence.

The present invention particularly encompasses humanized mGluR5 antibodies. Exemplary teachings related to humanization of rabbit-derived monoclonal antibodies and preferred sequence modifications to maintain antigen-binding affinity are disclosed in International Publication No. WO 2008/144757, entitled Novel Rabbit Antibody Humanization Methods and Humanized Rabbit Antibodies, filed May 21, 2008, the disclosure of which is herein incorporated by reference in its entirety. Humanized antibodies are engineered to contain more human-like immunoglobulin domains, and incorporate the complementarity determining regions of the animal-derived antibody into the framework regions of a human antibody. This is accomplished by carefully examining the sequence of the hyper-variable loops of the variable regions of the monoclonal antibody and fitting them to the structure of the human antibody chains. See, e.g., U.S. Pat. No. 6,187,287, incorporated fully herein by reference.

The antibody encoded by the altered antibody sequence(s) may retain one, some or all of the functional properties of the anti-mGluR5 antibodies produced by methods and with sequences provided herein, which functional properties include binding to variant mGluR5 or variant mGluR5 conjugate with a specific K_{D} level or less and/or modulating immune cell activity, and/or selectively binding to desired target cells such as, for example, active T cells or B cells. The functional properties of the altered antibodies may be assessed using standard assays available in the art and/or described herein.

Mutations may be introduced randomly or selectively along all or part of an anti-mGluR5 antibody coding sequence and the resulting modified anti-mGluR5 antibodies may be screened for binding activity and/or other desired functional properties.

### Definitions

Unless defined otherwise, all technical and scientific terms used herein have the same meaning as those commonly understood by one of ordinary skill in the art to which this invention belongs. Although methods and materials similar or equivalent to those described herein may be used in the invention or testing of the present invention, suitable methods and materials are described herein. The materials, methods and examples are illustrative only, and are not intended to be limiting. The nomenclatures utilized in connection with, and the laboratory procedures and techniques of, immunology, antibody design, and medicinal and pharmaceutical chemistry described herein are those well-known and commonly used in the art. Standard techniques may be used for antibody syntheses, antibody analyses, pharmaceutical preparation, formulation, and delivery, and treatment of patients.

As used in the description herein and throughout the claims that follow, the meaning of “a,” “an,” and “the” includes plural reference unless the context clearly dictates otherwise.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

An “acute pain” generally refers to a pain which is not prolonged, i.e., lasts less than 6 months and which may come on suddenly as a result of a specific cause and which may be sharp in quality. Generally acute pain goes away once the affect area has been treated. Some acute pain is temporary and short-lived. Other times, it can have a longer-lasting effect and may cause severe pain. Some exemplary causes of acute pain include headache, migraine, surgery, broken bones, dental work, cosmetic surgery, burns, cuts, labor, childbirth, et al.

An “affective disorder” or “mood disorder” is one in which a disturbance in the person's mood is the main underlying feature, as classified in the Diagnostic and Statistical Manual of Mental Disorders (DSM) and International Classification of Diseases (ICD). Mood disorders fall into the basic groups of elevated mood, such as mania or hypomania; depressed mood, of which the best-known and most researched is major depressive disorder (MDD) (commonly called clinical depression, unipolar depression, or major depression); and moods which cycle between mania and depression, known as bipolar disorder (BD) (formerly known as manic depression). There are several sub-types of depressive disorders or psychiatric syndromes featuring less severe symptoms such as dysthymic disorder and cyclothymic disorder. Mood disorders may also be substance induced or occur in response to a medical condition.

“Amino acid,” as used herein refers broadly to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified (e.g., hydroxyproline, γ-carboxyglutamate, and O-phosphoserine). Amino acid analogs refers to compounds that have the same basic chemical structure as a naturally occurring amino acid (i. e., a carbon that is bound to a hydrogen, a carboxyl group, an amino group), and an R group (e.g., homoserine, norleucine, methionine sulfoxide, methionine methyl sulfonium.) Analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “antibody,” as used herein, refers to an immunoglobulin molecule which specifically binds with an antigen. In one aspect, the antigen is mGluR5. Antibodies can be intact immunoglobulins derived from natural sources or from recombinant sources and can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The term “antigen-binding fragment” or “antibody fragment” refers to a portion of an intact antibody and refers to the antigenic determining variable regions of an intact antibody. Examples of antibody fragments include, but are not limited to, fragment antigen binding (Fab) fragments, F(ab′)2 fragments, Fab′ fragments, Fv fragments, recombinant IgG (rIgG) fragments, single chain antibody fragments, including single chain variable fragments (scFv), single domain antibodies (e.g., sdAb, sdFv, nanobody) fragments, diabodies, and multispecific antibodies formed from antibody fragments. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird, et al. (1988) Science 242: 423-426; Huston, et al. (1988) Proc Natl. Acad. Sci. USA 85: 5879-5883; and Osbourn, et al. (1998) Nat. Biotechnol. 16: 778. Single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab, Fv, or other fragments of immunoglobulins using either protein chemistry or recombinant DNA technology. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites. See e.g. Holliger, et al. (1993) Proc Natd. Acad. Sci. USA 90: 6444-6448; Poljak, et al. (1994) Structure 2: 1121-1123. Still further, an antibody or antigen-binding portion thereof (antigen-binding fragment, antibody fragment, antibody portion) may be part of a larger immunoadhesion molecules, formed by covalent or noncovalent association of the antibody or antibody portion with one or more other proteins or peptides. Examples of immunoadhesion molecules include use of the streptavidin core region to make a tetrameric scFv molecule (Kipriyanov, et al. (1995) Hum. Antibodies Hybridomas 6: 93-101) and use of a cysteine residue, a marker peptide and a C-terminal polyhistidine tag to make bivalent and biotinylated scFv molecules. Kipriyanov, et al. (1994) Mol. Immunol. 31: 1047-1058. Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Antibodies may be polyclonal, monoclonal, xenogeneic, allogeneic, syngeneic, or modified forms thereof, e.g., humanized, chimeric, bispecific or multispecific antibodies.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated, synthesized, or can be derived from a biological sample, or might be macromolecule besides a polypeptide. Such a biological sample can include, but is not limited to a tissue sample, a tumor sample, a cell or a fluid with other biological components. In one aspect, the antigen is mGluR5.

The term “bind” refers to an attractive interaction between two molecules that results in a stable association in which the molecules are in close proximity to each other. The result of molecular binding is sometimes the formation of a molecular complex in which the attractive forces holding the components together are generally non-covalent, and thus are normally energetically weaker than covalent bonds.

The term “centrally administered” or “central administration” herein means that an entity, typically an antibody, is administered under conditions whereby it reaches mGluR5 in the central nervous system and/or within the blood brain barrier. One means of achieving central administration is intrathecal administration.

A “chimeric antibody” is an antibody made by fusing the antigen binding region (variable domains of the heavy and light chains, V_{H} and V_{L}) from one species, like mouse or rabbit, with the constant domain (effector region) from another species, e.g., human. The chimeric antibodies retain the original antibody's antigen specificity and affinity, without the same immunogenicity. Chimeric antibodies may be made by recombinant means by combining the V_{L} and V_{H} regions, obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the invention may be selected from IgG1, IgG2, IgG3, and IgG4 constant regions.

A “chronic pain” refers to an ongoing pain that occurs for a prolonged duration, e.g., at least 6 months, 9 months, a year or longer. Chronic pain may be severe and may be associated with reduced likelihood of survival and depression. Conditions associated with chronic pain include by way of example cancers, heart disease, respiratory disease, spinal and muscle disorders, headache conditions, bone disorders, multiple sclerosis, arthritic conditions, nerve disorders, nerve damage (neuropathy), fibromyalgia, Lyme disease, IBD, IBS, acid reflux or ulcers, endometriosis, et al.

The term “compete,” as used herein with regard to an antibody, means that a first antibody, or an antigen binding fragment (or portion) thereof, binds to an epitope in a manner sufficiently similar to the binding of a second antibody, or an antigen binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein. In some embodiments, the antibody of the invention may compete or cross-compete with mGluR5 Ab1-Ab29 for binding to mGluR5. In a preferred embodiment, an antibody of the invention may compete with exemplary antibody AbA for binding to mGluR5. In a preferred embodiment, an antibody of the invention may compete with exemplary antibody AbB for binding to mGluR5. In a preferred embodiment, an antibody of the invention may compete with exemplary antibody AbC for binding to mGluR5.

“Complementarity determining region,” “HVR,” “hypervariable region,” or “CDR,” as used herein, refers broadly to one or more of the hyper-variable or complementarily determining regions (CDRs) found in the variable regions of light or heavy chains of an antibody, non-contiguous sequences of amino acids which confer antigen specificity and/or binding affinity. See Kabat, et al. (1987) Sequences of Proteins of Immunological Interest National Institutes of Health, Bethesda, Md. These expressions include the hypervariable regions as defined by Kabat, et al. (1983) Sequences of Proteins of Immunological Interest, U. S. Dept. of Health and Human Services or the hypervariable loops in 3-dimensional structures of antibodies. Chothia and Lesk (1987) J. Mol. Biol. 196: 901-917. The CDRs in each chain are held in close proximity by framework regions and, with the CDRs from the other chain, contribute to the formation of the antigen-binding site. Within the CDRs there are select amino acids that have been described as the selectivity determining regions (SDRs) which represent the critical contact residues used by the CDR in the antibody-antigen interaction. (Kashmiri Methods 36: 25-34(2005)). In general, there are three CDRs in each heavy chain variable region (CDR-H1, CDR-H2, CDR-H3) and three CDRs in each light chain variable region (CDR-L1, CDR-L2, CDR-L3).

The phrase “disease associated with expression of mGluR5” includes, but is not limited to, a disease associated with expression of mGluR5 or condition associated with cells which express mGluR5 including, e.g., migraine, pain, IBS, GERD, OAB, autism spectrum disorders, incontinence, neurological disorders, affective disorders, and psychiatric disorders.

The term “dyskinesia” fers to a category of movement disorders that are characterized by involuntary muscle movements, including movements similar to tics or chorea and diminished voluntary movements. Dyskinesia can be anything from a slight tremor of the hands to an uncontrollable movement of the upper body or lower extremities. Discoordination can also occur internally especially with the respiratory muscles and it often goes unrecognized. Dyskinesia is a symptom of several medical disorders including Parkinson's disease, Ataxia, Cervical dystonia. Chorea, Dystonia, Functional movement disorder, Huntington's disease, Multiple system atrophy, Myoclonus, Parkinson's disease, Parkinsonism, Progressive supranuclear palsy, Restless legs syndrome, Tardive dyskinesia, Tourette syndrome, Tremor, Wilson's disease, et al.

The term “dystonia” refers to a movement disorder in which a person's muscles contract uncontrollably. The contraction causes the affected body part to twist involuntarily, resulting in repetitive movements or abnormal postures. Dystonia can affect one muscle, a muscle group, or the entire body. Most cases of dystonia do not have a specific cause. Acquired dystonia may be caused by damage to the basal ganglia, such as the result of a brain trauma, stroke, tumor, oxygen deprivation, infection, drug reaction, or poisoning such as by lead or carbon monoxide.

The term “EC₅₀” as used herein refers to the dose of a test compound, e.g., anti-mGluR5 antibody or antigen-binding fragment thereof, which produces 50% of its maximum response or effect in an assay.

An “effective amount” or “an amount effective to treat” refers to a dose that is adequate to prevent or treat a disease, condition, or disorder in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the inventive antibodies in each or various rounds of administration.

An “epitope” or “binding site” is an area or region on an antigen to which an antigen-binding peptide (such as an antibody) specifically binds. A protein epitope may comprise amino acid residues directly involved in the binding (also called immunodominant component of the epitope) and other amino acid residues, which are not directly involved in the binding, such as amino acid residues that are effectively blocked by the specifically antigen binding peptide (in other words, the amino acid residue is within the “footprint” of the specifically antigen binding peptide). The term epitope herein includes both types of amino acid binding sites in any particular region of mGluR5 that specifically binds to an anti-mGluR5 antibody. mGluR5 may comprise a number of different epitopes, which may include, without limitation, (1) linear peptide antigenic determinants, (2) conformational antigenic determinants that consist of one or more noncontiguous amino acids located near each other in a mature mGluR5 conformation; and (3) post-translational antigenic determinants that consist, either in whole or part, of molecular structures covalently attached to a mGluR5 protein such as carbohydrate groups. In particular, the term “epitope” includes the specific residues in a protein or peptide, e.g., mGluR5, which are involved in the binding of an antibody to such protein or peptide as determined by known and accepted methods such as alanine scanning techniques. Such methods are exemplified herein.

An “expression vector” herein refers to DNA vectors containing elements that facilitate manipulation for the expression of a foreign protein within the target host cell, e.g., a bacterial, insect, yeast, plant, amphibian, reptile, avian, or mammalian cell, and most typically a yeast or mammalian cell, e.g., a CHO cell. Conveniently, manipulation of sequences and production of DNA for transformation is first performed in a bacterial host, e.g. E. coli, and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, (b) complement auxotrophic deficiencies, or (c) supply critical nutrients not available from complex media. Exemplary vectors and methods for transformation of yeast are described, for example, in Burke, D., Dawson, D., & Stearns, T., Methods in Yeast Genetics: a Cold Spring Harbor Laboratory Course Manual, Plainview, N.Y.: Cold Spring Harbor Laboratory Press (2000). Expression vectors for use in the methods of the invention may include yeast or mammalian specific sequences, including a selectable auxotrophic or drug marker for identifying transformed host strains. A drug marker may further be used to amplify copy number of the vector in a yeast host cell.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications. The expression/production of an antibody or antigen-binding fragment can be within the cytoplasm of the cell, and/or into the extracellular milieu such as the growth medium of a cell culture.

The terms “Fc receptor” and “FcR” describe a receptor that binds to the Fc region of an antibody. The preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, Ann. Rev. Immunol., 9:457-92 (1991); Capel et al., Immunomethods, 4:25-34 (1994); and de Haas et al, J. Lab. Clin. Med., 126:330-41 (1995). “FcR” also includes the neonatal receptor, FcRn, which is responsible for the transfer of maternal IgGs to the fetus (Guyer et al, J. Immunol., 117:587 (1976); and Kim et al., J. Immunol., 24:249 (1994)), and which primarily functions to modulate and/or extend the half-life of antibodies in circulation. To the extent that the disclosed anti-mGluR5 antibodies are aglycosylated, as a result of the expression system and/or sequence, the subject antibodies are expected to bind FcRn receptors, but not to bind (or to minimally bind) Fcγ receptors.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al, Sequences of Proteins of Immunological Interest, 5th edition, Bethesda, Md.: U.S. Dept. of Health and Human Services, Public Health Service, National Institutes of Health (1991). The Fc region of an immunoglobulin generally comprises two constant domains, CH2 and CH3.

The expressions “framework region” or “FR” refer to one or more of the framework regions within the variable regions of the light and heavy chains of an antibody (See Kabat et al, Sequences of Proteins of Immunological Interest, 4^{th} edition, Bethesda, Md.: U.S. Dept. of Health and Human Services, Public Health Service, National Institutes of Health (1987)). These expressions include those amino acid sequence regions interposed between the CDRs within the variable regions of the light and heavy chains of an antibody. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

A “functional Fc region” possesses at least one effector function of a native sequence Fc region. Exemplary “effector functions” include C1q binding; complement dependent cytotoxicity (“CDC”); Fc receptor binding; antibody-dependent cell-mediated cytotoxicity (“ADCC”); phagocytosis; down-regulation of cell surface receptors (e.g. B cell receptor (“BCR”)), etc. Such effector functions generally require the Fc region to be combined with a binding domain (e.g. an antibody variable domain) and can be assessed using various assays known in the art for evaluating such antibody effector functions. A “native sequence Fc region” comprises an amino acid sequence identical to the amino acid sequence of an Fc region found in nature. A “variant Fc region” comprises an amino acid sequence that differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95%, at least about 96%, at least about 97%, at least about 98%, or at least about 99% sequence identity therewith.

“Host cell,” as used herein, refers broadly to a cell into which a nucleic acid molecule of the invention, such as a recombinant expression vector of the invention, has been introduced. Host cells may be prokaryotic cells (e.g., E. coli), or eukaryotic cells such as yeast, insect (e.g., SF9), amphibian, or mammalian cells such as CHO, HeLa, HEK-293, e.g., cultured cells, explants, and cells in vivo. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, progeny may not, in fact, be identical to the parent cell, but are still included within the scope of the term as used herein.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; and Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

“Human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell. This includes fully human monoclonal antibodies and conjugates and variants thereof, e.g., which are bound to effector agents such as therapeutics or diagnostic agents.

“Humanized antibody,” as used herein, broadly includes antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “IC₅₀” as used herein refers to the dose of a test compound, e.g., anti-mGluR5 antibody or antigen-binding fragment thereof, which produces 50% inhibition in a biochemical assay.

The term “inflammatory pain” refers to pain resulting from the perception of and affective response to noxious stimuli that occur during an inflammatory or immune response. Non-limiting conditions which may be associated with inflammatory pain include various inflammatory and autoimmune conditions including by way of example cancer, heart disease, diabetes, Alzheimer's disease, arthritis conditions such as rheumatoid arthritis, psoriatic arthritis and gouty arthritis, conditions of the joints and musculoskeletal system such as osteoarthritis, fibromyalgia, muscular low back pain, and muscular neck pain, asthma, chronic peptic ulcer, tuberculosis, periodontitis, ulcerative colitis, Crohn's disease, sinusitis, active hepatitis, celiac disease, glomerulonephritis, hepatitis, inflammatory bowel disease, reperfusion injury, transplant rejection, inflammatory bowel disease (IBD), multiple sclerosis (MS), Type 1 diabetes mellitus, Guillain-Barre syndrome, Chronic inflammatory demyelinating polyneuropathy, Psoriasis, Graves' Disease, Hashimoto's thyroiditis, myasthenia gravis, vasculitis, et al.

The term “inhibitor” as used herein refers to a compound that binds to a target and renders it biologically inactive or less active. In a particular embodiment, the compound is an anti-mGluR5 antibody or antigen-binding fragment thereof. In some embodiments, the inhibitory effect of the compound is measured via inhibition of mGluR5-mediated pERK production.

An “isolated” biological component (such as an isolated antibody or cell or vector or protein or nucleic acid) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

“Isolated antibody,” as used herein, is intended to refer to an antibody that is substantially free of other antibodies having different antigenic specificities (e.g., an isolated antibody that specifically binds mGluR5 is substantially free of antibodies that specifically bind antigens other than mGluR5). Moreover, an isolated antibody may be substantially free of other cellular material and/or chemicals.

“Label” or a “detectable moiety” as used herein, refers broadly to a composition detectable by spectroscopic, photochemical, biochemical, immunochemical, chemical, or other physical means.

“Metabotropic glutamate receptor 1” or “mGluR1” is a member of the group C family of G-protein-coupled receptors, or GPCRs. Like all glutamate receptors, mGluR1 binds with glutamate, an amino acid that functions as an excitatory neurotransmitter. mGluR1 is encoded by the human gene GRM1. It is also known as GPRC1A, MGLU1, MGLUR1, PPP1R85, SCAR13, glutamate metabotropic receptor 1, and SCA44. Information for human mGluR1 may be found at the reference page for NCBI Accession No. NP_001264993.1 (SEQ ID NO:5).

“Metabotropic glutamate receptor 5” or “mGluR5” is a member of the group C family of G-protein-coupled receptors, or GPCRs. Like all glutamate receptors, mGluR5 binds with glutamate, an amino acid that functions as an excitatory neurotransmitter. mGluR5 performs a variety of functions in the central and peripheral nervous systems, and is implicated in, for example, learning, memory, anxiety, and the perception of pain. It is found in pre- and postsynaptic neurons in synapses of the hippocampus, cerebellum, and the cerebral cortex, as well as other parts of the brain and in peripheral tissues. It has been shown to activate phospholipase C. Like other metabotropic receptors, mGluR5 has seven transmembrane domains that span the cell membrane. Unlike ionotropic receptors, metabotropic glutamate receptors are not ion channels. Instead, they activate biochemical cascades, leading to the modification of other proteins, as for example ion channels. This can lead to changes in the synapse's excitability, for example by presynaptic inhibition of neurotransmission, or modulation and even induction of postsynaptic responses. mGluR5 is also known as GRM5, GPRC1E, MGLUR5, PPP1R86, mGlu5, and glutamate metabotropic receptor 5. mGluR5 has multiple isoforms, with isoform 2 or 5b being the canonical one. mGluR5a and mGluR5b both activate phospholipase C. Unless otherwise stated, “Metabotropic glutamate receptor 5” or “mGluR5” may refer to any of the known isoforms of mGluR5. mGluR5 sequences may be found at the following database reference pages: human mGluR5, UniProt Accession No. P41594; human mGluR5, NCBI Accession No. NP_001137303.1 and NP_000833.1 (SEQ ID NO:1 and 2); rat mGluR5, NCBI Accession No. NP_058708.1 (SEQ ID NO:3); and cynomolgus monkey mGluR5, NCBI Accession No. XP_005579366.1 (SEQ ID NO:4).

“Multispecific antibody” or “multispecific antigen-binding protein” refers to a polypeptide or antibody with 2 or more antigen binding regions. This includes bispecific antibodies. These antigen binding regions may bind to different antigens or to different epitopes of the same antigen.

A “neurodegenerative disease” is a disease involving the progressive loss of structure or function of neurons, including death of neurons. Many neurodegenerative diseases—including amyotrophic lfteral sclerosis, Parkinson's, Alzheimer's, and Huntington's—occur as a result of neurodegenerative processes. Such diseases are incurable, resulting in progressive degeneration and/or death of neuron cells, and are often characterized by atypical protein assemblies as well as induced cell death. Neurodegeneration can be found in many different levels of neuronal circuitry ranging from molecular to systemic. One aspect of the invention comprises a method for treating, preventing, or alleviating symptoms associated with a neurodegenerative disease, wherein the method comprises the administration of anti-human mGluR5 antibodies or antibody fragments. In a particular aspect, the antibody is selected from Ab1-Ab29. In a preferred aspect, the antibody is exemplary AbA. In another preferred embodiment, the antibody is exemplary AbB. In an additionally preferred embodiment, the antibody is exemplary AbC.

A “neurological disorder” is any disorder of the nervous system. Structural, biochemical or electrical abnormalities in the brain, spinal cord or other nerves can result in a range of symptoms. The specific causes of neurological problems vary, but can include genetic disorders, congenital abnormalities or disorders, infections, lifestyle or environmental health problems including malnutrition, and brain injury, spinal cord injury or nerve injury. The problem may start in another body system that interacts with the nervous system. These disorders can be separated into the categories of central nervous system disorders and peripheral nervous system disorders. In one aspect, the invention comprises a method to treat a neurological disorder through the administration of anti-mGluR5 antibodies or antigen-binding antibody fragments. In a particular aspect, the antibody is selected from Ab1-Ab29. In a preferred aspect, the antibody is exemplary AbA. In another preferred embodiment, the antibody is exemplary AbB. In an additionally preferred embodiment, the antibody is exemplary AbC.

A “neuropathic pain” generally refers to a pain caused by nerve damage or a disease affecting the somatosensory nervous system. Neuropathic pain may be associated with abnormal sensations called dysesthesia or pain from normally non-painful stimuli (allodynia). It may have continuous and/or episodic (paroxysmal) components. The latter resemble stabbings or electric shocks. Common qualities include burning or coldness, “pins and needles” sensations, numbness and itching. Neuropathic pain can be a symptom or complication of different diseases and conditions, Non-limiting examples of conditions, traumas and treatments which may be associated with neuropathic pain include multiple sclerosis, multiple myeloma, and other types of cancer, diabetes, long-term excessive alcohol intake, trigeminal neuralgia, cancer treatment such as chemotherapy and radiation, injuries to tissue, muscles, or joints, back, leg, and hip problems, accidents or injuries that affect the spine, herniated discs and spinal cord compression, shingles, syphilis infection, HIV, limb loss, phantom limb syndrome, vitamin B deficiency, carpal tunnel syndrome, thyroid problems, facial nerve problems, arthritis in the spine, et al.

A “neuropsychiatric” disease and/or disorder is one with psychiatric features associated with known nervous system injury, underdevelopment, biochemical, anatomical, or electrical malfunction, and/or disease pathology, e.g., Attention deficit hyperactivity disorder, Autism, Tourette's syndrome and some cases of obsessive compulsive disorder as well as the neurobehavioral associated symptoms of degeneration of the nervous system such as Parkinson's disease, essential tremor, Huntington's disease, Alzheimer's disease, multiple sclerosis and organic psychosis. One aspect of the invention comprises a method for treating, preventing, or alleviating symptoms associated with a neuropsychiatric disease or disorder, wherein the method comprises the administration of anti-human mGluR5 antibodies or antibody fragments. In a particular aspect, the antibody is selected from Ab1-Ab29. In a preferred aspect, the antibody is exemplary AbA. In another preferred embodiment, the antibody is exemplary AbB. In an additionally preferred embodiment, the antibody is exemplary AbC.

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism. The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences

A “nociceptive pain” generally refers to pain resulting from an injury to body tissues such as bruises, burns, cuts, fractures, overuse or joint damage, sports injury, dental procedure, cosmetic procedure, a sprain et al.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites or alternatively via a PCR/recombination method familiar to those skilled in the art (GATEWAY11 Technology; Invitrogen, Carlsbad Calif.). If such sites do not exist, the synthetic oligonucleotide adapters or linkers are used in accordance with conventional practice.

The term “peripherally administered” or “peripheral administration” herein means that an entity, typically an antibody, is administered under conditions whereby it reaches mGluR5 in the periphery, i.e., outside the blood brain barrier. Means of achieving peripheral administration of an antibody include by way of example intravenous, subcutaneous, intramuscular and intraperitoneal administration.

The term “peripherally and centrally administered” or “peripheral and central administration” herein means that an entity, typically an antibody, is administered under conditions whereby it reaches mGluR5 in the periphery, i.e., outside the blood brain barrier and mGluR5 in the CNS, i.e., inside the BBB. This may be achieved e.g., by administering an antibody intrathecally and further administering the antibody so that it reaches the periphery, e.g., via intravenous, subcutaneous, intramuscular or intraperitoneal administration.

A “pharmaceutically acceptable carrier” or “excipient” refers to compounds or materials conventionally used in pharmaceutical compositions during formulation and/or to permit storage. Excipients may be added to facilitate manufacture, enhance stability, control release, enhance product characteristics, enhance bioavailability drug absorption or solubility, or other pharmacokinetic considerations, enhance patient acceptability, etc. Pharmaceutical excipients include, for example, carriers, fillers, binders, disintegrants, lubricants, glidants, colors, preservatives, suspending agents, dispersing agents, film formers, buffer agents, pH adjusters, preservatives etc. The selection of appropriate excipients also depends upon the route of administration and the dosage form, as well as the active ingredient and other factors, and will be readily understood by one of ordinary skill in the art. A pharmaceutically acceptable carrier is intended to include any and all solvents, dispersion media, coatings, antibacterial and antifungal agents, isotonic and absorption delaying agents, and the like, compatible with pharmaceutical administration. Pharmaceutically acceptable carriers include a wide range of known diluents (i.e., solvents), fillers, extending agents, binders, suspending agents, disintegrates, surfactants, lubricants, excipients, wetting agents and the like commonly used in this field. These carriers may be used singly or in combination according to the form of the pharmaceutical preparation, and may further encompass any other component added to a pharmaceutical formulation other than the active ingredient and which is capable of bulking-up formulations that contain potent active ingredients (thus often referred to as “bulking agents,” “fillers,” or “diluents”) to allow convenient and accurate dispensation of a drug substance when producing a dosage form.

“Polypeptide,” “peptide” and “protein,” are used interchangeably and refer broadly to a polymer of amino acid residues s of any length, regardless of modification (e.g., phosphorylation or glycosylation). The terms apply to amino acid polymers in which one or more amino acid residue is an analog or mimetic of a corresponding naturally occurring amino acid, as well as to naturally occurring amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer. Polypeptides can be modified, e.g., by the addition of carbohydrate residues to form glycoproteins. The terms “polypeptide,” “peptide” and “protein” expressly include glycoproteins, as well as non-glycoproteins.

The term “promoter,” as used herein, is defined as a DNA sequence recognized by the synthetic machinery of the cell, or introduced synthetic machinery, required to initiate the specific transcription of a polynucleotide sequence.

“Prophylactically effective amount,” as used herein, refers broadly to the amount of a compound that, when administered to a patient for prophylaxis of a disease or prevention of the reoccurrence of a disease, is sufficient to effect such prophylaxis for the disease or reoccurrence. The prophylactically effective amount may be an amount effective to prevent the incidence of signs and/or symptoms. The “prophylactically effective amount” may vary depending on the disease and its severity and the age, weight, medical history, predisposition to conditions, preexisting conditions, of the patient to be treated.

A “psychiatric disorder,” also known as a “mental illness” or “mental disorder,” is a syndrome characterized by clinically significant disturbance in an individual's cognition, emotion regulation, or behavior that reflects a dysfunction in the psychological, biological, or developmental processes underlying mental functioning. See, e.g., Diagnostic and Statistical Manual of Mental Disorders (American Psychiatric Association) and International Classification of Diseases, Mental and Behavioral Disorders (World Health Organization). One aspect of the invention comprises a method for treating, preventing, or alleviating symptoms associated with a psychiatric disease or disorder, wherein the method comprises the administration of anti-human mGluR5 antibodies or antibody fragments. In a particular aspect, the antibody is selected from Ab1-Ab29. In a preferred aspect, the antibody is exemplary AbA. In another preferred embodiment, the antibody is exemplary AbB. In an additionally preferred embodiment, the antibody is exemplary AbC.

“Recombinant” as used herein, refers broadly to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

A “selectable marker” herein refers to a gene or gene fragment that confers a growth phenotype (physical growth characteristic) to a cell receiving that gene as, for example through a transformation event. The selectable marker allows that cell to survive and grow in a selective growth medium under conditions in which cells that do not receive that selectable marker gene cannot grow. Selectable marker genes generally fall into several types, including positive selectable marker genes such as a gene that confers on a cell resistance to an antibiotic or other drug, temperature when two temperature sensitive (“ts”) mutants are crossed or a ts mutant is transformed; negative selectable marker genes such as a biosynthetic gene that confers on a cell the ability to grow in a medium without a specific nutrient needed by all cells that do not have that biosynthetic gene, or a mutagenized biosynthetic gene that confers on a cell inability to grow by cells that do not have the wild type gene; and the like. Suitable markers include but are not limited to: ZEO; G418; LYS3; MET1; MET3a; ADE1; ADE3; URA3; and the like.

“Subject” or “patient” or “individual” in the context of therapy or diagnosis herein includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc., i.e., anyone suitable to be treated according to the present invention include, but are not limited to, avian and mammalian subjects, and are preferably mammalian. Any mammalian subject in need of being treated according to the present invention is suitable. Human subjects of both genders and at any stage of development (i. e., neonate, infant, juvenile, adolescent, and adult) can be treated according to the present invention. The present invention may also be carried out on animal subjects, particularly mammalian subjects such as mice, rats, dogs, cats, cattle, goats, sheep, and horses for veterinary purposes, and for drug screening and drug development purposes. “Subjects” is used interchangeably with “individuals” and “patients.”

The phrase that an antibody (e.g., first antibody) binds “substantially” or “at least partially” the same epitope as another antibody (e.g., second antibody) means that the epitope binding site for the first antibody comprises at least 10%, 20%, 30%, 40%, 50%, 60%, 70%, 80%, 90%, or more of the amino acid residues on the antigen that constitutes the epitope binding site of the second antibody. Also, that a first antibody binds substantially or partially the same or overlapping epitope as a second antibody means that the first and second antibodies compete in binding to the antigen, as described above. Thus, the term “binds to substantially the same epitope or determinant as” a monoclonal antibody means that an antibody “competes” with the antibody. The phrase “binds to the same or overlapping epitope or determinant as” an antibody of interest means that an antibody “competes” with said antibody of interest for at least one, (e.g., at least 2, at least 3, at least 4, at least 5) or all residues on mGluR5 to which said antibody of interest specifically binds. The identification of one or more antibodies that bind(s) to substantially or essentially the same epitope as the monoclonal antibodies described herein can be readily determined using alanine scanning. Additionally, any one of variety of immunological screening assays in which antibody competition can be assessed. A number of such assays are routinely practiced and well known in the art (see, e.g., U.S. Pat. No. 5,660,827, issued Aug. 26, 1997, which is specifically incorporated herein by reference). It will be understood that actually determining the epitope to which an antibody described herein binds is not in any way required to identify an antibody that binds to the same or substantially the same or overlapping epitope as the monoclonal antibody described herein.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

“Therapy,” “therapeutic,” “treating,” or “treatment,” as used herein, refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms (e.g., inflammation, pain). Therapy also encompasses “prophylaxis.” The term “reduced,” for purpose of therapy, refers broadly to the clinical significant reduction in signs and/or symptoms. Therapy includes treating relapses or recurrent signs and/or symptoms (e.g., inflammation, pain). Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms (e.g., inflammation, pain).

“Variable region” or “VR,” as used herein, refers broadly to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

A “vector” is a replicon, such as a plasmid, phage, cosmid, or virus in which a nucleic acid segment may be operably inserted so as to bring about the replication or expression of the segment. The vector may contain one or more additional sequences such as, but not limited to, regulatory sequences (e.g., promoter, enhancer), a selection marker, and a polyadenylation signal. Vectors for transforming a wide variety of host cells are well known to those of skill in the art. They include, but are not limited to, plasmids, phagemids, cosmids, baculoviruses, bacmids, bacterial artificial chromosomes (BACs), yeast artificial chromosomes (YACs), as well as other bacterial, yeast and viral vectors. The vectors described herein may be integrated into the host genome or maintained independently in the cell or nucleus.

### Use Embodiments

The invention further relates to an antibody or antigen-binding antibody fragment, or pharmaceutical composition according to the invention according to the following Embodiments 1-24
1. An antibody or antigen-binding antibody fragment, or pharmaceutical composition according to the invention for use in the treatment or prophylaxis of migraine, GERD, irritable bowel syndrome (IBS), overactive bladder (OAB), urinary incontinence, autism spectrum disorder, a neurological or psychiatric disorder, a behavior and dependence disorders, a neurological disorder, or pain.
2. The antibody or antigen-binding antibody fragment, or pharmaceutical composition according to Embodiment 1 for use in the treatments or prophylaxis of one or more symptoms associated with migraine, or reduces the frequency and/or severity of such one or more symptoms, optionally vasomotor symptoms (e.g. hot flashes, facial flushing, sweating, and night sweats), photophobia, phonophobia, sensitivity to smells, tearing/lacrimation, vertigo, dizziness, nausea, vomiting, headache pain, and aura.
3. The antibody or antigen-binding antibody fragment, or pharmaceutical composition according to Embodiment 1, wherein the migraine is selected from the group comprising episodic migraine, chronic migraine or cluster headache.
4. The antibody or antigen-binding antibody fragment, or pharmaceutical composition according to Embodiment 1 for use in the treatment of a patient that has not previously received prophylactic therapy for migraine headaches.
5. The antibody or antigen-binding antibody fragment, or pharmaceutical composition according to Embodiment 1 for use in the treatment or prophylaxis gastroesophageal reflux disease is nonerosive reflux disease or erosive esophagitis.
6. The antibody or antigen-binding antibody fragment, or pharmaceutical composition according to Embodiment 1 for use in the treatment or prophylaxis of diarrhea predominant IBS, constipation predominant IBS, or alternating bowel movement predominant IBS.
7. The antibody or antigen-binding antibody fragment, or pharmaceutical composition according to Embodiment 1 use in the treatment or prophylaxis urgency, urinary frequency, nocturia, or urge incontinence.
8. The antibody or antigen-binding antibody fragment, or pharmaceutical composition according to Embodiment 1 for use in the treatment or prophylaxis of stress incontinence, urge incontinence, overflow incontinence, mixed incontinence, structural incontinence, functional incontinence, nocturnal incontinence, transient incontinence, giggle incontinence, double incontinence, post-void dribbling, and coital incontinence.
9. The antibody or antigen-binding antibody fragment, or pharmaceutical composition according to Embodiment 1 for use in the treatment or prophylaxis of impaired social and functional communication, anxiety, inattention, hyperactivity, altered sensory reactivity, self-injury, aggression, impaired cognitive function, and compromised daily living skills.
10. The antibody or antigen-binding antibody fragment, or pharmaceutical composition according to Embodiment 1 for use in the treatment or prophylaxis of schizophrenia, schizophreniform disorder, schizoaffective disorder, delusional disorder, brief psychotic disorder, shared psychotic disorder, psychotic disorder due to a general medical condition, substance-induced psychotic disorder, psychotic disorder not otherwise specified, psychosis associated with dementia, major depressive disorder, dysthymic disorder, premenstrual dysphoric disorder, depressive disorder not otherwise specified, bipolar I disorder, bipolar 11 disorder, cyclothymic disorder, bipolar disorder not otherwise specified, mood disorder due to a general medical condition, substance-induced mood disorder, mood disorder not otherwise specified, generalized anxiety disorder, obsessive-compulsive disorder, panic disorder, acute stress disorder, post-traumatic stress disorder, mental retardation, pervasive developmental disorders, attention deficit disorders, attention-deficit/hyperactivity disorder, disruptive behavior disorders, personality disorder of the paranoid type, personality disorder of the schizoid type, personality disorder of the schizotypical type, tic disorders, Tourette's syndrome, substance dependence, substance abuse, substance withdrawal, trichotillomania, and conditions wherein cognition is impaired, Alzheimer's disease, Parkinson's disease, levodopa-induced dyskinesia in Parkinson's disease patients. Huntingdon's disease, Lewy Body Dementia, dementia due to HIV disease, dementia due to Creutzfeldt-Jakob disease, amnestic disorders, mild cognitive impairment, age-related cognitive decline, feeding disorders such as anorexia and bulimia, and obesity.
11. The antibody or antigen-binding antibody fragment, or pharmaceutical composition according to Embodiment 1 for use in the treatment or prophylaxis of ischemia, Parkinson's disease, memory impairment, Alzheimer's disease, dementia, and delirium tremens.
12. The antibody or antigen-binding antibody fragment, or pharmaceutical composition according to Embodiment 1 for use in the treatment or prophylaxis of neuropathic pain, central pain syndromes, postoperative pain, bone and joint pain, repetitive motion pain, dental pain, cancer pain, myofascial pain, perioperative pain, chronic pain, acute pain, dysmenorrhea, pain associated with angina, inflammatory pain, headache, migraine and cluster headache, primary hyperalgesia, secondary hyperalgesia, primary allodynia, secondary allodynia, or other pain.
13. Use of an antibody or antigen-binding antibody fragment, or pharmaceutical composition according to the invention for the manufacturing of a medicament for the treatment or prophylaxis of migraine, GERD, irritable bowel syndrome (IBS), overactive bladder (OAB), urinary incontinence, autism spectrum disorder, a neurological or psychiatric disorder, a behavior and dependence disorders, a neurological disorder, or pain.
14. The use according to Embodiment 13 wherein the treatment or prophylaxis is for symptoms associated with migraine, or reduces the frequency and/or severity of such one or more symptoms, optionally vasomotor symptoms (e.g. hot flashes, facial flushing, sweating, and night sweats), photophobia, phonophobia, sensitivity to smells, tearing/lacrimation, vertigo, dizziness, nausea, vomiting, headache pain, and aura.
15. The use according to Embodiment 13 wherein the migraine is selected from the group comprising episodic migraine, chronic migraine or cluster headache.
16. The use according to Embodiment 13 wherein the use is for the treatment or prophylaxis of a patient that has not previously received prophylactic therapy for migraine headaches.
17. The use according to Embodiment 13 wherein the treatment or prophylaxis is for treatment of gastroesophageal reflux disease is nonerosive reflux disease or erosive esophagitis.
18. The use according to Embodiment 13 wherein the treatment or prophylaxis is for diarrhea predominant IBS, constipation predominant IBS, or alternating bowel movement predominant IBS.
19. The use according to Embodiment 13 wherein the treatment or prophylaxis is for urgency, urinary frequency, nocturia, or urge incontinence.
20. The use according to Embodiment 13 wherein the treatment or prophylaxis is for stress incontinence, urge incontinence, overflow incontinence, mixed incontinence, structural incontinence, functional incontinence, nocturnal incontinence, transient incontinence, giggle incontinence, double incontinence, post-void dribbling, and coital incontinence.
21. The use according to Embodiment 13 wherein the treatment or prophylaxis is for impaired social and functional communication, anxiety, inattention, hyperactivity, altered sensory reactivity, self-injury, aggression, impaired cognitive function, and compromised daily living skills.
22. The use according to Embodiment 13 wherein the treatment or prophylaxis is for schizophrenia, schizophreniform disorder, schizoaffective disorder, delusional disorder, brief psychotic disorder, shared psychotic disorder, psychotic disorder due to a general medical condition, substance-induced psychotic disorder, psychotic disorder not otherwise specified, psychosis associated with dementia, major depressive disorder, dysthymic disorder, premenstrual dysphoric disorder, depressive disorder not otherwise specified, bipolar I disorder, bipolar II disorder, cyclothymic disorder, bipolar disorder not otherwise specified, mood disorder due to a general medical condition, substance-induced mood disorder, mood disorder not otherwise specified, generalized anxiety disorder, obsessive-compulsive disorder, panic disorder, acute stress disorder, post-traumatic stress disorder, mental retardation, pervasive developmental disorders, attention deficit disorders, attention-deficit/hyperactivity disorder, disruptive behavior disorders, personality disorder of the paranoid type, personality disorder of the schizoid type, personality disorder of the schizotypical type, tic disorders, Tourette's syndrome, substance dependence, substance abuse, substance withdrawal, trichotillomania, and conditions wherein cognition is impaired, Alzheimer's disease, Parkinson's disease, levodopa-induced dyskinesia in Parkinson's disease patients, Huntingdon's disease, Lewy Body Dementia, dementia due to HIV disease, dementia due to Creutzfeldt-Jakob disease, amnestic disorders, mild cognitive impairment, age-related cognitive decline, feeding disorders such as anorexia and bulimia, and obesity.
23. The use according to Embodiment 13 wherein the treatment or prophylaxis is for ischemia, Parkinson's disease, memory impairment, Alzheimer's disease, dementia, and delirium tremens.
24. The use according to Embodiment 13 wherein the treatment or prophylaxis is for neuropathic pain, central pain syndromes, postoperative pain, bone and joint pain, repetitive motion pain, dental pain, cancer pain, myofascial pain, perioperative pain, chronic pain, acute pain, dysmenorrhea, pain associated with angina, inflammatory pain, headache, migraine and cluster headache, primary hyperalgesia, secondary hyperalgesia, primary allodynia, secondary allodynia, or other pain.

Having described the invention above, the following examples are provided to further demonstrate the invention and its inherent advantages. These examples are offered to illustrate, but not to limit, the claimed invention.

### EXAMPLES

### Example 1: Generation, Selection, and Expression of Anti-mGluR5 Monoclonal Antibodies

Materials and Methods

Anti-mGluR5 mAb generation and selection. New Zealand White rabbits were immunized with mGluR5 sequences derived from the extracellular N-terminal domain. Spleen B cells from immunized rabbits expressing antibodies of interest were sorted and clonally expanded. Antibody sequences were recovered from clonal B cell wells using a combined RT-PCR method. Primers containing restriction enzymes were designed to anneal in conserved and constant regions of the target immunoglobulin genes (heavy and light), such as rabbit immunoglobulin sequences, and a two-step nested PCR recovery was used to amplify the antibody sequence. Amplicons from each well were sequenced and analyzed. Representative antibodies from the resulting sequence clusters are selected for recombinant protein expression. The original heavy and light variable regions amplified from rabbit cells are cloned into human heavy and light chain constant region expression vectors via restriction enzyme digestion and ligation, and via Gibson method. See Gibson et al., “Enzymatic assembly of DNA molecules up to several hundred kilobases,” Nature Methods 2009; 6(5):343-5. Vectors containing subcloned DNA fragments were amplified and purified. The sequences of the subcloned heavy and light chains were verified prior to expression. Seven additional antibodies were identified based on sequence homology to the representative antibodies.

Expression. Heavy and light chain plasmids were co-transfected to generate rabbit/human chimeric antibodies for testing. In particular, heavy and light chimeric plasmids were transiently transfected into HEK-293 cells. Transfections were allowed to incubate for 5-7 days, and upon harvest, cells were pelleted by centrifugation. Supernatants were purified via Protein A resin.

Results

From the above selection method, twenty-nine purified chimeric antibodies against human mGluR5 are exemplified here. These antibodies are designated Ab1-Ab29, with variable domain and complementarity-determining region (CDR) sequences contained in SEQ ID NOS:6-469.

### Example 2: Anti-Human mGluR5 mAbs Bind In Vitro to Human, Rat, and Monkey mGluR5, but not Human mGluR1

Materials and Methods

Design of mGluR5 and mGluR1 extracellular domain proteins. Expression plasmids were constructed to generate human mGluR5 extracellular domain (human-mGluR5-ECD) containing the first 505 amino acids of mGluR5 from NCBI Accession No. NM_000842 (SEQ ID NO:1 or 2)¹ with a C-terminal Flag tag DYKDDDDK using synthetic gBlock DNA (Integrated DNA Technologies). The mGluR5-ECD sequence was modified to contain a mutation C->S at amino acid position 238, similar to the mGluR5-ECD which was expressed to make the mGluR5 three-dimensional structure complexed with glutamate (EMBL-EBI Structure No. 3LMK). In a similar fashion, expression constructs for ECD of rat mGluR5 (NCBI Accession No. NM_017012; SEQ ID NO:3), cynomolgus monkey mGluR5 (NCBI Accession No. XM_005579309; SEQ ID NO:4), and human mGluR1 (NCBI Accession No. NM_001278064; SEQ ID NO:5) were generated. ¹ These amino acids are identical for mGluR5b and mGluR5a isoforms.

Expression andpurification ofECD proteins. HEK-293 cells were transfected with the expression constructs, and supernatants were harvested after 5-7 days. The expressed mGluR5-ECD proteins were purified using anti-Flag resin.

Binding of antibodies to mGluR5/mGluRJ measured via Homogeneous Time-Resolved Fluorescence (HTRF). Binding assays were performed using HTRF reagents from CisBio. Ten microliters of an antibody dilution series (highest final concentration of 30 nM) was incubated with 10 μl of mGluR-ECD (10 nM final concentration) in HTRF buffer (50 mM Phosphate buffer pH7, 0.8M KF (potassium fluoride), 0.2% BSA). Twenty microliters of Europium-labeled anti-hu-Fc donor (2 nM final concentration, CisBio Catalog No. 61HFCKLB) and 20 μl of XL665-labeled anti-Flag acceptor (60 nM final concentration, CisBio Catalog No. 61FG2XLB) were added to each well and incubated for 1 hour at room temperature. Alternatively, the antibody dilution series was incubated with 10 μl of biotinylated mGluR-ECD (10 nM final concentration), and 20 μl of Europium-labeled anti-hu-Fc donor (2 nM final concentration) and 20 μl of d2-labeled streptavidin acceptor (60 nM final concentration, CisBio Catalog No. 610SADLB) were added to each well. Fluorescence was measured at 620 and 665 nm with a delay of 300 μsec. Results were expressed as the ratio of signal at 665 nm over 620 nm. EC₅₀ values were calculated for binding of Ab1-Ab23 to human-mGluR5-ECD, of Ab1-Ab21 to rat-mGluR5-ECD, and of Ab1, Ab2 and Ab4-Ab12 to monkey-mGluR5-ECD and to human-mGluR1-ECD (TABLE 1).

mAb binding to mGluR5 measured via AlphaLISA. Binding assays were also performed using AlphaLISA reagents from Perkin Elmer. Twenty microliters of an antibody dilution series (highest final concentration of 1 μg/ml) was incubated with 20 sl of biotinylated human-mGluR5-ECD (0.2 μg/ml final concentration) in AlphaLISA stimulation buffer (Perkin Elmer Catalog No. AL000F). To each well was added 20 μl of anti-human-IgG acceptor beads (Perkin Elmer Catalog No. AL103M) and 20 μl of streptavidin donor beads (Perkin Elmer Catalog No. 6760002), each at 10 μg/ml final concentration. After incubation for 90 minutes at room temperature, the fluorescence signals were measured and EC₅₀ values for binding were calculated for Ab24-Ab29 (TABLE 1).

Results

Anti-mGluR5 antibodies Ab1-Ab29 each bind tightly and specifically to mGluR5, as summarized in Table 1. Each of these antibodies binds to human mGluR5 with an ECso of less than 2 nM. In addition to binding the human protein, antibodies Ab1-Ab7, Ab9-Ab19, and Ab21 were also shown to cross-react with rat-mGluR5. Further, Ab1, Ab2, and Ab4-Ab12 cross-reacted with cynomolgus monkey mGluR5, but none of these antibodies bound to human mGluR1. Exemplary antibodies AbA, AbB, and AbC were selected from among these 29 antibodies for further testing, as described below.

TABLE 1

| | mGluR-ECD binding EC50-pM | | | | |
|---|---|---|---|---|---|
| | | | rat- | cyno- | hu- |
| | hu-mGluR5 | | mGluR5 | mGluR5 | mGluR1 |
| Ab # | HTRF | AlphaLISA | HTRF | HTRF | HTRF |
| 1 | 268.5 | | 496.7 | 370.9 | ND |
| 2 | 341.3 | | 476.0 | 360.2 | ND |
| 3 | 536.2 | | 486.7 | | |
| 4 | 292.7 | | 332.1 | 286.3 | ND |
| 5 | 540.4 | | 311.1 | 310.1 | ND |
| 6 | 414.6 | | 771.4 | 424.3 | ND |
| 7 | 1020.0 | | 630.0 | 370.2 | ND |
| 8 | 748.4 | | ND | 322.4 | ND |
| 9 | 998.2 | | 519.0 | 398.2 | ND |
| 10 | 409.5 | | 848.0 | 338.5 | ND |
| 11 | 544.7 | | 422.9 | 254.6 | ND |
| 12 | 775.2 | | 991.0 | 359.4 | ND |
| 13 | 182.6 | | 283.3 | | |
| 14 | 370.8 | | 308.5 | | |
| 15 | 506.0 | | 448.3 | | |
| 16 | 439.3 | | 426.6 | | |
| 17 | 312.1 | | 304.6 | | |
| 18 | 490.9 | | 394.7 | | |
| 19 | 452.5 | | 327.2 | | |
| 20 | 364.4 | | ND | | |
| 21 | 684.2 | | 229.4 | | |
| 22 | 439.0 | | | | |
| 23 | 367.0 | | | | |
| 24 | | 94.7 | | | |
| 25 | | 91.5 | | | |
| 26 | | 101.3 | | | |
| 27 | | 77.2 | | | |
| 28 | | 91.8 | | | |
| 29 | | 89.1 | | | |

ND: not detected

### Example 3: Competition Binding Analysis of Exemplary Anti-mGluR5 mAbs Reveals Three Binding Epitope Bins

Materials and Methods

Octet96 Red system competition binding analysis. Experiments were performed to determine if mGluR5 monoclonal antibodies bound to different regions on the mGluR5-ECD using the Octet96 Red system from Pall ForteBio. Biotinylated mGluR5-ECD (1 μg/ml) was bound to a streptavidin sensor (Pall ForteBio Catalog No. 18-5019). After a brief wash step, an antibody (at 10 μg/ml) was added to the immobilized mGluR5-ECD and allowed to reach saturation with a plateau in binding signal. Saturation was confirmed by adding the same antibody again after a wash and seeing no additional signal. After another wash step, a second antibody (at 10 μg/ml) was added. A binding signal from the second antibody indicated that the two antibodies recognized different regions and could bind the target independently of each other.

Results

AbA, AbB, and AbC are three specific exemplary antibodies selected from Ab1-Ab29 which are representative of anti-mGluR5 antibodies having different binding epitopes. The results of these experiments indicated that the exemplary mGluR5 monoclonal antibodies AbA, AbB, and AbC did not compete with each other for binding to mGluR5-ECD and represent 3 different bins (FIG. 1). These exemplary antibodies are further employed in the following examples, to show representative results for each of these three bins.

### Example 4: Anti-mGluR5 mAbs Bind to Full-Length Human mGluR5 on the Cell Surface

Materials and Methods

Generation of mGluR5-transfected cell line. An expression vector for full-length (FL) human mGluR5 was obtained from Genecopoeia (Catalog No. EX-Z5904-M02). This was transfected into BA/F3 cells (DSMZ No. ACC-300) and stable clones were selected. Human mGluR5 surface expression was confirmed by flow cytometry using anti-mGluR5 exemplary antibody, AbA. BA/F3-mGluR5 cell clone 10C9 was determined to express mGluR5 on the cell surface and was used for binding and signaling assays.

Antibody binding by flow cytometry. Ab1-Ab29 were tested by flow cytometry on BA/F3-mGluR5 clone 10C9 cells in order to confirm binding of antibodies to full-length mGluR5 target expressed on the cell surface. Cells (2×10⁵) were incubated 1 hr on ice with 10 μg/ml antibody in FACS buffer (2% FBS in PBS). Cells were washed with FACS buffer and then incubated 30 min on ice with FITC-tagged anti-hu-Fc secondary antibody (Jackson ImmunoResearch Catalog No. 709-096-098, diluted 1:100 in FACS buffer). Cells were again washed in FACS buffer and then analyzed on a BD Accuri™ C6 flow cytometer. Results are expressed as fold mean fluorescence intensity (MFI) signal over background, which was defined by the MFI signal of the secondary antibody alone.

Antibody affinities determined via flow cytometry saturation binding assay. Antibodies were titrated with BA/F3-mGluR5 cell clone 10C9 (2×10⁵ cells per sample) in FACS buffer (PBS containing 2% fetal bovine serum) and incubated for 4 hours on ice with regular mixing. Samples were washed 2 times in FACS buffer and then incubated with FITC-labeled anti-huFc secondary antibody (1:100, Jackson ImmunoResearch Catalog No. 709-096-098) for 1 hour on ice. Cells were washed 2 more times in FACS buffer and MFI signal was determined using a BD Accuri™ C6 flow cytometer.

Results

The results of the flow cytometry assay are summarized in TABLE 2, showing a 10-42 fold signal over background for all of the tested antibodies. These results indicate that each of the anti-mGluR5 antibodies Ab1-Ab29 binds to full-length human mGluR5 on the cell surface.

Results of the binding affinity determination are shown in FIG. 2 for Exemplary antibodies AbA, AbB, and AbC. The EC₅₀ values determined for these antibodies were 0.14 nM, 1.68 nM, and 1.54 nM, respectively.

TABLE 2

| | mGluR5 (FL) cell binding-fold over background |
|---|---|
| Ab # | FACS |
| 1 | 40.8 |
| 2 | 40.3 |
| 3 | 31.1 |
| 4 | 41.8 |
| 5 | 36.3 |
| 6 | 24.4 |
| 7 | 28.9 |
| 8 | 32.1 |
| 9 | 28.4 |
| 10 | 27.9 |
| 11 | 37.0 |
| 12 | 31.9 |
| 13 | 37.4 |
| 14 | 40.6 |
| 15 | 41.3 |
| 16 | 40.0 |
| 17 | 40.4 |
| 18 | 39.6 |
| 19 | 38.3 |
| 20 | 26.1 |
| 21 | 34.2 |
| 22 | 10.9 |
| 23 | 37.9 |
| 24 | 37.2 |
| 25 | 37.9 |
| 26 | 39.4 |
| 27 | 37.8 |
| 28 | 38.9 |
| 29 | 39.6 |

### Example 5: Anti-Human mGluR5 Antibodies Successfully Inhibit mGluR5 In Vitro Via Non-Competitive Inhibition

Materials and Methods

p-ERK signaling assay and inhibition by mGluR5 antibodies. An mGluR5 signaling assay was established using the BA/F3-mGluR5 clone 10C9 cells. Upon incubation with quisqualate, a glutamate-like agonist of mGluR5, these cells generate production of cytosolic phospho-ERK (p-ERK) which was quantitated using an AlphaLISA SureFire Ultra p-ERK Assay Kit from Perkin Elmer (Catalog No. ALSU-PERK-A10K). To measure inhibition by mGluR5 antibodies, cells were stimulated with quisqualate in the presence of increasing amounts of antibody, and IC₅₀ values were obtained. Briefly, cells (2×10⁵/well) were pelleted in a V-bottom 96-well plate, then resuspended in 70 μl/well of an antibody dilution series in assay buffer (RPMI-1640+10% dialyzed FBS) and incubated for 1 hour at 37° C. Cells were pelleted, washed in stimulation buffer (CisBio Catalog No. 62IP1FDG), and re-pelleted. Cells were then resuspended in 70 μl/well of 10 μM quisqualate (Tocris Catalog No. 52809-07-01) in stimulation buffer and incubated 20 minutes at 37° C., at which time cells were again washed and pelleted. Fifty p./well of lysis buffer was then added and allowed to incubate 10 minutes with shaking at ˜350 RPM. Thirty μl of each lysate was transferred to a fresh plate where 15 μl of Acceptor mix was added and incubated for 1 hour at room temperature in the dark, followed by addition of 15 μl of Donor mix with incubation for an addition hour at room temperature in the dark. Fluorescence signals were measured and IC₅₀ values determined for Ab1-Ab29 (TABLE 3).

Exemplary mGluR5 antibodies do not compete for ligand binding. Experiments were performed to determine the mechanism of inhibition by the mGluR5 monoclonal antibodies. Utilizing an mGluR5 binding assay, the antibodies were tested for whether they acted by inhibiting the binding of the ligand molecule. Antibodies (3.33 μM final concentration) were pre-incubated with cell membrane homogenates (about 50 μg protein) from CHO cells transfected with human mGluR5 in an incubation buffer containing 20 mM Hepes/Na OH (pH 7.4), 2 mM MgCl2 and 2 mM CaCl2). [³H]Quisqualate (40 nM final concentration) was then added to the mixture and samples were incubated for 120 min at 22° C. Following incubation, the samples were filtered rapidly under vacuum through glass fiber filters (GF/B, Packard) presoaked with the incubation buffer and rinsed several times with ice-cold 50 mM Tris-HCl using a 96-sample cell harvester (Unifilter, Packard). The filters were dried then counted for radioactivity in a scintillation counter (Topcount, Packard) using a scintillation cocktail (Microscint 0, Packard). The results for Exemplary antibodies AbA, AbB and AbC are shown in FIG. 3 and are expressed as a percent inhibition of the control radioligand specific binding. As a positive control for the assay, unlabeled quisqualate was used to inhibit binding of the [³H]Quisqualate and was found to inhibit with an IC₅₀ of 48 nM.

Results

TABLE 3 shows the results of the cell-based mGluR5 inhibition assay for Ab1-Ab29, revealing that each antibody inhibited mGluR5 with an IC₅₀ of 0.06 nM-8.33 nM. In a follow-up study, exemplary antibodies AbA, AbB, and AbC, representative of the three epitope bins described in Example 3, were tested to determine the mode of inhibition. As shown in FIG. 3, none of these mGluR5 antibodies inhibited the binding of [³H]Quisqualate to the mGluR5-expressing membrane preparations.

TABLE 3

| Ab # | pERK Assay Inhibition IC50-nM |
|---|---|
| 1 | 0.14 |
| 2 | 0.16 |
| 3 | 3.91 |
| 4 | 0.34 |
| 5 | 1.51 |
| 6 | 4.29 |
| 7 | 1.34 |
| 8 | 2.29 |
| 9 | 2.97 |
| 10 | 1.07 |
| 11 | 0.14 |
| 12 | 3.73 |
| 13 | 0.22 |
| 14 | 0.15 |
| 15 | 0.18 |
| 16 | 0.17 |
| 17 | 0.15 |
| 18 | 0.15 |
| 19 | 1.07 |
| 20 | 2.36 |
| 21 | 1.41 |
| 22 | 8.33 |
| 23 | 0.14 |
| 24 | 0.10 |
| 25 | 0.11 |
| 26 | 0.10 |
| 27 | 0.06 |
| 28 | 0.11 |
| 29 | 0.09 |

### Example 6: mGluR5 Antibodies Show Efficacy in an In Vivo Model of Umbellulone-Driven Migraine Symptoms

Materials and Methods

Test of inhibition ofumbellulone-induced lacrimation by anti-mGluR5 monoclonal antibodies. Lacrimation (tearing) is regulated by the parasympathetic nervous system. Noxious chemical stimulation of rat facial mucosa, using chemicals such as umbellulone (an extract from the “headache tree” Umbellularia californica), has been shown to increase facial and intracranial blood flow, as well as lacrimation, through activation of the trigemino-parasympathetic reflex, an experimental model for vascular dysfunctions in cluster headache and migraine (Gottselig R, Messlinger K. Cephalalgia. 2004 March; 24(3):206-14; Nassini R et al. Brain. 2012; 135(2):376-90). For this experiment, umbellulone oil (Sigma Aldrich, Lot No. 083M4714V) was diluted to 0.2 μmol/kg in 0.5% DMSO in PBS in an amber glass vial. Male Sprague Dawley rats (n=64) aged approx. 6 weeks upon arrival were obtained from Envigo, Inc., Indianapolis, Ind. Rats weighed approximately 276-310 g (mean approx. 288 g) on Study Day −1. Twenty-four hours after test article administration (IV dosing, 20 mg/kg), animals were anesthetized with inhaled Isoflurane (VetOne, Catalog No. 502017) and administered umbellulone (at 0.2 μmol/kg) or vehicle intranasally (IN, 50 μl/rat) 2 mm into the right nostril over a 5 second period. Sixty minutes post IN dosing, animals were anesthetized and a modified Schirmers test strip was placed on the medial side of the right lower eyelid for a period of 5 minutes. After 5 minutes the test strip was read for tear production using the pre-printed millimeter hash marks on the strip. Results for Exemplary AbA and AbB are shown in FIG. 4.

Test of inhibition of umbellulone-induced facial temperature increase by anti-mGluR5 monoclonal antibodies. Noxious chemicals such as umbellulone administered intranasally have been shown to stimulate rat facial mucosa and increase facial and intracranial blood flow through activation of the trigeminal-parasympathetic reflex. Nose temperature increases that result from increased facial blood flow can be measured using an infrared (IR) thermometer (Gottselig R, Messlinger K. Cephalalgia. 2004 March; 24(3):206-14; Nassini R et al. Brain. 2012; 135(2):376-90). In this experiment, umbellulone oil (Sigma Aldrich, Lot No. 083M4714V) was diluted to 0.2 μmol/kg in 0.5% DMSO in PBS in an amber glass vial. Male Sprague Dawley rats were obtained from Envigo, Indianapolis, Ind. The rats weighed 276-335 grams (mean of 303 g). Antibody treatment groups were administered antibodies approximately 24 hours prior to testing (IV dosing, 15 mg/kg). One hour prior to testing, small molecule treatment groups received IV injections of test article (20 mg/kg) or vehicle. For the IV injections the tail was dipped in warm water. For umbellulone administration, animals were briefly anesthetized with inhaled Isoflurane (VetOne, Catalog No. 502017) and then had 50 μl of irritant or vehicle administered intranasally (IN) 2 mm into the right nostril over a 5 second period. At pre-dose and 5 minutes post IN dosing, animals had their nose and right foot temperature taken with a Thermoworks TW2 IR thermometer with emissivity set to 0.97. The thermometer was held within approximately 2.5 cm of the nose or footpad for the reading. Results for Exemplary AbA, AbB, and AbC are shown in FIG. 5, compared to results for the mGluR5 small molecule inhibitor ADX10059 (Tocris).

Results

FIG. 4 demonstrates that exemplary mGluR5 blocking mAbs AbA, AbB, and AbC prevented umbellulone-induced lacrimation in rats. In addition, results in FIG. 5 show that mGluR5 blocking mAbs AbA, AbB, and AbC, and a previously described small molecule mGluR5 inhibitor (ADX10059), prevented headache tree extract umbellulone-induced facial temperature increase in rats.

Thus, the results indicate that the mGluR5 antibodies exemplified herein were effective in preventing symptoms of cluster headache and migraine in an in vivo model.

### Example 7: mGluR5 Antibody Administration does not Lead to Deficiencies in Motor Coordination

Materials and Methods

Motor coordination test. A rotarod test is used to calculate motor dysfunction produced by centrally acting drugs to determine possible alterations in the motor coordination of the animal, based on the assumption that a rodent with normal motor efficiency is able to maintain its equilibrium on a rotating rod. The difference in the fall time from the rotating rod is taken as an index of muscle relaxation (Kudagi B et al. J Dent Med Sci. 2012; 1(4):42-7). A small molecule inhibitor that penetrates the brain may cause unwanted side effects that reduce muscle coordination, while a blocking antibody against the same target, which does not significantly partition into the brain may have little to none of the unwanted muscle coordination side effects. The rotarod test was used to test these differences between small molecule and antibody side effects. Male Sprague Dawley rats were obtained from Envigo, Indianapolis, Ind. The rats weighed 276-335 grams (mean of 303 g). Prior to the start of the study, the animals were trained on the rotarod. On Study Day −1, the animals were weighed and the baseline rotarod measurements were taken. The animals were randomized based on rotarod results. Antibody treatment groups were administered antibodies approximately 24 hours prior to rotarod testing (IV dosing, 15 mg/kg). One hour prior to rotarod testing, small molecule treatment groups received IV injections of test article (20 mg/kg) or vehicle. At each testing event, three trials were performed with a 6-minute cut off of each trial and a minimum 5-minute inter-trial interval. Animals were placed on the rotarod and the rod began rotating at 4 RPM and increased up to 40 RPM over the course of 5 minutes. Latency to fall off the rotarod and final RPM was recorded for each animal. If an animal fell off prior to 10 seconds, this was not counted as a trial and the animal was placed on the rotarod to retry. Results for Exemplary AbA, AbB, and AbC are shown in FIG. 6, compared to results for the mGluR5 small molecule inhibitor ADX10059 (Tocris).

Results

As a measure of dizziness (a reported side effect of ADX10059), a motor coordination model in rats was employed, using performance on a rotarod as the readout. The results, shown in FIG. 6, indicate that mGluR5 mAbs do not impair motor coordination while ADX10059 significantly impaired motor coordination. Thus, mGluR5 mAbs demonstrate efficacy in relevant in vivo models of cluster headache/migraine symptoms with mGluR5 blocking mAbs of all three bins, while showing a lack of the “motor coordination/dizziness side effect observed with mGluR5 small molecule in human clinical trials.

mGluR5 antibodies may be tested for efficacy in a variety of models for various disease indications, based on the known biology of mGluR5 and the effects of other mGluR5 small molecule inhibitors. Antibodies may be delivered either prophylactically or therapeutically in a variety of routes in these models to improve symptoms, including intravenous, subcutaneous, or intrathecal administrations.

### Example 8: mGluR5 mAb Efficacy in an Irritable Bowel Syndrome Model

mGluR5 mAbs are tested in a rodent model involving colonic spasm/pain readouts in response to colonic distension, with or without additional irritant such as TNBS (2,4,6-Trinitrobenzenesulfonic acid) or acetic acid.

Materials and Methods:

Colorectal distension (CRD) is performed in rats treated with test or control antibodies by applying graded colorectal distensions according to a modified method by Tarrerias A et al., Pain 2002; 100:91-97. Abdominal striated muscle contractions induced by graded colorectal distension (10-60 mmHg, 10 mmHg increments, 3 min duration with 1 min deflation using a barostat) indicate the degree of visceral nociception. In some experiments, trinitrobenzene sulfonic acid (TNBS, 30 mg/kg in ethanol 25%) is instilled in the colon of rats to induce visceral hyperalgesia 7 days prior to experiments applying graded colorectal distensions. Alternatively, intracolonic acetic acid (1.5%) is instilled 3 days prior to the colorectal distensions.

Results:

The anti-mGluR5 monoclonal antibodies Ab1-Ab29 are expected to reduce muscle contractions in a dose-dependent manner.

### Example 9: mGluR5 mAb Efficacy in an Incontinence/Overactive Bladder Model

mGluR5 mAbs are tested in a rodent model involving bladder spasm/pain readouts in response to bladder distension.

Materials and Methods:

In anesthetized rats treated with test or control antibodies, the urinary bladder is catheterized by use of a PE50 polyethylene tubing filled with physiological saline. Intravesical pressure is measured by a pressure transducer. Cystometry is performed during constant infusion (0.06 ml/min) of saline into the bladder to elicit bladder contractions (Tagaki-Matzumoto et al., J Pharmacol Sci 2004; 95:458-465).

Results:

The anti-mGluR5 monoclonal antibodies Ab1-Ab29 are expected to increase threshold volumes eliciting bladder contractions in a dose-dependent manner.

### Example 10: mGluR5 mAb Efficacy in a Gastro-Esophageal Reflux Disease Model

mGluR5 mAbs are tested in a rodent model involving esophageal pH changes after acidic meal challenge.

Materials and Methods:

In ferrets, the measure of esophageal pH changes above the sphincter after acidic meals is used to predict effects in gastro-esophageal reflux disease treatment. See, e.g., Frisby C L et al. Gastroenterology. 2005; 129:995-1004. For this experiment, left lateral cervical esophagostomies are fashioned in young adult female ferrets (Mustela putorius furo L) under 2%-4% halothane anesthesia as described previously (Blackshaw L A et al. Neurogastroenterol Motil. 1998; 10:49-56). Ferrets are conditioned to experimental equipment and environmental settings at least 1 month following the esophagostomy surgery using reward-based training.

Esophageal, LES (lower esophageal sphincter), and gastric pressures are recorded using a custom-designed multilumen micromanometric assembly (Dentsleeve, Wayville, South Australia). LES pressure is measured using a 3-cm reverse-perfused sleeve sensor incorporated into the micromanometric assembly. Four side holes positioned 1.25, 2.25, 4.75, and 7.25 cm proximal to midsleeve are used for esophageal pressure recording, with a fifth side hole 2.25 cm distal to midsleeve for gastric pressure monitoring. The central channel of the assembly is used for administration of gastric loads. A constant perfusion manometric pump (Dentsleeve) is used to perfuse the manometric assembly at 0.02 mL/min for esophageal side holes and 0.04 mL/min for the sleeve and gastric side hole. To enable measurement of concurrent pH events, a single-sensor multiuse pH catheter (Flexilog; Oakfield Instruments Ltd, Eynsham, Oxfordshire, England) together with a KCl reference electrode are secured to the micromanometric assembly with 2-mm-wide strips of Parafilm (American National Can, Chicago, Ill.), with the sensor 2 cm proximal to midsleeve to ensure it records esophageal pH in situ. To protect the manometric/pH assembly from bite damage during the study, the assembly is passed through a coil spring that is attached to a purpose-built Neoprene harness (Clark Rubber, Adelaide, Australia). Animals are manually restrained during intubation, and positioning of the sleeve sensor at the LES is achieved by overintroducing the assembly so that the sleeve lays within the stomach and slowly withdrawing it until the highpressure zone is clearly recorded. A microphone sewn into the harness is positioned under the hyoid bone to allow detection of the onset of swallows. Ferrets are free to move around a cylindrical housing chamber 30 cm in diameter mounted on a freely rotating bearing for the duration of the experiment. Visual inspection of ferret posture and movements during the experimental period is facilitated through a small clear plastic window. Manometric and pH recordings are amplified using Synectics Medical Polygraf amplifiers (Stockholm, Sweden) and acquired with Labview-based Trace! Software (version 2.1; Dr G. S. Hebbard, Heidelberg, Victoria, Australia).

Experimental studies are conducted after an overnight fast. Ferrets treated with test or control antibodies are intubated and then secured into the experimental apparatus both to allow an acclimatization period and to enable appropriate positioning of the micromanometric/pH monitoring assembly. Data acquisition is commenced 25 minutes before gastric loads. The gastric load designed to trigger TLESR (transient lower esophageal sphincter relaxation) consists of 25 mL 10% acidified glucose (pH 3.5) administered over 2 minutes, followed by three 10-minute air infusions (1 mL/min), each separated by 5-minute rest periods.

Micromanometric recordings are analyzed with Trace! software using manometric indices previously established for this model (Blackshaw L A et al. Neurogastroenterol Motil. 1998; 10:49-56). TLESRs are identified as rapid (>1 mm Hg/s) decreases in LES pressure to 2 mm Hg or less above gastric pressure for more than 5 seconds, with no associated swallowing or esophageal peristalsis at their onset. Basal LES pressure is determined from the mean LES pressure above gastric pressure during the 3 rest periods between gastric air infusions.

Reflux of acid into the esophagus is scored as a reflux episode if the intraesophageal pH decreased to a value of at least pH 4 for more than 5 seconds while the position of the pH probe could be verified as above the high-pressure zone of the LES.

Results:

The anti-mGluR5 monoclonal antibodies Ab1-Ab29 are expected to attenuate TLESRs, reflux episodes, and intraesophageal pH decreases in a dose-dependent manner.

### Example 11: mGluR5 mAb Efficacy in a Neurogenic Dural Vasodilation Migraine Model

mGluR5 mAbs are tested in a rodent model measuring vasodilation in the dura after electrical stimulation near the vessel.

Materials and Methods:

In rats, the neurogenic dural vasodilation model is used to predict effects in migraine treatment. See, e.g., Waung M W et al. Annals of clinical and translational neurology 2016; 3(8):560-71. For this experiment, a thinned parietal bone window is created over the dural meningeal artery until the vessel is clearly visualized (Akerman S et al. J Neurosci 2013; 33:14869-77.). Images of the artery are captured by microscope and changes in vessel diameter are measured over time. A bipolar stimulating electrode is positioned on the cranial window surface within 200 μm of the vessel of interest in animals treated with test or control antibodies. A 10 sec train of 5 Hz stimulation is administered with 1 msec pulses between 10 and 40 V to achieve maximal vessel dilation. This maximal response voltage is used in the same animal throughout the experiment. Electrical stimulation is repeated at 5-15 min intervals over 1 h in the presence of test or control antibodies. Effects of electrical stimulation on dural vessel diameter are calculated as a percentage increase from prestimulation baseline diameters.

Results:

The anti-mGluR5 monoclonal antibodies Ab1-Ab29 are expected to attenuate neurogenic dural vasodilation in response to meningeal stimulation in a dose-dependent manner.

### Example 12: mGluR5 mAb Efficacy in an Excitatory TCC (Trigeminal-Cervical Complex) Nociceptive Stimulation Migraine Model

mGluR5 mAbs are tested in a rodent model measuring electrical recordings from neurons in the TCC, either spontaneous or after electrical stimulation near the middle meningeal artery in the dura.

Materials and Methods:

In rats, the TCC neuronal activity model is used to predict effects in migraine treatment. See, e.g., Waung M W et al. Annals of Clinical and Translational Neurology 2016; 3(8):560-71. For this experiment, a craniotomy is performed over the parietal bone with exposure of the dura mater overlying the middle meningeal artery (MMA). Cervical spinal cord hemi-laminectomy is also performed, and the dura mater incised to expose the brainstem at the level of the caudal medulla. A tungsten recording electrode (0.5 MΩ, tip diameter 0.5 μm) is lowered into the brainstem with a piezoelectric motor controller. Placement of the recording electrode into the V1 region of the trigeminal nucleus caudalis is guided by direct neuronal firing in response to cutaneous brush and pinch in the V1 ophthalmic dermatome. A bipolar stimulating electrode is placed on the dura mater adjacent to the MMA and square-wave stimuli (0.5 Hz) of 0.1-0.3 msec duration, 10-25 V are applied to activate trigeminal afferents. Extracellular recordings are made from neurons in the TCC activated by MMA stimulation in animals treated with test or control antibodies. An average of three baselines for comparisons is used or compared to vehicle control recordings. The signal is amplified and passed through filters and a 60-Hz noise eliminator to a second-stage amplifier. This signal is fed to a gated amplitude discriminator and analog-to-digital converter and to a microprocessor-based computer for analysis.

Results:

The anti-mGluR5 monoclonal antibodies Ab1-Ab29 are expected to attenuate TCC neuronal activity in response to meningeal stimulation in a dose-dependent manner.

### Example 13: mGluR5 mAb Efficacy in an Anxiety Model

mGluR5 mAbs are tested in a rodent model measuring the number of marbles buried in the cage bedding in a set period of time.

Materials and Methods:

In mice, the marble burying model is used to predict effects in anxiety treatment. See, e.g., Spooren WPJM et al. J Pharmacol Exp Ther 2000; 295:1267-1275. For this experiment, the test procedure for marble burying is adopted with minor modifications from the original description of Broekkamp et al. Eur J Pharmacol 1986; 126:223-229. Briefly, mice treated with test or control antibodies are removed from the cage and individually placed in small cages (22×16×14 cm) in which 10 marbles have been equally distributed on top of a 5-cm sawdust bedding. The mice are left undisturbed in these cages for 60 min; after removal of the mouse the number of visible, nonburied marbles (i.e., less than two-thirds covered by sawdust) is counted.

Results:

The anti-mGluR5 monoclonal antibodies Ab1-Ab29 are expected to attenuate the marble burying behavior in a dose-dependent manner.

### Example 14: mGluR5 mAb Efficacy in an Addictive Disorder Model

mGluR5 mAbs are tested in a rodent model measuring drug self-administration after training to self-administer drugs such as morphine.

Materials and Methods:

The rodent drug self-administration test is used for evaluation of drug addiction treatments. The apparatus for the drug self-administration test is a standard, commercially available operant conditioning chamber. Before drug trials begin, rats are trained to press a lever for a food reward. After stable lever pressing behavior is acquired, rats are tested for acquisition of lever pressing for drug reward. Rats are trained to self-administer a known drug of abuse, such as morphine. Rats are then presented with two levers, an “active” lever and an “inactive” lever. Pressing of the active lever results in drug infusion on a fixed ratio schedule followed by a 20 second time out period. Pressing of the inactive lever results in infusion of excipient. Training continues until the total number of morphine infusions stabilizes to within 10% per session. Trained rats treated with test or control antibodies are allowed to self-administer drug as usual. Data is analyzed as the change in number of drug infusions per testing session compared to training session.

Results:

The anti-mGluR5 monoclonal antibodies Ab1-Ab29 are expected to induce a lower rate of response to active drug in a dose-dependent manner.

### Example 15: mGluR5 mAb Efficacy in a Levodopa-Induced Dyskinesia Model

mGluR5 mAbs are tested in an animal model measuring involuntary movements induced by MPTP (1-methyl-4-phenyl-1,2,3,6-tetrahydropyridine) or 6-OHDA (6-hydroxydopamine).

Materials and Methods:

The rodent model of Parkinson's disease induced with 6-OHDA and treated with levodopa is used to test treatments for the involuntary movements associated with this condition. Experimental parkinsonism is achieved through unilateral injection of 6-hydroxydopamine (6-OHDA, 3 μg/μL; Sigma) into the striatum as described previously (Lundblad M et al., Neurobiol Dis 2004; 16:110-123). 6-OHDA is dissolved in saline containing 0.02% ascorbic acid and unilaterally injected in the striatum at the following two coordinates from bregma: 2 μL at +0.3 mm anteroposterior, +2.2 mm lateral, and −3.0 mm dorsoventral below dura and 2 μL at +1.1 mm anteroposterior, +1.7 mm lateral, and −2.9 mm dorsoventral. After 3 wk of recovery, animals treated with test or control antibodies undergo chronic injection of L-DOPA (20 mg/kg, s.c.) once daily for 14 d for development of AIMs (abnormal involuntary movements). The four AIMs categories (limb, axial, orolingual, and locomotive) are scored using a validated rating scale (Lundblad M et al., Eur J Neurosci 2002; 15:120-132) for 1 min every 20 min for 2 h by a blinded trained investigator.

Results:

The anti-mGluR5 monoclonal antibodies Ab1-Ab29 are expected to induce a lower number of AIMs in a dose-dependent manner.

### Example 16: mGluR5 mAb Efficacy in a Dystonia Model

mGluR5 mAbs are tested in a rodent model measuring abnormal excitatory nerve and behavioral responses due to overexpression of Torsin A protein.

Materials and Methods:

The rodent model overexpressing TorsinA protein is used to identify treatments for dystonia. See Sciamanna G et al., Neuropharm 2014; 85:440-50. Human TorsinA transgenic mice (8- to 10-weeks old) are generated as previously described (Sharma N et al., J Neurosci 2005; 25:5351-5). Mice are sacrificed by cervical dislocation, then brain slices are prepared and transferred into a recording chamber. Current clamp recordings in response to various stimuli are performed in the presence of test or control antibodies in the perfusion solution.

Results:

The anti-mGluR5 monoclonal antibodies Ab1-Ab29 are expected to reduce the abnormal membrane responses in a dose-dependent manner.

### Example 17: mGluR5 mAb Efficacy in an Acute Pain Model

mGluR5 mAbs are tested in a rodent model measuring tail flick responses to heat.

Materials and Methods:

The tail flick method is utilized to study the antinociceptive activity in rodents. See Rezaee-Asl M et al., Int Sch Res Notices 2014; Article ID 687697:5 pages. A radiant heat automatic tail flick analgesiometer is applied to measure reaction latencies. Basal reaction time to radiant heat of animals treated with test or control antibodies is recorded by locating the tip (last 1-2 cm) of the tail on radiant heat source. The tail removal from the radiant warmth is taken as end point. The cutoff time of 15 seconds is used to avoid tail injury by heat.

Results:

The anti-mGluR5 monoclonal antibodies Ab1-Ab29 are expected to attenuate the pain response in a dose-dependent manner.

### Example 18: mGluR5 mAb Efficacy in a Chronic Pain Model

mGluR5 mAbs are tested in a rodent model measuring pain responses using Von Frey filaments after injection of acid saline solution over several days in the calf muscle, often used as a model of fibromyalgia.

Materials and Methods:

Repeated acid injection into the calf muscle is used as a model of chronic pain and is used to test analgesics. See Nielsen A N et al., Eur J Pharmacol 2004; 287:93-103. Mice treated with test or control antibodies are injected twice with 20 μL of pH 4 sterile saline into one gastrocnemius muscle 5 days apart. This results in a bilateral mechanical hyperalgesia that lasts through 4 weeks, does not depend on continued primary afferent input and has no damage to the injected muscle (Sluka K A et al., Muscle Nerve 2001; 24:37-46). Mechanical hyperalgesia is measured as the number of responses out of five to repeated applications of a 0.4 mN von Frey filament to the plantar surface of the ipsilateral and contralateral hind paws. A response to the von Frey stimuli is defined as an abrupt foot lift upon application of the von Frey filament. Each trial of the five von Frey stimuli are applied at approximately 1/s. Ten trials are averaged to give one response per animal with 5 min between each of the 10 trials.

Results:

The anti-mGluR5 monoclonal antibodies Ab1-Ab29 are expected to attenuate the pain response in a dose-dependent manner.

### Example 19: mGluR5 mAb Efficacy in a Postoperative Pain Model

mGluR5 mAbs are tested in a rodent model measuring weight-bearing ability after a paw incision.

Materials and Methods:

Weight-bearing after paw incision in rodents is used to analyze treatments for postoperative pain. See Zhu C Z et al., Pain 2005; 114:195-202. Paw incision is performed using halothane (2-3%) anesthesia and follows procedures previously described (Brennan T J et al., Pain 1996; 64:493-501). Briefly, the plantar aspect of the left hind paw is placed through a hole in a sterile plastic drape. A 1-cm longitudinal incision is made through the skin and fascia, starting 0.5 cm from the proximal edge of the heel and extending towards the toes, the plantar muscle is elevated and injured longitudinally leaving the muscle origin and insertion points intact. After hemostasis with gentle pressure, the skin is apposed with two mattress sutures (5-0 nylon). Hind paw weight-bearing response is assessed using the Incapacitance Analgesia Meter (Stoelting, Wood Dale, Ill.), which is a dual channel scale that separately measures the weight of the animal distributed to each hind paw. While normal rats distribute their body weight equally between the two hind paws (50-50), the discrepancy of weight distribution between an injured and noninjured paw is a natural reflection of the discomfort level in the injured paw. Rats treated with test or control antibodies are placed in the plastic chamber designed so that each hind paw rests on a separate transducer pad. The averager is set to record the load on the transducer over 5 s time period and two numbers displayed represent the distribution of the rat's body weight on each paw in grams (g). For each rat, three readings from each paw are taken and then averaged. Side-to-side weight-bearing difference is calculated as the average of the absolute value of the difference between two hind paws from three trials (right paw reading-left paw reading).

Results:

The anti-mGluR5 monoclonal antibodies Ab1-Ab29 are expected to attenuate the pain response and reduce the differences in weight-bearing between the 2 paws in a dose-dependent manner.

### Example 20: mGluR5 mAb Efficacy in a Neuropathic Pain Model

mGluR5 mAbs are tested in a rodent model measuring paw sensitivity to Von Frey filaments induced by the chemotherapy agent paclitaxel.

Materials and Methods:

The paclitaxel-induced paw sensitivity model is used to test treatments for painful peripheral neuropathy. See Xie J-D et al., J Biol Chem 2016; 291:19364-19373. To induce peripheral neuropathy, rats treated with test or control antibodies are injected intraperitoneally with paclitaxel (2 mg/kg) on four alternate days (days 1, 3, 5, and 7; total cumulative dose of 8 mg/kg). After 10 days, rats are individually placed on a mesh floor within suspended chambers and allowed to acclimate for at least 30 min. To determine their tactile sensitivity, we apply a series of calibrated von Frey filaments perpendicularly to the plantar surface of both hindpaws with sufficient force to bend the filament for 6 s. Brisk withdrawal or flinching of the paw is considered a positive response. In the absence of a response, we apply the filament of the next greater force. After a response, we apply the filament of the next lower force. The tactile stimulus producing a 50% likelihood of withdrawal response is calculated.

Results:

The anti-mGluR5 monoclonal antibodies Ab1-Ab29 are expected to attenuate the pain response and increase paw withdrawal threshold in a dose-dependent manner.

### Example 21: mGluR5 mAb Efficacy in an Inflammatory Pain Model

mGluR5 mAbs are tested in a rodent model measuring paw withdrawal after inducing inflammation by injection of Freund's complete adjuvant.

Materials and Methods:

The Freund's complete adjuvant (FCA) model of inflammatory pain is used to measure effects of drugs on established inflammatory hyperalgesia in rats. See Walker K et al., Neuropharmacology 2001; 40:109. FCA-induced inflammation of the rat hind paw is associated with the development of persistent inflammatory mechanical hyperalgesia (decrease in paw withdrawal threshold or PWT) and provides reliable prediction of the anti-hyperalgesic effects of clinically useful analgesic drugs (Bartho L et al., Naunyn Schmiedebergs Arch Pharmacol 1990; 342(6):666-70). The left hind paw of animals treated with test or control antibodies is injected, subplantar, with 25 sL of Freund's complete adjuvant (Sigma). PWT are determined prior to FCA treatment (pretreatment) and then again 24 h following FCA treatment (pre-dose). PWT are measured using the paw pressure technique (Stein C et al., Pharmacol Biochem Behav 1988; 31:451-455). The analgesymeter (7200, Ugo Basile, Italy) employs a wedge-shaped probe (area 1.75 mm2). Cutoff is set at 250 g and the end point is taken as paw withdrawal.

Results:

The anti-mGluR5 monoclonal antibodies Ab1-Ab29 are expected to attenuate the pain response and increase PWT in a dose-dependent manner.

All of the references cited in this application are incorporated by reference in their entirety.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

SEQUENCE LISTING

Human mGluR5b
SEQ ID NO: 1
MVLLLILSVLLLKEDVRGSAQSSERRVVAHMPGDIIIGALFSVHHQPTVDKVHERKCGAVREQYGIQRVEAMLHT
LERINSDPTLLPNITLGCEIRDSCWHSAVALEQSIEFIRDSLISSEEEEGLVRCVDGSSSSFRSKKPIVGVIGPG
SSSVAIQVQNLLQLFNIPQIAYSATSMDLSDKTLFKYFMRVVPSDAQQARAMVDIVKRYNWTYVSAVHTEGNYGE
SGMEAFKDMSAKEGICIAHSYKIYSNAGEQSFDKLLKKLTSHLPKARVVACFCEGMTVRGLLMAMRRLGLAGEFL
LLGSDGWADRYDVIDGYQREAVGGITIKLQSPDVKWFDDYYLKLRPETNHRNPWFQEFWQHRFQCRLEGFPQENS
KYNKTCNSSLILKTHHVQDSKMGFVINAIYSMAYGLHNMQMSLCPGYAGLCDAMKPIDGRKLLESLMKTNFTGVS
GDTILFDENGDSPGRYEIMNFKEMGKDYFDYINVGSWDNGELKMDDDEVWSKKSNIIRSVCSEPCEKGQIKVIRK
GEVSCCWTCTPCKENEYVFDEYTCKACQLGSWPTDDLTGCDLIPVQYLRWGDPEPIAAVVFACLGLLATLFVTVV
FIIYRDTPVVKSSSRELCYIILAGICLGYLCIFCLIAKPKQIYCYLQRIGIGLSPAMSYSALVTKTNRIARILAG
SKKKICTKKPRFMSACAQLVIAFILICIQLGIIVALFIMEPPDIMHDYPSIREVYLICNTTNLGVVTPLGYNGLL
ILSCTFYAFKTRNVPANFNEAKYIAFTMYTTCIIWLAFVPIYFGSNYKIITMCFSVSLSATVALGCMFVPKVYII
LAKPERNVRSAFTTSTVVRMHVGDGKSSSAASRSSSLVNLWKRRGSSGETLRYKDRRLAQHKSEIECFTPKGSMG
NGGRATMSSSNGKSVTWAQNEKSSRGQHLWQRLSIHINKKENPNQTAVIKPFPKSTESRGLGAGAGAGGSAGGVG
ATGGAGCAGAGPGGPESPDAGPKALYDVAEAEEHFPAPARPRSPSPISTLSHRAGSASRTDDDVPSLHSEPVARS
SSSQGSLMEQISSVVTRFTANISELNSMMLSTAAPSPGVGAPLCSSYLIPKEIQLPTTMTTFAEIQPLPAIEVTG
GAQPAAGAQAAGDAARESPAAGPEAAAAKPDLEELVALTPPSPFRDSVDSGSTIPNSPVSESALCIPSSPKYDTL
IIRDYTQSSSSL
Human mGluR5a
SEQ ID NO: 2
MVLLLILSVLLLKEDVRGSAQSSERRVVAHMPGDIIIGALFSVHHQPIVDKVHERKCGAVREQYGIQRVEAMLHT
LERINSDPTLLPNITLGCEIRDSCWHSAVALEQSIEFIRDSLISSEEEEGLVRCVDGSSSSFRSKKPIVGVIGPG
SSSVAIQVQNLLQLFNIPQIAYSATSMDLSDKTLFKYFMRVVPSDAQQARAMVDIVKRYNWTYVSAVHTEGNYGE
SGMEAFKDMSAKEGICIAHSYKIYSNAGEQSFDKLLKKLTSHLPKARVVACFCEGMTVRGLLMAMRRLGLAGEFL
LLGSDGWADRYDVTDGYQREAVGGITIKLQSPDVKWFDDYYLKLRPETNHRNPWFQEFWQHRFQCRLEGFPQENS
KYNKTCNSSLTLKTHHVQDSKMGFVINAIYSMAYGLHNMQMSLCPGYAGLCDAMKPIDGRKLLESLMKTNFTGVS
GDTILFDENGDSPGRYEIMNFKEMGKDYFDYINVGSWDNGELKMDDDEVWSKKSNIIRSVCSEPCEKGQIKVIRK
GEVSCCWTCTPCKENEYVFDEYTCKACQLGSWPTDDLIGCDLIPVQYLRWGDPEPIAAVVFACLGLLATLFVTVV
FIIYRDTPVVKSSSRELCYIILAGICLGYLCTFCLIAKPKQIYCYLQRIGIGLSPAMSYSALVTKTNRIARILAG
SKKKICIKKPRFMSACAQLVIAFILICIQLGIIVALFIMEPPDIMHDYPSIREVYLICNTTNLGVVIPLGYNGLL
ILSCIFYAFKTRNVPANFNEAKYIAFTMYTTCIIWLAFVPIYFGSNYKIITMCFSVSLSATVALGCMFVPKVYII
LAKPERNVRSAFTTSTVVRMHVGDGKSSSAASRSSSLVNLWKRRGSSGETLSSNGKSVTWAQNEKSSRGQHLWQR
LSIHINKKENPNQTAVIKPFPKSTESRGLGAGAGAGGSAGGVGATGGAGCAGAGPGGPESPDAGPKALYDVAEAE
EHFPAPARPRSPSPISTLSHRAGSASRTDDDVPSLHSEPVARSSSSQGSLMEQISSVVTRETANISELNSMMLST
AAPSPGVGAPLCSSYLIPKEIQLPTTMTTFAEIQPLPAIEVIGGAQPAAGAQAAGDAARESPAAGPEAAAAKPDL
EELVALTPPSPFRDSVDSGSTTPNSPVSESALCIPSSPKYDTLIIRDYTQSSSSL
Rat mGluR5
SEQ ID NO: 3
MVLLLILSVLLLKEDVRGSAQSSERRVVAHMPGDIIIGALFSVHHQPTVDKVHERKCGAVREQYGIQRVEAMLHT
LERINSDPILLPNITLGCEIRDSCWHSAVALEQSIEFIRDSLISSEEEEGLVRCVDGSSSFRSKKPIVGVIGPGS
SSVAIQVQNLLQLFNIPQTAYSATSMDLSDKTLFKYFMRVVPSDAQQARAMVDIVKRYNWTYVSAVHTEGNYGES
GMEAFKDMSAKEGICIAHSYKIYSNAGEQSFDKLLKKLRSHLPKARVVACFCEGMTVRGLLMAMRRLGLAGEFLL
LGSDGWADRYDVIDGYQREAVGGITIKLQSPDVKWFDDYYLKLRPETNLRNPWFQEFWQHRFQCRLEGFAQENSK
YNKTCNSSLTLRTHHVQDSKMGFVINAIYSMAYGLHNMQMSLCPGYAGLCDAMKPIDGRKLLDSLMKTNFTGVSG
DMILFDENGDSPGRYEIMNFKEMGKDYFDYINVGSWDNGELKMDDDEVWSKKNNIIRSVCSEPCEKGQIKVIRKG
EVSCCWICTPCKENEYVFDEYTCKACQLGSWPTDDLTGCDLIPVQYLRWGDPEPIAAVVFACLGLLATLFVTVIF
IIYRDTPVVKSSSRELCYTILAGICLGYLCTFCLIAKPKQIYCYLQRIGIGLSPAMSYSALVTKTNRIARILAGS
KKKICTKKPRFMSACAQLVIAFILICIQLGIIVALFIMEPPDIMHDYPSIREVYLICNTTNLGVVTPLGYNGLLI
LSCTFYAFKTRNVPANFNEAKYIAFTMYTTCIIWLAFVPIYFGSNYKIITMCFSVSLSATVALGCMFVPKVYIIL
AKPERNVRSAFTTSTVVRMHVGDGKSSSAASRSSSLVNLWKRRGSSGETLSSNGKSVTWAQNEKSTRGQHLWQRL
SVHINKKENPNQTAVIKPFPKSTENRGPGAAAGGGSGPGVAGAGNAGCTATGGPEPPDAGPKALYDVAEAEESFP
AAARPRSPSPISTLSHLAGSAGRTDDDAPSLHSETAARSSSSQGSLMEQISSVVTRFTANISELNSMMLSTAATP
GPPGTPICSSYLIPKEIQLPTTMTTFAEIQPLPAIEVTGGAQGATGVSPAQETPTGAESAPGKPDLEELVALTPP
SPFRDSVDSGSTTPNSPVSESALCIPSSPKYDTLIIRDYTQSSSSL
Cyno mGluR5
SEQ ID NO: 4
MVLLLILSVLLLKEDVRGSAQSSERRVVAHMPGDIIIGALFSVHHQPTVDKVHERKCGAVREQYGIQRVEAMLHT
LERINSDPTLLPNITLGCEIRDSCWHSAVALEQSIEFIRDSLISSEEEEGLVRCVDGSSSSERSKKPIVGVIGPG
SSSVAIQVQNLLQLFNIPQTAYSATSMDLSDKTLFKYFMRVVPSDAQQARAMVDIVKRYNWTYVSAVHTEGNYGE
SGMEAFKDMSAKEGICIAHSYKIYSNAGEQSFDKLLKKLTSHLPKARVVACFCEGMTVRGLLMAMRRLGLAGEFL
LLGSDGWADRYDVTDGYQREAVGGITIKLQSPDVKWFDDYYLKLRPETNHRNPWFQEFWQHRFQCRLEGFPQENS
KYNKTCDSSLTLKTHHVQDSKMGFVINAIYSMAYGLHNMQMSLCPGYAGLCDAMKPIDGRKLLESLMKTNFTGVS
GDMILFDENGDSPGRYEIMNFKEMGKDYFDYINVGSWDNGELKMDDDEVWSKKSNIIRSVCSEPCEKGQIKVIRK
GEVSCCWTCTPCKENEYVFDEYTCKACQLGSWPTDDLTGCDLIPVQYLRWGDPEPIAAVVFACLGLLATLFVTVV
FIIYRDTPVVKSSSRELCYIILAGICLGYLCTFCLIAKPKINYCYLQRIGIGLSPAMSYSALVTKTNRIARILAG
SKKKICTKKPREMSACAQLVIAFILICIQLGIIVALFIMEPPDIMHDYPSIREVYLICNTTNLGVVTPLGYNGLL
ILSCTFYAFKTRNVPANFNEAKYIAFTMYTTCIIWLAFVPIYEGSNYKIITMCFSVSLSATVALGCMFVPKVYII
LAKPERNVRSAFTTSTVVRMHVGDGKSSSAASRSSSLVNLWKRRGSSGETLRYKDRRLAQHKSEIECFTPKGSMG
NGGRATMSSSNGKSVTWAQNEKSSRGQHLWQRLSIHINKKENPNQTAVIKPFPKSTESRGLGAGAGAGGSAGGMG
ATGGAGCAGAGPGGPEPPDASPKGLYDVAETEEHFPAPTRPRSPSPISTLSHRAGSASRTDDDVPSLHSEPAARS
SSSQGSLMEQISSVVTRFTANISELNSMMLSTAAPSPGVGAPLCSSYLIPKEIQLPTTMTTFAEIQPLPAIEVTG
GAQPAAGAQAAGDAARESPAAGPKAAAAKPDLEELVALTPPSPFRDSVDSGSTTPNSPVSESALCIPSSPKYDTL
IIRDYTQSSSSL
Human mGluR1
SEQ ID NO: 5
MVGLLLEFFPAIFLEVSLLPRSPGRKVLLAGASSQRSVARMDGDVIIGALFSVHHQPPAEKVPERKCGEIREQYG
IQRVEAMFHTLDKINADPVLLPNITLGSEIRDSCWHSSVALEQSIEFIRDSLISIRDEKDGINRCLPDGQSLPPG
RTKKPIAGVIGPGSSSVAIQVQNLLQLFDIPQIAYSATSIDLSDKTLYKYFLRVVPSDTLQARAMLDIVKRYNWT
YVSAVHTEGNYGESGMDAFKELAAQEGLCIAHSDKIYSNAGEKSFDRLLRKLRERLPKARVVVCFCEGMTVRGLL
SAMRRLGVVGEFSLIGSDGWADRDEVIEGYEVEANGGITIKLQSPEVRSFDDYFLKLRLDTNTRNPWFPEFWQHR
FQCRLPGHLLENPNFKRICTGNESLEENYVQDSKMGFVINAIYAMAHGLQNMHHALCPGHVGLCDAMKPIDGSKL
LDFLIKSSFIGVSGEEVWFDEKGDAPGRYDIMNLQYTEANRYDYVHVGTWHEGVLNIDDYKIQMNKSGVVRSVCS
EPCLKGQIKVIRKGEVSCCWICTACKENEYVQDEFTCKACDLGWWPNADLTGCEPIPVRYLEWSNIESIIAIAFS
CLGILVTLFVTLIFVLYRDTPVVKSSSRELCYIILAGIFLGYVCPFTLIAKPTTTSCYLQRLLVGLSSAMCYSAL
VTKTNRIARILAGSKKKICTRKPRFMSAWAQVIIASILISVQLTLVVTLIIMEPPMPILSYPSIKEVYLICNTSN
LGVVAPLGYNGLLIMSCTYYAFKTRNVPANFNEAKYIAFTMYTTCIIWLAFVPIYEGSNYKIITTCFAVSLSVTV
ALGCMFTPKMYIIIAKPERNVRSAFTTSDVVRMHVGDGKLPCRSNTFLNIFRRKKAGAGNANSNGKSVSWSEPGG
GQVPKGQHMWHRLSVHVKTNETACNQTAVIKPLTKSYQGSGKSLTFSDTSTKTLYNVEEEEDAQPIRFSPPGSPS
MVVHRRVPSAATTPPLPSHLTAEETPLFLAEPALPKGLPPPLQQQQQPPPQQKSLMDQLQGVVSNFSTAIPDFHA
VLAGPGGPGNGLRSLYPPPPPPQHLQMLPLQLSTFGEELVSPPADDDDDSERFKLLQEYVYEHEREGNTEEDELE
EEEEDLQAASKLTPDDSPALTPPSPFRDSVASGSSVPSSPVSESVLCTPPNVSYASVILRDYKQSSSTL
Ab1_VH VH
SEQ ID NO: 6
CAGCAGCAGCTGGAGGAGTCCGGGGGAGGCCTGGTCAAGCCTGGAGGAACCCTGACACTCACCTGCAAAGTCTCT
GGGATCGACTTCAGTAGCTACTACTACATGTGCTGGGTCCGCCAGGCTCCAGGGAAGGGGCTGGAGTGGATCGCA
TGCATTTATGCTGGTGATGGTGGCACTTACTACGCGAGCTGGGCGAAAGGCCGATTCACCAGCTCCAAAACCTCG
TCGACCACGGTGACTCTGCAAATGACCAGTCTGACAGCCGCGGACACGGCCACCTATTTCTGTGCGAGGGGTAGT
GGTGTTGGTAGTTATGGTTACAGCTTGTGGGGCCCAGGCACCCTCGTCACCGTCTCGAGC
Ab1_VH VH
SEQ ID NO: 7
QQQLEESGGGLVKPGGTLTLTCKVSGIDESSYYYMCWVRQAPGKGLEWIACIYAGDGGTYYASWAKGRFTSSKTS
STTVTLQMTSLTAADTATYFCARGSGVGSYGYSLWGPGTLVTVSS
Ab1_VH CDRH1
SEQ ID NO: 8
AGCTACTACTACATGTGC
Ab1_VH CDRH1
SEQ ID NO: 9
SYYYMC
Ab1_VH CDRH2
SEQ ID NO: 10
TGCATTTATGCTGGTGATGGTGGCACTTACTACGCGAGCTGGGCGAAAGGC
Ab1_VH CDRH2
SEQ ID NO: 11
CIYAGDGGTYYASWAKG
Ab1_VH CDRH3
SEQ ID NO: 12
GGTAGTGGTGTTGGTAGTTATGGTTACAGCTTG
Ab1_VH CDRH3
SEQ ID NO: 13
GSGVGSYGYSL
Ab1_VL VL
SEQ ID NO: 14
GCTGACATTGTGATGACCCAGACTCCAGCCTCCGTGGAGGCAGCTGTGGGAGGCACAGTCACCATCAAGTGCCAG
GCCAGTGAGAGCATTTATAGCAATTTAGCCTGGTATCAGCAGAAACCAGGGCAGCCTCCCAAGCTCCTAATCTAC
AGGGCATCCAATCTGGAATCTGGGGTCCCATCGCGGTTCAAAGGCAGTGGATCTGGGACAGAATTTACTCTCACC
ATCAGCGACCTGGAGTGTGCCGATGCTGCCACTTACTACTGTCAAGGCTATTATGCCAGTACTACTACTAATGCT
TTCGGCGGAGGGACCGAGGTGGTGGTCAAACGT
Ab1_VL VL
SEQ ID NO: 15
ADIVMTQTPASVEAAVGGTVTIKCQASESIYSNLAWYQQKPGQPPKLLIYRASNLESGVPSRFKGSGSGTEFTLT
ISDLECADAATYYCQGYYASTTTNAFGGGTEVVVKR
Ab1_VL CDRL1
SEQ ID NO: 16
CAGGCCAGTGAGAGCATTTATAGCAATTTAGCC
Ab1_VL CDRL1
SEQ ID NO: 17
QASESIYSNLA
Ab1_VL CDRL2
SEQ ID NO: 18
AGGGCATCCAATCTGGAATCT
Ab1_VL CDRL2
SEQ ID NO: 19
RASNLES
Ab1_VL CDRL3
SEQ ID NO: 20
CAAGGCTATTATGCCAGTACTACTACTAATGCT
Ab1_VL CDRL3
SEQ ID NO: 21
QGYYASTTTNA
Ab2_VH VH
SEQ ID NO: 22
CAGCAGCAGCTGGAGGAGTCCGGGGGAGGCCTGGTCAAGCCTGGAGGAACCCTGACACTCACCTGCAAAGCCTCT
GGAATCGACTTCAGTGGCTACTACTACATGTGCTGGGTCCGCCAGGCTCCAGGGAAGGGGCTGGAGTGGATCGCA
TGCGTTTATACTGGTGATGGTGGCACTTACTACGCGAGCTGGGCGAAAGGCCGATTCACCATCTCCAAAACCTCG
TCGACCACGGTGACTCTGCAAATGACCAGTCTGACAGCCGCGGACACGGCCACCTATTTCTGTGCGAGGGGTAGT
GGTGTTGGTAGTTATGGTTACAACTTGTGGGGCCAAGGGACCCTCGTCACCGTCTCGAGC
Ab2_VH VH
SEQ ID NO: 23
QQQLEESGGGLVKPGGTLTLTCKASGIDFSGYYYMCWVRQAPGKGLEWIACVYTGDGGTYYASWAKGRFTISKTS
STTVTLQMTSLTAADTATYFCARGSGVGSYGYNLWGQGTLVTVSS
Ab2_VH CDRH1
SEQ ID NO: 24
GGCTACTACTACATGTGC
Ab2_VH CDRH1
SEQ ID NO: 25
GYYYMC
Ab2_VH CDRH2
SEQ ID NO: 26
TGCGTTTATACTGGTGATGGTGGCACTTACTACGCGAGCTGGGCGAAAGGC
Ab2_VH CDRH2
SEQ ID NO: 27
CVYTGDGGTYYASWAKG
Ab2_VH CDRH3
SEQ ID NO: 28
GGTAGTGGTGTTGGTAGTTATGGTTACAACTTG
Ab2_VH CDRH3
SEQ ID NO: 29
GSGVGSYGYNL
Ab2_VL VL
SEQ ID NO: 30
GCTGACATTGTGATGACCCAGACTCCAGCCTCCGTGGAGGCAGCTGTGGGAGGCACAGTCACCATCAAGTGCCAG
GCCAGTGAGAGCATTTATAGCAATTTAGCCTGGTATCAGCAGAAACCAGGGCAGCCTCCCAAGCTCCTAATCTAC
AGGGCATCCAATCTGGAATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTATGCCATTACTACTACTAATGCT
TTCGGCGGAGGGACCGAGGTGGTGGTCAAACGT
Ab2_VL VL
SEQ ID NO: 31
ADIVMTQTPASVEAAVGGTVTIKCQASESIYSNLAWYQQKPGQPPKLLIYRASNLESGVPSRFKGSGSGTEFTLT
ISDLECADAATYYCQGYYAITTTNAFGGGTEVVVKR
Ab2_VL CDRL1
SEQ ID NO: 32
CAGGCCAGTGAGAGCATTTATAGCAATTTAGCC
Ab2_VL CDRL1
SEQ ID NO: 33
QASESIYSNLA
Ab2_VL CDRL2
SEQ ID NO: 34
AGGGCATCCAATCTGGAATCT
Ab2_VL CDRL2
SEQ ID NO: 35
RASNLES
Ab2_VL CDRL3
SEQ ID NO: 36
CAAGGCTATTATGCCATTACTACTACTAATGCT
Ab2_VL CDRL3
SEQ ID NO: 37
QGYYAITTTNA
Ab3_VH VH
SEQ ID NO: 38
CAGTCGCTGGAGGAGTCCGGGGGTCGCCTGGTCACGCCTGGGACACCCCTGACACTCACCTGCACAGTCTCTGGA
ATCGACCTCAGTAGCTATGCAGTGGGCTGGGTCCGCCAGGCTCCAGGGAAGGGGCTGGAATACATCGGAATCATT
TATGCTAGTGGTAACACATGGTACGCGAGCTGGGTGAAAGGCCGATTCACCATCTCCAAAACCTCGACCACGGTG
GATCTGAAGTTGGCCAGTCTGACAACCGAGGACACGGCCACCTATTTCTGTGCCAGAGGTCATGGTGCTACTTAT
GACTACTTTAATTTGTGGGGCCCGGGCACCCTCGTCACCGTCTCGAGC
Ab3_VH VH
SEQ ID NO: 39
QSLEESGGRLVTPGTPLTLTCTVSGIDLSSYAVGWVRQAPGKGLEYIGIIYASGNTWYASWVKGRFTISKTSTTV
DLKLASLTTEDTATYFCARGHGATYDYFNLWGPGTLVTVSS
Ab3_VH CDRH1
SEQ ID NO: 40
AGCTATGCAGTGGGC
Ab3_VH CDRH1
SEQ ID NO: 41
SYAVG
Ab3_VH CDRH2
SEQ ID NO: 42
ATCATTTATGCTAGTGGTAACACATGGTACGCGAGCTGGGTGAAAGGC
Ab3_VH CDRH2
SEQ ID NO: 43
IIYASGNTWYASWVKG
Ab3_VH CDRH3
SEQ ID NO: 44
GGTCATGGTGCTACTTATGACTACTTTAATTTG
Ab3_VH CDRH3
SEQ ID NO: 45
GHGATYDYFNL
Ab3_VL VL
SEQ ID NO: 46
GCCTATGATATGACCCAGACTCCAGCCTCTGTGGAGGTAGCTGTGGGAGGCACAGTCACCATCAAGTGCCAGGCC
AGTCAGAGCATTAGTAGCTACTTAGCCTGGTATCAGCAGAAACCAGGGCAGCCTCCCAAGCTCCTGATCTATGAT
GCATCCGATCTGGCATCTGGGGTCCCATCGCGGTTCAAAGGCAGTGGATCTGGGACAGAGTACACTCTCACCATC
AGCGGCGTGCAGTGTGACGATGCTGCCACTTACTACTGTCAACAGGGTCTTGCTACTGCTAATGTTGATAATGTT
TTCGGCGGAGGGACCGAGGTGGTGGTCAAACGT
Ab3_VL VL
SEQ ID NO: 47
AYDMTQTPASVEVAVGGTVTIKCQASQSISSYLAWYQQKPGQPPKLLIYDASDLASGVPSRFKGSGSGTEYTLTI
SGVQCDDAATYYCQQGLATANVDNVEGGGTEVVVKR
Ab3_VL CDRL1
SEQ ID NO: 48
CAGGCCAGTCAGAGCATTAGTAGCTACTTAGCC
Ab3_VL CDRL1
SEQ ID NO: 49
QASQSISSYLA
Ab3_VL CDRL2
SEQ ID NO: 50
GATGCATCCGATCTGGCATCT
Ab3_VL CDRL2
SEQ ID NO: 51
DASDLAS
Ab3_VL CDRL3
SEQ ID NO: 52
CAACAGGGTCTTGCTACTGCTAATGTTGATAATGTT
Ab3_VL CDRL3
SEQ ID NO: 53
QQGLATANVDNV
Ab4_VH VH
SEQ ID NO: 54
CAGCAGCAGCTGGAGGAGTCCGGGGGAGGCCTGGTCAAGCCTGGAGGAACCCTGACACTCACCTGCAAAGGCTCT
GGAATCGACTTCAATGACTACCAGTACATGTGCTGGGTCCGCCAGGCTCCAGGGAAGGGGCTGGAGTGGATCGCG
TGCATTTATAATGGGGATGGTGAGACTTACTACGCGCACTGGGCGAAAGGCCGATTCACCATCTCCAAAACCTCG
TCGACCACGGTGACTCTGCAGATGACCAGTCTGACAGCCGCGGACACGGCCACCTATTTCTGTGCGAGGGGTAGT
GGTGTTGGGAGTTATGGTTACAACTTTTGGGGCCCGGGCACCCTCGTCACCGTCTCGAGC
Ab4_VH VH
SEQ ID NO: 55
QQQLEESGGGLVKPGGTLTLTCKGSGIDENDYQYMCWVRQAPGKGLEWIACIYNGDGETYYAHWAKGRFTISKTS
STTVTLQMTSLTAADTATYFCARGSGVGSYGYNFWGPGTLVTVSS
Ab4_VH CDRH1
SEQ ID NO: 56
GACTACCAGTACATGTGC
Ab4_VH CDRH1
SEQ ID NO: 57
DYQYMC
Ab4_VH CDRH2
SEQ ID NO: 58
TGCATTTATAATGGGGATGGTGAGACTTACTACGCGCACTGGGCGAAAGGC
Ab4_VH CDRH2
SEQ ID NO: 59
CIYNGDGETYYAHWAKG
Ab4_VH CDRH3
SEQ ID NO: 60
GGTAGTGGTGTTGGGAGTTATGGTTACAACTTT
Ab4_VH CDRH3
SEQ ID NO: 61
GSGVGSYGYNF
Ab4_VL VL
SEQ ID NO: 62
GCTGACATTGTGATGACCCAGACTCCAGCCTCCGTGGAGGCAGCTGTGGGAGGCACAGTCACCATCAAGTGCCAG
GCCAGTGAGAGCATTTACAGCAATTTAGCCTGGTATCAGCAGAAAGCAGGGCAGCCTCCCAAGCTCCTAATCTAC
AGGGCATCCAATCTGGCATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTATGCCAGTACTGTTACTAATGGT
TTCGGCGGAGGGACCGAGGTGGTGGTCAAACGT
Ab4_VL VL
SEQ ID NO: 63
ADIVMTQTPASVEAAVGGTVTIKCQASESIYSNLAWYQQKAGQPPKLLIYRASNLASGVPSRFKGSGSGTEFTLT
ISDLECADAATYYCQGYYASTVTNGFGGGTEVVVKR
Ab4_VL CDRL1
SEQ ID NO: 64
CAGGCCAGTGAGAGCATTTACAGCAATTTAGCC
Ab4_VL CDRL1
SEQ ID NO: 65
QASESIYSNLA
Ab4_VL CDRL2
SEQ ID NO: 66
AGGGCATCCAATCTGGCATCT
Ab4_VL CDRL2
SEQ ID NO: 67
RASNLAS
Ab4_VL CDRL3
SEQ ID NO: 68
CAAGGCTATTATGCCAGTACTGTTACTAATGGT
Ab4_VL CDRL3
SEQ ID NO: 69
QGYYASTVTNG
Ab5_VH VH
SEQ ID NO: 70
CAGGAGCAGCTGGTGGAGTCCGGGGGAGGCCTGGTCCAGCCTGAGGGATCCCTGACACTCACCTGCACAGCCTCT
GGAATCGACTTCAGTAGCGGCTATTTCATGTGCTGGGTCCGCCAGGCTCCAGGGAAGGGGCTGGAGTGGATCGCA
TGCATTTCGCCTTATAGTGATAATACATGGTACGCGAACTGGGCGAAAGGCCGATTCACCATCTCCAAAACCTCG
TCGACCACGGTGACTCTGCAAATGACCAGTCTGACAGCCGCGGACACGGCCGCCTATCGCTGTGCGAGAACTATG
GCTATTGGCGCTTATAACCCCTTTAAGTTGTGGGGCCAGGGCACCCTGGTCACCGTCTCGAGC
Ab5_VH VH
SEQ ID NO: 71
QEQLVESGGGLVQPEGSLTLTCTASGIDFSSGYFMCWVRQAPGKGLEWIACISPYSDNTWYANWAKGRFTISKTS
STTVTLQMTSLTAADTAAYRCARTMAIGAYNPFKLWGQGTLVTVSS
Ab5_VH CDRH1
SEQ ID NO: 72
AGCGGCTATTTCATGTGC
Ab5_VH CDRH1
SEQ ID NO: 73
SGYFMC
Ab5_VH CDRH2
SEQ ID NO: 74
TGCATTTCGCCTTATAGTGATAATACATGGTACGCGAACTGGGCGAAAGGC
Ab5_VH CDRH2
SEQ ID NO: 75
CISPYSDNTWYANWAKG
Ab5_VH CDRH3
SEQ ID NO: 76
ACTATGGCTATTGGCGCTTATAACCCCTTTAAGTTG
Ab5_VH CDRH3
SEQ ID NO: 77
TMAIGAYNPFKL
Ab5_VL VL
SEQ ID NO: 78
GCCCTTGTGATGACCCAGACTCCATCCTCCGTGTCTGCAGCTGTGGGAGGCACAGTCACCATCAATTGCCAGGCC
AGTCAGAACATTTACAGCAATTTAGCCTGGTATCAGCAGAAACCAGGGCAGCGTCCCAAGCTCCTGATCTATGAT
GCATCCGATCTGGCATCTGGGGTCTCATCGCGGGTCAAAGGCAGTGGATCTGGGACAGAGTTCACTCTCACCATC
AGCGACCTGGAGTGTGCCGATGCTGCCACTTACTACTGTCAAGGCTATTATTCTGGTAATGCTTTCGGCGGAGGG
ACCGAGGTGGTGGTCAAACGT
Ab5_VL VL
SEQ ID NO: 79
ALVMTQTPSSVSAAVGGTVTINCQASQNIYSNLAWYQQKPGQRPKLLIYDASDLASGVSSRVKGSGSGTEFTLTI
SDLECADAATYYCQGYYSGNAFGGGTEVVVKR
Ab5_VL CDRL1
SEQ ID NO: 80
CAGGCCAGTCAGAACATTTACAGCAATTTAGCC
Ab5_VL CDRL1
SEQ ID NO: 81
QASQNIYSNLA
Ab5_VL CDRL2
SEQ ID NO: 82
GATGCATCCGATCTGGCATCT
Ab5_VL CDRL2
SEQ ID NO: 83
DASDLAS
Ab5_VL CDRL3
SEQ ID NO: 84
CAAGGCTATTATTCTGGTAATGCT
Ab5_VL CDRL3
SEQ ID NO: 85
QGYYSGNA
Ab6_VH VH
SEQ ID NO: 86
CAGTCGCTGGAGGAGTCCGGGGGTCGCCTGGTCACGCCTGGAGGAACCCTAACACTCACCTGCACAGTCTCTGGA
ATCGACCTCAGTAGCTATGCAATGAGCTGGGTCCGCCAGGCTCCAGGGAAGGGGCTGGAATGGATCGGAATCATT
TATGCTAGTGGTAACACATGGTACGCGAACTGGGTGAAAGGCCGATTCACCATCTCCAAAACCTCGACCACGGTG
GATCTGAAAATGACCAGTCCGACAACCGAGGACACGGCCACCTATTTCTGTGCCAGAGATAGTAGTGCTACTTTT
AATTATCTAGACTTGTGGGGCCAAGGCACCCTCGTCACCGTCTCGAGC
Ab6_VH VH
SEQ ID NO: 87
QSLEESGGRLVTPGGTLTLTCTVSGIDLSSYAMSWVRQAPGKGLEWIGIIYASGNTWYANWVKGRFTISKTSTTV
DLKMTSPTTEDTATYFCARDSSATFNYLDLWGQGTLVTVSS
Ab6_VH CDRH1
SEQ ID NO: 88
AGCTATGCAATGAGC
Ab6_VH CDRH1
SEQ ID NO: 89
SYAMS
Ab6_VH CDRH2
SEQ ID NO: 90
ATCATTTATGCTAGTGGTAACACATGGTACGCGAACTGGGTGAAAGGC
Ab6_VH CDRH2
SEQ ID NO: 91
IIYASGNTWYANWVKG
Ab6_VH CDRH3
SEQ ID NO: 92
GATAGTAGTGCTACTTTTAATTATCTAGACTTG
Ab6_VH CDRH3
SEQ ID NO: 93
DSSATFNYLDL
Ab6_VL VL
SEQ ID NO: 94
GCCTTTCATATGACCCAGACTCCAGCCTCTATGGAGGTAGCTGTGGGAGGCACAGTCACCATGAAGTGCCAGGCC
AGTGAGAGCATTGATACCTACTTAAACTGGTATCAGGAGAAACCAGGGCAGCCTCCCAAGCTCCTGATCTACCAG
GCATCCAAACTGGCATCTGGGGTCCCATCGCGGTTCAAAGGCAGTGGATCTGGGACAGAGTTCACTCTCACCATC
AGCGGCGTGGAGTGTGCCGATGCTGCCACTTACTACTGTCAACAGGGTTGGAGTACTAATGATGTTGATAATGTT
TTCGGCGGAGGGACCGAGGTGGTGGTCAAACGT
Ab6_VL VL
SEQ ID NO: 95
AFHMTQTPASMEVAVGGTVTMKCQASESIDTYLNWYQQKPGQPPKLLIYQASKLASGVPSRFKGSGSGTEFTLTI
SGVECADAATYYCQQGWSTNDVDNVFGGGTEVVVKR
Ab6_VL CDRL1
SEQ ID NO: 96
CAGGCCAGTGAGAGCATTGATACCTACTTAAAC
Ab6_VL CDRL1
SEQ ID NO: 97
QASESIDTYLN
Ab6_VL CDRL2
SEQ ID NO: 98
CAGGCATCCAAACTGGCATCT
Ab6_VL CDRL2
SEQ ID NO: 99
QASKLAS
Ab6_VL CDRL3
SEQ ID NO: 100
CAACAGGGTTGGAGTACTAATGATGTTGATAATGTT
Ab6_VL CDRL3
SEQ ID NO: 101
QQGWSTNDVDNV
Ab7_VH VH
SEQ ID NO: 102
CAGTCGGTGGAGGAGTCCGGGGGTCGCCTGGTCACGCCTGGGACACCCCTGACACTCACCTGCACAGTCTCTGGA
TTCTCCCTCAGTCGCTATGCAATGGGCTGGGTCCGCCACGTTCCAGGGAAGGGGCTGGAATGGATCGGAATCATT
TATGCTAGTGGTAGCACATACTACGCGAGCTGGGCGAAGGGCCGATTCACCATCTCCAAAACCTCGACCACGGTG
GATCTGAAAATCACCAGTCCGACAACCGAGGACACGGCCACCTATTTCTGTGCCAGATCGCGTACTGCTACTTAT
GATGCTGTTGATCCCTGGGGCCCGGGCACCCTCGTCACCGTCTCGAGC
Ab7_VH VH
SEQ ID NO: 103
QSVEESGGRLVTPGTPLTLTCTVSGFSLSRYAMGWVRHVPGKGLEWIGIIYASGSTYYASWAKGRFTISKTSTTV
DLKITSPTTEDTATYFCARSRTATYDAVDPWGPGTLVTVSS
Ab7_VH CDRH1
SEQ ID NO: 104
CGCTATGCAATGGGC
Ab7_VH CDRH1
SEQ ID NO: 105
RYAMG
Ab7_VH CDRH2
SEQ ID NO: 106
ATCATTTATGCTAGTGGTAGCACATACTACGCGAGCTGGGCGAAGGGC
Ab7_VH CDRH2
SEQ ID NO: 107
IIYASGSTYYASWAKG
Ab7_VH CDRH3
SEQ ID NO: 108
TCGCGTACTGCTACTTATGATGCTGTTGATCCC
Ab7_VH CDRH3
SEQ ID NO: 109
SRTATYDAVDP
Ab7_VL VL
SEQ ID NO: 110
GCCTATGATATGACCCAGACTCCAGCCTCCGTGTCTGAACCTGTGGGAGGCACAGTCACCATCAAGTGCCAGGCC
AGTCAGAGCATTAGTAGTTACTTATCCTGGTATCAGCAGAAACCAGGGCAGCCTCCCAAGCTCCTGATCTACGAA
GCATCCACTCTGGCATCTGGGGTCTCATCGCGGTTCAAAGGCAGTGGATCTGGGACAGAGTACACTCTCACCATC
AGCGACCTGGAGTGTGACGATGCTGCCGCTTACTACTGTCAACAGGATCTTAGTGGTAGTAATCTTGATAATTCT
TTCGGCGGAGGGACCGAGGTGGTGGTCAAACGT
Ab7_VL VL
SEQ ID NO: 111
AYDMTQTPASVSEPVGGTVTIKCQASQSISSYLSWYQQKPGQPPKLLIYEASTLASGVSSRFKGSGSGTEYTLTI
SDLECDDAAAYYCQQDLSGSNLDNSFGGGTEVVVKR
Ab7_VL CDRL1
SEQ ID NO: 112
CAGGCCAGTCAGAGCATTAGTAGTTACTTATCC
Ab7_VL CDRL1
SEQ ID NO: 113
QASQSISSYLS
Ab7_VL CDRL2
SEQ ID NO: 114
GAAGCATCCACTCTGGCATCT
Ab7_VL CDRL2
SEQ ID NO: 115
EASTLAS
Ab7_VL CDRL3
SEQ ID NO: 116
CAACAGGATCTTAGTGGTAGTAATCTTGATAATTCT
Ab7_VL CDRL3
SEQ ID NO: 117
QQDLSGSNLDNS
Ab8_VH VH
SEQ ID NO: 118
CAGTCGGTGGAGGAGTCCGGGGGTCGCCTGGTCACGCCTGGGACACCCCTGACACTCACCTGCTCCGTCTCTGGA
ATCGACCTCAGTAGCTATGCAATGAGCTGGGTCCGCCAGGCTCCAGGGAAGGGGCTGGAATGGATCGGAGTCATT
TATGCTAGTGGTAGCACATACTACGCGAGCTGGGCGAAAGGCCGATTCACCATCTCCAAGACCTCGACCACGGTG
GATCTGAAAATCACCAGTCCGACAACCGGGGACACGGCCACCTATTTCTGTGTCAGAGATACTACTGCAACTTAT
AACTACTTTAACTTGTGGGGCCAGGGCACCCTCGTCACCGTCTCGAGC
Ab8_VH VH
SEQ ID NO: 119
QSVEESGGRLVTPGTPLTLTCSVSGIDLSSYAMSWVRQAPGKGLEWIGVIYASGSTYYASWAKGRFTISKTSTTV
DLKITSPTTGDTATYFCVRDTTATYNYFNLWGQGTLVTVSS
Ab8_VH CDRH1
SEQ ID NO: 120
AGCTATGCAATGAGC
Ab8_VH CDRH1
SEQ ID NO: 121
SYAMS
Ab8_VH CDRH2
SEQ ID NO: 122
GTCATTTATGCTAGTGGTAGCACATACTACGCGAGCTGGGCGAAAGGC
Ab8_VH CDRH2
SEQ ID NO: 123
VIYASGSTYYASWAKG
Ab8_VH CDRH3
SEQ ID NO: 124
GATACTACTGCAACTTATAACTACTTTAACTTG
Ab8_VH CDRH3
SEQ ID NO: 125
DTTATYNYFNL
Ab8_VL VL
SEQ ID NO: 126
GCCTATGATATGACCCAGACTCCAGCCTCTGTGGAGGTAGCTGTGGGAGGCACAGCCACCATCAAGTGCCAGGCC
AGTCAGAGCATTTACAACTACTTAGCCTGGTATCAGCAGAAACCAGGGCAGCCTCCCAAGCTCCTGATCTATGAA
GCATCCACTCTGGCCTCCGGGGTCCCATCGCGGTTCAAAGGCAGTGGATCTGGGACAGAGTTCACTCTCACCATC
AGCGACCTGGAGTGTGCCGATGCTGCCACTTACTACTGTCAACAGAGTTTGAGTCCTAGTAATGTTGGTCGAAGG
GTTTTCGGCGGAGGGACCGAGGTGGTGGTCAAACGT
Ab8_VL VL
SEQ ID NO: 127
AYDMTQTPASVEVAVGGTATIKCQASQSIYNYLAWYQQKPGQPPKLLIYEASTLASGVPSRFKGSGSGTEFTLTI
SDLECADAATYYCQQSLSPSNVGRRVFGGGTEVVVKR
Ab8_VL CDRL1
SEQ ID NO: 128
CAGGCCAGTCAGAGCATTTACAACTACTTAGCC
Ab8_VL CDRL1
SEQ ID NO: 129
QASQSIYNYLA
Ab8_VL CDRL2
SEQ ID NO: 130
GAAGCATCCACTCTGGCCTCC
Ab8_VL CDRL2
SEQ ID NO: 131
EAS TLAS
Ab8_VL CDRL3
SEQ ID NO: 132
CAACAGAGTTTGAGTCCTAGTAATGTTGGTCGAAGGGTT
Ab8_VL CDRL3
SEQ ID NO: 133
QQSLSPSNVGRRV
Ab9_VH VH
SEQ ID NO: 134
CAGTCGGTGGAGGAGTCCGGGGGTCGCCTGGTCACGCCTGGGACACCCCTGACACTCACCTGCACAGTCTCTGGA
TTCTCCCTCAATAACTATGCAATGAACTGGATCCGCCAGGCTCCAGGGAAGGGACTGGAATGGATCGGAATCATT
TATGGTAGTGGTAGCACATACTACGCGAGCTGGGCGAAAGGCCGATTCACCGCCTCCAAAACCTCGACCACGGTG
GATCTGAAAATGACCAGTCTGACAACCGAGGACACGGCCACGTATTTCTGTGCCAGAGATGAACTAGCTGCTGGT
ATTAATTACTTTACCTTGTGGGGCCAAGGGACCCTGGTCACCGTCTCGAGC
Ab9_VH VH
SEQ ID NO: 135
QSVEESGGRLVTPGTPLTLTCTVSGFSLNNYAMNWIRQAPGKGLEWIGIIYGSGSTYYASWAKGRFTASKTSTTV
DLKMTSLTTEDTATYFCARDELAAGINYFTLWGQGTLVTVSS
Ab9_VH CDRH1
SEQ ID NO: 136
AACTATGCAATGAAC
Ab9_VH CDRH1
SEQ ID NO: 137
NYAMN
Ab9_VH CDRH2
SEQ ID NO: 138
ATCATTTATGGTAGTGGTAGCACATACTACGCGAGCTGGGCGAAAGGC
Ab9_VH CDRH2
SEQ ID NO: 139
IIYGSGSTYYASWAKG
Ab9_VH CDRH3
SEQ ID NO: 140
GATGAACTAGCTGCTGGTATTAATTACTTTACCTTG
Ab9_VH CDRH3
SEQ ID NO: 141
DELAAGINYFTL
Ab9_VL VL
SEQ ID NO: 142
GCCCTTGTGATGACCCAGACTCCAGCCTCCGTGGAGGTAGCTGTGGGAGGCACAGTCACCATCAAGTGCCAGGCC
AGTCAGAATATTATTGATTGGTGCTCCTGGTATCAGGAGAAACTAGGGCAGCCTCCCAAACTCCTGATCTACGAA
GCATCTAAACTGGCATCTGGGGTCCCATCGCGGTTCAAAGGCAGTGGATCTGGGACACAGTTCACTCTCACCATC
AGCGGCGTGGAGTGTGCCGATGCTGCCACTTACTATTGTCAACAGGGTTATAGTCCTAGTGGTGTTGATAATGTT
TTCGGCGGAGGGACCGAGGTGGTGGTCAAACGT
Ab9_VL VL
SEQ ID NO: 143
ALVMTQTPASVEVAVGGTVTIKCQASQNIIDWCSWYQQKLGQPPKLLIYEASKLASGVPSRFKGSGSGTQFTLTI
SGVECADAATYYCQQGYSPSGVDNVFGGGTEVVVKR
Ab9_VL CDRL1
SEQ ID NO: 144
CAGGCCAGTCAGAATATTATTGATTGGTGCTCC
Ab9_VL CDRL1
SEQ ID NO: 145
QASQNIIDWCS
Ab9_VL CDRL2
SEQ ID NO: 146
GAAGCATCTAAACTGGCATCT
Ab9_VL CDRL2
SEQ ID NO: 147
EASKLAS
Ab9_VL CDRL3
SEQ ID NO: 148
CAACAGGGTTATAGTCCTAGTGGTGTTGATAATGTT
Ab9_VL CDRL3
SEQ ID NO: 149
QQGYSPSGVDNV
Ab10_VH VH
SEQ ID NO: 150
CAGTCGCTGGAGGAGTCCGGGGGTCGCCTGGTCACGCCTGGGACACCCCTGACACTCACCTGCACAGTCTCTGGA
ATCGACCTCAGTAGCTATGCAGTGGGCTGGGTCCGCCAGGCTCCAGGGAAGGGGCTGGAATACATCGGAATCATT
TATGCTAGTGGTAACACATGGTACGCGAGCTGGGTGAAAGGCCGATTCACCATCTCCAAAACCTCGACCACGGTG
GATCTGAAAATGACCAGTCTGACAACCGAGGACACGGCCACCTATTTCTGTGCCAGAGGTCATGGTGCTACTTAT
GACTACTTTAATTTGTGGGGCCCAGGCACCCTCGTCACCGTCTCGAGC
Ab10_VH VH
SEQ ID NO: 151
QSLEESGGRLVTPGTPLTLTCTVSGIDLSSYAVGWVRQAPGKGLEYIGIIYASGNTWYASWVKGRFTISKTSTTV
DLKMTSLTTEDTATYFCARGHGATYDYFNLWGPGTLVTVSS
Ab10_VH CDRH1
SEQ ID NO: 152
AGCTATGCAGTGGGC
Ab10_VH CDRH1
SEQ ID NO: 153
SYAVG
Ab10_VH CDRH2
SEQ ID NO: 154
ATCATTTATGCTAGTGGTAACACATGGTACGCGAGCTGGGTGAAAGGC
Ab10_VH CDRH2
SEQ ID NO: 155
IIYASGNTWYASWVKG
Ab10_VH CDRH3
SEQ ID NO: 156
GGTCATGGTGCTACTTATGACTACTTTAATTTG
Ab10_VH CDRH3
SEQ ID NO: 157
GHGATYDYFNL
AB10_VL VL
SEQ ID NO: 158
GCCTATGATATGACCCAGACTCCAGCCTCTGTGGAGGTAGCTGTGGGAGGCACAGTCACCATCAAGTGCCAGGCC
AGTCAGAGCATTAGCAGTTGGTTAGCCTGGTATCAGCAGAAACCAGGGCAGCCTCCCAAGCTCCTGATCTATGAT
GCATCCGATCTGGCATCTGGGGTCCCATCGCGGTTCAAAGGCAGTGGATCTGGGACAGAGTACACTCTCACCATC
AGCGGCGTGCAGTGTGACGATGCTGCCACTTACTACTGTCAACAGGGTCTTGCTACTGCTAATGTTGATAATGTT
TTCGGCGGAGGGACCGAGGTGGTGGTCAAACGT
AB10_VL VL
SEQ ID NO: 159
AYDMTQTPASVEVAVGGTVTIKCQASQSISSWLAWYQQKPGQPPKLLIYDASDLASGVPSRFKGSGSGTEYTLTI
SGVQCDDAATYYCQQGLATANVDNVFGGGTEVVVKR
AB10_VL CDRL1
SEQ ID NO: 160
CAGGCCAGTCAGAGCATTAGCAGTTGGTTAGCC
AB10_VL CDRL1
SEQ ID NO: 161
QASQSISSWLA
AB10_VL CDRL2
SEQ ID NO: 162
GATGCATCCGATCTGGCATCT
AB10_VL CDRL2
SEQ ID NO: 163
DASDLAS
AB10_VL CDRL3
SEQ ID NO: 164
CAACAGGGTCTTGCTACTGCTAATGTTGATAATGTT
AB10_VL CDRL3
SEQ ID NO: 165
QQGLATANVDNV
Ab11_VH VH
SEQ ID NO: 166
CAGTCGTTGGAGGAGTCCGGGGGAGGCCTGGTCAAGCCTGGAGGAACCCTGACACTCACCTGCACAGCCTCTGGA
TTCTCCTTCAGTAGCAGCTACTGGATATGCTGGGTCCGCCAGGCTCCAGGGAAGGGGCTGGAGTGGATCGGATGC
ATTTACGGAAGTACTGATGCCTATAACGCGAACTGGGCGAAAGGCCGATTCGCCATCTCCCAAACCTCGTCGACC
ACGGTGACTCTGCAAATGACCAGTCTGCCAGCCGCGGACACGGCCACCTATTTCTGTGCGCGACATGATGGTGAT
ATTGGTTGGACCTTGGACTTGTGGGGCCCGGGGACCCTGGTCACCGTCTCGAGC
Ab11_VH VH
SEQ ID NO: 167
QSLEESGGGLVKPGGTLTLTCTASGESFSSSYWICWVRQAPGKGLEWIGCIYGSTDAYNANWAKGRFAISQTSST
TVTLQMTSLPAADTATYFCARHDGDIGWTLDLWGPGTLVTVSS
Ab11_VH CDRH1
SEQ ID NO: 168
AGCAGCTACTGGATATGC
Ab11_VH CDRH1
SEQ ID NO: 169
SSYWIC
Ab11_VH CDRH2
SEQ ID NO: 170
TGCATTTACGGAAGTACTGATGCCTATAACGCGAACTGGGCGAAAGGC
Ab11_VH CDRH2
SEQ ID NO: 171
CIYGSTDAYNANWAKG
Ab11_VH CDRH3
SEQ ID NO: 172
CATGATGGTGATATTGGTTGGACCTTGGACTTG
Ab11_VH CDRH3
SEQ ID NO: 173
HDGDIGWTLDL
Ab11_VL VL
SEQ ID NO: 174
GCCTATGATATGACCCAGACTCCAGCCTCTGTGGAGGTCGCTGTGGGAGGCACAGTCACCATCAAGTGCCAGGCC
AGTCTGACCATTGGTACTCGGTTAGCCTGGTATCAGCAGAAACCAGGGCAGCCTCCCAAGCTCCTGATCTACAAG
GCATCCACTCTGGCATCTGGGGTCTCATCGCGGTTCAAAGGCAGTGGATCTGGGACACAGTTCGCTCTCACCATC
AGCGACCTGGAATGTGQCGATGCTGCCACTTACTACTGTCAACAGGCTGCTTATGTTACTGATATAGATAATGCT
TTCGGCGGAGGGACCGAGGTGGTGGTCAAACGT
Ab11_VL VL
SEQ ID NO: 175
AYDMTQTPASVEVAVGGTVTIKCQASLTIGTRLAWYQQKPGQPPKLLIYKASTLASGVSSRFKGSGSGTQFALTI
SDLECADAATYYCQQAAYVTDIDNAFGGGTEVVVKR
Ab11_VL CDRL1
SEQ ID NO: 176
CAGGCCAGTCTGACCATTGGTACTCGGTTAGCC
Ab11_VL CDRL1
SEQ ID NO: 177
QASLTIGTRLA
Ab11_VL CDRL2
SEQ ID NO: 178
AAGGCATCCACTCTGGCATCT
Ab11_VL CDRL2
SEQ ID NO: 179
KASTLAS
Ab11_VL CDRL3
SEQ ID NO: 180
CAACAGGCTGCTTATGTTACTGATATAGATAATGCT
Ab11_VL CDRL3
SEQ ID NO: 181
QQAAYVTDIDNA
Ab12_VH VH
SEQ ID NO: 182
CAGTCGGTGGAGGAGTCCGGGGGTCGCCTGGTCACGCCTGGGACACCCCTGACACTCACCTGCACAGTCTCTGGA
ATCGACCTCAGTAGCAATGCAATGGGCTGGGTCCGCCAGGCTCCAGGGAAGGGGCTGGAATGGATCGGGATCATT
TATGCTAGTGGTAACGCATGGTACGCGAACTGGGTGAAAGGCCGATTCACCATCTCCAAAACCTCGACCACGGTG
GATCTGAAAATCACCAGTCCGACAACCGAGGACACGGCCACCTATTTCTGTGCCAGGGATGCTACTGCTACTTGG
GACTACTTTAACTTGTGGGGCCAAGGCACCCTCGTCACCGTCTCGAGC
Ab12_VH VH
SEQ ID NO: 183
QSVEESGGRLVTPGTPLTLTCTVSGIDLSSNAMGWVRQAPGKGLEWIGIIYASGNAWYANWVKGRFTISKTSTTV
DLKITSPTTEDTATYFCARDATATWDYFNLWGQGTLVTVSS
Ab12_VH CDRH1
SEQ ID NO: 184
AGCAATGCAATGGGC
Ab12_VH CDRH1
SEQ ID NO: 185
SNAMG
Ab12_VH CDRH2
SEQ ID NO: 186
ATCATTTATGCTAGTGGTAACGCATGGTACGCGAACTGGGTGAAAGGC
Ab12_VH CDRH2
SEQ ID NO: 187
IIYASGNAWYANWVKG
Ab12_VH CDRH3
SEQ ID NO: 188
GATGCTACTGCTACTTGGGACTACTTTAACTTG
Ab12_VH CDRH3
SEQ ID NO: 189
DATATWDYFNL
Ab12_VL VL
SEQ ID NO: 190
GCCTATGATATGACCCAGACTCCATCCTCCGTGTCTGCAGCTGTGGGAGGCACAGTCACCATCAAGTGCCAGGCC
AGTCAGAGCATTAGTAATTGGTTATCCTGGTATCAGCAGAAACCAGGGCAGCCTCCCAAGCTCCTGATCTACAGG
GCGTCCACTCTGGCATCTGGGGTCTCATCGCGGTTCAGCGGCAGTGGATCTGGGACAGAGTTCACTCTCACCATC
AGCGACCTGGAGTGTGCCGATGCTGCCACTTACTACTGTCAACAGGGTTATAGTGTTGCTAATGTGGATAATGCT
TTCGGCGGAGGGACCGAGGTGGTGGTCAAACGT
Ab12_VL VL
SEQ ID NO: 191
AYDMTQTPSSVSAAVGGTVTIKCQASQSISNWLSWYQQKPGQPPKLLIYRASTLASGVSSRFSGSGSGTEFTLTI
SDLECADAATYYCQQGYSVANVDNAFGGGTEVVVKR
Ab12_VL CDRL1
SEQ ID NO: 192
CAGGCCAGTCAGAGCATTAGTAATTGGTTATCC
Ab12_VL CDRL1
SEQ ID NO: 193
QASQSISNWLS
Ab12_VL CDRL2
SEQ ID NO: 194
AGGGCGTCCACTCTGGCATCT
Ab12_VL CDRL2
SEQ ID NO: 195
RASTLAS
Ab12_VL CDRL3
SEQ ID NO: 196
CAACAGGGTTATAGTGTTGCTAATGTGGATAATGCT
Ab12_VL CDRL3
SEQ ID NO: 197
QQGYSVANVDNA
Ab13_VH VH
SEQ ID NO: 198
CAGTCGTTGGAGGAGTCCGGGGGAGACCTGGTCAAGCCTGGGGCATCCCTGACACTCACCTGCACAGCCTCTGGA
TTCTCCTTCAGTAGCAGCGACTACATGTGTTGGGTCCGCCAGGCTCCAGGGAAGGGGCTGGAGTGGATCGGATGC
ATTTATACTGGTGATGGTACCACATACTACGCGAGCTGGGCGAAAGGCCGATTCACCATCTCCAAAACCTCGTCG
ACCACGGTGACTCTGCAAATGACCAGTCTGACAGCCGCGGACACGGCCACCTATTTCTGTGCGAGAGGGGCTGGT
GTTGGTTCTTATGGTTATAATTTGTGGGGCCCGGGCACCCTCGTCACCGTCTCGAGC
Ab13_VH VH
SEQ ID NO: 199
QSLEESGGDLVKPGASLTLTCTASGESESSSDYMCWVRQAPGKGLEWIGCIYTGDGTTYYASWAKGRFTISKTSS
TTVTLQMTSLTAADTATYFCARGAGVGSYGYNLWGPGTLVTVSS
Ab13_VH CDRH1
SEQ ID NO: 200
AGCAGCGACTACATGTGT
Ab13_VH CDRH1
SEQ ID NO: 201
SSDYMC
Ab13_VH CDRH2
SEQ ID NO: 202
TGCATTTATACTGGTGATGGTACCACATACTACGCGAGCTGGGCGAAAGGC
Ab13_VH CDRH2
SEQ ID NO: 203
CIYTGDGTTYYASWAKG
Ab13_VH CDRH3
SEQ ID NO: 204
GGGGCTGGTGTTGGTTCTTATGGTTATAATTTG
Ab13_VH CDRH3
SEQ ID NO: 205
GAGVGSYGYNL
Ab13_VL VL
SEQ ID NO: 206
GTTGACATTGTGATGACCCAGACTCCAGCCTCCGTGGAGGCAGCTGTGGGAGACACAGTCACCATCAATTGCCAG
GCCAGTCAGAACATTTACAGCAATTTAGCCTGGTATCAACAGAAACCAGGGCAGCCTCCCAAGCTCCTGATCTAC
AGGGCATCCACTCTGGCATCTGGGGTCCCATCGCGGTTCAAAGGCAGTGGATCTGGGACACAGTTCACTCTCACC
ATCAGCGACCTGGAGTGTGCCGATGCTGCCACTTACTACTGTCAAAGCTATTATGCGAGTAGTATTAGTAATTAT
TTCGGCGGAGGGACCGAGGTGGTGGTCAAACGT
Ab13_VL VL
SEQ ID NO: 207
VDIVMTQTPASVEAAVGDTVTINCQASQNIYSNLAWYQQKPGQPPKLLIYRASTLASGVPSRFKGSGSGTQFTLT
ISDLECADAATYYCQSYYASSISNYFGGGTEVVVKR
Ab13_VL CDRL1
SEQ ID NO: 208
CAGGCCAGTCAGAACATTTACAGCAATTTAGCC
Ab13_VL CDRL1
SEQ ID NO: 209
QASQNIYSNLA
Ab13_VL CDRL2
SEQ ID NO: 210
AGGGCATCCACTCTGGCATCT
Ab13_VL CDRL2
SEQ ID NO: 211
RASTLAS
Ab13_VL CDRL3
SEQ ID NO: 212
CAAAGCTATTATGCGAGTAGTATTAGTAATTAT
Ab13_VL CDRL3
SEQ ID NO: 213
QSYYASSISNY
Ab14_VH VH
SEQ ID NO: 214
CAGCAGCAGCTGGAGGAGTCCGGGGGAGGCCTGGTCAAGCCTGGAGGAACCCTGACACTCACCTGCAAAGGCTCT
GGAATCGACTTCAATGATTACTACTACATGTGCTGGGTCCGCCAGGCTCCAGGGAAGGGGCTGGAGTGGATCGCG
TGCATTTATAATGGTGATGGTGAGACTTACTACGCGAACTGGGCGAAAGGCCGATTCACCATCTCCAAAACCTCG
TCGACCACGGTGACTCTGCAGATGACCAGTCTGACAGCCGCGGACACGGCCACCTATTTCTGTGCGAGGGGTAGT
GGTGTTGGGAGTTATGGTTACAACTTTTGGGGCCAGGGGACCCTGGTCACCGTCTCGAGC
Ab14_VH VH
SEQ ID NO: 215
QQQLEESGGGLVKPGGTLTLTCKGSGIDFNDYYYMCWVRQAPGKGLEWIACIYNGDGETYYANWAKGRFTISKTS
STTVTLQMTSLTAADTATYFCARGSGVGSYGYNFWGQGTLVTVSS
Ab14_VH CDRH1
SEQ ID NO: 216
GATTACTACTACATGTGC
Ab14_VH CDRH1
SEQ ID NO: 217
DYYYMC
Ab14_VH CDRH2
SEQ ID NO: 218
TGCATTTATAATGGTGATGGTGAGACTTACTACGCGAACTGGGCGAAAGGC
Ab14_VH CDRH2
SEQ ID NO: 219
CIYNGDGETYYANWAKG
Ab14_VH CDRH3
SEQ ID NO: 220
GGTAGTGGTGTTGGGAGTTATGGTTACAACTTT
Ab14_VH CDRH3
SEQ ID NO: 221
GSGVGSYGYNF
Ab14_VL VL
SEQ ID NO: 222
GCTGACATTGTGATGACCCAGACTCCAGCCTCCGTGGAGGCAGCTGTGGGAGGCACAGTCACCATCAAGTGCCAG
GCCAGTGAGAGCATTTACAGCAATTTAGCCTGGTATCAGGAGAAACCAGGGCAGCCTCCCAAGCTCCTAATCTAC
AGGGCATCCAATCTGGCATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTATGCCAGTACTGTTACTAATGCT
TTCGGCGGAGGGACCGAGGTGGTGGTCAAACGT
Ab14_VL VL
SEQ ID NO: 223
ADIVMTQTPASVEAAVGGTVTIKCQASESIYSNLAWYQQKPGQPPKLLIYRASNLASGVPSRFKGSGSGTEFTLT
ISDLECADAATYYCQGYYASTVTNAFGGGTEVVVKR
Ab14_VL CDRL1
SEQ ID NO: 224
CAGGCCAGTGAGAGCATTTACAGCAATTTAGCC
Ab14_VL CDRL1
SEQ ID NO: 225
QASESIYSNLA
Ab14_VL CDRL2
SEQ ID NO: 226
AGGGCATCCAATCTGGCATCT
Ab14_VL CDRL2
SEQ ID NO: 227
RASNLAS
Ab14_VL CDRL3
SEQ ID NO: 228
CAAGGCTATTATGCCAGTACTGTTACTAATGCT
Ab14_VL CDRL3
SEQ ID NO: 229
QGYYASTVTNA
Ab15_VH VH
SEQ ID NO: 230
CAGCAGCTGGTGGAGTCCGGGGGAGGCCTGGTCAAGCCTGGAGGAACCCTGACACTCACCTGCAAAGGCTCTGGA
ATCGACTTCAATGACTACTACTACATATGCTGGGTCCGCCAGGCTCCAGGGAAGGGGCTGGAGTGGATCCTATGC
ATTTATAATGGCGATGGTGAGACTTACTACGCGAACTGGGCGAAAGGCCGATTCACCATCTCCAAAACCTCGTCG
ACCACGGTGACTCTGCAGATGACCAGTCTGACAGGCGCGGACACGGCCACCTATTTCTGTGCGAGGGGTAGTGGT
GTTGGGAGTTATGGTTACAACTTTTGGGGCCAGGGGACCCTGGTCACCGTCTCGAGC
Ab15_VH VH
SEQ ID NO: 231
QQLVESGGGLVKPGGTLTLTCKGSGIDFNDYYYICWVRQAPGKGLEWILCIYNGDGETYYANWAKGRFTISKTSS
TTVTLQMTSLTGADTATYFCARGSGVGSYGYNFWGQGTLVTVSS
Ab15_VH CDRH1
SEQ ID NO: 232
GACTACTACTACATATGC
Ab15_VH CDRH1
SEQ ID NO: 233
DYYYIC
Ab15_VH CDRH2
SEQ ID NO: 234
TGCATTTATAATGGCGATGGTGAGACTTACTACGCGAACTGGGCGAAAGGC
Ab15_VH CDRH2
SEQ ID NO: 235
CIYNGDGETYYANWAKG
Ab15_VH CDRH3
SEQ ID NO: 236
GGTAGTGGTGTTGGGAGTTATGGTTACAACTTT
Ab15_VH CDRH3
SEQ ID NO: 237
GSGVGSYGYNF
Ab15_VL VL
SEQ ID NO: 238
GCTGACATTGTGATGACCCAGACTCCAGCCTCCGTGGAGGCAGCTGTGGGAGGCACAGTCACCATCAAGTGCCAG
GCCAGTGAGAACATTTACAGCAATTTAGCCTGGTATCAGCAGAAACCAGGGCAGCCTCCCAAGCTCCTAATTTAC
AGGGCATCCAATCTGGCATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTATGCCAGTACTGTTACTAATGGT
TTCGGCGGAGGGACCGAGGTGGTGGTCAAACGT
Ab15_VL VL
SEQ ID NO: 239
ADIVMTQTPASVEAAVGGTVTIKCQASENTYSNLAWYQQKPGQPPKLLIYRASNLASGVPSRFKGSGSGTEFTLT
ISDLECADAATYYCQGYYASTVTNGFGGGTEVVVKR
Ab15_VL CDRL1
SEQ ID NO: 240
CAGGCCAGTGAGAACATTTACAGCAATTTAGCC
Ab15_VL CDRL1
SEQ ID NO: 241
QASENIYSNLA
Ab15_VL CDRL2
SEQ ID NO: 242
AGGGCATCCAATCTGGCATCT
Ab15_VL CDRL2
SEQ ID NO: 243
RASNLAS
Ab15_VL CDRL3
SEQ ID NO: 244
CAAGGCTATTATGCCAGTACTGTTACTAATGGT
Ab15_VL CDRL3
SEQ ID NO: 245
QGYYASTVTNG
Ab16_VH VH
SEQ ID NO: 246
CAGTCGGTGGAGGAGTCCGGGGGTCGCCTGGTCACGCCTGGGACACCCCTGACACTCACCTGCACAGCCTCTGGA
TTCACCATCAGTAGCTATTCAATGATCTGGGTCCGCCAGGCTCCAGGGAAGGGGCTGGAATCCATCGGATGGATT
AATACTGGTGGTAGCGCATACTACGCGAACTGGGCAAAAGGCCGATTCACCATCTCCAGAACCTCGACCACGGTG
GATCTGAAAATGACCAGTCTGACAACCGAGGACACGGCCACCTATTTCTGTGCCAGAGACAATAACTGGTCTTTT
AACTTGTGGGGCCAAGGCACCCTGGTCACCGTCTCGAGC
Ab16_VH VH
SEQ ID NO: 247
QSVEESGGRLVTPGTPLTLTCTASGFTISSYSMIWVRQAPGKGLESIGWINTGGSAYYANWAKGRFTISRTSTTV
DLKMTSLTTEDTATYFCARDNNWSFNLWGQGTLVTVSS
Ab16_VH CDRH1
SEQ ID NO: 248
AGCTATTCAATGATC
Ab16_VH CDRH1
SEQ ID NO: 249
SYSMI
Ab16_VH CDRH2
SEQ ID NO: 250
TGGATTAATACTGGTGGTAGCGCATACTACGCGAACTGGGCAAAAGGC
Ab16_VH CDRH2
SEQ ID NO: 251
WINTGGSAYYANWAKG
Ab16_VH CDRH3
SEQ ID NO: 252
GACAATAACTGGTCTTTTAACTTG
Ab16_VH CDRH3
SEQ ID NO: 253
DNNWSFNL
AB16_VL VL
SEQ ID NO: 254
GCTGACATTGTGATGACCCAGACTCCATCCTCCGTGTCTGCAGCTGTGGGAGGCACAGTCACCATCAAGTGCCAG
GCCAGTCAGAGCATTGGTACTAATTTAGCCTGGTATCAGCAGAAATCAGGGCAGCCTCCCAAGCGCCTGACCTAC
AGGGCATCCAATCTGGAATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCGGTTATTATCGTGAGGGTAATAGTCCC
ACTTATCCTTTCGGCGGAGGGACCGAGGTGGTGGTCAAACGT
AB16_VL VL
SEQ ID NO: 255
ADIVMTQTPSSVSAAVGGTVTIKCQASQSIGTNLAWYQQKSGQPPKRLTYRASNLESGVPSRFKGSGSGTEFTLT
ISDLECADAATYYCQGGYYREGNSPTYPFGGGTEVVVKR
AB16_VL CDRL1
SEQ ID NO: 256
CAGGCCAGTCAGAGCATTGGTACTAATTTAGCC
AB16_VL CDRL1
SEQ ID NO: 257
QASQSIGTNLA
AB16_VL CDRL2
SEQ ID NO: 258
AGGGCATCCAATCTGGAATCT
AB16_VL CDRL2
SEQ ID NO: 259
RASNLES
AB16_VL CDRL3
SEQ ID NO: 260
CAAGGCGGTTATTATCGTGAGGGTAATAGTCCCACTTATCCT
AB16_VL CDRL3
SEQ ID NO: 261
QGGYYREGNSPTYP
Ab17_VH VH
SEQ ID NO: 262
CAGCGGCAGCTGGAGGAGTCCGGGGGAGGCCTGGTCAAGCCTGGAGGAGCCCTGACACTCACCTGCAAAGCCTCT
GGAATCGACTTCAGTAGCTACTACTACATATGTTGGGTCCGCCAGGCTCCAGGGAAGGGGCTGGAGTGGATCGCA
TGCATTTATGCTGGTGATGGTACCACTTACTACGCGACCTGGGCGGAAGGCCGATTCACCAGCTCCAAAACCTCG
TCGACCACGGTGACTCTGCAAATGACCAGTCTGACAGCCGCGGACACGGCCACCTATTTCTGTGCGAGGGGTAGT
GGTGTTGGTAGTTATGGTTACAACTTGTGGGGCCAAGGCACCCTCGTCACCGTCTCGAGC
Ab17_VH VH
SEQ ID NO: 263
QRQLEESGGGLVKPGGALTLTCKASGIDFSSYYYICWVRQAPGKGLEWIACIYAGDGTTYYATWAEGRFTSSKTS
STTVTLQMTSLTAADTATYFCARGSGVGSYGYNLWGQGTLVTVSS
Ab17_VH CDRH1
SEQ ID NO: 264
AGCTACTACTACATATGT
Ab17_VH CDRH1
SEQ ID NO: 265
SYYYIC
Ab17_VH CDRH2
SEQ ID NO: 266
TGCATTTATGCTGGTGATGGTACCACTTACTACGCGACCTGGGCGGAAGGC
Ab17_VH CDRH2
SEQ ID NO: 267
CIYAGDGTTYYATWAEG
Ab17_VH CDRH3
SEQ ID NO: 268
GGTAGTGGTGTTGGTAGTTATGGTTACAACTTG
Ab17_VH CDRH3
SEQ ID NO: 269
GSGVGSYGYNL
Ab17_VL VL
SEQ ID NO: 270
GCTGACATTGTGATGACCCAGACTCCAGCCTCCGTGGAGGCAGCTGTGGGAGGCACAGTCACCATCAAGTGCCAG
GCCAGTGAGTACATTTATAGCAATTTAGCCTGGTATCAGCAAAAACCAGGGCAGCCTCCCAAGCTCCTGATCTAC
AGGGCATCCACTCTGGAATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTTTCCCAGTATTATTACTAATGCT
TTCGGCGGAGGGACCGAGGTGGTGGTCAAACGT
Ab17_VL VL
SEQ ID NO: 271
ADIVMTQTPASVEAAVGGTVTIKCQASEYIYSNLAWYQQKPGQPPKLLIYRASTLESGVPSRFKGSGSGTEFTLT
ISDLECADAATYYCQGYFPSIITNAFGGGTEVVVKR
Ab17_VL CDRL1
SEQ ID NO: 272
CAGGCCAGTGAGTACATTTATAGCAATTTAGCC
Ab17_VL CDRL1
SEQ ID NO: 273
QASEYIYSNLA
Ab17_VL CDRL2
SEQ ID NO: 274
AGGGCATCCACTCTGGAATCT
Ab17_VL CDRL2
SEQ ID NO: 275
RASTLES
Ab17_VL CDRL3
SEQ ID NO: 276
CAAGGCTATTTTCCCAGTATTATTACTAATGCT
Ab17_VL CDRL3
SEQ ID NO: 277
QGYFPSIITNA
Ab18_VH VH
SEQ ID NO: 278
CAGCAGCAGCTGGAGGAGTCCGGGGGAGGCCTGGTCAAGCCTGGAGGAACCCTGACACTCACCTGCAAAGCCTCT
GGATTAGACTTCAGTAACTACCACTACATGTGCTGGGTCCGCCAGGCTCCAGGGAAGAGGCCTGAGTGGATCGCA
TGCATTTATAATGGTGATGGTACCACTTACGACGCGAGCTGGGCGAAAGGCCGATTCACCAGCTCCAAAACCTCG
TCGACCACGGTGACTCTGCAAATGACCAGTCTGACAGCCGCGGACACGGCCACCTATTTCTGTGCGAGGGGTAGT
GGTGTTGGTAGTTATGGTTACAACTTGTGGGGCCAAGGCACCCTCGTCACCGTCTCGAGC
Ab18_VH VH
SEQ ID NO: 279
QQQLEESGGGLVKPGGTLTLTCKASGLDFSNYHYMCWVRQAPGKRPEWIACIYNGDGTTYDASWAKGRFTSSKTS
STTVTLQMTSLTAADTATYFCARGSGVGSYGYNLWGQGTLVTVSS
Ab18_VH CDRH1
SEQ ID NO: 280
AACTACCACTACATGTGC
Ab18_VH CDRH1
SEQ ID NO: 281
NYHYMC
Ab18_VH CDRH2
SEQ ID NO: 282
TGCATTTATAATGGTGATGGTACCACTTACGACGCGAGCTGGGCGAAAGGC
Ab18_VH CDRH2
SEQ ID NO: 283
CIYNGDGTTYDASWAKG
Ab18_VH CDRH3
SEQ ID NO: 284
GGTAGTGGTGTTGGTAGTTATGGTTACAACTTG
Ab18_VH CDRH3
SEQ ID NO: 285
GSGVGSYGYNL
Ab18_VL VL
SEQ ID NO: 286
GCTGACATTGTGATGACCCAGACTCCAGCCTCCGTGGAGGCAGCTGTGGGAGGCACAGTCACCATCAAGTGCCAG
GCCAGTGAGAGCATTTATAGCAATTTAGCCTGGTATCAGCAGAAACCAGGGCAGCCTCCCAAGCTCCTGATCTAC
AGGGCATCCACTCTGGAATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTATGCCAGTATTATTACTAATGCT
TTCGGCGGAGGGACCGAGGTGGTGGTCAAACGT
Ab18_VL VL
SEQ ID NO: 287
ADIVMTQTPASVEAAVGGTVTIKCQASESIYSNLAWYQQKPGQPPKLLIYRASTLESGVPSRFKGSGSGTEFTLT
ISDLECADAATYYCQGYYASIITNAFGGGTEVVVKR
Ab18_VL CDRL1
SEQ ID NO: 288
CAGGCCAGTGAGAGCATTTATAGCAATTTAGCC
Ab18_VL CDRL1
SEQ ID NO: 289
QASESIYSNLA
Ab18_VL CDRL2
SEQ ID NO: 290
AGGGCATCCACTCTGGAATCT
Ab18_VL CDRL2
SEQ ID NO: 291
RASTLES
Ab18_VL CDRL3
SEQ ID NO: 292
CAAGGCTATTATGCCAGTATTATTACTAATGCT
Ab18_VL CDRL3
SEQ ID NO: 293
QGYYASIITNA
Ab19_VH VH
SEQ ID NO: 294
CAGGAGCAGCTGGAGGAGTCCGGGGGAGACCTGGTCAAGCCTGAGGGATCCCTGACACTCACCTGCACAGCCTCT
GGATTCTCCTTCAGTAACTACTACTACATGTGCTGGGTCCGCCAGGCTCCAGGGAAGGGGCTGGAGTGGATCGCA
TGCATTTATACTAGTAGTGGTAGCACTTACTACGCGAACTGGGTGAATGGCCGATTCACCATCTCCAGAAGCACC
AGCCTAAACACGGTGACTCTGCAAGTGACCAGTCTGACAGCCGCGGACACGGCCACCTATTTCTGTGCGAAATCT
TTTGGTGTTGGTGGTTACTCCTTTAACTTGTGGGGCCAAGGCACCCTGGTCACCGTCTCGAGC
Ab19_VH VH
SEQ ID NO: 295
QEQLEESGGDLVKPEGSLTLTCTASGESFSNYYYMCWVRQAPGKGLEWIACIYTSSGSTYYANWVNGRFTISRST
SLNTVTLQVTSLTAADTATYFCAKSFGVGGYSFNLWGQGTLVTVSS
Ab19_VH CDRH1
SEQ ID NO: 296
AACTACTACTACATGTGC
Ab19_VH CDRH1
SEQ ID NO: 297
NYYYMC
Ab19_VH CDRH2
SEQ ID NO: 298
TGCATTTATACTAGTAGTGGTAGCACTTACTACGCGAACTGGGTGAATGGC
Ab19_VH CDRH2
SEQ ID NO: 299
CIYTSSGSTYYANWVNG
Ab19_VH CDRH3
SEQ ID NO: 300
TCTTTTGGTGTTGGTGGTTACTCCTTTAACTTG
Ab19_VH CDRH3
SEQ ID NO: 301
SFGVGGYSFNL
Ab19_VL VL
SEQ ID NO: 302
GCCTATGATATGACCCAGACTCCAGCCTCTGTGGAGGTAGCTGTGGGAGGCACAGTCACCATCAAGTGCCAGGCC
AGTCAGAGTATTAGTAGCTACTTATCCTGGTATCAGCAGAAACCAGGGCAGCGTCCCAAGCTCCTGATCTACAGG
GCATCCACTCTGGCATCTGGGGTCTCATCGCGGTTCAAAGGCAGTGGATCTGGGACAGAGTACAGTCTCTCCATC
AGCGGCATGGAGTGTGACGATGCTGCCACTTACTACTGTCAGCAGGGTACTACTATTAGTGATGTTGATAATGTT
TTCGGCGGAGGGACCGAGGTGGTGGTCAAACGT
Ab19_VL VL
SEQ ID NO: 303
AYDMTQTPASVEVAVGGTVTIKCQASQSISSYLSWYQQKPGQRPKLLIYRASTLASGVSSREKGSGSGTEYSLSI
SGMECDDAATYYCQQGTTISDVDNVFGGGTEVVVKR
Ab19_VL CDRL1
SEQ ID NO: 304
CAGGCCAGTCAGAGTATTAGTAGCTACTTATCC
Ab19_VL CDRL1
SEQ ID NO: 305
QASQSISSYLS
Ab19_VL CDRL2
SEQ ID NO: 306
AGGGCATCCACTCTGGCATCT
Ab19_VL CDRL2
SEQ ID NO: 307
RASTLAS
Ab19_VL CDRL3
SEQ ID NO: 308
CAGCAGGGTACTACTATTAGTGATGTTGATAATGTT
Ab19_VL CDRL3
SEQ ID NO: 309
QQGTTISDVDNV
Ab20_VH VH
SEQ ID NO: 310
CAGTCGGTGGAGGAGTCCGGGGGTCGCCTGGTCACGCCTGGGACACCCCTGACACTCACCTGCACAGTCTCTGGA
TTCTCCCTCAGTGATTATGGATTGAACTGGGTCCGCCAGGCTCCAGGGAAGGGGCTGGAGTGGATCGGAATCATT
TATGCTAGTGGTAGCACATACTACGCGAACTGGGCGAAAGGCCGATTCACCATCTCCAAAACCTCGACCACGGTG
GATCTGAAAATGACCAGTCCGACAACCGAGGACACGGCCACCTATTTCTGTGTCAGAGATAGTTCTGCTACTTAT
GACTACTTTAACTTGTGGGGCCCGGGCACCCTGGTCACCGTCTCGAGC
Ab20_VH VH
SEQ ID NO: 311
QSVEESGGRLVTPGTPLTLTCTVSGESLSDYGLNWVRQAPGKGLEWIGITYASGSTYYANWAKGRFTISKTSTTV
DLKMTSPTTEDTATYFCVRDSSATYDYFNLWGPGTLVTVSS
Ab20_VH CDRH1
SEQ ID NO: 312
GATTATGGATTGAAC
Ab20_VH CDRH1
SEQ ID NO: 313
DYGLN
Ab20_VH CDRH2
SEQ ID NO: 314
ATCATTTATGCTAGTGGTAGCACATACTACGCGAACTGGGCGAAAGGC
Ab20_VH CDRH2
SEQ ID NO: 315
IIYASGSTYYANWAKG
Ab20 _VH CDRH3
SEQ ID NO: 316
GATAGTTCTGCTACTTATGACTACTTTAACTTG
Ab20_VH CDRH3
SEQ ID NO: 317
DSSATYDYFNL
Ab20_VL VL
SEQ ID NO: 318
TATGATATGACCCAGACTCCAGCCTCTGTGGAGGTAGCTGTGGGAGGCACAGTCACCATCAAGTGCCAGGCCAGT
CAGAGCATTAGTAGCTACTTAGCCTGGTATCAGCAGAAACCGGGGCAGCCTCCCCGGCTCCTGATCTATGGTGCA
TCCAATCTGGCGTCTGGGGTCCCATCGCGGTTCAAAGGCAGTGGATCTGGGACAGAGTACACTCTCACCATCAGC
GACCTGGAGTGTGCCGATGCTGCCACTTACTACTGTCAACAAGCCTTGAGTCCTAGTAATGTTGATAATGCTTTC
GGCGGAGGGACCGAGGTGGTGGTCAAACGT
Ab20_VL VL
SEQ ID NO: 319
YDMTQTPASVEVAVGGTVTIKCQASQSISSYLAWYQQKPGQPPRLLIYGASNLASGVPSRFKGSGSGTEYTLTIS
DLECADAATYYCQQALSPSNVDNAFGGGTEVVVKR
Ab20_VL CDRL1
SEQ ID NO: 320
CAGGCCAGTCAGAGCATTAGTAGCTACTTAGCC
Ab20_VL CDRL1
SEQ ID NO: 321
QASQSISSYLA
Ab20_VL CDRL2
SEQ ID NO: 322
GGTGCATCCAATCTGGCGTCT
Ab20_VL CDRL2
SEQ ID NO: 323
GASNLAS
Ab20_VL CDRL3
SEQ ID NO: 324
CAACAAGCCTTGAGTCCTAGTAATGTTGATAATGCT
Ab20_VL CDRL3
SEQ ID NO: 325
QQALSPSNVDNA
Ab21_VH VH
SEQ ID NO: 326
CAGTCGTTGGAGGAGTCCGGGGGAGGCCTGGTCCAGCCTGGGGCATCCCTGACACTCACCTGCACAGCTTCTGGA
TTCTCCTTCAGTAGCAACTACTGGATATGCTGGGTCCGCCAGGCTCCAGGGAAGGGGCTGGAGTGGATCGGATGC
ATTTATACTGGTACTGGTATCACATGGTACGCGAGCTGGGTGAATGGCCGAGTCACCATCTCCAGCACCTCGTCG
ACCACGGTGACTCTGCAAGTGAACAGTCTGACAGCCGCGGACACGGCCACTTATTTCTGTGCGAGAGGAAGTGGT
GATATCGCCTGGGCTTGGGATTTGTGGGGCCAAGGCACCCTGGTCACCGTCTCGAGC
Ab21_VH VH
SEQ ID NO: 327
QSLEESGGGLVQPGASLTLTCTASGFSFSSNYWICWVRQAPGKGLEWICCIYTGTGITWYASWVNGRVTISSTSS
TTVTLQVNSLTAADTATYFCARGSGDIAWAWDLWGQGTLVTVSS
Ab21_VH CDRH1
SEQ ID NO: 328
AGCAACTACTGGATATGC
Ab21_VH CDRH1
SEQ ID NO: 329
SNYWIC
Ab21_VH CDRH2
SEQ ID NO: 330
TGCATTTATACTGGTACTGGTATCACATGGTACGCGAGCTGGGTGAATGGC
Ab21_VH CDRH2
SEQ ID NO: 331
CIYTGTGITWYASWVNG
Ab21_VH CDRH3
SEQ ID NO: 332
GGAAGTGGTGATATCGCCTGGGCTTGGGATTTG
Ab21_VH CDRH3
SEQ ID NO: 333
GSGDIAWAWDL
Ab21_VL VL
SEQ ID NO: 334
GATGTTGTGATGACCCAGACTCCAGCCTCCGTGTCTGCAGCTGTGGGAGGCACAGTCACCATCAAGTGCCAGGCC
AGTCAGAGCATTTATACCAATTTAGCCTGGTATCAGCAGAAACCAGGGCAGCCTCCCAAGCTCCTGATCTACAAG
GCATCCACTCTGGCATCTGGGGTCTCATCGCGGTTCAAAGGCAGTGGATCTGGGACAGAGTTCACTCTCACCATC
AGCGGCGTGCAGTGTGCCGATGCAGCCACTTACTACTGTCAATGTACTGGTTATGGTAGTAGTGGATGGACTTTC
GGCGGAGGGACCGAGGTGGTGGTCAAACGT
Ab21_VL VL
SEQ ID NO: 335
DVVMTQTPASVSAAVGGTVTIKCQASQSIYTNLAWYQQKPGQPPKLLIYKASTLASGVSSRFKGSGSGTEFTLTI
SGVQCADAATYYCQCTGYGSSGWTFGGGTEVVVKR
Ab21_VL CDRL1
SEQ ID NO: 336
CAGGCCAGTCAGAGCATTTATACCAATTTAGCC
Ab21_VL CDRL1
SEQ ID NO: 337
QASQSIYTNLA
Ab21_VL CDRL2
SEQ ID NO: 338
AAGGCATCCACTCTGGCATCT
Ab21_VL CDRL2
SEQ ID NO: 339
KASTLAS
Ab21_VL CDRL3
SEQ ID NO: 340
CAATGTACTGGTTATGGTAGTAGTGGATGGACT
Ab21_VL CDRL3
SEQ ID NO: 341
QCTGYGSSGWT
Ab22_VH VH
SEQ ID NO: 342
CAGTCGTTGGAGGAGTCCGGGGGAGACCTGGTCAAGCCTGGGGCATCCCTGACACTCACCTGCACAGCCTCTGGA
TTCTCCTTCAGTACCAGCTATTACATGTGCTGGGTCCGCCAGGCTCCAGGGAAGGGGCTGGAGTGGATCGGATGC
ATACATACTAGTACGTTTTCCACATGGTACGCGAGCTGGGCGAAAGGCCGATTCACCATCTCCAAAACCTCGTCG
ACCACGGTGACTCTGCAAATGACCAGTCTGACAGCCGCGGACACGGCCACCTATTTCTGTGCGAGAAGAGATGCT
TATAATTATGCTGGTTATGCTTATAACTTGTGGGGCCAGGGGACCCTGGTCACCGTCTCGAGC
Ab22_VH VH
SEQ ID NO: 343
QSLEESGGDLVKPGASLTLTCTASGESFSTSYYMCWVRQAPGKGLEWIGCIHTSTESTWYASWAKGRFTISKTSS
TTVTLQMTSLTAADTATYFCARRDAYNYAGYAYNLWGQGTLVTVSS
Ab22_VH CDRH1
SEQ ID NO: 344
ACCAGCTATTACATGTGC
Ab22_VH CDRH1
SEQ ID NO: 345
TSYYMC
Ab22_VH CDRH2
SEQ ID NO: 346
TGCATACATACTAGTACGTTTTCCACATGGTACGCGAGCTGGGCGAAAGGC
Ab22_VH CDRH2
SEQ ID NO: 347
CIHTSTFSTWYASWAKG
Ab22_VH CDRH3
SEQ ID NO: 348
AGAGATGCTTATAATTATGCTGGTTATGCTTATAACTTG
Ab22_VH CDRH3
SEQ ID NO: 349
RDAYNYAGYAYNL
Ab22_VL VL
SEQ ID NO: 350
GATGTTGTGATGACCCAGACTCCAGCCTCGGTGTCTGCCGCTGTGGGAGGCACAGTCACCATCAATTGCCAGGCC
AGTGAGGACATTTATAGCAATTTGGCCTGGTATCAGCAGAAACCAGGGCAGCCTCCCAAGCTCCTGATCTATGGT
GCATCCACTCTGGCATCTGGGGTCCCATCGCGGTTCAAAGGCAGTGGATCTGGGACAGAGTTCACTCTCACCATC
AGCGACCTGGAGTGTGCCGATGCTGCCACTTACTACTGTCAATGCACTTTTGGTTGTTTTAGTGGTAGTAGTAAC
AACATTTTCGGCGGAGGGACCGAGGTGGTGGTCAAACGT
Ab22_VL VL
SEQ ID NO: 351
DVVMTQTPASVSAAVGGTVTINCQASEDIYSNLAWYQQKPGQPPKLLIYGASTLASGVPSRFKGSGSGTEFTLTI
SDLECADAATYYCQCTFGCFSGSSNNIFGGGTEVVVKR
Ab22_VL CDRL1
SEQ ID NO: 352
CAGGCCAGTGAGGACATTTATAGCAATTTGGCC
Ab22_VL CDRL1
SEQ ID NO: 353
QASEDIYSNLA
Ab22_VL CDRL2
SEQ ID NO: 354
GGTGCATCCACTCTGGCATCT
Ab22_VL CDRL2
SEQ ID NO: 355
GASTLAS
Ab22_VL CDRL3
SEQ ID NO: 356
CAATGCACTTTTGGTTGTTTTAGTGGTAGTAGTAACAACATT
Ab22_VL CDRL3
SEQ ID NO: 357
QCTFGCFSGSSNNI
Ab23_VH VH
SEQ ID NO: 358
CAGCAGCAGCTGGAGGAGTCCGGGGGAGGCCTGGTCAAGCCTGGAGGAACCCTGACACTCACCTGCAAAGCCTCT
GGAATCGACTTCAATGACTACTACTACATGTGCTGGGTCCGCCAGGCTCCAGGGAAGGGGCCTGAGTGGATCGCA
TGCATTTATAATGGTGATGGTGACACTTACTACGCGAACTGGGCGAAAGGCCGATTCACCATCTCCAAAACCTCG
TCGACCACGGTGACTCTGCAAATGACCAGTCTGACAGCCGCGGACACGGCCACCTATTTCTGTGCGAGGGGTAGT
GGTGTTGGTAGTTATGGTTACAACTTGTGGGGCCAAGGCACCCTGGTCACCGTCTCGAGC
Ab23 VH VH
SEQ ID NO: 359
QQQLEESGGGLVKPGGTLTLTCKASGIDFNDYYYMCWVRQAPGKGPEWIACIYNGDGDTYYANWAKGRFTISKTS
STTVTLQMTSLTAADTATYFCARGSGVGSYGYNLWGQGTLVTVSS
Ab23_VH CDRH1
SEQ ID NO: 360
GACTACTACTACATGTGC
Ab23_VH CDRH1
SEQ ID NO: 361
DYYYMC
Ab23_VH CDRH2
SEQ ID NO: 362
TGCATTTATAATGGTGATGGTGACACTTACTACGCGAACTGGGCGAAAGGC
Ab23_VH CDRH2
SEQ ID NO: 363
CIYNGDGDTYYANWAKG
Ab23_VH CDRH3
SEQ ID NO: 364
GGTAGTGGTGTTGGTAGTTATGGTTACAACTTG
Ab23_VH CDRH3
SEQ ID NO: 365
GSGVGSYGYNL
Ab23_VL VL
SEQ ID NO: 366
GCTGACATTGTGATGACCCAGACTCCAGCCTCCGTGGAGGCAGCTGTGGGAGGCACAGTCACCATCAAGTGCCAG
GCCAGTGAGAGCATTTACAGCAATTTAGCCTGGTATCAGCAGAAACCAGGGCAGCCTCCCAAGCTCCTAATCTAC
AGGGCATCTACTCTGGCATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTATGCCAGTACTATTACTAATGTT
TTCGGCGGAGGGACCGAGGTGGTGGTCAAACGT
Ab23_VL VL
SEQ ID NO: 367
ADIVMTQTPASVEAAVGGTVTIKCQASESIYSNLAWYQQKPGQPPKLLIYRASTLASGVPSRFKGSGSGTEFTLT
ISDLECADAATYYCQGYYASTITNVFGGGTEVVVKR
Ab23_VL CDRL1
SEQ ID NO: 368
CAGGCCAGTGAGAGCATTTACAGCAATTTAGCC
Ab23_VL CDRL1
SEQ ID NO: 369
QASESIYSNLA
Ab23_VL CDRL2
SEQ ID NO: 370
AGGGCATCTACTCTGGCATCT
Ab23_VL CDRL2
SEQ ID NO: 371
RASTLAS
Ab23_VL CDRL3
SEQ ID NO: 372
CAAGGCTATTATGCCAGTACTATTACTAATGTT
Ab23_VL CDRL3
SEQ ID NO: 373
QGYYASTITNV
Ab24_VH VH
SEQ ID NO: 374
CAGCAGCAGCTGGAGGAGTCCGGGGGAGGCCTGGTCAAGCCTGGAGGAACCCTGACACTCACCTGCAAAGCCTCT
GGAATCGACTTCAGTAACGTCTACTACATGTGCTGGGTCCGCCAGGCTCCAGGGAAGGGGCCGGAGTGGATCGCA
TGCATTTATGTTGGTGATGGCAGCACATACGACGCGAGCTGGGCGAGAGGCCGATTCACCATCTCCAAAACCTCG
TCGACCACGGTGACTCTGCAGATGATTAGTCTGACAGCCGCGGACACGGCCACCTATTTCTGTGCGGGGGGTACT
GGTGTTGGTAGCTATGGTTACAGCTTGTGGGGCCCAGGCACCCTGGTCACCGTCTCGAGC
Ab24_VH VH
SEQ ID NO: 375
QQQLEESGGGLVKPGGTLTLTCKASGIDESNVYYMCWVRQAPGKGPEWIACIYVGDGSTYDASWARGRFTISKTS
STTVTLQMISLTAADTATYFCAGGTGVGSYGYSLWGPGTLVTVSS
Ab24_VH CDRH1
SEQ ID NO: 376
AACGTCTACTACATGTGC
Ab24_VH CDRH1
SEQ ID NO: 377
NVYYMC
Ab24_VH CDRH2
SEQ ID NO: 378
TGCATTTATGTTGGTGATGGCAGCACATACGACGCGAGCTGGGCGAGAGGC
Ab24_VH CDRH2
SEQ ID NO: 379
CIYVGDGSTYDASWARG
Ab24_VH CDRH3
SEQ ID NO: 380
GGTACTGGTGTTGGTAGCTATGGTTACAGCTTG
Ab24_VH CDRH3
SEQ ID NO: 381
GTGVGSYGYSL
Ab24_VL VL
SEQ ID NO: 382
GCTGACATTGTGATGACCCAGACTCCAGCCTCCGTGGAGGCAGCTGTGGGAGGCACAGTCACCATCAAGTGCCAG
GCCAGTGAGAGCATTTATAGCAATTTAGCCTGGTATCATCAGAAACCAGGGCAGCCTCCCAAGCTCCTGATCTAC
AGGGCATCCAATCTGGAATCTGGGGTCCCATCGCGGTTCAAAGGCAGTGGATCTGGGACAGATTTCACTCTCACC
ATCAGCGACCTGGAGTGTGCCGATGCTGCCACTTACTACTGTCAAGGCTATTATGCCAGTGTTGTTACAAATGGT
TTCGGCGGAGGGACCGAGGTGGTGGTCAAACGT
Ab24_VL VL
SEQ ID NO: 383
ADIVMTQTPASVEAAVGGTVTIKCQASESIYSNLAWYHQKPGQPPKLLIYRASNLESGVPSRFKGSGSGTDFTLT
ISDLECADAATYYCQGYYASVVTNGFGGGTEVVVKR
Ab24_VL CDRL1
SEQ ID NO: 384
CAGGCCAGTGAGAGCATTTATAGCAATTTAGCC
Ab24_VL CDRL1
SEQ ID NO: 385
QASESIYSNLA
Ab24_VL CDRL2
SEQ ID NO: 386
AGGGCATCCAATCTGGAATCT
Ab24_VL CDRL2
SEQ ID NO: 387
RASNLES
Ab24_VL CDRL3
SEQ ID NO: 388
CAAGGCTATTATGCCAGTGTTGTTACAAATGGT
Ab24_VL CDRL3
SEQ ID NO: 389
QGYYASVVTNG
Ab25_VH VH
SEQ ID NO: 390
CAGCGGCAGCTGGAGGAGTCCGGGGGAGGCCTGGTCAAGCCTGGAGGAGCCCTGACACTCACCTGCAAAGCCTCT
GGAATCGACTTCAGTAGCTACTACTACAGATGCTGGGTCCGCCAGGCTCCAGGGAAGGGGCTGGAGTGGATCGCA
TGCATTTATACTGGTGATGGTACCACTTACTACGCGAGCTGGGCGAAAGGCCGATTCACCACCTCCAAAACCTCG
TCGACCACGGTGACTCTGCAAATGACCAGTCTGACAGCCGCGGACACGGCCACCTATTTCTGTGCGAGGGGTAGT
GGTGTTGGTAGTTATGGTTACAACTTCTGGGGCCCAGGCACCCTGGTCACCGTCTCGAGC
Ab25_VH VH
SEQ ID NO: 391
QRQLEESGGGLVKPGGALTLTCKASGIDFSSYYYRCWVRQAPGKGLEWIACIYTGDGTTYYASWAKGRFTTSKTS
STTVTLQMTSLTAADTATYFCARGSGVGSYGYNFWGPGTLVTVSS
Ab25_VH CDRH1
SEQ ID NO: 392
AGCTACTACTACAGATGC
Ab25_VH CDRH1
SEQ ID NO: 393
SYYYRC
Ab25_VH CDRH2
SEQ ID NO: 394
TGCATTTATACTGGTGATGGTACCACTTACTACGCGAGCTGGGCGAAAGGC
Ab25_VH CDRH2
SEQ ID NO: 395
CIYTGDGTTYYASWAKG
Ab25_VH CDRH3
SEQ ID NO: 396
GGTAGTGGTGTTGGTAGTTATGGTTACAACTTC
Ab25_VH CDRH3
SEQ ID NO: 397
GSGVGSYGYNF
Ab25_VL VL
SEQ ID NO: 398
GCTGACATTGTGATGACCCAGACTCCAGCCTCCGTGGAGGCAGCTGTGGGAGGCACAGTCACCATCAAGTGCCAG
GCCAGTGAGAGCATTTATAGCAATTTAGCCTGGTATCAGCAGAAACCAGGGCAGCCTCCCAAGCTCCTGATCTAC
AGGGCATCCACTCTGGAATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTATGCCAGTATTATTACTAATGCT
TTCGGCGGAGGGACCGAGGTGGTGGTCAAACGT
Ab25_VL VL
SEQ ID NO: 399
ADIVMTQTPASVEAAVGGTVTIKCQASESIYSNLAWYQQKPGQPPKLLIYRASTLESGVPSRFKGSGSGTEFTLT
ISDLECADAATYYCQGYYASIITNAFGGGTEVVVKR
Ab25_VL CDRL1
SEQ ID NO: 400
CAGGCCAGTGAGAGCATTTATAGCAATTTAGCC
Ab25_VL CDRL1
SEQ ID NO: 401*
QASESIYSNLA
Ab25_VL CDRL2
SEQ ID NO: 402
AGGGCATCCACTCTGGAATCT
Ab25_VL CDRL2
SEQ ID NO: 403
RASTLES
Ab25_VL CDRL3
SEQ ID NO: 404
CAAGGCTATTATGCCAGTATTATTACTAATGCT
Ab25_VL CDRL3
SEQ ID NO: 405
QGYYASIITNA
Ab26_VH VH
SEQ ID NO: 406
CAGTCGGTGGAGGAGTCCGGGGGAGGCCTGGTCAAGCCTGAGGGATCCCTGACACTCACCTGCAAAGCCTCTGGA
TTCGACCTCAGTAGCTACTACTACATGTGCTGGGTCCGCCAGGCTCCAGGGAAGGGGCCGGAGTGGATCGCATGC
ATTTATGCTGGTGATGGCACCACATACTACGCGAGCTGGGCGAAAGGCCGATTCACCATCTCCAAAACCTCGTCG
ACCACGGTGACTCTGCAAATGACCAGTCTGACAGCCGCGGACACGGCCACCTATTTCTGTGCGAGGGGTACTGGT
GTTGGTAGCTATGGTTACAACTTGTGGGGCCCAGGCACCCTGGTCACCGTCTCGAGC
Ab26_VH VH
SEQ ID NO: 407
QSVEESGGGLVKPEGSLTLTCKASGFDLSSYYYMCWVRQAPGKGPEWIACIYAGDGTTYYASWAKGRFTISKTSS
TTVTLQMTSLTAADTATYFCARGTGVGSYGYNLWGPGTLVTVSS
Ab26_VH CDRH1
SEQ ID NO: 408
AGCTACTACTACATGTGC
Ab26_VH CDRH1
SEQ ID NO: 409
SYYYMC
Ab26_VH CDRH2
SEQ ID NO: 410
TGCATTTATGCTGGTGATGGCACCACATACTACGCGAGCTGGGCGAAAGGC
Ab26_VH CDRH2
SEQ ID NO: 411
CIYAGDGTTYYASWAKG
Ab26_VH CDRH3
SEQ ID NO: 412
GGTACTGGTGTTGGTAGCTATGGTTACAACTTG
Ab26_VH CDRH3
SEQ ID NO: 413
GTGVGSYGYNL
Ab26_VL VL
SEQ ID NO: 414
GCTGACATTGTGATGACCCAGACTCCAGCCTCCGTGGAGGCAGCTGTGGGAGGCACAGTCACCATCAAGTGCCAG
GCCAGTGAGAACATTTACAGCAATTTAGCCTGGTATCAGCAGAAACCAGGGCAGCCTCCCAAGCTCCTAATCTAC
AGGGCATCCAATCTGGAATCTGGGGTCCCATCGCGGTTCAAAGGCAGTGGATCTGGGACAGAGTTCACTCTCACC
ATCAGCGACCTGGAGTGTGACGATGCTGCCACTTACTACTGTCAAGGCTATTATCCCAGTGCTGTTACTAATGCT
TTCGGCGGAGGGACCGAGGTGGTGGTCAAACGT
Ab26_VL VL
SEQ ID NO: 415
ADIVMTQTPASVEAAVGGTVTIKCQASENIYSNLAWYQQKPGQPPKLLIYRASNLESGVPSRFKGSGSGTEFTLT
ISDLECDDAATYYCQGYYPSAVTNAFGGGTEVVVKR
Ab26_VL CDRL1
SEQ ID NO: 416
CAGGCCAGTGAGAACATTTACAGCAATTTAGCC
Ab26_VL CDRL1
SEQ ID NO: 417
QASENIYSNLA
Ab26_VL CDRL2
SEQ ID NO: 418
AGGGCATCCAATCTGGAATCT
Ab26_VL CDRL2
SEQ ID NO: 419
RASNLES
Ab26_VL CDRL3
SEQ ID NO: 420
CAAGGCTATTATCCCAGTGCTGTTACTAATGCT
Ab26_VL CDRL3
SEQ ID NO: 421
QGYYPSAVTNA
Ab27_VH VH
SEQ ID NO: 422
CAGTCGGTGGAGGAGTCCGGGGGAGGCCTGGTCAAGCCTGGAGGAACCCTGACACTCACCTGCAAAGCCTCTGGA
ATCGACTTCAGTAGTTACCACTACATGTGCTGGGTCCGCCAGGCTCCAGGGAAGGGGCTGGAGTGGATCGCGTGC
ATTTATAATGGTGATGGTGACACTTACTACGCGAGCTGGGCGAAAGGCCGATTCACCATCTCCAAAACCTCGTCG
ACCACGGTGACTCTGCAAATGACCAGTCTGACAGCCGCGGACACGGCCACCTATTTCTGTGCGAGGGGTAGTGGT
GTTGGTAGTTATGGTTACAATTTGTGGGGCCCAGGCACCCTGGTCACCGTCTCGAGC
Ab27 VH VH
SEQ ID NO: 423
QSVEESGGGLVKPGGTLTLTCKASGIDFSSYHYMCWVRQAPGKGLEWIACIYNGDGDTYYASWAKGRFTISKTSS
TTVTLQMTSLTAADTATYFCARGSGVGSYGYNLWGPGTLVTVSS
Ab27_VH CDRH1
SEQ ID NO: 424
AGTTACCACTACATGTGC
Ab27_VH CDRH1
SEQ ID NO: 425
SYHYMC
Ab27_VH CDRH2
SEQ ID NO: 426
TGCATTTATAATGGTGATGGTGACACTTACTACGCGAGCTGGGCGAAAGGC
Ab27_VH CDRH2
SEQ ID NO: 427
CIYNGDGDTYYASWAKG
Ab27_VH CDRH3
SEQ ID NO: 428
GGTAGTGGTGTTGGTAGTTATGGTTACAATTTG
Ab27_VH CDRH3
SEQ ID NO: 429
GSGVGSYGYNL
Ab27_VL VL
SEQ ID NO: 430
GCTGACATTGTGATGACCCAGACTCCAGCCTCCGTGGAGGCAGCTGTGGGAGGCACAGTCACCATCAAGTGCCAG
GCCAGTGAGAGCATTTACAGCAATTTAGCCTGGTATCAGCAGAAACCAGGGCAGCCTCCCAAGCTCCTAATCTAC
AGGGCATCCAATCTGGAATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTATGCCAGTACTACTACTAATGCT
TTCGGCGGAGGGACCGAGGTGGTGGTCAAACGT
Ab27_VL VL
SEQ ID NO: 431
ADIVMTQTPASVEAAVGGTVTIKCQASESIYSNLAWYQQKPGQPPKLLIYRASNLESGVPSRFKGSGSGTEFTLT
ISDLECADAATYYCQGYYASTTTNAFGGGTEVVVKR
Ab27_VL CDRL1
SEQ ID NO: 432
CAGGCCAGTGAGAGCATTTACAGCAATTTAGCC
Ab27_VL CDRL1
SEQ ID NO: 433
QASESIYSNLA
Ab27_VL CDRL2
SEQ ID NO: 434
AGGGCATCCAATCTGGAATCT
Ab27_VL CDRL2
SEQ ID NO: 435
RASNLES
Ab27_VL CDRL3
SEQ ID NO: 436
CAAGGCTATTATGCCAGTACTACTACTAATGCT
Ab27_VL CDRL3
SEQ ID NO: 437
QGYYASTTTNA
Ab28_VH VH
SEQ ID NO: 438
CAGCAGCAGCTGGAGGAGTCCGGGGGAGGCCTGGTCAAGCCTGGAGGAACCCTGACACTCACCTGCAAAGGCTCT
GGAATCGACTTCAATGACTACTACTACATGTGCTGGGTCCGCCAGGCTCCAGGGAAGGGGCTGGAGTGGATCGCA
TGCATTTATAATGGTGATGGTGAGACTTACTACGCGAACTGGGCGAAAGGCCGATTCACCATCTCCAAAACCTCG
TCGACCACGGTGACTCTGCAGATGACCAGTCTGACAGCCGCGGACACGGCCACCTATTTCTGTGCGAGGGGTAGT
GGTGTTGGGAGTTATGGTTACAACTTTTGGGGCCCAGGCACCCTGGTCACCGTCTCGAGC
Ab28_VH VH
SEQ ID NO: 439
QQQLEESGGGLVKPGGTLTLTCKGSGIDENDYYYMCWVRQAPGKGLEWIACIYNGDGETYYANWAKGRFTISKTS
STTVTLQMTSLTAADTATYFCARGSGVGSYGYNFWGPGTLVTVSS
Ab28_VH CDRH1
SEQ ID NO: 440
GACTACTACTACATGTGC
Ab28_VH CDRH1
SEQ ID NO: 441
DYYYMC
Ab28_VH CDRH2
SEQ ID NO: 442
TGCATTTATAATGGTGATGGTGAGACTTACTACGCGAACTGGGCGAAAGGC
Ab28_VH CDRH2
SEQ ID NO: 443
CIYNGDGETYYANWAKG
Ab28_VH CDRH3
SEQ ID NO: 444
GGTAGTGGTGTTGGGAGTTATGGTTACAACTTT
Ab28_VH CDRH3
SEQ ID NO: 445
GSGVGSYGYNF
Ab28_VL VL
SEQ ID NO: 446
GCTGACATTGTGATGACCCAGACTCCAGCCTCCGTGGAGGCAGCTGTGGGAGGCACAGTCACCATCAAGTGCCAG
GCCAGTGAGAGCATTTACAGCAATTTAGCCTGGTATCAGCAGAAACCAGGGCAGCCTCCCAAGCTCCTAATCTAC
AGGGCATCCAATCTGGCATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTCAAGGCTATTATGCCAGTACTGTTACTAATGCT
TTCGGCGGAGGGACCGAGGTGGTGGTCAAACGT
Ab28_VL VL
SEQ ID NO: 447
ADIVMTQTPASVEAAVGGTVTIKCQASESIYSNLAWYQQKPGQPPKLLIYRASNLASGVPSRFKGSGSGTEFTLT
ISDLECADAATYYCQGYYASTVTNAFGGGTEVVVKR
Ab28_VL CDRL1
SEQ ID NO: 448
CAGGCCAGTGAGAGCATTTACAGCAATTTAGCC
Ab28_VL CDRL1
SEQ ID NO: 449
QASESIYSNLA
Ab28_VL CDRL2
SEQ ID NO: 450
AGGGCATCCAATCTGGCATCT
Ab28_VL CDRL2
SEQ ID NO: 451
RASNLAS
Ab28_VL CDRL3
SEQ ID NO: 452
CAAGGCTATTATGCCAGTACTGTTACTAATGCT
Ab28_VL CDRL3
SEQ ID NO: 453
QGYYASTVTNA
Ab29_VH VH
SEQ ID NO: 454
CAGCAGCAGCTGGAGGAGTCCGGGGGAGGCCTGGTCAAGCCTGGAGGAACCCTGACACTCACCTGCAAAGCCTCT
GGAATCGACTTCAGTAGTTACCACTACATGTGCTGGGTCCGCCAGGCTCCAGGGAAGGGGCTGGAGTGGATCGCG
TGCATTTATAATGGTGATGGTGACACTTACTACGCGAGCTGGGCGAAAGGCCGATTCACCATCTCCAAAACCTCG
TCGACCACGGTGACTCTGCAAATGACCAGTCTGACAGCCGCGGACACGGCCACCTATTTCTGTGCGAGGGGTAGT
GGTGTTGGTAGTTATGGTTACAATTTGTGGGGCCCAGGCACCCTGGTCACCGTCTCGAGC
Ab29_VH VH
SEQ ID NO: 455
QQQLEESGGGLVKPGGTLTLTCKASGIDFSSYHYMCWVRQAPGKGLEWIACIYNGDGDTYYASWAKGRFTISKTS
STTVTLQMTSLTAADTATYFCARGSGVGSYGYNLWGPGTLVTVSS
Ab29_VH CDRH1
SEQ ID NO: 456
AGTTACCACTACATGTGC
Ab29_VH CDRH1
SEQ ID NO: 457
SYHYMC
Ab29_VH CDRH2
SEQ ID NO: 458
TGCATTTATAATGGTGATGGTGACACTTACTACGCGAGCTGGGCGAAAGGC
Ab29_VH CDRH2
SEQ ID NO: 459
CIYNGDGDTYYASWAKG
Ab29_VH CDRH3
SEQ ID NO: 460
GGTAGTGGTGTTGGTAGTTATGGTTACAATTTG
Ab29_VH CDRH3
SEQ ID NO: 461
GSGVGSYGYNL
Ab29_VL VL
SEQ ID NO: 462
GCTGACATTGTGATGACCCAGACTCCAGCCTCCGTGGAGGCAGCTGTGGGAGGCACAGTCACCATCAAGTGCCAG
GCCAGTGAGAACATTTACAGCAATTTAGCCTGGTATCAGCAGAAACCAGGGCAGCCTCCCAAGCTCCTAATCTAC
AGGGCATCCAATCTGGAATCTGGGGTCCCATCGCGGTTCAAAGGCAGTGGATCTGGGACAGAGTTCACTCTCACC
ATCAGCGACCTGGAGTGTGACGATGCTGCCACTTACTACTGTCAAGGCTATTATCCCAGTGCTGTTACTAATGCT
TTCGGCGGAGGGACCGAGGTGGTGGTCAAACGT
Ab29_VL VL
SEQ ID NO: 463
ADIVMTQTPASVEAAVGGTVTIKCQASENTYSNLAWYQQKPGQPPKLLIYRASNLESGVPSRFKGSGSGTEFTLT
ISDLECDDAATYYCQGYYPSAVTNAFGGGTEVVVKR
Ab29_VL CDRL1
SEQ ID NO: 464
CAGGCCAGTGAGAACATTTACAGCAATTTAGCC
Ab29_VL CDRL1
SEQ ID NO: 465
QASENIYSNLA
Ab29_VL CDRL2
SEQ ID NO: 466
AGGGCATCCAATCTGGAATCT
Ab29_VL CDRL2
SEQ ID NO: 467
RASNLES
Ab29_VL CDRL3
SEQ ID NO: 468
CAAGGCTATTATCCCAGTGCTGTTACTAATGCT
Ab29_VL CDRL3
SEQ ID NO: 469
QGYYPSAVTNA

What is claimed is:

1. An isolated antibody, or antigen-binding antibody fragment, that binds to human mGluR5 and antagonizes, inhibits, neutralizes or blocks at least one biological effect associated with human mGluR5, wherein the isolated antibody or antigen-binding antibody fragment is selected from the group consisting of a monoclonal antibody; a monospecific antibody; a polyspecific antibody; a human antibody; a humanized antibody; a chimeric antibody; a tetrameric antibody; a tetravalent antibody; a bispecific antibody; a multispecific antibody; a single chain antibody; a domain-specific antibody; a single domain antibody; a domain-deleted antibody; an scFc fusion protein; a synthetic antibody; a recombinant antibody; a hybrid antibody; a mutated antibody; CDR-grafted antibodies; an antigen-binding antibody fragment; an Fab; an F(ab′)2; an Fab′ fragment; an Fv fragment; a single-chain Fv (scFv) fragment; an Fd fragment; a dAb fragment; a diabody; a nanobody; a bivalent nanobody; a shark variable IgNAR domain; a V_{H}H antibody; a camelid antibody; a chimeric antigen receptor (CAR), a Bispecific T cell Engager (BiTE), and a minibody.

2-3. (canceled)

4. The isolated antibody or antigen-binding antibody fragment according to claim 1, wherein the antibody or antigen-binding antibody fragment is not N-glycosylated.

5. The isolated antibody or antigen-binding antibody fragment according to claim 1, which allosterically inhibits human mGluR5; which does not compete with quisqualate for binding to human mGluR5, optionally as measured via a radioligand binding inhibition assay; which does not bind to human mGluR1; which cross-reacts with rat and/or cynomolgus monkey mGluR5; which inhibits the production of cytosolic phospho-ERK (pERK) in a pERK signaling assay, optionally wherein the IC₅₀ of the antibody or antigen-binding antibody fragment in the pERK signaling assay is less than 100 nM, less than 50 nM, or less than 10 nM; which binds to the extracellular domain of human mGluR5 with an EC₅₀ of less than 10 nM, less than 5 nM, or less than 2 nM, as measured via an HTRF or AlphaLISA fluorescence assay; which inhibits one or more migraine associated symptoms; which inhibits umbellulone-induced lacrimation and/or umbellulone-induced facial temperature increase when administered to a subject; or which does not elicit the adverse side-effects associated with small molecule mGluR5 antagonists, e.g., liver toxicity and/or impaired motor coordination and/or dizziness, optionally when peripherally administered.

6-13. (canceled)

14. The isolated antibody or antigen-binding antibody fragment according to claim 1, which comprises:
(i) a V_{H} chain comprising a CDR1 sequence consisting of SEQ ID NO: 9; a CDR2 sequence consisting of SEQ ID NO: 11; and a CDR3 sequence consisting of SEQ ID NO: 13; and/or a V_{L} chain comprising a CDR1 sequence consisting of SEQ ID NO: 17; a CDR2 sequence consisting of SEQ ID NO: 19; and a CDR3 sequence consisting of SEQ ID NO: 21;
(ii) a V_{H} chain comprising a CDR1 sequence consisting of SEQ ID NO: 25; a CDR2 sequence consisting of SEQ ID NO: 27; and a CDR3 sequence consisting of SEQ ID NO: 29; and/or a V_{L} chain comprising a CDR1 sequence consisting of SEQ ID NO: 33; a CDR2 sequence consisting of SEQ ID NO: 35; and a CDR3 sequence consisting of SEQ ID NO: 37;
(iii) a V_{H} chain comprising a CDR1 sequence consisting of SEQ ID NO: 41; a CDR2 sequence consisting of SEQ ID NO: 43; and a CDR3 sequence consisting of SEQ ID NO: 45; and/or a V_{L} chain comprising a CDR1 sequence consisting of SEQ ID NO: 49; a CDR2 sequence consisting of SEQ ID NO: 51; and a CDR3 sequence consisting of SEQ ID NO: 53;
(iv) a V_{H} chain comprising a CDR1 sequence consisting of SEQ ID NO: 57; a CDR2 sequence consisting of SEQ ID NO: 59; and a CDR3 sequence consisting of SEQ ID NO: 61; and/or a V_{L} chain comprising a CDR1 sequence consisting of SEQ ID NO: 65; a CDR2 sequence consisting of SEQ ID NO: 67; and a CDR3 sequence consisting of SEQ ID NO: 69;
(v) a V_{H} chain comprising a CDR1 sequence consisting of SEQ ID NO: 73; a CDR2 sequence consisting of SEQ ID NO: 75; and a CDR3 sequence consisting of SEQ ID NO: 77; and/or a V_{L} chain comprising a CDR1 sequence consisting of SEQ ID NO: 81; a CDR2 sequence consisting of SEQ ID NO: 83; and a CDR3 sequence consisting of SEQ ID NO: 85;
(vi) a V_{H} chain comprising a CDR1 sequence consisting of SEQ ID NO: 89; a CDR2 sequence consisting of SEQ ID NO: 91; and a CDR3 sequence consisting of SEQ ID NO: 93; and/or a V_{L} chain comprising a CDR1 sequence consisting of SEQ ID NO: 97; a CDR2 sequence consisting of SEQ ID NO: 99; and a CDR3 sequence consisting of SEQ ID NO: 101;
(vii) a V_{H} chain comprising a CDR1 sequence consisting of SEQ ID NO: 105; a CDR2 sequence consisting of SEQ ID NO: 107; and a CDR3 sequence consisting of SEQ ID NO: 109; and/or a V_{L} chain comprising a CDR1 sequence consisting of SEQ ID NO: 113; a CDR2 sequence consisting of SEQ ID NO: 115; and a CDR3 sequence consisting of SEQ ID NO: 117;
(viii) a V_{H} chain comprising a CDR1 sequence consisting of SEQ ID NO: 121; a CDR2 sequence consisting of SEQ ID NO: 123; and a CDR3 sequence consisting of SEQ ID NO: 125; and/or a V_{L} chain comprising a CDR1 sequence consisting of SEQ ID NO: 129; a CDR2 sequence consisting of SEQ ID NO: 131; and a CDR3 sequence consisting of SEQ ID NO: 133;
(ix) a V_{H} chain comprising a CDR1 sequence consisting of SEQ ID NO: 137; a CDR2 sequence consisting of SEQ ID NO: 139; and a CDR3 sequence consisting of SEQ ID NO: 141; and/or a V_{L} chain comprising a CDR1 sequence consisting of SEQ ID NO: 145; a CDR2 sequence consisting of SEQ ID NO: 147; and a CDR3 sequence consisting of SEQ ID NO: 149;
(x) a V_{H} chain comprising a CDR1 sequence consisting of SEQ ID NO: 153; a CDR2 sequence consisting of SEQ ID NO: 155; and a CDR3 sequence consisting of SEQ ID NO: 157; and/or a V_{L} chain comprising a CDR1 sequence consisting of SEQ ID NO: 161; a CDR2 sequence consisting of SEQ ID NO: 163; and a CDR3 sequence consisting of SEQ ID NO: 165;
(xi) a V_{H} chain comprising a CDR1 sequence consisting of SEQ ID NO: 169; a CDR2 sequence consisting of SEQ ID NO: 171; and a CDR3 sequence consisting of SEQ ID NO: 173; and/or a V_{L} chain comprising a CDR1 sequence consisting of SEQ ID NO: 177; a CDR2 sequence consisting of SEQ ID NO: 179; and a CDR3 sequence consisting of SEQ ID NO: 181;
(xii) a V_{H} chain comprising a CDR1 sequence consisting of SEQ ID NO: 185; a CDR2 sequence consisting of SEQ ID NO: 187; and a CDR3 sequence consisting of SEQ ID NO: 189; and/or a V_{L} chain comprising a CDR1 sequence consisting of SEQ ID NO: 193; a CDR2 sequence consisting of SEQ ID NO: 195; and a CDR3 sequence consisting of SEQ ID NO: 197;
(xiii) a V_{H} chain comprising a CDR1 sequence consisting of SEQ ID NO: 201; a CDR2 sequence consisting of SEQ ID NO: 203; and a CDR3 sequence consisting of SEQ ID NO: 205; and/or a V_{L} chain comprising a CDR1 sequence consisting of SEQ ID NO: 209; a CDR2 sequence consisting of SEQ ID NO: 211; and a CDR3 sequence consisting of SEQ ID NO: 213;
(xiv) a V_{H} chain comprising a CDR1 sequence consisting of SEQ ID NO: 217; a CDR2 sequence consisting of SEQ ID NO: 219; and a CDR3 sequence consisting of SEQ ID NO: 221; and/or a V_{L} chain comprising a CDR1 sequence consisting of SEQ ID NO: 225; a CDR2 sequence consisting of SEQ ID NO: 227; and a CDR3 sequence consisting of SEQ ID NO: 229;
(xv) a V_{H} chain comprising a CDR1 sequence consisting of SEQ ID NO: 233; a CDR2 sequence consisting of SEQ ID NO: 235; and a CDR3 sequence consisting of SEQ ID NO: 237; and/or a V_{L} chain comprising a CDR1 sequence consisting of SEQ ID NO: 241; a CDR2 sequence consisting of SEQ ID NO: 243; and a CDR3 sequence consisting of SEQ ID NO: 245;
(xvi) a V_{H} chain comprising a CDR1 sequence consisting of SEQ ID NO: 249; a CDR2 sequence consisting of SEQ ID NO: 251; and a CDR3 sequence consisting of SEQ ID NO: 253; and/or a V_{L} chain comprising a CDR1 sequence consisting of SEQ ID NO: 257; a CDR2 sequence consisting of SEQ ID NO: 259; and a CDR3 sequence consisting of SEQ ID NO: 261;
(xvii) a V_{H} chain comprising a CDR1 sequence consisting of SEQ ID NO: 265; a CDR2 sequence consisting of SEQ ID NO: 267; and a CDR3 sequence consisting of SEQ ID NO: 269; and/or a V_{L} chain comprising a CDR1 sequence consisting of SEQ ID NO: 273; a CDR2 sequence consisting of SEQ ID NO: 275; and a CDR3 sequence consisting of SEQ ID NO: 277;
(xviii) a V_{H} chain comprising a CDR1 sequence consisting of SEQ ID NO: 281; a CDR2 sequence consisting of SEQ ID NO: 283; and a CDR3 sequence consisting of SEQ ID NO: 285; and/or a V_{L} chain comprising a CDR1 sequence consisting of SEQ ID NO: 289; a CDR2 sequence consisting of SEQ ID NO: 291; and a CDR3 sequence consisting of SEQ ID NO: 293;
(xix) a V_{H} chain comprising a CDR1 sequence consisting of SEQ ID NO: 297; a CDR2 sequence consisting of SEQ ID NO: 299; and a CDR3 sequence consisting of SEQ ID NO: 301; and/or a V_{L} chain comprising a CDR1 sequence consisting of SEQ ID NO: 305; a CDR2 sequence consisting of SEQ ID NO: 307; and a CDR3 sequence consisting of SEQ ID NO: 309;
(xx) a V_{H} chain comprising a CDR1 sequence consisting of SEQ ID NO: 313; a CDR2 sequence consisting of SEQ ID NO: 315; and a CDR3 sequence consisting of SEQ ID NO: 317; and/or a V_{L} chain comprising a CDR1 sequence consisting of SEQ ID NO: 321; a CDR2 sequence consisting of SEQ ID NO: 323; and a CDR3 sequence consisting of SEQ ID NO: 325;
(xxi) a V_{H} chain comprising a CDR1 sequence consisting of SEQ ID NO: 329; a CDR2 sequence consisting of SEQ ID NO: 331; and a CDR3 sequence consisting of SEQ ID NO: 333; and/or a V_{L} chain comprising a CDR1 sequence consisting of SEQ ID NO: 337; a CDR2 sequence consisting of SEQ ID NO: 339; and a CDR3 sequence consisting of SEQ ID NO: 341;
(xxii) a V_{H} chain comprising a CDR1 sequence consisting of SEQ ID NO: 345; a CDR2 sequence consisting of SEQ ID NO: 347; and a CDR3 sequence consisting of SEQ ID NO: 349; and/or a V_{L} chain comprising a CDR1 sequence consisting of SEQ ID NO: 353; a CDR2 sequence consisting of SEQ ID NO: 355; and a CDR3 sequence consisting of SEQ ID NO: 357;
(xxiii) a V_{H} chain comprising a CDR1 sequence consisting of SEQ ID NO: 361; a CDR2 sequence consisting of SEQ ID NO: 363; and a CDR3 sequence consisting of SEQ ID NO: 365; and/or a V_{L} chain comprising a CDR1 sequence consisting of SEQ ID NO: 369; a CDR2 sequence consisting of SEQ ID NO: 371; and a CDR3 sequence consisting of SEQ ID NO: 373;
(xxiv) a V_{H} chain comprising a CDR1 sequence consisting of SEQ ID NO: 377; a CDR2 sequence consisting of SEQ ID NO: 379; and a CDR3 sequence consisting of SEQ ID NO: 381; and/or a V_{L} chain comprising a CDR1 sequence consisting of SEQ ID NO: 385; a CDR2 sequence consisting of SEQ ID NO: 387; and a CDR3 sequence consisting of SEQ ID NO: 389;
(xxv) a V_{H} chain comprising a CDR1 sequence consisting of SEQ ID NO: 393; a CDR2 sequence consisting of SEQ ID NO: 395; and a CDR3 sequence consisting of SEQ ID NO: 397; and/or a V_{L} chain comprising a CDR1 sequence consisting of SEQ ID NO: 401; a CDR2 sequence consisting of SEQ ID NO: 403; and a CDR3 sequence consisting of SEQ ID NO: 405;
(xxvi) a V_{H} chain comprising a CDR1 sequence consisting of SEQ ID NO: 409; a CDR2 sequence consisting of SEQ ID NO: 411; and a CDR3 sequence consisting of SEQ ID NO: 413; and/or a V_{L} chain comprising a CDR1 sequence consisting of SEQ ID NO: 417; a CDR2 sequence consisting of SEQ ID NO: 419; and a CDR3 sequence consisting of SEQ ID NO: 421;
(xxvii) a V_{H} chain comprising a CDR1 sequence consisting of SEQ ID NO: 425; a CDR2 sequence consisting of SEQ ID NO: 427; and a CDR3 sequence consisting of SEQ ID NO: 429; and/or a V_{L} chain comprising a CDR1 sequence consisting of SEQ ID NO: 433; a CDR2 sequence consisting of SEQ ID NO: 435; and a CDR3 sequence consisting of SEQ ID NO: 437;
(xxviii) a V_{H} chain comprising a CDR1 sequence consisting of SEQ ID NO: 441; a CDR2 sequence consisting of SEQ ID NO: 443; and a CDR3 sequence consisting of SEQ ID NO: 445; and/or a V_{L} chain comprising a CDR1 sequence consisting of SEQ ID NO: 449; a CDR2 sequence consisting of SEQ ID NO: 451; and a CDR3 sequence consisting of SEQ ID NO: 453; or
(xxix) a V_{H} chain comprising a CDR1 sequence consisting of SEQ ID NO: 457; a CDR2 sequence consisting of SEQ ID NO: 459; and a CDR3 sequence consisting of SEQ ID NO: 461; and/or a V_{L} chain comprising a CDR1 sequence consisting of SEQ ID NO: 465; a CDR2 sequence consisting of SEQ ID NO: 467; and a CDR3 sequence consisting of SEQ ID NO: 469.

15. The isolated antibody or antigen-binding antibody fragment according to claim 14, which comprises:
(i) a V_{H} chain comprising an amino acid sequence with at least 80, 85, 90, 95, 96, 97, 98, 99, or 100% sequence identity to SEQ ID NO: 7, and/or a V_{L} chain comprising an amino acid sequence with at least 80, 85, 90, 95, 96, 97, 98, 99, or 100% sequence identity to SEQ ID NO: 15;
(ii) a V_{H} chain comprising an amino acid sequence with at least 80, 85, 90, 95, 96, 97, 98, 99, or 100% sequence identity to SEQ ID NO: 23, and/or a V_{L} chain comprising an amino acid sequence with at least 80, 85, 90, 95, 96, 97, 98, 99, or 100% sequence identity to SEQ ID NO: 31;
(iii) a V_{H} chain comprising an amino acid sequence with at least 80, 85, 90, 95, 96, 97, 98, 99, or 100% sequence identity to SEQ ID NO: 39, and/or a V_{L} chain comprising an amino acid sequence with at least 80, 85, 90, 95, 96, 97, 98, 99, or 100% sequence identity to SEQ ID NO: 47;
(iv) a V_{H} chain comprising an amino acid sequence with at least 80, 85, 90, 95, 96, 97, 98, 99, or 100% sequence identity to SEQ ID NO: 55, and/or a V_{L} chain comprising an amino acid sequence with at least 80, 85, 90, 95, 96, 97, 98, 99, or 100% sequence identity to SEQ ID NO: 63;
(v) a V_{H} chain comprising an amino acid sequence with at least 80, 85, 90, 95, 96, 97, 98, 99, or 100% sequence identity to SEQ ID NO: 71, and/or a V_{L} chain comprising an amino acid sequence with at least 80, 85, 90, 95, 96, 97, 98, 99, or 100% sequence identity to SEQ ID NO: 79;
(vi) a V_{H} chain comprising an amino acid sequence with at least 80, 85, 90, 95, 96, 97, 98, 99, or 100% sequence identity to SEQ ID NO: 87, and/or a V_{L} chain comprising an amino acid sequence with at least 80, 85, 90, 95, 96, 97, 98, 99, or 100% sequence identity to SEQ ID NO: 95;
(vii) a V_{H} chain comprising an amino acid sequence with at least 80, 85, 90, 95, 96, 97, 98, 99, or 100% sequence identity to SEQ ID NO: 103, and/or a V_{L} chain comprising an amino acid sequence with at least 80, 85, 90, 95, 96, 97, 98, 99, or 100% sequence identity to SEQ ID NO: 111;
(viii) a V_{H} chain comprising an amino acid sequence with at least 80, 85, 90, 95, 96, 97, 98, 99, or 100% sequence identity to SEQ ID NO: 119, and/or a V_{L} chain comprising an amino acid sequence with at least 80, 85, 90, 95, 96, 97, 98, 99, or 100% sequence identity to SEQ ID NO: 127;
(ix) a V_{H} chain comprising an amino acid sequence with at least 80, 85, 90, 95, 96, 97, 98, 99, or 100% sequence identity to SEQ ID NO: 135, and/or a V_{L} chain comprising an amino acid sequence with at least 80, 85, 90, 95, 96, 97, 98, 99, or 100% sequence identity to SEQ ID NO: 143;
(x) a V_{H} chain comprising an amino acid sequence with at least 80, 85, 90, 95, 96, 97, 98, 99, or 100% sequence identity to SEQ ID NO: 151, and/or a V_{L} chain comprising an amino acid sequence with at least 80, 85, 90, 95, 96, 97, 98, 99, or 100% sequence identity to SEQ ID NO: 159;
(xi) a V_{H} chain comprising an amino acid sequence with at least 80, 85, 90, 95, 96, 97, 98, 99, or 100% sequence identity to SEQ ID NO: 167, and/or a V_{L} chain comprising an amino acid sequence with at least 80, 85, 90, 95, 96, 97, 98, 99, or 100% sequence identity to SEQ ID NO: 175;
(xii) a V_{H} chain comprising an amino acid sequence with at least 80, 85, 90, 95, 96, 97, 98, 99, or 100% sequence identity to SEQ ID NO: 183, and/or a V_{L} chain comprising an amino acid sequence with at least 80, 85, 90, 95, 96, 97, 98, 99, or 100% sequence identity to SEQ ID NO: 191;
(xiii) a V_{H} chain comprising an amino acid sequence with at least 80, 85, 90, 95, 96, 97, 98, 99, or 100% sequence identity to SEQ ID NO: 199, and/or a V_{L} chain comprising an amino acid sequence with at least 80, 85, 90, 95, 96, 97, 98, 99, or 100% sequence identity to SEQ ID NO: 207;
(xiv) a V_{H} chain comprising an amino acid sequence with at least 80, 85, 90, 95, 96, 97, 98, 99, or 100% sequence identity to SEQ ID NO: 215, and/or a V_{L} chain comprising an amino acid sequence with at least 80, 85, 90, 95, 96, 97, 98, 99, or 100% sequence identity to SEQ ID NO: 223;
(xv) a V_{H} chain comprising an amino acid sequence with at least 80, 85, 90, 95, 96, 97, 98, 99, or 100% sequence identity to SEQ ID NO: 231, and/or a V_{L} chain comprising an amino acid sequence with at least 80, 85, 90, 95, 96, 97, 98, 99, or 100% sequence identity to SEQ ID NO: 239;
(xvi) a V_{H} chain comprising an amino acid sequence with at least 80, 85, 90, 95, 96, 97, 98, 99, or 100% sequence identity to SEQ ID NO: 247, and/or a V_{L} chain comprising an amino acid sequence with at least 80, 85, 90, 95, 96, 97, 98, 99, or 100% sequence identity to SEQ ID NO: 255;
(xvii) a V_{H} chain comprising an amino acid sequence with at least 80, 85, 90, 95, 96, 97, 98, 99, or 100% sequence identity to SEQ ID NO: 263, and/or a V_{L} chain comprising an amino acid sequence with at least 80, 85, 90, 95, 96, 97, 98, 99, or 100% sequence identity to SEQ ID NO: 271;
(xviii) a V_{H} chain comprising an amino acid sequence with at least 80, 85, 90, 95, 96, 97, 98, 99, or 100% sequence identity to SEQ ID NO: 279, and/or a V_{L} chain comprising an amino acid sequence with at least 80, 85, 90, 95, 96, 97, 98, 99, or 100% sequence identity to SEQ ID NO: 287;
(xix) a V_{H} chain comprising an amino acid sequence with at least 80, 85, 90, 95, 96, 97, 98, 99, or 100% sequence identity to SEQ ID NO: 295, and/or a V_{L} chain comprising an amino acid sequence with at least 80, 85, 90, 95, 96, 97, 98, 99, or 100% sequence identity to SEQ ID NO: 303;
(xx) a V_{H} chain comprising an amino acid sequence with at least 80, 85, 90, 95, 96, 97, 98, 99, or 100% sequence identity to SEQ ID NO: 311, and/or a V_{L} chain comprising an amino acid sequence with at least 80, 85, 90, 95, 96, 97, 98, 99, or 100% sequence identity to SEQ ID NO: 319;
(xxi) a V_{H} chain comprising an amino acid sequence with at least 80, 85, 90, 95, 96, 97, 98, 99, or 100% sequence identity to SEQ ID NO: 327, and/or a V_{L} chain comprising an amino acid sequence with at least 80, 85, 90, 95, 96, 97, 98, 99, or 100% sequence identity to SEQ ID NO: 335;
(xxii) a V_{H} chain comprising an amino acid sequence with at least 80, 85, 90, 95, 96, 97, 98, 99, or 100% sequence identity to SEQ ID NO: 343, and/or a V_{L} chain comprising an amino acid sequence with at least 80, 85, 90, 95, 96, 97, 98, 99, or 100% sequence identity to SEQ ID NO: 351;
(xxiii) a V_{H} chain comprising an amino acid sequence with at least 80, 85, 90, 95, 96, 97, 98, 99, or 100% sequence identity to SEQ ID NO: 359, and/or a V_{L} chain comprising an amino acid sequence with at least 80, 85, 90, 95, 96, 97, 98, 99, or 100% sequence identity to SEQ ID NO: 367;
(xxiv) a V_{H} chain comprising an amino acid sequence with at least 80, 85, 90, 95, 96, 97, 98, 99, or 100% sequence identity to SEQ ID NO: 375, and/or a V_{L} chain comprising an amino acid sequence with at least 80, 85, 90, 95, 96, 97, 98, 99, or 100% sequence identity to SEQ ID NO: 383;
(xxv) a V_{H} chain comprising an amino acid sequence with at least 80, 85, 90, 95, 96, 97, 98, 99, or 100% sequence identity to SEQ ID NO: 391, and/or a V_{L} chain comprising an amino acid sequence with at least 80, 85, 90, 95, 96, 97, 98, 99, or 100% sequence identity to SEQ ID NO: 399;
(xxvi) a V_{H} chain comprising an amino acid sequence with at least 80, 85, 90, 95, 96, 97, 98, 99, or 100% sequence identity to SEQ ID NO: 407, and/or a V_{L} chain comprising an amino acid sequence with at least 80, 85, 90, 95, 96, 97, 98, 99, or 100% sequence identity to SEQ ID NO: 415;
(xxvii) a V_{H} chain comprising an amino acid sequence with at least 80, 85, 90, 95, 96, 97, 98, 99, or 100% sequence identity to SEQ ID NO: 423, and/or a V_{L} chain comprising an amino acid sequence with at least 80, 85, 90, 95, 96, 97, 98, 99, or 100% sequence identity to SEQ ID NO: 431;
(xxviii) a V_{H} chain comprising an amino acid sequence with at least 80, 85, 90, 95, 96, 97, 98, 99, or 100% sequence identity to SEQ ID NO: 439, and/or a V_{L} chain comprising an amino acid sequence with at least 80, 85, 90, 95, 96, 97, 98, 99, or 100% sequence identity to SEQ ID NO: 447; or
(xxix) a V_{H} chain comprising an amino acid sequence with at least 80, 85, 90, 95, 96, 97, 98, 99, or 100% sequence identity to SEQ ID NO: 455, and/or a V_{L} chain comprising an amino acid sequence with at least 80, 85, 90, 95, 96, 97, 98, 99, or 100% sequence identity to SEQ ID NO: 463.

16-17. (canceled)

18. The isolated antibody or antigen-binding antibody fragment according to claim 1, which comprises a human IgG1, IgG2, IgG3, or IgG4 Fc region; or which comprises an Fc region that has been modified to alter at least one of effector function, half-life, proteolysis, or glycosylation, and/or decrease Fc receptor binding.

19-21. (canceled)

22. The isolated antibody or antigen-binding antibody fragment according to claim 1, which additionally has one or more of the following modifications:
(i) is conjugated to a cytotoxic agent;
(ii) is conjugated to a label;
(iii) is conjugated to a chemiluminescent label;
(iv) is conjugated to a paramagnetic label;
(v) is conjugated to an MRI contrast agent;
(vi) is conjugated to a fluorescent label;
(vii) is conjugated to a bioluminescent label,
(viii) is conjugated to a radioactive label; and
(ix) is conjugated to another therapeutic agent.

23-24. (canceled)

25. An anti-idiotypic antibody or antigen-binding antibody fragment produced against an anti-mGluR5 antibody or antigen-binding antibody fragment according to claim 1, which optionally neutralizes one or more biological effects of the anti-mGluR5 antibody or antigen-binding antibody fragment to which it binds.

26. (canceled)

27. A pharmaceutical composition comprising a pharmaceutically effective amount of an isolated antibody or antigen-binding antibody fragment according to claim 1, or a cell which expresses such antibody or antigen-binding antibody fragment, optionally an immune cell, e.g., a T, Treg, or NK cell, further comprising a pharmaceutical diluent, carrier, or excipient.

28. A method of using the anti-idiotypic antibody or antibody fragment of claim 25 to monitor the in vivo levels of said anti-mGluR5 antibody or antigen-binding antibody fragment in a subject or to neutralize the in vivo effects of said anti-mGluR5 antibody or antigen-binding antibody fragment in a subject, wherein said anti-idiotypic antibody or antibody fragment is peripherally administered, centrally administered, or peripherally and centrally administered.

29-31. (canceled)

32. An isolated polynucleotide encoding the antibody or antigen-binding antibody fragment according to claim 1 or an expression vector comprising said polynucleotide.

33. (canceled)

34. A host cell comprising the expression vector of claim 32.

35. A method of producing an isolated anti-mGluR5 antibody or antigen-binding antibody fragment comprising culturing the host cell of claim 34 under conditions that allow expression of the antibody or antigen-binding antibody fragment; and recovering the antibody or antigen-binding antibody fragment from the culture medium or host cell.

36. A method for treating or preventing a disorder associated with the peripheral or central nervous system; or a condition, disease, or disorder associated with mGluR5 activity comprising administering to a subject in need thereof; wherein the subject is a mammal, a human, a non-human primate, or a rodent, a therapeutically or prophylactically effective amount of an antibody or antigen-binding antibody fragment according to claim 1, wherein said antibody, antigen-binding antibody fragment, or pharmaceutical composition is peripherally administered, centrally administered, or peripherally and centrally administered, peripherally, centrally, or peripherally and centrally, enterally, parenterally, intravenously, intrathecally, or topically, preferably wherein the administration is parenteral.

37-39. (canceled)

40. The method according to claim 36, wherein administration of the antibody gr antigen-binding antibody fragment:
(i) inhibits mGluR5 signaling;
(ii) does not inhibit mGluR1;
(iii) inhibits the production of cytosolic pERK;
(iv) does not result in a dizziness and/or other motor function side effect.
(v) does not substantially cause an adverse central nervous system side effect in the patient;
(vi) does not elicit side-effects associated with small molecule mGluR5 antagonists.

41. The method according to claim 36, which is used to treat or prevent a peripheral nervous system disorder; a central nervous system disorder; migraine; episodic migraine; chronic migraine; cluster headache; or one or more symptoms associated with migraine, or reducing the frequency and/or severity of such one or more symptoms, optionally vasomotor symptoms (e.g. hot flashes, facial flushing, sweating, and night sweats), photophobia, phonophobia, sensitivity to smells, tearing/lacrimation, vertigo, dizziness, nausea, vomiting, headache pain, and aura; pain; GERD; nonerosive reflux disease, erosive esophagitis; irritable bowel syndrome (IBS); diarrhea predominant IBS; constipation predominant IBS; alternating bowel movement predominant IBS; overactive bladder (OAB); incontinence; urinary incontinence, urgency, urinary frequency, nocturia, urge incontinence, stress incontinence, overflow incontinence, mixed incontinence, structural incontinence, functional incontinence, nocturnal incontinence, transient incontinence, giggle incontinence, double incontinence, post-void dribbling, or coital incontinence; a neurological disorder; an autism spectrum disorder; symptoms of an autism spectrum disorder, including impaired social and functional communication, anxiety, inattention, hyperactivity, altered sensory reactivity, self-injury, aggression, impaired cognitive function, or compromised daily living skills; a psychiatric disorder; Parkinson's disease and/or levodopa-induced dyskinesia; schizophrenia; schizophreniform disorder; schizoaffective disorder; delusional disorder; brief psychotic disorder; shared psychotic disorder; psychotic disorder due to a general medical condition; substance-induced psychotic disorder; psychotic disorder not otherwise specified; psychosis associated with dementia; major depressive disorder; dysthymic disorder, premenstrual dysphoric disorder; depressive disorder not otherwise specified; bipolar I disorder; bipolar II disorder; cyclothymic disorder; bipolar disorder not otherwise specified; mood disorder due to a general medical condition; substance-induced mood disorder; mood disorder not otherwise specified; generalized anxiety disorder; obsessive-compulsive disorder; panic disorder; acute stress disorder; post-traumatic stress disorder; mental retardation; pervasive developmental disorders; attention deficit disorders; attention-deficit/hyperactivity disorder; disruptive behavior disorders; personality disorder of the paranoid type; personality disorder of the schizoid type; personality disorder of the schizotypical type; tic disorders; Tourette's syndrome; substance dependence; substance abuse; substance withdrawal; trichotillomania; conditions wherein cognition is impaired; Alzheimer's disease; Parkinson's disease; levodopa-induced dyskinesia in Parkinson's disease patients; Huntingdon's disease; Lewy Body Dementia; dementia due to HIV disease; dementia due to Creutzfeldt-Jakob disease; amnestic disorders; mild cognitive impairment; age-related cognitive decline; feeding disorders such as anorexia and bulimia; and obesity; alcohol, nicotine, cocaine, amphetamine, benzodiazepine, analgesics, opiate or other substance tolerance or dependence; bulimia nervosa; anorexia nervosa; gambling dependence; sex dependence; obsessive compulsive disorders; neurodegeneration; neurotoxicity; ischemia; Parkinson's disease; memory impairment; Alzheimer's disease; dementia; delirium tremens; pain; acute and/or chronic pain; neuropathic pain; central pain syndromes; postoperative pain; bone and joint pain; repetitive motion pain; dental pain; cancer pain; myofascial pain; perioperative pain; chronic pain; acute pain; dysmenorrhea; pain associated with angina; inflammatory pain; headache; migraine and cluster headache; primary hyperalgesia; secondary hyperalgesia; primary allodynia; secondary allodynia; or other pain.

42-51. (canceled)

52. The method according to claim 41, wherein the monthly incidence of migraine is reduced following administration of the antibody pr antigen-binding antibody fragment.

53-54. (canceled)

55. The method according to claim 41; wherein the patient has not previously received prophylactic therapy for migraine headaches; wherein the patient has failed or is intolerant to at least one other migraine headache prophylactic therapy, optionally an antiepileptic, a tricyclic antidepressant, or a beta-blocker; wherein the antibody; or antigen-binding antibody fragment is administered as a monotherapy; or wherein the antibody or antigen-binding antibody fragment is administered in combination with a second therapeutic agent, optionally another biologic, further optionally an anti-CGRP antibody and/or anti-PACAP antibody.

56-89. (canceled)

90. A method for diagnosing a condition associated with upregulation of mGluR5 expression, said method comprising:
(i) isolating the cells or tissues responsible for mediating the condition;
(ii) contacting said cells with an anti-mGluR5 antibody or antigen-binding antibody fragment according to claim 1; and
(iii) detecting the level of anti-mGluR5 antibody or antigen-binding antibody fragment bound to said cells.

91. An antibody or antigen-binding antibody fragment according to claim 1 for use in the treatment or prophylaxis of migraine; one or more symptoms associated with migraine; episodic migraine; chronic migraine; cluster headache; or reduces the frequency and/or severity of such one or more symptoms, optionally vasomotor symptoms (e.g. hot flashes, facial flushing, sweating, and night sweats), photophobia, phonophobia, sensitivity to smells, tearing/lacrimation, vertigo, dizziness, nausea, vomiting, headache pain, or aura; GERD; nonerosive reflux disease, erosive esophagitis; irritable bowel syndrome (IBS); diarrhea predominant IBS; constipation predominant IBS, or alternating bowel movement predominant IBS; overactive bladder (OAB); urinary incontinence; urgency; urinary frequency; nocturia; urge incontinence; stress incontinence; urge incontinence; overflow incontinence; mixed incontinence; structural incontinence; functional incontinence; nocturnal incontinence; transient incontinence; giggle incontinence; double incontinence; post-void dribbling; coital incontinence; autism spectrum disorder; impaired social and functional communication; anxiety; inattention; hyperactivity; altered sensory reactivity; self-injury; aggression; impaired cognitive function; compromised daily living skills; a neurological or psychiatric disorder; schizophrenia, schizophreniform disorder; schizoaffective disorder; delusional disorder; brief psychotic disorder; shared psychotic disorder; psychotic disorder due to a general medical condition; substance-induced psychotic disorder; psychotic disorder not otherwise specified; psychosis associated with dementia; major depressive disorder; dysthymic disorder; premenstrual dysphoric disorder; depressive disorder not otherwise specified; bipolar I disorder; bipolar II disorder; cyclothymic disorder; bipolar disorder not otherwise specified; mood disorder due to a general medical condition; substance-induced mood disorder; mood disorder not otherwise specified; generalized anxiety disorder; obsessive-compulsive disorder; panic disorder; acute stress disorder; post-traumatic stress disorder; mental retardation; pervasive developmental disorders; attention deficit disorders; attention-deficit/hyperactivity disorder; disruptive behavior disorders; personality disorder of the paranoid type; personality disorder of the schizoid type; personality disorder of the schizotypical type; tic disorders; Tourette's syndrome; substance dependence; substance abuse; substance withdrawal; trichotillomania; conditions wherein cognition is impaired; Alzheimer's disease; Parkinson's disease; levodopa-induced dyskinesia in Parkinson's disease patients; Huntingdon's disease; Lewy Body Dementia; dementia due to HIV disease; dementia due to Creutzfeldt-Jakob disease; amnestic disorders; mild cognitive impairment; age-related cognitive decline; feeding disorders such as anorexia and bulimia, and obesity; a behavior or dependence disorder; a neurological disorder; ischemia; Parkinson's disease; memory impairment; Alzheimer's disease; dementia; and delirium tremens; pain; neuropathic pain; central pain syndromes; postoperative pain; bone and joint pain; repetitive motion pain; dental pain; cancer pain; myofascial pain; perioperative pain; chronic pain, acute pain; dysmenorrhea; pain associated with angina; inflammatory pain; headache, migraine and cluster headache, primary hyperalgesia, secondary hyperalgesia, primary allodynia, secondary allodynia, or other pain.

92-114. (canceled)


SEQUENCE LISTING (469 sequences)

SEQ ID NO: 1 - protein, 1212 aa, Artificial Sequence
  Human mGluR5b
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCNSSLTLKTHHVQDSKMGFVINAIYSMAYGLHNMQMSLCPGYAGLCDAMKPIDGRKLLESLMKTNFTGVSGDTILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPKGSMGNGGRATMSSSNGKSVTWAQNEKSSRGQHLWQRLSIHINKKENPNQTAVIKPFPKSTESRGLGAGAGAGGSAGGVGATGGAGCAGAGPGGPESPDAGPKALYDVAEAEEHFPAPARPRSPSPISTLSHRAGSASRTDDDVPSLHSEPVARSSSSQGSLMEQISSVVTRFTANISELNSMMLSTAAPSPGVGAPLCSSYLIPKEIQLPTTMTTFAEIQPLPAIEVTGGAQPAAGAQAAGDAARESPAAGPEAAAAKPDLEELVALTPPSPFRDSVDSGSTTPNSPVSESALCIPSSPKYDTLIIRDYTQSSSSL

SEQ ID NO: 2 - protein, 1180 aa, Artificial Sequence
  Human mGluR5a
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCNSSLTLKTHHVQDSKMGFVINAIYSMAYGLHNMQMSLCPGYAGLCDAMKPIDGRKLLESLMKTNFTGVSGDTILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLSSNGKSVTWAQNEKSSRGQHLWQRLSIHINKKENPNQTAVIKPFPKSTESRGLGAGAGAGGSAGGVGATGGAGCAGAGPGGPESPDAGPKALYDVAEAEEHFPAPARPRSPSPISTLSHRAGSASRTDDDVPSLHSEPVARSSSSQGSLMEQISSVVTRFTANISELNSMMLSTAAPSPGVGAPLCSSYLIPKEIQLPTTMTTFAEIQPLPAIEVTGGAQPAAGAQAAGDAARESPAAGPEAAAAKPDLEELVALTPPSPFRDSVDSGSTTPNSPVSESALCIPSSPKYDTLIIRDYTQSSSSL

SEQ ID NO: 3 - protein, 1171 aa, Artificial Sequence
  Rat mGluR5
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLRSHLPKARVVACFCEGMTVRGLLMAMRRLGLAGEFLLLGSDGWADRYDVTDGYQREAVGGITIKLQSPDVKWFDDYYLKLRPETNLRNPWFQEFWQHRFQCRLEGFAQENSKYNKTCNSSLTLRTHHVQDSKMGFVINAIYSMAYGLHNMQMSLCPGYAGLCDAMKPIDGRKLLDSLMKTNFTGVSGDMILFDENGDSPGRYEIMNFKEMGKDYFDYINVGSWDNGELKMDDDEVWSKKNNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVIFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLSSNGKSVTWAQNEKSTRGQHLWQRLSVHINKKENPNQTAVIKPFPKSTENRGPGAAAGGGSGPGVAGAGNAGCTATGGPEPPDAGPKALYDVAEAEESFPAAARPRSPSPISTLSHLAGSAGRTDDDAPSLHSETAARSSSSQGSLMEQISSVVTRFTANISELNSMMLSTAATPGPPGTPICSSYLIPKEIQLPTTMTTFAEIQPLPAIEVTGGAQGATGVSPAQETPTGAESAPGKPDLEELVALTPPSPFRDSVDSGSTTPNSPVSESALCIPSSPKYDTLIIRDYTQSSSSL

SEQ ID NO: 4 - protein, 1212 aa, Artificial Sequence
  Cyno mGluR5
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCDSSLTLKTHHVQDSKMGFVINAIYSMAYGLHNMQMSLCPGYAGLCDAMKPIDGRKLLESLMKTNFTGVSGDMILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPKGSMGNGGRATMSSSNGKSVTWAQNEKSSRGQHLWQRLSIHINKKENPNQTAVIKPFPKSTESRGLGAGAGAGGSAGGMGATGGAGCAGAGPGGPEPPDASPKGLYDVAETEEHFPAPTRPRSPSPISTLSHRAGSASRTDDDVPSLHSEPAARSSSSQGSLMEQISSVVTRFTANISELNSMMLSTAAPSPGVGAPLCSSYLIPKEIQLPTTMTTFAEIQPLPAIEVTGGAQPAAGAQAAGDAARESPAAGPKAAAAKPDLEELVALTPPSPFRDSVDSGSTTPNSPVSESALCIPSSPKYDTLIIRDYTQSSSSL

SEQ ID NO: 5 - protein, 1194 aa, Artificial Sequence
  Human mGluR1
MVGLLLFFFPAIFLEVSLLPRSPGRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGINRCLPDGQSLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKRICTGNESLEENYVQDSKMGFVINAIYAMAHGLQNMHHALCPGHVGLCDAMKPIDGSKLLDFLIKSSFIGVSGEEVWFDEKGDAPGRYDIMNLQYTEANRYDYVHVGTWHEGVLNIDDYKIQMNKSGVVRSVCSEPCLKGQIKVIRKGEVSCCWICTACKENEYVQDEFTCKACDLGWWPNADLTGCEPIPVRYLEWSNIESIIAIAFSCLGILVTLFVTLIFVLYRDTPVVKSSSRELCYIILAGIFLGYVCPFTLIAKPTTTSCYLQRLLVGLSSAMCYSALVTKTNRIARILAGSKKKICTRKPRFMSAWAQVIIASILISVQLTLVVTLIIMEPPMPILSYPSIKEVYLICNTSNLGVVAPLGYNGLLIMSCTYYAFKTRNVPANFNEAKYIAFTMYTTCIIWLAFVPIYFGSNYKIITTCFAVSLSVTVALGCMFTPKMYIIIAKPERNVRSAFTTSDVVRMHVGDGKLPCRSNTFLNIFRRKKAGAGNANSNGKSVSWSEPGGGQVPKGQHMWHRLSVHVKTNETACNQTAVIKPLTKSYQGSGKSLTFSDTSTKTLYNVEEEEDAQPIRFSPPGSPSMVVHRRVPSAATTPPLPSHLTAEETPLFLAEPALPKGLPPPLQQQQQPPPQQKSLMDQLQGVVSNFSTAIPDFHAVLAGPGGPGNGLRSLYPPPPPPQHLQMLPLQLSTFGEELVSPPADDDDDSERFKLLQEYVYEHEREGNTEEDELEEEEEDLQAASKLTPDDSPALTPPSPFRDSVASGSSVPSSPVSESVLCTPPNVSYASVILRDYKQSSSTL

SEQ ID NO: 6 - DNA, 360 bp, Artificial Sequence
  Ab1_VH VH
cagcagcagctggaggagtccgggggaggcctggtcaagcctggaggaaccctgacactcacctgcaaagtctctgggatcgacttcagtagctactactacatgtgctgggtccgccaggctccagggaaggggctggagtggatcgcatgcatttatgctggtgatggtggcacttactacgcgagctgggcgaaaggccgattcaccagctccaaaacctcgtcgaccacggtgactctgcaaatgaccagtctgacagccgcggacacggccacctatttctgtgcgaggggtagtggtgttggtagttatggttacagcttgtggggcccaggcaccctcgtcaccgtctcgagc

SEQ ID NO: 7 - protein, 120 aa, Artificial Sequence
  Ab1_VH VH
QQQLEESGGGLVKPGGTLTLTCKVSGIDFSSYYYMCWVRQAPGKGLEWIACIYAGDGGTYYASWAKGRFTSSKTSSTTVTLQMTSLTAADTATYFCARGSGVGSYGYSLWGPGTLVTVSS

SEQ ID NO: 8 - DNA, 18 bp, Artificial Sequence
  Ab1_VH CDRH1
agctactactacatgtgc

SEQ ID NO: 9 - protein, 6 aa, Artificial Sequence
  Ab1_VH CDRH1
SYYYMC

SEQ ID NO: 10 - DNA, 51 bp, Artificial Sequence
  Ab1_VH CDRH2
tgcatttatgctggtgatggtggcacttactacgcgagctgggcgaaaggc

SEQ ID NO: 11 - protein, 17 aa, Artificial Sequence
  Ab1_VH CDRH2
CIYAGDGGTYYASWAKG

SEQ ID NO: 12 - DNA, 33 bp, Artificial Sequence
  Ab1_VH CDRH3
ggtagtggtgttggtagttatggttacagcttg

SEQ ID NO: 13 - protein, 11 aa, Artificial Sequence
  Ab1_VH CDRH3
GSGVGSYGYSL

SEQ ID NO: 14 - DNA, 333 bp, Artificial Sequence
  Ab1_VL VL
gctgacattgtgatgacccagactccagcctccgtggaggcagctgtgggaggcacagtcaccatcaagtgccaggccagtgagagcatttatagcaatttagcctggtatcagcagaaaccagggcagcctcccaagctcctaatctacagggcatccaatctggaatctggggtcccatcgcggttcaaaggcagtggatctgggacagaatttactctcaccatcagcgacctggagtgtgccgatgctgccacttactactgtcaaggctattatgccagtactactactaatgctttcggcggagggaccgaggtggtggtcaaacgt

SEQ ID NO: 15 - protein, 111 aa, Artificial Sequence
  Ab1_VL VL
ADIVMTQTPASVEAAVGGTVTIKCQASESIYSNLAWYQQKPGQPPKLLIYRASNLESGVPSRFKGSGSGTEFTLTISDLECADAATYYCQGYYASTTTNAFGGGTEVVVKR

SEQ ID NO: 16 - DNA, 33 bp, Artificial Sequence
  Ab1_VL CDRL1
caggccagtgagagcatttatagcaatttagcc

SEQ ID NO: 17 - protein, 11 aa, Artificial Sequence
  Ab1_VL CDRL1
QASESIYSNLA

SEQ ID NO: 18 - DNA, 21 bp, Artificial Sequence
  Ab1_VL CDRL2
agggcatccaatctggaatct

SEQ ID NO: 19 - protein, 7 aa, Artificial Sequence
  Ab1_VL CDRL2
RASNLES

SEQ ID NO: 20 - DNA, 33 bp, Artificial Sequence
  Ab1_VL CDRL3
caaggctattatgccagtactactactaatgct

SEQ ID NO: 21 - protein, 11 aa, Artificial Sequence
  Ab1_VL CDRL3
QGYYASTTTNA

SEQ ID NO: 22 - DNA, 360 bp, Artificial Sequence
  Ab2_VH VH
cagcagcagctggaggagtccgggggaggcctggtcaagcctggaggaaccctgacactcacctgcaaagcctctggaatcgacttcagtggctactactacatgtgctgggtccgccaggctccagggaaggggctggagtggatcgcatgcgtttatactggtgatggtggcacttactacgcgagctgggcgaaaggccgattcaccatctccaaaacctcgtcgaccacggtgactctgcaaatgaccagtctgacagccgcggacacggccacctatttctgtgcgaggggtagtggtgttggtagttatggttacaacttgtggggccaagggaccctcgtcaccgtctcgagc

SEQ ID NO: 23 - protein, 120 aa, Artificial Sequence
  Ab2_VH VH
QQQLEESGGGLVKPGGTLTLTCKASGIDFSGYYYMCWVRQAPGKGLEWIACVYTGDGGTYYASWAKGRFTISKTSSTTVTLQMTSLTAADTATYFCARGSGVGSYGYNLWGQGTLVTVSS

SEQ ID NO: 24 - DNA, 18 bp, Artificial Sequence
  Ab2_VH CDRH1
ggctactactacatgtgc

SEQ ID NO: 25 - protein, 6 aa, Artificial Sequence
  Ab2_VH CDRH1
GYYYMC

SEQ ID NO: 26 - DNA, 51 bp, Artificial Sequence
  Ab2_VH CDRH2
tgcgtttatactggtgatggtggcacttactacgcgagctgggcgaaaggc

SEQ ID NO: 27 - protein, 17 aa, Artificial Sequence
  Ab2_VH CDRH2
CVYTGDGGTYYASWAKG

SEQ ID NO: 28 - DNA, 33 bp, Artificial Sequence
  Ab2_VH CDRH3
ggtagtggtgttggtagttatggttacaacttg

SEQ ID NO: 29 - protein, 11 aa, Artificial Sequence
  Ab2_VH CDRH3
GSGVGSYGYNL

SEQ ID NO: 30 - DNA, 333 bp, Artificial Sequence
  Ab2_VL VL
gctgacattgtgatgacccagactccagcctccgtggaggcagctgtgggaggcacagtcaccatcaagtgccaggccagtgagagcatttatagcaatttagcctggtatcagcagaaaccagggcagcctcccaagctcctaatctacagggcatccaatctggaatctggggtcccatcgcggttcaaaggcagtggatctgggacagagttcactctcaccatcagcgacctggagtgtgccgatgctgccacttactactgtcaaggctattatgccattactactactaatgctttcggcggagggaccgaggtggtggtcaaacgt

SEQ ID NO: 31 - protein, 111 aa, Artificial Sequence
  Ab2_VL VL
ADIVMTQTPASVEAAVGGTVTIKCQASESIYSNLAWYQQKPGQPPKLLIYRASNLESGVPSRFKGSGSGTEFTLTISDLECADAATYYCQGYYAITTTNAFGGGTEVVVKR

SEQ ID NO: 32 - DNA, 33 bp, Artificial Sequence; identical to SEQ ID NO: 16
  Ab2_VL CDRL1

SEQ ID NO: 33 - protein, 11 aa, Artificial Sequence
  Ab2_VL CDRL1
QASESIYSNLA

SEQ ID NO: 34 - DNA, 21 bp, Artificial Sequence
  Ab2_VL CDRL2
agggcatccaatctggaatct

SEQ ID NO: 35 - protein, 7 aa, Artificial Sequence
  Ab2_VL CDRL2
RASNLES

SEQ ID NO: 36 - DNA, 33 bp, Artificial Sequence
  Ab2_VL CDRL3
caaggctattatgccattactactactaatgct

SEQ ID NO: 37 - protein, 11 aa, Artificial Sequence
  Ab2_VL CDRL3
QGYYAITTTNA

SEQ ID NO: 38 - DNA, 348 bp, Artificial Sequence
  Ab3_VH VH
cagtcgctggaggagtccgggggtcgcctggtcacgcctgggacacccctgacactcacctgcacagtctctggaatcgacctcagtagctatgcagtgggctgggtccgccaggctccagggaaggggctggaatacatcggaatcatttatgctagtggtaacacatggtacgcgagctgggtgaaaggccgattcaccatctccaaaacctcgaccacggtggatctgaagttggccagtctgacaaccgaggacacggccacctatttctgtgccagaggtcatggtgctacttatgactactttaatttgtggggcccgggcaccctcgtcaccgtctcgagc

SEQ ID NO: 39 - protein, 116 aa, Artificial Sequence
  Ab3_VH VH
QSLEESGGRLVTPGTPLTLTCTVSGIDLSSYAVGWVRQAPGKGLEYIGIIYASGNTWYASWVKGRFTISKTSTTVDLKLASLTTEDTATYFCARGHGATYDYFNLWGPGTLVTVSS

SEQ ID NO: 40 - DNA, 15 bp, Artificial Sequence
  Ab3_VH CDRH1
agctatgcagtgggc

SEQ ID NO: 41 - protein, 5 aa, Artificial Sequence
  Ab3_VH CDRH1
SYAVG

SEQ ID NO: 42 - DNA, 48 bp, Artificial Sequence
  Ab3_VH CDRH2
atcatttatgctagtggtaacacatggtacgcgagctgggtgaaaggc

SEQ ID NO: 43 - protein, 16 aa, Artificial Sequence
  Ab3_VH CDRH2
IIYASGNTWYASWVKG

SEQ ID NO: 44 - DNA, 33 bp, Artificial Sequence
  Ab3_VH CDRH3
ggtcatggtgctacttatgactactttaatttg

SEQ ID NO: 45 - protein, 11 aa, Artificial Sequence
  Ab3_VH CDRH3
GHGATYDYFNL

SEQ ID NO: 46 - DNA, 333 bp, Artificial Sequence
  Ab3_VL VL
gcctatgatatgacccagactccagcctctgtggaggtagctgtgggaggcacagtcaccatcaagtgccaggccagtcagagcattagtagctacttagcctggtatcagcagaaaccagggcagcctcccaagctcctgatctatgatgcatccgatctggcatctggggtcccatcgcggttcaaaggcagtggatctgggacagagtacactctcaccatcagcggcgtgcagtgtgacgatgctgccacttactactgtcaacagggtcttgctactgctaatgttgataatgttttcggcggagggaccgaggtggtggtcaaacgt

SEQ ID NO: 47 - protein, 111 aa, Artificial Sequence
  Ab3_VL VL
AYDMTQTPASVEVAVGGTVTIKCQASQSISSYLAWYQQKPGQPPKLLIYDASDLASGVPSRFKGSGSGTEYTLTISGVQCDDAATYYCQQGLATANVDNVFGGGTEVVVKR

SEQ ID NO: 48 - DNA, 33 bp, Artificial Sequence
  Ab3_VL CDRL1
caggccagtcagagcattagtagctacttagcc

SEQ ID NO: 49 - protein, 11 aa, Artificial Sequence
  Ab3_VL CDRL1
QASQSISSYLA

SEQ ID NO: 50 - DNA, 21 bp, Artificial Sequence
  Ab3_VL CDRL2
gatgcatccgatctggcatct

SEQ ID NO: 51 - protein, 7 aa, Artificial Sequence
  Ab3_VL CDRL2
DASDLAS

SEQ ID NO: 52 - DNA, 36 bp, Artificial Sequence
  Ab3_VL CDRL3
caacagggtcttgctactgctaatgttgataatgtt

SEQ ID NO: 53 - protein, 12 aa, Artificial Sequence
  Ab3_VL CDRL3
QQGLATANVDNV

SEQ ID NO: 54 - DNA, 360 bp, Artificial Sequence
  Ab4_VH VH
cagcagcagctggaggagtccgggggaggcctggtcaagcctggaggaaccctgacactcacctgcaaaggctctggaatcgacttcaatgactaccagtacatgtgctgggtccgccaggctccagggaaggggctggagtggatcgcgtgcatttataatggggatggtgagacttactacgcgcactgggcgaaaggccgattcaccatctccaaaacctcgtcgaccacggtgactctgcagatgaccagtctgacagccgcggacacggccacctatttctgtgcgaggggtagtggtgttgggagttatggttacaacttttggggcccgggcaccctcgtcaccgtctcgagc

SEQ ID NO: 55 - protein, 120 aa, Artificial Sequence
  Ab4_VH VH
QQQLEESGGGLVKPGGTLTLTCKGSGIDFNDYQYMCWVRQAPGKGLEWIACIYNGDGETYYAHWAKGRFTISKTSSTTVTLQMTSLTAADTATYFCARGSGVGSYGYNFWGPGTLVTVSS

SEQ ID NO: 56 - DNA, 18 bp, Artificial Sequence
  Ab4_VH CDRH1
gactaccagtacatgtgc

SEQ ID NO: 57 - protein, 6 aa, Artificial Sequence
  Ab4_VH CDRH1
DYQYMC

SEQ ID NO: 58 - DNA, 51 bp, Artificial Sequence
  Ab4_VH CDRH2
tgcatttataatggggatggtgagacttactacgcgcactgggcgaaaggc

SEQ ID NO: 59 - protein, 17 aa, Artificial Sequence
  Ab4_VH CDRH2
CIYNGDGETYYAHWAKG

SEQ ID NO: 60 - DNA, 33 bp, Artificial Sequence
  Ab4_VH CDRH3
ggtagtggtgttgggagttatggttacaacttt

SEQ ID NO: 61 - protein, 11 aa, Artificial Sequence
  Ab4_VH CDRH3
GSGVGSYGYNF

SEQ ID NO: 62 - DNA, 333 bp, Artificial Sequence
  Ab4_VL VL
gctgacattgtgatgacccagactccagcctccgtggaggcagctgtgggaggcacagtcaccatcaagtgccaggccagtgagagcatttacagcaatttagcctggtatcagcagaaagcagggcagcctcccaagctcctaatctacagggcatccaatctggcatctggggtcccatcgcggttcaaaggcagtggatctgggacagagttcactctcaccatcagcgacctggagtgtgccgatgctgccacttactactgtcaaggctattatgccagtactgttactaatggtttcggcggagggaccgaggtggtggtcaaacgt

SEQ ID NO: 63 - protein, 111 aa, Artificial Sequence
  Ab4_VL VL
ADIVMTQTPASVEAAVGGTVTIKCQASESIYSNLAWYQQKAGQPPKLLIYRASNLASGVPSRFKGSGSGTEFTLTISDLECADAATYYCQGYYASTVTNGFGGGTEVVVKR

SEQ ID NO: 64 - DNA, 33 bp, Artificial Sequence
  Ab4_VL CDRL1
caggccagtgagagcatttacagcaatttagcc

SEQ ID NO: 65 - protein, 11 aa, Artificial Sequence
  Ab4_VL CDRL1
QASESIYSNLA

SEQ ID NO: 66 - DNA, 21 bp, Artificial Sequence
  Ab4_VL CDRL2
agggcatccaatctggcatct

SEQ ID NO: 67 - protein, 7 aa, Artificial Sequence
  Ab4_VL CDRL2
RASNLAS

SEQ ID NO: 68 - DNA, 33 bp, Artificial Sequence
  Ab4_VL CDRL3
caaggctattatgccagtactgttactaatggt

SEQ ID NO: 69 - protein, 11 aa, Artificial Sequence
  Ab4_VL CDRL3
QGYYASTVTNG

SEQ ID NO: 70 - DNA, 363 bp, Artificial Sequence
  Ab5_VH VH
caggagcagctggtggagtccgggggaggcctggtccagcctgagggatccctgacactcacctgcacagcctctggaatcgacttcagtagcggctatttcatgtgctgggtccgccaggctccagggaaggggctggagtggatcgcatgcatttcgccttatagtgataatacatggtacgcgaactgggcgaaaggccgattcaccatctccaaaacctcgtcgaccacggtgactctgcaaatgaccagtctgacagccgcggacacggccgcctatcgctgtgcgagaactatggctattggcgcttataacccctttaagttgtggggccagggcaccctggtcaccgtctcgagc

SEQ ID NO: 71 - protein, 121 aa, Artificial Sequence
  Ab5_VH VH
QEQLVESGGGLVQPEGSLTLTCTASGIDFSSGYFMCWVRQAPGKGLEWIACISPYSDNTWYANWAKGRFTISKTSSTTVTLQMTSLTAADTAAYRCARTMAIGAYNPFKLWGQGTLVTVSS

SEQ ID NO: 72 - DNA, 18 bp, Artificial Sequence
  Ab5_VH CDRH1
agcggctatttcatgtgc

SEQ ID NO: 73 - protein, 6 aa, Artificial Sequence
  Ab5_VH CDRH1
SGYFMC

SEQ ID NO: 74 - DNA, 51 bp, Artificial Sequence
  Ab5_VH CDRH2
tgcatttcgccttatagtgataatacatggtacgcgaactgggcgaaaggc

SEQ ID NO: 75 - protein, 17 aa, Artificial Sequence
  Ab5_VH CDRH2
CISPYSDNTWYANWAKG

SEQ ID NO: 76 - DNA, 36 bp, Artificial Sequence
  Ab5_VH CDRH3
actatggctattggcgcttataacccctttaagttg

SEQ ID NO: 77 - protein, 12 aa, Artificial Sequence
  Ab5_VH CDRH3
TMAIGAYNPFKL

SEQ ID NO: 78 - DNA, 321 bp, Artificial Sequence
  Ab5_VL VL
gcccttgtgatgacccagactccatcctccgtgtctgcagctgtgggaggcacagtcaccatcaattgccaggccagtcagaacatttacagcaatttagcctggtatcagcagaaaccagggcagcgtcccaagctcctgatctatgatgcatccgatctggcatctggggtctcatcgcgggtcaaaggcagtggatctgggacagagttcactctcaccatcagcgacctggagtgtgccgatgctgccacttactactgtcaaggctattattctggtaatgctttcggcggagggaccgaggtggtggtcaaacgt

SEQ ID NO: 79 - protein, 107 aa, Artificial Sequence
  Ab5_VL VL
ALVMTQTPSSVSAAVGGTVTINCQASQNIYSNLAWYQQKPGQRPKLLIYDASDLASGVSSRVKGSGSGTEFTLTISDLECADAATYYCQGYYSGNAFGGGTEVVVKR

SEQ ID NO: 80 - DNA, 33 bp, Artificial Sequence
  Ab5_VL CDRL1
caggccagtcagaacatttacagcaatttagcc

SEQ ID NO: 81 - protein, 11 aa, Artificial Sequence
  Ab5_VL CDRL1
QASQNIYSNLA

SEQ ID NO: 82 - DNA, 21 bp, Artificial Sequence
  Ab5_VL CDRL2
gatgcatccgatctggcatct

SEQ ID NO: 83 - protein, 7 aa, Artificial Sequence
  Ab5_VL CDRL2
DASDLAS

SEQ ID NO: 84 - DNA, 24 bp, Artificial Sequence
  Ab5_VL CDRL3
caaggctattattctggtaatgct

SEQ ID NO: 85 - protein, 8 aa, Artificial Sequence
  Ab5_VL CDRL3
QGYYSGNA

SEQ ID NO: 86 - DNA, 348 bp, Artificial Sequence
  Ab6_VH VH
cagtcgctggaggagtccgggggtcgcctggtcacgcctggaggaaccctaacactcacctgcacagtctctggaatcgacctcagtagctatgcaatgagctgggtccgccaggctccagggaaggggctggaatggatcggaatcatttatgctagtggtaacacatggtacgcgaactgggtgaaaggccgattcaccatctccaaaacctcgaccacggtggatctgaaaatgaccagtccgacaaccgaggacacggccacctatttctgtgccagagatagtagtgctacttttaattatctagacttgtggggccaaggcaccctcgtcaccgtctcgagc

SEQ ID NO: 87 - protein, 116 aa, Artificial Sequence
  Ab6_VH VH
QSLEESGGRLVTPGGTLTLTCTVSGIDLSSYAMSWVRQAPGKGLEWIGIIYASGNTWYANWVKGRFTISKTSTTVDLKMTSPTTEDTATYFCARDSSATFNYLDLWGQGTLVTVSS

SEQ ID NO: 88 - DNA, 15 bp, Artificial Sequence
  Ab6_VH CDRH1
agctatgcaatgagc

SEQ ID NO: 89 - protein, 5 aa, Artificial Sequence
  Ab6_VH CDRH1
SYAMS

SEQ ID NO: 90 - DNA, 48 bp, Artificial Sequence
  Ab6_VH CDRH2
atcatttatgctagtggtaacacatggtacgcgaactgggtgaaaggc

SEQ ID NO: 91 - protein, 16 aa, Artificial Sequence
  Ab6_VH CDRH2
IIYASGNTWYANWVKG

SEQ ID NO: 92 - DNA, 33 bp, Artificial Sequence
  Ab6_VH CDRH3
gatagtagtgctacttttaattatctagacttg

SEQ ID NO: 93 - protein, 11 aa, Artificial Sequence
  Ab6_VH CDRH3
DSSATFNYLDL

SEQ ID NO: 94 - DNA, 333 bp, Artificial Sequence
  Ab6_VL VL
gcctttcatatgacccagactccagcctctatggaggtagctgtgggaggcacagtcaccatgaagtgccaggccagtgagagcattgatacctacttaaactggtatcagcagaaaccagggcagcctcccaagctcctgatctaccaggcatccaaactggcatctggggtcccatcgcggttcaaaggcagtggatctgggacagagttcactctcaccatcagcggcgtggagtgtgccgatgctgccacttactactgtcaacagggttggagtactaatgatgttgataatgttttcggcggagggaccgaggtggtggtcaaacgt

SEQ ID NO: 95 - protein, 111 aa, Artificial Sequence
  Ab6_VL VL
AFHMTQTPASMEVAVGGTVTMKCQASESIDTYLNWYQQKPGQPPKLLIYQASKLASGVPSRFKGSGSGTEFTLTISGVECADAATYYCQQGWSTNDVDNVFGGGTEVVVKR

SEQ ID NO: 96 - DNA, 33 bp, Artificial Sequence
  Ab6_VL CDRL1
caggccagtgagagcattgatacctacttaaac

SEQ ID NO: 97 - protein, 11 aa, Artificial Sequence
  Ab6_VL CDRL1
QASESIDTYLN

SEQ ID NO: 98 - DNA, 21 bp, Artificial Sequence
  Ab6_VL CDRL2
caggcatccaaactggcatct

SEQ ID NO: 99 - protein, 7 aa, Artificial Sequence
  Ab6_VL CDRL2
QASKLAS

SEQ ID NO: 100 - DNA, 36 bp, Artificial Sequence
  Ab6_VL CDRL3
caacagggttggagtactaatgatgttgataatgtt

SEQ ID NO: 101 - protein, 12 aa, Artificial Sequence
  Ab6_VL CDRL3
QQGWSTNDVDNV

SEQ ID NO: 102 - DNA, 348 bp, Artificial Sequence
  Ab7_VH VH
cagtcggtggaggagtccgggggtcgcctggtcacgcctgggacacccctgacactcacctgcacagtctctggattctccctcagtcgctatgcaatgggctgggtccgccacgttccagggaaggggctggaatggatcggaatcatttatgctagtggtagcacatactacgcgagctgggcgaagggccgattcaccatctccaaaacctcgaccacggtggatctgaaaatcaccagtccgacaaccgaggacacggccacctatttctgtgccagatcgcgtactgctacttatgatgctgttgatccctggggcccgggcaccctcgtcaccgtctcgagc

SEQ ID NO: 103 - protein, 116 aa, Artificial Sequence
  Ab7_VH VH
QSVEESGGRLVTPGTPLTLTCTVSGFSLSRYAMGWVRHVPGKGLEWIGIIYASGSTYYASWAKGRFTISKTSTTVDLKITSPTTEDTATYFCARSRTATYDAVDPWGPGTLVTVSS

SEQ ID NO: 104 - DNA, 15 bp, Artificial Sequence
  Ab7_VH CDRH1
cgctatgcaatgggc

SEQ ID NO: 105 - protein, 5 aa, Artificial Sequence
  Ab7_VH CDRH1
RYAMG

SEQ ID NO: 106 - DNA, 48 bp, Artificial Sequence
  Ab7_VH CDRH2
atcatttatgctagtggtagcacatactacgcgagctgggcgaagggc

SEQ ID NO: 107 - protein, 16 aa, Artificial Sequence
  Ab7_VH CDRH2
IIYASGSTYYASWAKG

SEQ ID NO: 108 - DNA, 33 bp, Artificial Sequence
  Ab7_VH CDRH3
tcgcgtactgctacttatgatgctgttgatccc

SEQ ID NO: 109 - protein, 11 aa, Artificial Sequence
  Ab7_VH CDRH3
SRTATYDAVDP

SEQ ID NO: 110 - DNA, 333 bp, Artificial Sequence
  Ab7_VL VL
gcctatgatatgacccagactccagcctccgtgtctgaacctgtgggaggcacagtcaccatcaagtgccaggccagtcagagcattagtagttacttatcctggtatcagcagaaaccagggcagcctcccaagctcctgatctacgaagcatccactctggcatctggggtctcatcgcggttcaaaggcagtggatctgggacagagtacactctcaccatcagcgacctggagtgtgacgatgctgccgcttactactgtcaacaggatcttagtggtagtaatcttgataattctttcggcggagggaccgaggtggtggtcaaacgt

SEQ ID NO: 111 - protein, 111 aa, Artificial Sequence
  Ab7_VL VL
AYDMTQTPASVSEPVGGTVTIKCQASQSISSYLSWYQQKPGQPPKLLIYEASTLASGVSSRFKGSGSGTEYTLTISDLECDDAAAYYCQQDLSGSNLDNSFGGGTEVVVKR

SEQ ID NO: 112 - DNA, 33 bp, Artificial Sequence
  Ab7_VL CDRL1
caggccagtcagagcattagtagttacttatcc

SEQ ID NO: 113 - protein, 11 aa, Artificial Sequence
  Ab7_VL CDRL1
QASQSISSYLS

SEQ ID NO: 114 - DNA, 21 bp, Artificial Sequence
  Ab7_VL CDRL2
gaagcatccactctggcatct

SEQ ID NO: 115 - protein, 7 aa, Artificial Sequence
  Ab7_VL CDRL2
EASTLAS

SEQ ID NO: 116 - DNA, 36 bp, Artificial Sequence
  Ab7_VL CDRL3
caacaggatcttagtggtagtaatcttgataattct

SEQ ID NO: 117 - protein, 12 aa, Artificial Sequence
  Ab7_VL CDRL3
QQDLSGSNLDNS

SEQ ID NO: 118 - DNA, 348 bp, Artificial Sequence
  Ab8_VH VH
cagtcggtggaggagtccgggggtcgcctggtcacgcctgggacacccctgacactcacctgctccgtctctggaatcgacctcagtagctatgcaatgagctgggtccgccaggctccagggaaggggctggaatggatcggagtcatttatgctagtggtagcacatactacgcgagctgggcgaaaggccgattcaccatctccaagacctcgaccacggtggatctgaaaatcaccagtccgacaaccggggacacggccacctatttctgtgtcagagatactactgcaacttataactactttaacttgtggggccagggcaccctcgtcaccgtctcgagc

SEQ ID NO: 119 - protein, 116 aa, Artificial Sequence
  Ab8_VH VH
QSVEESGGRLVTPGTPLTLTCSVSGIDLSSYAMSWVRQAPGKGLEWIGVIYASGSTYYASWAKGRFTISKTSTTVDLKITSPTTGDTATYFCVRDTTATYNYFNLWGQGTLVTVSS

SEQ ID NO: 120 - DNA, 15 bp, Artificial Sequence
  Ab8_VH CDRH1
agctatgcaatgagc

SEQ ID NO: 121 - protein, 5 aa, Artificial Sequence
  Ab8_VH CDRH1
SYAMS

SEQ ID NO: 122 - DNA, 48 bp, Artificial Sequence
  Ab8_VH CDRH2
gtcatttatgctagtggtagcacatactacgcgagctgggcgaaaggc

SEQ ID NO: 123 - protein, 16 aa, Artificial Sequence
  Ab8_VH CDRH2
VIYASGSTYYASWAKG

SEQ ID NO: 124 - DNA, 33 bp, Artificial Sequence
  Ab8_VH CDRH3
gatactactgcaacttataactactttaacttg

SEQ ID NO: 125 - protein, 11 aa, Artificial Sequence
  Ab8_VH CDRH3
DTTATYNYFNL

SEQ ID NO: 126 - DNA, 336 bp, Artificial Sequence
  Ab8_VL VL
gcctatgatatgacccagactccagcctctgtggaggtagctgtgggaggcacagccaccatcaagtgccaggccagtcagagcatttacaactacttagcctggtatcagcagaaaccagggcagcctcccaagctcctgatctatgaagcatccactctggcctccggggtcccatcgcggttcaaaggcagtggatctgggacagagttcactctcaccatcagcgacctggagtgtgccgatgctgccacttactactgtcaacagagtttgagtcctagtaatgttggtcgaagggttttcggcggagggaccgaggtggtggtcaaacgt

SEQ ID NO: 127 - protein, 112 aa, Artificial Sequence
  Ab8_VL VL
AYDMTQTPASVEVAVGGTATIKCQASQSIYNYLAWYQQKPGQPPKLLIYEASTLASGVPSRFKGSGSGTEFTLTISDLECADAATYYCQQSLSPSNVGRRVFGGGTEVVVKR

SEQ ID NO: 128 - DNA, 33 bp, Artificial Sequence
  Ab8_VL CDRL1
caggccagtcagagcatttacaactacttagcc

SEQ ID NO: 129 - protein, 11 aa, Artificial Sequence
  Ab8_VL CDRL1
QASQSIYNYLA

SEQ ID NO: 130 - DNA, 21 bp, Artificial Sequence
  Ab8_VL CDRL2
gaagcatccactctggcctcc

SEQ ID NO: 131 - protein, 7 aa, Artificial Sequence
  Ab8_VL CDRL2
EASTLAS

SEQ ID NO: 132 - DNA, 39 bp, Artificial Sequence
  Ab8_VL CDRL3
caacagagtttgagtcctagtaatgttggtcgaagggtt

SEQ ID NO: 133 - protein, 13 aa, Artificial Sequence
  Ab8_VL CDRL3
QQSLSPSNVGRRV

SEQ ID NO: 134 - DNA, 351 bp, Artificial Sequence
  Ab9_VH VH
cagtcggtggaggagtccgggggtcgcctggtcacgcctgggacacccctgacactcacctgcacagtctctggattctccctcaataactatgcaatgaactggatccgccaggctccagggaagggactggaatggatcggaatcatttatggtagtggtagcacatactacgcgagctgggcgaaaggccgattcaccgcctccaaaacctcgaccacggtggatctgaaaatgaccagtctgacaaccgaggacacggccacgtatttctgtgccagagatgaactagctgctggtattaattactttaccttgtggggccaagggaccctggtcaccgtctcgagc

SEQ ID NO: 135 - protein, 117 aa, Artificial Sequence
  Ab9_VH VH
QSVEESGGRLVTPGTPLTLTCTVSGFSLNNYAMNWIRQAPGKGLEWIGIIYGSGSTYYASWAKGRFTASKTSTTVDLKMTSLTTEDTATYFCARDELAAGINYFTLWGQGTLVTVSS

SEQ ID NO: 136 - DNA, 15 bp, Artificial Sequence
  Ab9_VH CDRH1
aactatgcaatgaac

SEQ ID NO: 137 - protein, 5 aa, Artificial Sequence
  Ab9_VH CDRH1
NYAMN

SEQ ID NO: 138 - DNA, 48 bp, Artificial Sequence
  Ab9_VH CDRH2
atcatttatggtagtggtagcacatactacgcgagctgggcgaaaggc

SEQ ID NO: 139 - protein, 16 aa, Artificial Sequence
  Ab9_VH CDRH2
IIYGSGSTYYASWAKG

SEQ ID NO: 140 - DNA, 36 bp, Artificial Sequence
  Ab9_VH CDRH3
gatgaactagctgctggtattaattactttaccttg

SEQ ID NO: 141 - protein, 12 aa, Artificial Sequence
  Ab9_VH CDRH3
DELAAGINYFTL

SEQ ID NO: 142 - DNA, 333 bp, Artificial Sequence
  Ab9_VL VL
gcccttgtgatgacccagactccagcctccgtggaggtagctgtgggaggcacagtcaccatcaagtgccaggccagtcagaatattattgattggtgctcctggtatcagcagaaactagggcagcctcccaaactcctgatctacgaagcatctaaactggcatctggggtcccatcgcggttcaaaggcagtggatctgggacacagttcactctcaccatcagcggcgtggagtgtgccgatgctgccacttactattgtcaacagggttatagtcctagtggtgttgataatgttttcggcggagggaccgaggtggtggtcaaacgt

SEQ ID NO: 143 - protein, 111 aa, Artificial Sequence
  Ab9_VL VL
ALVMTQTPASVEVAVGGTVTIKCQASQNIIDWCSWYQQKLGQPPKLLIYEASKLASGVPSRFKGSGSGTQFTLTISGVECADAATYYCQQGYSPSGVDNVFGGGTEVVVKR

SEQ ID NO: 144 - DNA, 33 bp, Artificial Sequence
  Ab9_VL CDRL1
caggccagtcagaatattattgattggtgctcc

SEQ ID NO: 145 - protein, 11 aa, Artificial Sequence
  Ab9_VL CDRL1
QASQNIIDWCS

SEQ ID NO: 146 - DNA, 21 bp, Artificial Sequence
  Ab9_VL CDRL2
gaagcatctaaactggcatct

SEQ ID NO: 147 - protein, 7 aa, Artificial Sequence
  Ab9_VL CDRL2
EASKLAS

SEQ ID NO: 148 - DNA, 36 bp, Artificial Sequence
  Ab9_VL CDRL3
caacagggttatagtcctagtggtgttgataatgtt

SEQ ID NO: 149 - protein, 12 aa, Artificial Sequence
  Ab9_VL CDRL3
QQGYSPSGVDNV

SEQ ID NO: 150 - DNA, 348 bp, Artificial Sequence
  Ab10_VH VH
cagtcgctggaggagtccgggggtcgcctggtcacgcctgggacacccctgacactcacctgcacagtctctggaatcgacctcagtagctatgcagtgggctgggtccgccaggctccagggaaggggctggaatacatcggaatcatttatgctagtggtaacacatggtacgcgagctgggtgaaaggccgattcaccatctccaaaacctcgaccacggtggatctgaaaatgaccagtctgacaaccgaggacacggccacctatttctgtgccagaggtcatggtgctacttatgactactttaatttgtggggcccaggcaccctcgtcaccgtctcgagc

SEQ ID NO: 151 - protein, 116 aa, Artificial Sequence
  Ab10_VH VH
QSLEESGGRLVTPGTPLTLTCTVSGIDLSSYAVGWVRQAPGKGLEYIGIIYASGNTWYASWVKGRFTISKTSTTVDLKMTSLTTEDTATYFCARGHGATYDYFNLWGPGTLVTVSS

SEQ ID NO: 152 - DNA, 15 bp, Artificial Sequence
  Ab10_VH CDRH1
agctatgcagtgggc

SEQ ID NO: 153 - protein, 5 aa, Artificial Sequence
  Ab10_VH CDRH1
SYAVG

SEQ ID NO: 154 - DNA, 48 bp, Artificial Sequence; identical to SEQ ID NO: 42
  Ab10_VH CDRH2

SEQ ID NO: 155 - protein, 16 aa, Artificial Sequence
  Ab10_VH CDRH2
IIYASGNTWYASWVKG

SEQ ID NO: 156 - DNA, 33 bp, Artificial Sequence; identical to SEQ ID NO: 44
  Ab10_VH CDRH3

SEQ ID NO: 157 - protein, 11 aa, Artificial Sequence
  Ab10_VH CDRH3
GHGATYDYFNL

SEQ ID NO: 158 - DNA, 333 bp, Artificial Sequence
  AB10_VL VL
gcctatgatatgacccagactccagcctctgtggaggtagctgtgggaggcacagtcaccatcaagtgccaggccagtcagagcattagcagttggttagcctggtatcagcagaaaccagggcagcctcccaagctcctgatctatgatgcatccgatctggcatctggggtcccatcgcggttcaaaggcagtggatctgggacagagtacactctcaccatcagcggcgtgcagtgtgacgatgctgccacttactactgtcaacagggtcttgctactgctaatgttgataatgttttcggcggagggaccgaggtggtggtcaaacgt

SEQ ID NO: 159 - protein, 111 aa, Artificial Sequence
  AB10_VL VL
AYDMTQTPASVEVAVGGTVTIKCQASQSISSWLAWYQQKPGQPPKLLIYDASDLASGVPSRFKGSGSGTEYTLTISGVQCDDAATYYCQQGLATANVDNVFGGGTEVVVKR

SEQ ID NO: 160 - DNA, 33 bp, Artificial Sequence
  AB10_VL CDRL1
caggccagtcagagcattagcagttggttagcc

SEQ ID NO: 161 - protein, 11 aa, Artificial Sequence
  AB10_VL CDRL1
QASQSISSWLA

SEQ ID NO: 162 - DNA, 21 bp, Artificial Sequence
  AB10_VL CDRL2
gatgcatccgatctggcatct

SEQ ID NO: 163 - protein, 7 aa, Artificial Sequence
  AB10_VL CDRL2
DASDLAS

SEQ ID NO: 164 - DNA, 36 bp, Artificial Sequence; identical to SEQ ID NO: 52
  AB10_VL CDRL3

SEQ ID NO: 165 - protein, 12 aa, Artificial Sequence
  AB10_VL CDRL3
QQGLATANVDNV

SEQ ID NO: 166 - DNA, 354 bp, Artificial Sequence
  Ab11_VH VH
cagtcgttggaggagtccgggggaggcctggtcaagcctggaggaaccctgacactcacctgcacagcctctggattctccttcagtagcagctactggatatgctgggtccgccaggctccagggaaggggctggagtggatcggatgcatttacggaagtactgatgcctataacgcgaactgggcgaaaggccgattcgccatctcccaaacctcgtcgaccacggtgactctgcaaatgaccagtctgccagccgcggacacggccacctatttctgtgcgcgacatgatggtgatattggttggaccttggacttgtggggcccggggaccctggtcaccgtctcgagc

SEQ ID NO: 167 - protein, 118 aa, Artificial Sequence
  Ab11_VH VH
QSLEESGGGLVKPGGTLTLTCTASGFSFSSSYWICWVRQAPGKGLEWIGCIYGSTDAYNANWAKGRFAISQTSSTTVTLQMTSLPAADTATYFCARHDGDIGWTLDLWGPGTLVTVSS

SEQ ID NO: 168 - DNA, 18 bp, Artificial Sequence
  Ab11_VH CDRH1
agcagctactggatatgc

SEQ ID NO: 169 - protein, 6 aa, Artificial Sequence
  Ab11_VH CDRH1
SSYWIC

SEQ ID NO: 170 - DNA, 48 bp, Artificial Sequence
  Ab11_VH CDRH2
tgcatttacggaagtactgatgcctataacgcgaactgggcgaaaggc

SEQ ID NO: 171 - protein, 16 aa, Artificial Sequence
  Ab11_VH CDRH2
CIYGSTDAYNANWAKG

SEQ ID NO: 172 - DNA, 33 bp, Artificial Sequence
  Ab11_VH CDRH3
catgatggtgatattggttggaccttggacttg

SEQ ID NO: 173 - protein, 11 aa, Artificial Sequence
  Ab11_VH CDRH3
HDGDIGWTLDL

SEQ ID NO: 174 - DNA, 333 bp, Artificial Sequence
  Ab11_VL VL
gcctatgatatgacccagactccagcctctgtggaggtcgctgtgggaggcacagtcaccatcaagtgccaggccagtctgaccattggtactcggttagcctggtatcagcagaaaccagggcagcctcccaagctcctgatctacaaggcatccactctggcatctggggtctcatcgcggttcaaaggcagtggatctgggacacagttcgctctcaccatcagcgacctggaatgtgccgatgctgccacttactactgtcaacaggctgcttatgttactgatatagataatgctttcggcggagggaccgaggtggtggtcaaacgt

SEQ ID NO: 175 - protein, 111 aa, Artificial Sequence
  Ab11_VL VL
AYDMTQTPASVEVAVGGTVTIKCQASLTIGTRLAWYQQKPGQPPKLLIYKASTLASGVSSRFKGSGSGTQFALTISDLECADAATYYCQQAAYVTDIDNAFGGGTEVVVKR

SEQ ID NO: 176 - DNA, 33 bp, Artificial Sequence
  Ab11_VL CDRL1
caggccagtctgaccattggtactcggttagcc

SEQ ID NO: 177 - protein, 11 aa, Artificial Sequence
  Ab11_VL CDRL1
QASLTIGTRLA

SEQ ID NO: 178 - DNA, 21 bp, Artificial Sequence
  Ab11_VL CDRL2
aaggcatccactctggcatct

SEQ ID NO: 179 - protein, 7 aa, Artificial Sequence
  Ab11_VL CDRL2
KASTLAS

SEQ ID NO: 180 - DNA, 36 bp, Artificial Sequence
  Ab11_VL CDRL3
caacaggctgcttatgttactgatatagataatgct

SEQ ID NO: 181 - protein, 12 aa, Artificial Sequence
  Ab11_VL CDRL3
QQAAYVTDIDNA

SEQ ID NO: 182 - DNA, 348 bp, Artificial Sequence
  Ab12_VH VH
cagtcggtggaggagtccgggggtcgcctggtcacgcctgggacacccctgacactcacctgcacagtctctggaatcgacctcagtagcaatgcaatgggctgggtccgccaggctccagggaaggggctggaatggatcgggatcatttatgctagtggtaacgcatggtacgcgaactgggtgaaaggccgattcaccatctccaaaacctcgaccacggtggatctgaaaatcaccagtccgacaaccgaggacacggccacctatttctgtgccagggatgctactgctacttgggactactttaacttgtggggccaaggcaccctcgtcaccgtctcgagc

SEQ ID NO: 183 - protein, 116 aa, Artificial Sequence
  Ab12_VH VH
QSVEESGGRLVTPGTPLTLTCTVSGIDLSSNAMGWVRQAPGKGLEWIGIIYASGNAWYANWVKGRFTISKTSTTVDLKITSPTTEDTATYFCARDATATWDYFNLWGQGTLVTVSS

SEQ ID NO: 184 - DNA, 15 bp, Artificial Sequence
  Ab12_VH CDRH1
agcaatgcaatgggc

SEQ ID NO: 185 - protein, 5 aa, Artificial Sequence
  Ab12_VH CDRH1
SNAMG

SEQ ID NO: 186 - DNA, 48 bp, Artificial Sequence
  Ab12_VH CDRH2
atcatttatgctagtggtaacgcatggtacgcgaactgggtgaaaggc

SEQ ID NO: 187 - protein, 16 aa, Artificial Sequence
  Ab12_VH CDRH2
IIYASGNAWYANWVKG

SEQ ID NO: 188 - DNA, 33 bp, Artificial Sequence
  Ab12_VH CDRH3
gatgctactgctacttgggactactttaacttg

SEQ ID NO: 189 - protein, 11 aa, Artificial Sequence
  Ab12_VH CDRH3
DATATWDYFNL

SEQ ID NO: 190 - DNA, 333 bp, Artificial Sequence
  Ab12_VL VL
gcctatgatatgacccagactccatcctccgtgtctgcagctgtgggaggcacagtcaccatcaagtgccaggccagtcagagcattagtaattggttatcctggtatcagcagaaaccagggcagcctcccaagctcctgatctacagggcgtccactctggcatctggggtctcatcgcggttcagcggcagtggatctgggacagagttcactctcaccatcagcgacctggagtgtgccgatgctgccacttactactgtcaacagggttatagtgttgctaatgtggataatgctttcggcggagggaccgaggtggtggtcaaacgt

SEQ ID NO: 191 - protein, 111 aa, Artificial Sequence
  Ab12_VL VL
AYDMTQTPSSVSAAVGGTVTIKCQASQSISNWLSWYQQKPGQPPKLLIYRASTLASGVSSRFSGSGSGTEFTLTISDLECADAATYYCQQGYSVANVDNAFGGGTEVVVKR

SEQ ID NO: 192 - DNA, 33 bp, Artificial Sequence
  Ab12_VL CDRL1
caggccagtcagagcattagtaattggttatcc

SEQ ID NO: 193 - protein, 11 aa, Artificial Sequence
  Ab12_VL CDRL1
QASQSISNWLS

SEQ ID NO: 194 - DNA, 21 bp, Artificial Sequence
  Ab12_VL CDRL2
agggcgtccactctggcatct

SEQ ID NO: 195 - protein, 7 aa, Artificial Sequence
  Ab12_VL CDRL2
RASTLAS

SEQ ID NO: 196 - DNA, 36 bp, Artificial Sequence
  Ab12_VL CDRL3
caacagggttatagtgttgctaatgtggataatgct

SEQ ID NO: 197 - protein, 12 aa, Artificial Sequence
  Ab12_VL CDRL3
QQGYSVANVDNA

SEQ ID NO: 198 - DNA, 357 bp, Artificial Sequence
  Ab13_VH VH
cagtcgttggaggagtccgggggagacctggtcaagcctggggcatccctgacactcacctgcacagcctctggattctccttcagtagcagcgactacatgtgttgggtccgccaggctccagggaaggggctggagtggatcggatgcatttatactggtgatggtaccacatactacgcgagctgggcgaaaggccgattcaccatctccaaaacctcgtcgaccacggtgactctgcaaatgaccagtctgacagccgcggacacggccacctatttctgtgcgagaggggctggtgttggttcttatggttataatttgtggggcccgggcaccctcgtcaccgtctcgagc

SEQ ID NO: 199 - protein, 119 aa, Artificial Sequence
  Ab13_VH VH
QSLEESGGDLVKPGASLTLTCTASGFSFSSSDYMCWVRQAPGKGLEWIGCIYTGDGTTYYASWAKGRFTISKTSSTTVTLQMTSLTAADTATYFCARGAGVGSYGYNLWGPGTLVTVSS

SEQ ID NO: 200 - DNA, 18 bp, Artificial Sequence
  Ab13_VH CDRH1
agcagcgactacatgtgt

SEQ ID NO: 201 - protein, 6 aa, Artificial Sequence
  Ab13_VH CDRH1
SSDYMC

SEQ ID NO: 202 - DNA, 51 bp, Artificial Sequence
  Ab13_VH CDRH2
tgcatttatactggtgatggtaccacatactacgcgagctgggcgaaaggc

SEQ ID NO: 203 - protein, 17 aa, Artificial Sequence
  Ab13_VH CDRH2
CIYTGDGTTYYASWAKG

SEQ ID NO: 204 - DNA, 33 bp, Artificial Sequence
  Ab13_VH CDRH3
ggggctggtgttggttcttatggttataatttg

SEQ ID NO: 205 - protein, 11 aa, Artificial Sequence
  Ab13_VH CDRH3
GAGVGSYGYNL

SEQ ID NO: 206 - DNA, 333 bp, Artificial Sequence
  Ab13_VL VL
gttgacattgtgatgacccagactccagcctccgtggaggcagctgtgggagacacagtcaccatcaattgccaggccagtcagaacatttacagcaatttagcctggtatcaacagaaaccagggcagcctcccaagctcctgatctacagggcatccactctggcatctggggtcccatcgcggttcaaaggcagtggatctgggacacagttcactctcaccatcagcgacctggagtgtgccgatgctgccacttactactgtcaaagctattatgcgagtagtattagtaattatttcggcggagggaccgaggtggtggtcaaacgt

SEQ ID NO: 207 - protein, 111 aa, Artificial Sequence
  Ab13_VL VL
VDIVMTQTPASVEAAVGDTVTINCQASQNIYSNLAWYQQKPGQPPKLLIYRASTLASGVPSRFKGSGSGTQFTLTISDLECADAATYYCQSYYASSISNYFGGGTEVVVKR

SEQ ID NO: 208 - DNA, 33 bp, Artificial Sequence; identical to SEQ ID NO: 80
  Ab13_VL CDRL1

SEQ ID NO: 209 - protein, 11 aa, Artificial Sequence
  Ab13_VL CDRL1
QASQNIYSNLA

SEQ ID NO: 210 - DNA, 21 bp, Artificial Sequence
  Ab13_VL CDRL2
agggcatccactctggcatct

SEQ ID NO: 211 - protein, 7 aa, Artificial Sequence
  Ab13_VL CDRL2
RASTLAS

SEQ ID NO: 212 - DNA, 33 bp, Artificial Sequence
  Ab13_VL CDRL3
caaagctattatgcgagtagtattagtaattat

SEQ ID NO: 213 - protein, 11 aa, Artificial Sequence
  Ab13_VL CDRL3
QSYYASSISNY

SEQ ID NO: 214 - DNA, 360 bp, Artificial Sequence
  Ab14_VH VH
cagcagcagctggaggagtccgggggaggcctggtcaagcctggaggaaccctgacactcacctgcaaaggctctggaatcgacttcaatgattactactacatgtgctgggtccgccaggctccagggaaggggctggagtggatcgcgtgcatttataatggtgatggtgagacttactacgcgaactgggcgaaaggccgattcaccatctccaaaacctcgtcgaccacggtgactctgcagatgaccagtctgacagccgcggacacggccacctatttctgtgcgaggggtagtggtgttgggagttatggttacaacttttggggccaggggaccctggtcaccgtctcgagc

SEQ ID NO: 215 - protein, 120 aa, Artificial Sequence
  Ab14_VH VH
QQQLEESGGGLVKPGGTLTLTCKGSGIDFNDYYYMCWVRQAPGKGLEWIACIYNGDGETYYANWAKGRFTISKTSSTTVTLQMTSLTAADTATYFCARGSGVGSYGYNFWGQGTLVTVSS

SEQ ID NO: 216 - DNA, 18 bp, Artificial Sequence
  Ab14_VH CDRH1
gattactactacatgtgc

SEQ ID NO: 217 - protein, 6 aa, Artificial Sequence
  Ab14_VH CDRH1
DYYYMC

SEQ ID NO: 218 - DNA, 51 bp, Artificial Sequence
  Ab14_VH CDRH2
tgcatttataatggtgatggtgagacttactacgcgaactgggcgaaaggc

SEQ ID NO: 219 - protein, 17 aa, Artificial Sequence
  Ab14_VH CDRH2
CIYNGDGETYYANWAKG

SEQ ID NO: 220 - DNA, 33 bp, Artificial Sequence; identical to SEQ ID NO: 60
  Ab14_VH CDRH3

SEQ ID NO: 221 - protein, 11 aa, Artificial Sequence
  Ab14_VH CDRH3
GSGVGSYGYNF

SEQ ID NO: 222 - DNA, 333 bp, Artificial Sequence
  Ab14_VL VL
gctgacattgtgatgacccagactccagcctccgtggaggcagctgtgggaggcacagtcaccatcaagtgccaggccagtgagagcatttacagcaatttagcctggtatcagcagaaaccagggcagcctcccaagctcctaatctacagggcatccaatctggcatctggggtcccatcgcggttcaaaggcagtggatctgggacagagttcactctcaccatcagcgacctggagtgtgccgatgctgccacttactactgtcaaggctattatgccagtactgttactaatgctttcggcggagggaccgaggtggtggtcaaacgt

SEQ ID NO: 223 - protein, 111 aa, Artificial Sequence
  Ab14_VL VL
ADIVMTQTPASVEAAVGGTVTIKCQASESIYSNLAWYQQKPGQPPKLLIYRASNLASGVPSRFKGSGSGTEFTLTISDLECADAATYYCQGYYASTVTNAFGGGTEVVVKR

SEQ ID NO: 224 - DNA, 33 bp, Artificial Sequence; identical to SEQ ID NO: 64
  Ab14_VL CDRL1

SEQ ID NO: 225 - protein, 11 aa, Artificial Sequence
  Ab14_VL CDRL1
QASESIYSNLA

SEQ ID NO: 226 - DNA, 21 bp, Artificial Sequence
  Ab14_VL CDRL2
agggcatccaatctggcatct

SEQ ID NO: 227 - protein, 7 aa, Artificial Sequence
  Ab14_VL CDRL2
RASNLAS

SEQ ID NO: 228 - DNA, 33 bp, Artificial Sequence
  Ab14_VL CDRL3
caaggctattatgccagtactgttactaatgct

SEQ ID NO: 229 - protein, 11 aa, Artificial Sequence
  Ab14_VL CDRL3
QGYYASTVTNA

SEQ ID NO: 230 - DNA, 357 bp, Artificial Sequence
  Ab15_VH VH
cagcagctggtggagtccgggggaggcctggtcaagcctggaggaaccctgacactcacctgcaaaggctctggaatcgacttcaatgactactactacatatgctgggtccgccaggctccagggaaggggctggagtggatcctatgcatttataatggcgatggtgagacttactacgcgaactgggcgaaaggccgattcaccatctccaaaacctcgtcgaccacggtgactctgcagatgaccagtctgacaggcgcggacacggccacctatttctgtgcgaggggtagtggtgttgggagttatggttacaacttttggggccaggggaccctggtcaccgtctcgagc

SEQ ID NO: 231 - protein, 119 aa, Artificial Sequence
  Ab15_VH VH
QQLVESGGGLVKPGGTLTLTCKGSGIDFNDYYYICWVRQAPGKGLEWILCIYNGDGETYYANWAKGRFTISKTSSTTVTLQMTSLTGADTATYFCARGSGVGSYGYNFWGQGTLVTVSS

SEQ ID NO: 232 - DNA, 18 bp, Artificial Sequence
  Ab15_VH CDRH1
gactactactacatatgc

SEQ ID NO: 233 - protein, 6 aa, Artificial Sequence
  Ab15_VH CDRH1
DYYYIC

SEQ ID NO: 234 - DNA, 51 bp, Artificial Sequence
  Ab15_VH CDRH2
tgcatttataatggcgatggtgagacttactacgcgaactgggcgaaaggc

SEQ ID NO: 235 - protein, 17 aa, Artificial Sequence
  Ab15_VH CDRH2
CIYNGDGETYYANWAKG

SEQ ID NO: 236 - DNA, 33 bp, Artificial Sequence; identical to SEQ ID NO: 60
  Ab15_VH CDRH3

SEQ ID NO: 237 - protein, 11 aa, Artificial Sequence
  Ab15_VH CDRH3
GSGVGSYGYNF

SEQ ID NO: 238 - DNA, 333 bp, Artificial Sequence
  Ab15_VL VL
gctgacattgtgatgacccagactccagcctccgtggaggcagctgtgggaggcacagtcaccatcaagtgccaggccagtgagaacatttacagcaatttagcctggtatcagcagaaaccagggcagcctcccaagctcctaatttacagggcatccaatctggcatctggggtcccatcgcggttcaaaggcagtggatctgggacagagttcactctcaccatcagcgacctggagtgtgccgatgctgccacttactactgtcaaggctattatgccagtactgttactaatggtttcggcggagggaccgaggtggtggtcaaacgt

SEQ ID NO: 239 - protein, 111 aa, Artificial Sequence
  Ab15_VL VL
ADIVMTQTPASVEAAVGGTVTIKCQASENIYSNLAWYQQKPGQPPKLLIYRASNLASGVPSRFKGSGSGTEFTLTISDLECADAATYYCQGYYASTVTNGFGGGTEVVVKR

SEQ ID NO: 240 - DNA, 33 bp, Artificial Sequence
  Ab15_VL CDRL1
caggccagtgagaacatttacagcaatttagcc

SEQ ID NO: 241 - protein, 11 aa, Artificial Sequence
  Ab15_VL CDRL1
QASENIYSNLA

SEQ ID NO: 242 - DNA, 21 bp, Artificial Sequence
  Ab15_VL CDRL2
agggcatccaatctggcatct

SEQ ID NO: 243 - protein, 7 aa, Artificial Sequence
  Ab15_VL CDRL2
RASNLAS

SEQ ID NO: 244 - DNA, 33 bp, Artificial Sequence; identical to SEQ ID NO: 68
  Ab15_VL CDRL3

SEQ ID NO: 245 - protein, 11 aa, Artificial Sequence
  Ab15_VL CDRL3
QGYYASTVTNG

SEQ ID NO: 246 - DNA, 339 bp, Artificial Sequence
  Ab16_VH VH
cagtcggtggaggagtccgggggtcgcctggtcacgcctgggacacccctgacactcacctgcacagcctctggattcaccatcagtagctattcaatgatctgggtccgccaggctccagggaaggggctggaatccatcggatggattaatactggtggtagcgcatactacgcgaactgggcaaaaggccgattcaccatctccagaacctcgaccacggtggatctgaaaatgaccagtctgacaaccgaggacacggccacctatttctgtgccagagacaataactggtcttttaacttgtggggccaaggcaccctggtcaccgtctcgagc

SEQ ID NO: 247 - protein, 113 aa, Artificial Sequence
  Ab16_VH VH
QSVEESGGRLVTPGTPLTLTCTASGFTISSYSMIWVRQAPGKGLESIGWINTGGSAYYANWAKGRFTISRTSTTVDLKMTSLTTEDTATYFCARDNNWSFNLWGQGTLVTVSS

SEQ ID NO: 248 - DNA, 15 bp, Artificial Sequence
  Ab16_VH CDRH1
agctattcaatgatc

SEQ ID NO: 249 - protein, 5 aa, Artificial Sequence
  Ab16_VH CDRH1
SYSMI

SEQ ID NO: 250 - DNA, 48 bp, Artificial Sequence
  Ab16_VH CDRH2
tggattaatactggtggtagcgcatactacgcgaactgggcaaaaggc

SEQ ID NO: 251 - protein, 16 aa, Artificial Sequence
  Ab16_VH CDRH2
WINTGGSAYYANWAKG

SEQ ID NO: 252 - DNA, 24 bp, Artificial Sequence
  Ab16_VH CDRH3
gacaataactggtcttttaacttg

SEQ ID NO: 253 - protein, 8 aa, Artificial Sequence
  Ab16_VH CDRH3
DNNWSFNL

SEQ ID NO: 254 - DNA, 342 bp, Artificial Sequence
  AB16_VL VL
gctgacattgtgatgacccagactccatcctccgtgtctgcagctgtgggaggcacagtcaccatcaagtgccaggccagtcagagcattggtactaatttagcctggtatcagcagaaatcagggcagcctcccaagcgcctgacctacagggcatccaatctggaatctggggtcccatcgcggttcaaaggcagtggatctgggacagagttcactctcaccatcagcgacctggagtgtgccgatgctgccacttactactgtcaaggcggttattatcgtgagggtaatagtcccacttatcctttcggcggagggaccgaggtggtggtcaaacgt

SEQ ID NO: 255 - protein, 114 aa, Artificial Sequence
  AB16_VL VL
ADIVMTQTPSSVSAAVGGTVTIKCQASQSIGTNLAWYQQKSGQPPKRLTYRASNLESGVPSRFKGSGSGTEFTLTISDLECADAATYYCQGGYYREGNSPTYPFGGGTEVVVKR

SEQ ID NO: 256 - DNA, 33 bp, Artificial Sequence
  AB16_VL CDRL1
caggccagtcagagcattggtactaatttagcc

SEQ ID NO: 257 - protein, 11 aa, Artificial Sequence
  AB16_VL CDRL1
QASQSIGTNLA

SEQ ID NO: 258 - DNA, 21 bp, Artificial Sequence
  AB16_VL CDRL2
agggcatccaatctggaatct

SEQ ID NO: 259 - protein, 7 aa, Artificial Sequence
  AB16_VL CDRL2
RASNLES

SEQ ID NO: 260 - DNA, 42 bp, Artificial Sequence
  AB16_VL CDRL3
caaggcggttattatcgtgagggtaatagtcccacttatcct

SEQ ID NO: 261 - protein, 14 aa, Artificial Sequence
  AB16_VL CDRL3
QGGYYREGNSPTYP

SEQ ID NO: 262 - DNA, 360 bp, Artificial Sequence
  Ab17_VH VH
cagcggcagctggaggagtccgggggaggcctggtcaagcctggaggagccctgacactcacctgcaaagcctctggaatcgacttcagtagctactactacatatgttgggtccgccaggctccagggaaggggctggagtggatcgcatgcatttatgctggtgatggtaccacttactacgcgacctgggcggaaggccgattcaccagctccaaaacctcgtcgaccacggtgactctgcaaatgaccagtctgacagccgcggacacggccacctatttctgtgcgaggggtagtggtgttggtagttatggttacaacttgtggggccaaggcaccctcgtcaccgtctcgagc

SEQ ID NO: 263 - protein, 120 aa, Artificial Sequence
  Ab17_VH VH
QRQLEESGGGLVKPGGALTLTCKASGIDFSSYYYICWVRQAPGKGLEWIACIYAGDGTTYYATWAEGRFTSSKTSSTTVTLQMTSLTAADTATYFCARGSGVGSYGYNLWGQGTLVTVSS

SEQ ID NO: 264 - DNA, 18 bp, Artificial Sequence
  Ab17_VH CDRH1
agctactactacatatgt

SEQ ID NO: 265 - protein, 6 aa, Artificial Sequence
  Ab17_VH CDRH1
SYYYIC

SEQ ID NO: 266 - DNA, 51 bp, Artificial Sequence
  Ab17_VH CDRH2
tgcatttatgctggtgatggtaccacttactacgcgacctgggcggaaggc

SEQ ID NO: 267 - protein, 17 aa, Artificial Sequence
  Ab17_VH CDRH2
CIYAGDGTTYYATWAEG

SEQ ID NO: 268 - DNA, 33 bp, Artificial Sequence; identical to SEQ ID NO: 28
  Ab17_VH CDRH3

SEQ ID NO: 269 - protein, 11 aa, Artificial Sequence
  Ab17_VH CDRH3
GSGVGSYGYNL

SEQ ID NO: 270 - DNA, 333 bp, Artificial Sequence
  Ab17_VL VL
gctgacattgtgatgacccagactccagcctccgtggaggcagctgtgggaggcacagtcaccatcaagtgccaggccagtgagtacatttatagcaatttagcctggtatcagcaaaaaccagggcagcctcccaagctcctgatctacagggcatccactctggaatctggggtcccatcgcggttcaaaggcagtggatctgggacagagttcactctcaccatcagcgacctggagtgtgccgatgctgccacttactactgtcaaggctattttcccagtattattactaatgctttcggcggagggaccgaggtggtggtcaaacgt

SEQ ID NO: 271 - protein, 111 aa, Artificial Sequence
  Ab17_VL VL
ADIVMTQTPASVEAAVGGTVTIKCQASEYIYSNLAWYQQKPGQPPKLLIYRASTLESGVPSRFKGSGSGTEFTLTISDLECADAATYYCQGYFPSIITNAFGGGTEVVVKR

SEQ ID NO: 272 - DNA, 33 bp, Artificial Sequence
  Ab17_VL CDRL1
caggccagtgagtacatttatagcaatttagcc

SEQ ID NO: 273 - protein, 11 aa, Artificial Sequence
  Ab17_VL CDRL1
QASEYIYSNLA

SEQ ID NO: 274 - DNA, 21 bp, Artificial Sequence
  Ab17_VL CDRL2
agggcatccactctggaatct

SEQ ID NO: 275 - protein, 7 aa, Artificial Sequence
  Ab17_VL CDRL2
RASTLES

SEQ ID NO: 276 - DNA, 33 bp, Artificial Sequence
  Ab17_VL CDRL3
caaggctattttcccagtattattactaatgct

SEQ ID NO: 277 - protein, 11 aa, Artificial Sequence
  Ab17_VL CDRL3
QGYFPSIITNA

SEQ ID NO: 278 - DNA, 360 bp, Artificial Sequence
  Ab18_VH VH
cagcagcagctggaggagtccgggggaggcctggtcaagcctggaggaaccctgacactcacctgcaaagcctctggattagacttcagtaactaccactacatgtgctgggtccgccaggctccagggaagaggcctgagtggatcgcatgcatttataatggtgatggtaccacttacgacgcgagctgggcgaaaggccgattcaccagctccaaaacctcgtcgaccacggtgactctgcaaatgaccagtctgacagccgcggacacggccacctatttctgtgcgaggggtagtggtgttggtagttatggttacaacttgtggggccaaggcaccctcgtcaccgtctcgagc

SEQ ID NO: 279 - protein, 120 aa, Artificial Sequence
  Ab18_VH VH
QQQLEESGGGLVKPGGTLTLTCKASGLDFSNYHYMCWVRQAPGKRPEWIACIYNGDGTTYDASWAKGRFTSSKTSSTTVTLQMTSLTAADTATYFCARGSGVGSYGYNLWGQGTLVTVSS

SEQ ID NO: 280 - DNA, 18 bp, Artificial Sequence
  Ab18_VH CDRH1
aactaccactacatgtgc

SEQ ID NO: 281 - protein, 6 aa, Artificial Sequence
  Ab18_VH CDRH1
NYHYMC

SEQ ID NO: 282 - DNA, 51 bp, Artificial Sequence
  Ab18_VH CDRH2
tgcatttataatggtgatggtaccacttacgacgcgagctgggcgaaaggc

SEQ ID NO: 283 - protein, 17 aa, Artificial Sequence
  Ab18_VH CDRH2
CIYNGDGTTYDASWAKG

SEQ ID NO: 284 - DNA, 33 bp, Artificial Sequence; identical to SEQ ID NO: 28
  Ab18_VH CDRH3

SEQ ID NO: 285 - protein, 11 aa, Artificial Sequence
  Ab18_VH CDRH3
GSGVGSYGYNL

SEQ ID NO: 286 - DNA, 333 bp, Artificial Sequence
  Ab18_VL VL
gctgacattgtgatgacccagactccagcctccgtggaggcagctgtgggaggcacagtcaccatcaagtgccaggccagtgagagcatttatagcaatttagcctggtatcagcagaaaccagggcagcctcccaagctcctgatctacagggcatccactctggaatctggggtcccatcgcggttcaaaggcagtggatctgggacagagttcactctcaccatcagcgacctggagtgtgccgatgctgccacttactactgtcaaggctattatgccagtattattactaatgctttcggcggagggaccgaggtggtggtcaaacgt

SEQ ID NO: 287 - protein, 111 aa, Artificial Sequence
  Ab18_VL VL
ADIVMTQTPASVEAAVGGTVTIKCQASESIYSNLAWYQQKPGQPPKLLIYRASTLESGVPSRFKGSGSGTEFTLTISDLECADAATYYCQGYYASIITNAFGGGTEVVVKR

SEQ ID NO: 288 - DNA, 33 bp, Artificial Sequence; identical to SEQ ID NO: 16
  Ab18_VL CDRL1

SEQ ID NO: 289 - protein, 11 aa, Artificial Sequence
  Ab18_VL CDRL1
QASESIYSNLA

SEQ ID NO: 290 - DNA, 21 bp, Artificial Sequence
  Ab18_VL CDRL2
agggcatccactctggaatct

SEQ ID NO: 291 - protein, 7 aa, Artificial Sequence
  Ab18_VL CDRL2
RASTLES

SEQ ID NO: 292 - DNA, 33 bp, Artificial Sequence
  Ab18_VL CDRL3
caaggctattatgccagtattattactaatgct

SEQ ID NO: 293 - protein, 11 aa, Artificial Sequence
  Ab18_VL CDRL3
QGYYASIITNA

SEQ ID NO: 294 - DNA, 363 bp, Artificial Sequence
  Ab19_VH VH
caggagcagctggaggagtccgggggagacctggtcaagcctgagggatccctgacactcacctgcacagcctctggattctccttcagtaactactactacatgtgctgggtccgccaggctccagggaaggggctggagtggatcgcatgcatttatactagtagtggtagcacttactacgcgaactgggtgaatggccgattcaccatctccagaagcaccagcctaaacacggtgactctgcaagtgaccagtctgacagccgcggacacggccacctatttctgtgcgaaatcttttggtgttggtggttactcctttaacttgtggggccaaggcaccctggtcaccgtctcgagc

SEQ ID NO: 295 - protein, 121 aa, Artificial Sequence
  Ab19_VH VH
QEQLEESGGDLVKPEGSLTLTCTASGFSFSNYYYMCWVRQAPGKGLEWIACIYTSSGSTYYANWVNGRFTISRSTSLNTVTLQVTSLTAADTATYFCAKSFGVGGYSFNLWGQGTLVTVSS

SEQ ID NO: 296 - DNA, 18 bp, Artificial Sequence
  Ab19_VH CDRH1
aactactactacatgtgc

SEQ ID NO: 297 - protein, 6 aa, Artificial Sequence
  Ab19_VH CDRH1
NYYYMC

SEQ ID NO: 298 - DNA, 51 bp, Artificial Sequence
  Ab19_VH CDRH2
tgcatttatactagtagtggtagcacttactacgcgaactgggtgaatggc

SEQ ID NO: 299 - protein, 17 aa, Artificial Sequence
  Ab19_VH CDRH2
CIYTSSGSTYYANWVNG

SEQ ID NO: 300 - DNA, 33 bp, Artificial Sequence
  Ab19_VH CDRH3
tcttttggtgttggtggttactcctttaacttg

SEQ ID NO: 301 - protein, 11 aa, Artificial Sequence
  Ab19_VH CDRH3
SFGVGGYSFNL

SEQ ID NO: 302 - DNA, 333 bp, Artificial Sequence
  Ab19_VL VL
gcctatgatatgacccagactccagcctctgtggaggtagctgtgggaggcacagtcaccatcaagtgccaggccagtcagagtattagtagctacttatcctggtatcagcagaaaccagggcagcgtcccaagctcctgatctacagggcatccactctggcatctggggtctcatcgcggttcaaaggcagtggatctgggacagagtacagtctctccatcagcggcatggagtgtgacgatgctgccacttactactgtcagcagggtactactattagtgatgttgataatgttttcggcggagggaccgaggtggtggtcaaacgt

SEQ ID NO: 303 - protein, 111 aa, Artificial Sequence
  Ab19_VL VL
AYDMTQTPASVEVAVGGTVTIKCQASQSISSYLSWYQQKPGQRPKLLIYRASTLASGVSSRFKGSGSGTEYSLSISGMECDDAATYYCQQGTTISDVDNVFGGGTEVVVKR

SEQ ID NO: 304 - DNA, 33 bp, Artificial Sequence
  Ab19_VL CDRL1
caggccagtcagagtattagtagctacttatcc

SEQ ID NO: 305 - protein, 11 aa, Artificial Sequence
  Ab19_VL CDRL1
QASQSISSYLS

SEQ ID NO: 306 - DNA, 21 bp, Artificial Sequence
  Ab19_VL CDRL2
agggcatccactctggcatct

SEQ ID NO: 307 - protein, 7 aa, Artificial Sequence
  Ab19_VL CDRL2
RASTLAS

SEQ ID NO: 308 - DNA, 36 bp, Artificial Sequence
  Ab19_VL CDRL3
cagcagggtactactattagtgatgttgataatgtt

SEQ ID NO: 309 - protein, 12 aa, Artificial Sequence
  Ab19_VL CDRL3
QQGTTISDVDNV

SEQ ID NO: 310 - DNA, 348 bp, Artificial Sequence
  Ab20_VH VH
cagtcggtggaggagtccgggggtcgcctggtcacgcctgggacacccctgacactcacctgcacagtctctggattctccctcagtgattatggattgaactgggtccgccaggctccagggaaggggctggagtggatcggaatcatttatgctagtggtagcacatactacgcgaactgggcgaaaggccgattcaccatctccaaaacctcgaccacggtggatctgaaaatgaccagtccgacaaccgaggacacggccacctatttctgtgtcagagatagttctgctacttatgactactttaacttgtggggcccgggcaccctggtcaccgtctcgagc

SEQ ID NO: 311 - protein, 116 aa, Artificial Sequence
  Ab20_VH VH
QSVEESGGRLVTPGTPLTLTCTVSGFSLSDYGLNWVRQAPGKGLEWIGIIYASGSTYYANWAKGRFTISKTSTTVDLKMTSPTTEDTATYFCVRDSSATYDYFNLWGPGTLVTVSS

SEQ ID NO: 312 - DNA, 15 bp, Artificial Sequence
  Ab20_VH CDRH1
gattatggattgaac

SEQ ID NO: 313 - protein, 5 aa, Artificial Sequence
  Ab20_VH CDRH1
DYGLN

SEQ ID NO: 314 - DNA, 48 bp, Artificial Sequence
  Ab20_VH CDRH2
atcatttatgctagtggtagcacatactacgcgaactgggcgaaaggc

SEQ ID NO: 315 - protein, 16 aa, Artificial Sequence
  Ab20_VH CDRH2
IIYASGSTYYANWAKG

SEQ ID NO: 316 - DNA, 33 bp, Artificial Sequence
  Ab20_VH CDRH3
gatagttctgctacttatgactactttaacttg

SEQ ID NO: 317 - protein, 11 aa, Artificial Sequence
  Ab20_VH CDRH3
DSSATYDYFNL

SEQ ID NO: 318 - DNA, 330 bp, Artificial Sequence
  Ab20_VL VL
tatgatatgacccagactccagcctctgtggaggtagctgtgggaggcacagtcaccatcaagtgccaggccagtcagagcattagtagctacttagcctggtatcagcagaaaccggggcagcctccccggctcctgatctatggtgcatccaatctggcgtctggggtcccatcgcggttcaaaggcagtggatctgggacagagtacactctcaccatcagcgacctggagtgtgccgatgctgccacttactactgtcaacaagccttgagtcctagtaatgttgataatgctttcggcggagggaccgaggtggtggtcaaacgt

SEQ ID NO: 319 - protein, 110 aa, Artificial Sequence
  Ab20_VL VL
YDMTQTPASVEVAVGGTVTIKCQASQSISSYLAWYQQKPGQPPRLLIYGASNLASGVPSRFKGSGSGTEYTLTISDLECADAATYYCQQALSPSNVDNAFGGGTEVVVKR

SEQ ID NO: 320 - DNA, 33 bp, Artificial Sequence; identical to SEQ ID NO: 48
  Ab20_VL CDRL1

SEQ ID NO: 321 - protein, 11 aa, Artificial Sequence
  Ab20_VL CDRL1
QASQSISSYLA

SEQ ID NO: 322 - DNA, 21 bp, Artificial Sequence
  Ab20_VL CDRL2
ggtgcatccaatctggcgtct

SEQ ID NO: 323 - protein, 7 aa, Artificial Sequence
  Ab20_VL CDRL2
GASNLAS

SEQ ID NO: 324 - DNA, 36 bp, Artificial Sequence
  Ab20_VL CDRL3
caacaagccttgagtcctagtaatgttgataatgct

SEQ ID NO: 325 - protein, 12 aa, Artificial Sequence
  Ab20_VL CDRL3
QQALSPSNVDNA

SEQ ID NO: 326 - DNA, 357 bp, Artificial Sequence
  Ab21_VH VH
cagtcgttggaggagtccgggggaggcctggtccagcctggggcatccctgacactcacctgcacagcttctggattctccttcagtagcaactactggatatgctgggtccgccaggctccagggaaggggctggagtggatcggatgcatttatactggtactggtatcacatggtacgcgagctgggtgaatggccgagtcaccatctccagcacctcgtcgaccacggtgactctgcaagtgaacagtctgacagccgcggacacggccacttatttctgtgcgagaggaagtggtgatatcgcctgggcttgggatttgtggggccaaggcaccctggtcaccgtctcgagc

SEQ ID NO: 327 - protein, 119 aa, Artificial Sequence
  Ab21_VH VH
QSLEESGGGLVQPGASLTLTCTASGFSFSSNYWICWVRQAPGKGLEWIGCIYTGTGITWYASWVNGRVTISSTSSTTVTLQVNSLTAADTATYFCARGSGDIAWAWDLWGQGTLVTVSS

SEQ ID NO: 328 - DNA, 18 bp, Artificial Sequence
  Ab21_VH CDRH1
agcaactactggatatgc

SEQ ID NO: 329 - protein, 6 aa, Artificial Sequence
  Ab21_VH CDRH1
SNYWIC

SEQ ID NO: 330 - DNA, 51 bp, Artificial Sequence
  Ab21_VH CDRH2
tgcatttatactggtactggtatcacatggtacgcgagctgggtgaatggc

SEQ ID NO: 331 - protein, 17 aa, Artificial Sequence
  Ab21_VH CDRH2
CIYTGTGITWYASWVNG

SEQ ID NO: 332 - DNA, 33 bp, Artificial Sequence
  Ab21_VH CDRH3
ggaagtggtgatatcgcctgggcttgggatttg

SEQ ID NO: 333 - protein, 11 aa, Artificial Sequence
  Ab21_VH CDRH3
GSGDIAWAWDL

SEQ ID NO: 334 - DNA, 330 bp, Artificial Sequence
  Ab21_VL VL
gatgttgtgatgacccagactccagcctccgtgtctgcagctgtgggaggcacagtcaccatcaagtgccaggccagtcagagcatttataccaatttagcctggtatcagcagaaaccagggcagcctcccaagctcctgatctacaaggcatccactctggcatctggggtctcatcgcggttcaaaggcagtggatctgggacagagttcactctcaccatcagcggcgtgcagtgtgccgatgcagccacttactactgtcaatgtactggttatggtagtagtggatggactttcggcggagggaccgaggtggtggtcaaacgt

SEQ ID NO: 335 - protein, 110 aa, Artificial Sequence
  Ab21_VL VL
DVVMTQTPASVSAAVGGTVTIKCQASQSIYTNLAWYQQKPGQPPKLLIYKASTLASGVSSRFKGSGSGTEFTLTISGVQCADAATYYCQCTGYGSSGWTFGGGTEVVVKR

SEQ ID NO: 336 - DNA, 33 bp, Artificial Sequence
  Ab21_VL CDRL1
caggccagtcagagcatttataccaatttagcc

SEQ ID NO: 337 - protein, 11 aa, Artificial Sequence
  Ab21_VL CDRL1
QASQSIYTNLA

SEQ ID NO: 338 - DNA, 21 bp, Artificial Sequence
  Ab21_VL CDRL2
aaggcatccactctggcatct

SEQ ID NO: 339 - protein, 7 aa, Artificial Sequence
  Ab21_VL CDRL2
KASTLAS

SEQ ID NO: 340 - DNA, 33 bp, Artificial Sequence
  Ab21_VL CDRL3
caatgtactggttatggtagtagtggatggact

SEQ ID NO: 341 - protein, 11 aa, Artificial Sequence
  Ab21_VL CDRL3
QCTGYGSSGWT

SEQ ID NO: 342 - DNA, 363 bp, Artificial Sequence
  Ab22_VH VH
cagtcgttggaggagtccgggggagacctggtcaagcctggggcatccctgacactcacctgcacagcctctggattctccttcagtaccagctattacatgtgctgggtccgccaggctccagggaaggggctggagtggatcggatgcatacatactagtacgttttccacatggtacgcgagctgggcgaaaggccgattcaccatctccaaaacctcgtcgaccacggtgactctgcaaatgaccagtctgacagccgcggacacggccacctatttctgtgcgagaagagatgcttataattatgctggttatgcttataacttgtggggccaggggaccctggtcaccgtctcgagc

SEQ ID NO: 343 - protein, 121 aa, Artificial Sequence
  Ab22_VH VH
QSLEESGGDLVKPGASLTLTCTASGFSFSTSYYMCWVRQAPGKGLEWIGCIHTSTFSTWYASWAKGRFTISKTSSTTVTLQMTSLTAADTATYFCARRDAYNYAGYAYNLWGQGTLVTVSS

SEQ ID NO: 344 - DNA, 18 bp, Artificial Sequence
  Ab22_VH CDRH1
accagctattacatgtgc

SEQ ID NO: 345 - protein, 6 aa, Artificial Sequence
  Ab22_VH CDRH1
TSYYMC

SEQ ID NO: 346 - DNA, 51 bp, Artificial Sequence
  Ab22_VH CDRH2
tgcatacatactagtacgttttccacatggtacgcgagctgggcgaaaggc

SEQ ID NO: 347 - protein, 17 aa, Artificial Sequence
  Ab22_VH CDRH2
CIHTSTFSTWYASWAKG

SEQ ID NO: 348 - DNA, 39 bp, Artificial Sequence
  Ab22_VH CDRH3
agagatgcttataattatgctggttatgcttataacttg

SEQ ID NO: 349 - protein, 13 aa, Artificial Sequence
  Ab22_VH CDRH3
RDAYNYAGYAYNL

SEQ ID NO: 350 - DNA, 339 bp, Artificial Sequence
  Ab22_VL VL
gatgttgtgatgacccagactccagcctcggtgtctgccgctgtgggaggcacagtcaccatcaattgccaggccagtgaggacatttatagcaatttggcctggtatcagcagaaaccagggcagcctcccaagctcctgatctatggtgcatccactctggcatctggggtcccatcgcggttcaaaggcagtggatctgggacagagttcactctcaccatcagcgacctggagtgtgccgatgctgccacttactactgtcaatgcacttttggttgttttagtggtagtagtaacaacattttcggcggagggaccgaggtggtggtcaaacgt

SEQ ID NO: 351 - protein, 113 aa, Artificial Sequence
  Ab22_VL VL
DVVMTQTPASVSAAVGGTVTINCQASEDIYSNLAWYQQKPGQPPKLLIYGASTLASGVPSRFKGSGSGTEFTLTISDLECADAATYYCQCTFGCFSGSSNNIFGGGTEVVVKR

SEQ ID NO: 352 - DNA, 33 bp, Artificial Sequence
  Ab22_VL CDRL1
caggccagtgaggacatttatagcaatttggcc

SEQ ID NO: 353 - protein, 11 aa, Artificial Sequence
  Ab22_VL CDRL1
QASEDIYSNLA

SEQ ID NO: 354 - DNA, 21 bp, Artificial Sequence
  Ab22_VL CDRL2
ggtgcatccactctggcatct

SEQ ID NO: 355 - protein, 7 aa, Artificial Sequence
  Ab22_VL CDRL2
GASTLAS

SEQ ID NO: 356 - DNA, 42 bp, Artificial Sequence
  Ab22_VL CDRL3
caatgcacttttggttgttttagtggtagtagtaacaacatt

SEQ ID NO: 357 - protein, 14 aa, Artificial Sequence
  Ab22_VL CDRL3
QCTFGCFSGSSNNI

SEQ ID NO: 358 - DNA, 360 bp, Artificial Sequence
  Ab23_VH VH
cagcagcagctggaggagtccgggggaggcctggtcaagcctggaggaaccctgacactcacctgcaaagcctctggaatcgacttcaatgactactactacatgtgctgggtccgccaggctccagggaaggggcctgagtggatcgcatgcatttataatggtgatggtgacacttactacgcgaactgggcgaaaggccgattcaccatctccaaaacctcgtcgaccacggtgactctgcaaatgaccagtctgacagccgcggacacggccacctatttctgtgcgaggggtagtggtgttggtagttatggttacaacttgtggggccaaggcaccctggtcaccgtctcgagc

SEQ ID NO: 359 - protein, 120 aa, Artificial Sequence
  Ab23_VH VH
QQQLEESGGGLVKPGGTLTLTCKASGIDFNDYYYMCWVRQAPGKGPEWIACIYNGDGDTYYANWAKGRFTISKTSSTTVTLQMTSLTAADTATYFCARGSGVGSYGYNLWGQGTLVTVSS

SEQ ID NO: 360 - DNA, 18 bp, Artificial Sequence
  Ab23_VH CDRH1
gactactactacatgtgc

SEQ ID NO: 361 - protein, 6 aa, Artificial Sequence
  Ab23_VH CDRH1
DYYYMC

SEQ ID NO: 362 - DNA, 51 bp, Artificial Sequence
  Ab23_VH CDRH2
tgcatttataatggtgatggtgacacttactacgcgaactgggcgaaaggc

SEQ ID NO: 363 - protein, 17 aa, Artificial Sequence
  Ab23_VH CDRH2
CIYNGDGDTYYANWAKG

SEQ ID NO: 364 - DNA, 33 bp, Artificial Sequence; identical to SEQ ID NO: 28
  Ab23_VH CDRH3

SEQ ID NO: 365 - protein, 11 aa, Artificial Sequence
  Ab23_VH CDRH3
GSGVGSYGYNL

SEQ ID NO: 366 - DNA, 333 bp, Artificial Sequence
  Ab23_VL VL
gctgacattgtgatgacccagactccagcctccgtggaggcagctgtgggaggcacagtcaccatcaagtgccaggccagtgagagcatttacagcaatttagcctggtatcagcagaaaccagggcagcctcccaagctcctaatctacagggcatctactctggcatctggggtcccatcgcggttcaaaggcagtggatctgggacagagttcactctcaccatcagcgacctggagtgtgccgatgctgccacttactactgtcaaggctattatgccagtactattactaatgttttcggcggagggaccgaggtggtggtcaaacgt

SEQ ID NO: 367 - protein, 111 aa, Artificial Sequence
  Ab23_VL VL
ADIVMTQTPASVEAAVGGTVTIKCQASESIYSNLAWYQQKPGQPPKLLIYRASTLASGVPSRFKGSGSGTEFTLTISDLECADAATYYCQGYYASTITNVFGGGTEVVVKR

SEQ ID NO: 368 - DNA, 33 bp, Artificial Sequence; identical to SEQ ID NO: 64
  Ab23_VL CDRL1

SEQ ID NO: 369 - protein, 11 aa, Artificial Sequence
  Ab23_VL CDRL1
QASESIYSNLA

SEQ ID NO: 370 - DNA, 21 bp, Artificial Sequence
  Ab23_VL CDRL2
agggcatctactctggcatct

SEQ ID NO: 371 - protein, 7 aa, Artificial Sequence
  Ab23_VL CDRL2
RASTLAS

SEQ ID NO: 372 - DNA, 33 bp, Artificial Sequence
  Ab23_VL CDRL3
caaggctattatgccagtactattactaatgtt

SEQ ID NO: 373 - protein, 11 aa, Artificial Sequence
  Ab23_VL CDRL3
QGYYASTITNV

SEQ ID NO: 374 - DNA, 360 bp, Artificial Sequence
  Ab24_VH VH
cagcagcagctggaggagtccgggggaggcctggtcaagcctggaggaaccctgacactcacctgcaaagcctctggaatcgacttcagtaacgtctactacatgtgctgggtccgccaggctccagggaaggggccggagtggatcgcatgcatttatgttggtgatggcagcacatacgacgcgagctgggcgagaggccgattcaccatctccaaaacctcgtcgaccacggtgactctgcagatgattagtctgacagccgcggacacggccacctatttctgtgcggggggtactggtgttggtagctatggttacagcttgtggggcccaggcaccctggtcaccgtctcgagc

SEQ ID NO: 375 - protein, 120 aa, Artificial Sequence
  Ab24_VH VH
QQQLEESGGGLVKPGGTLTLTCKASGIDFSNVYYMCWVRQAPGKGPEWIACIYVGDGSTYDASWARGRFTISKTSSTTVTLQMISLTAADTATYFCAGGTGVGSYGYSLWGPGTLVTVSS

SEQ ID NO: 376 - DNA, 18 bp, Artificial Sequence
  Ab24_VH CDRH1
aacgtctactacatgtgc

SEQ ID NO: 377 - protein, 6 aa, Artificial Sequence
  Ab24_VH CDRH1
NVYYMC

SEQ ID NO: 378 - DNA, 51 bp, Artificial Sequence
  Ab24_VH CDRH2
tgcatttatgttggtgatggcagcacatacgacgcgagctgggcgagaggc

SEQ ID NO: 379 - protein, 17 aa, Artificial Sequence
  Ab24_VH CDRH2
CIYVGDGSTYDASWARG

SEQ ID NO: 380 - DNA, 33 bp, Artificial Sequence
  Ab24_VH CDRH3
ggtactggtgttggtagctatggttacagcttg

SEQ ID NO: 381 - protein, 11 aa, Artificial Sequence
  Ab24_VH CDRH3
GTGVGSYGYSL

SEQ ID NO: 382 - DNA, 333 bp, Artificial Sequence
  Ab24_VL VL
gctgacattgtgatgacccagactccagcctccgtggaggcagctgtgggaggcacagtcaccatcaagtgccaggccagtgagagcatttatagcaatttagcctggtatcatcagaaaccagggcagcctcccaagctcctgatctacagggcatccaatctggaatctggggtcccatcgcggttcaaaggcagtggatctgggacagatttcactctcaccatcagcgacctggagtgtgccgatgctgccacttactactgtcaaggctattatgccagtgttgttacaaatggtttcggcggagggaccgaggtggtggtcaaacgt

SEQ ID NO: 383 - protein, 111 aa, Artificial Sequence
  Ab24_VL VL
ADIVMTQTPASVEAAVGGTVTIKCQASESIYSNLAWYHQKPGQPPKLLIYRASNLESGVPSRFKGSGSGTDFTLTISDLECADAATYYCQGYYASVVTNGFGGGTEVVVKR

SEQ ID NO: 384 - DNA, 33 bp, Artificial Sequence; identical to SEQ ID NO: 16
  Ab24_VL CDRL1

SEQ ID NO: 385 - protein, 11 aa, Artificial Sequence
  Ab24_VL CDRL1
QASESIYSNLA

SEQ ID NO: 386 - DNA, 21 bp, Artificial Sequence
  Ab24_VL CDRL2
agggcatccaatctggaatct

SEQ ID NO: 387 - protein, 7 aa, Artificial Sequence
  Ab24_VL CDRL2
RASNLES

SEQ ID NO: 388 - DNA, 33 bp, Artificial Sequence
  Ab24_VL CDRL3
caaggctattatgccagtgttgttacaaatggt

SEQ ID NO: 389 - protein, 11 aa, Artificial Sequence
  Ab24_VL CDRL3
QGYYASVVTNG

SEQ ID NO: 390 - DNA, 360 bp, Artificial Sequence
  Ab25_VH VH
cagcggcagctggaggagtccgggggaggcctggtcaagcctggaggagccctgacactcacctgcaaagcctctggaatcgacttcagtagctactactacagatgctgggtccgccaggctccagggaaggggctggagtggatcgcatgcatttatactggtgatggtaccacttactacgcgagctgggcgaaaggccgattcaccacctccaaaacctcgtcgaccacggtgactctgcaaatgaccagtctgacagccgcggacacggccacctatttctgtgcgaggggtagtggtgttggtagttatggttacaacttctggggcccaggcaccctggtcaccgtctcgagc

SEQ ID NO: 391 - protein, 120 aa, Artificial Sequence
  Ab25_VH VH
QRQLEESGGGLVKPGGALTLTCKASGIDFSSYYYRCWVRQAPGKGLEWIACIYTGDGTTYYASWAKGRFTTSKTSSTTVTLQMTSLTAADTATYFCARGSGVGSYGYNFWGPGTLVTVSS

SEQ ID NO: 392 - DNA, 18 bp, Artificial Sequence
  Ab25_VH CDRH1
agctactactacagatgc

SEQ ID NO: 393 - protein, 6 aa, Artificial Sequence
  Ab25_VH CDRH1
SYYYRC

SEQ ID NO: 394 - DNA, 51 bp, Artificial Sequence
  Ab25_VH CDRH2
tgcatttatactggtgatggtaccacttactacgcgagctgggcgaaaggc

SEQ ID NO: 395 - protein, 17 aa, Artificial Sequence
  Ab25_VH CDRH2
CIYTGDGTTYYASWAKG

SEQ ID NO: 396 - DNA, 33 bp, Artificial Sequence
  Ab25_VH CDRH3
ggtagtggtgttggtagttatggttacaacttc

SEQ ID NO: 397 - protein, 11 aa, Artificial Sequence
  Ab25_VH CDRH3
GSGVGSYGYNF

SEQ ID NO: 398 - DNA, 333 bp, Artificial Sequence; identical to SEQ ID NO: 286
  Ab25_VL VL

SEQ ID NO: 399 - protein, 111 aa, Artificial Sequence; identical to SEQ ID NO: 287
  Ab25_VL VL

SEQ ID NO: 400 - DNA, 33 bp, Artificial Sequence; identical to SEQ ID NO: 16
  Ab25_VL CDRL1

SEQ ID NO: 401 - protein, 11 aa, Artificial Sequence
  Ab25_VL CDRL1
QASESIYSNLA

SEQ ID NO: 402 - DNA, 21 bp, Artificial Sequence
  Ab25_VL CDRL2
agggcatccactctggaatct

SEQ ID NO: 403 - protein, 7 aa, Artificial Sequence
  Ab25_VL CDRL2
RASTLES

SEQ ID NO: 404 - DNA, 33 bp, Artificial Sequence; identical to SEQ ID NO: 292
  Ab25_VL CDRL3

SEQ ID NO: 405 - protein, 11 aa, Artificial Sequence
  Ab25_VL CDRL3
QGYYASIITNA

SEQ ID NO: 406 - DNA, 357 bp, Artificial Sequence
  Ab26_VH VH
cagtcggtggaggagtccgggggaggcctggtcaagcctgagggatccctgacactcacctgcaaagcctctggattcgacctcagtagctactactacatgtgctgggtccgccaggctccagggaaggggccggagtggatcgcatgcatttatgctggtgatggcaccacatactacgcgagctgggcgaaaggccgattcaccatctccaaaacctcgtcgaccacggtgactctgcaaatgaccagtctgacagccgcggacacggccacctatttctgtgcgaggggtactggtgttggtagctatggttacaacttgtggggcccaggcaccctggtcaccgtctcgagc

SEQ ID NO: 407 - protein, 119 aa, Artificial Sequence
  Ab26_VH VH
QSVEESGGGLVKPEGSLTLTCKASGFDLSSYYYMCWVRQAPGKGPEWIACIYAGDGTTYYASWAKGRFTISKTSSTTVTLQMTSLTAADTATYFCARGTGVGSYGYNLWGPGTLVTVSS

SEQ ID NO: 408 - DNA, 18 bp, Artificial Sequence
  Ab26_VH CDRH1
agctactactacatgtgc

SEQ ID NO: 409 - protein, 6 aa, Artificial Sequence
  Ab26_VH CDRH1
SYYYMC

SEQ ID NO: 410 - DNA, 51 bp, Artificial Sequence
  Ab26_VH CDRH2
tgcatttatgctggtgatggcaccacatactacgcgagctgggcgaaaggc

SEQ ID NO: 411 - protein, 17 aa, Artificial Sequence
  Ab26_VH CDRH2
CIYAGDGTTYYASWAKG

SEQ ID NO: 412 - DNA, 33 bp, Artificial Sequence
  Ab26_VH CDRH3
ggtactggtgttggtagctatggttacaacttg

SEQ ID NO: 413 - protein, 11 aa, Artificial Sequence
  Ab26_VH CDRH3
GTGVGSYGYNL

SEQ ID NO: 414 - DNA, 333 bp, Artificial Sequence
  Ab26_VL VL
gctgacattgtgatgacccagactccagcctccgtggaggcagctgtgggaggcacagtcaccatcaagtgccaggccagtgagaacatttacagcaatttagcctggtatcagcagaaaccagggcagcctcccaagctcctaatctacagggcatccaatctggaatctggggtcccatcgcggttcaaaggcagtggatctgggacagagttcactctcaccatcagcgacctggagtgtgacgatgctgccacttactactgtcaaggctattatcccagtgctgttactaatgctttcggcggagggaccgaggtggtggtcaaacgt

SEQ ID NO: 415 - protein, 111 aa, Artificial Sequence
  Ab26_VL VL
ADIVMTQTPASVEAAVGGTVTIKCQASENIYSNLAWYQQKPGQPPKLLIYRASNLESGVPSRFKGSGSGTEFTLTISDLECDDAATYYCQGYYPSAVTNAFGGGTEVVVKR

SEQ ID NO: 416 - DNA, 33 bp, Artificial Sequence; identical to SEQ ID NO: 240
  Ab26_VL CDRL1

SEQ ID NO: 417 - protein, 11 aa, Artificial Sequence
  Ab26_VL CDRL1
QASENIYSNLA

SEQ ID NO: 418 - DNA, 21 bp, Artificial Sequence
  Ab26_VL CDRL2
agggcatccaatctggaatct

SEQ ID NO: 419 - protein, 7 aa, Artificial Sequence
  Ab26_VL CDRL2
RASNLES

SEQ ID NO: 420 - DNA, 33 bp, Artificial Sequence
  Ab26_VL CDRL3
caaggctattatcccagtgctgttactaatgct

SEQ ID NO: 421 - protein, 11 aa, Artificial Sequence
  Ab26_VL CDRL3
QGYYPSAVTNA

SEQ ID NO: 422 - DNA, 357 bp, Artificial Sequence
  Ab27_VH VH
cagtcggtggaggagtccgggggaggcctggtcaagcctggaggaaccctgacactcacctgcaaagcctctggaatcgacttcagtagttaccactacatgtgctgggtccgccaggctccagggaaggggctggagtggatcgcgtgcatttataatggtgatggtgacacttactacgcgagctgggcgaaaggccgattcaccatctccaaaacctcgtcgaccacggtgactctgcaaatgaccagtctgacagccgcggacacggccacctatttctgtgcgaggggtagtggtgttggtagttatggttacaatttgtggggcccaggcaccctggtcaccgtctcgagc

SEQ ID NO: 423 - protein, 119 aa, Artificial Sequence
  Ab27_VH VH
QSVEESGGGLVKPGGTLTLTCKASGIDFSSYHYMCWVRQAPGKGLEWIACIYNGDGDTYYASWAKGRFTISKTSSTTVTLQMTSLTAADTATYFCARGSGVGSYGYNLWGPGTLVTVSS

SEQ ID NO: 424 - DNA, 18 bp, Artificial Sequence
  Ab27_VH CDRH1
agttaccactacatgtgc

SEQ ID NO: 425 - protein, 6 aa, Artificial Sequence
  Ab27_VH CDRH1
SYHYMC

SEQ ID NO: 426 - DNA, 51 bp, Artificial Sequence
  Ab27_VH CDRH2
tgcatttataatggtgatggtgacacttactacgcgagctgggcgaaaggc

SEQ ID NO: 427 - protein, 17 aa, Artificial Sequence
  Ab27_VH CDRH2
CIYNGDGDTYYASWAKG

SEQ ID NO: 428 - DNA, 33 bp, Artificial Sequence
  Ab27_VH CDRH3
ggtagtggtgttggtagttatggttacaatttg

SEQ ID NO: 429 - protein, 11 aa, Artificial Sequence
  Ab27_VH CDRH3
GSGVGSYGYNL

SEQ ID NO: 430 - DNA, 333 bp, Artificial Sequence
  Ab27_VL VL
gctgacattgtgatgacccagactccagcctccgtggaggcagctgtgggaggcacagtcaccatcaagtgccaggccagtgagagcatttacagcaatttagcctggtatcagcagaaaccagggcagcctcccaagctcctaatctacagggcatccaatctggaatctggggtcccatcgcggttcaaaggcagtggatctgggacagagttcactctcaccatcagcgacctggagtgtgccgatgctgccacttactactgtcaaggctattatgccagtactactactaatgctttcggcggagggaccgaggtggtggtcaaacgt

SEQ ID NO: 431 - protein, 111 aa, Artificial Sequence; identical to SEQ ID NO: 15
  Ab27_VL VL

SEQ ID NO: 432 - DNA, 33 bp, Artificial Sequence; identical to SEQ ID NO: 64
  Ab27_VL CDRL1

SEQ ID NO: 433 - protein, 11 aa, Artificial Sequence
  Ab27_VL CDRL1
QASESIYSNLA

SEQ ID NO: 434 - DNA, 21 bp, Artificial Sequence
  Ab27_VL CDRL2
agggcatccaatctggaatct

SEQ ID NO: 435 - protein, 7 aa, Artificial Sequence
  Ab27_VL CDRL2
RASNLES

SEQ ID NO: 436 - DNA, 33 bp, Artificial Sequence; identical to SEQ ID NO: 20
  Ab27_VL CDRL3

SEQ ID NO: 437 - protein, 11 aa, Artificial Sequence
  Ab27_VL CDRL3
QGYYASTTTNA

SEQ ID NO: 438 - DNA, 360 bp, Artificial Sequence
  Ab28_VH VH
cagcagcagctggaggagtccgggggaggcctggtcaagcctggaggaaccctgacactcacctgcaaaggctctggaatcgacttcaatgactactactacatgtgctgggtccgccaggctccagggaaggggctggagtggatcgcatgcatttataatggtgatggtgagacttactacgcgaactgggcgaaaggccgattcaccatctccaaaacctcgtcgaccacggtgactctgcagatgaccagtctgacagccgcggacacggccacctatttctgtgcgaggggtagtggtgttgggagttatggttacaacttttggggcccaggcaccctggtcaccgtctcgagc

SEQ ID NO: 439 - protein, 120 aa, Artificial Sequence
  Ab28_VH VH
QQQLEESGGGLVKPGGTLTLTCKGSGIDFNDYYYMCWVRQAPGKGLEWIACIYNGDGETYYANWAKGRFTISKTSSTTVTLQMTSLTAADTATYFCARGSGVGSYGYNFWGPGTLVTVSS

SEQ ID NO: 440 - DNA, 18 bp, Artificial Sequence
  Ab28_VH CDRH1
gactactactacatgtgc

SEQ ID NO: 441 - protein, 6 aa, Artificial Sequence
  Ab28_VH CDRH1
DYYYMC

SEQ ID NO: 442 - DNA, 51 bp, Artificial Sequence; identical to SEQ ID NO: 218
  Ab28_VH CDRH2

SEQ ID NO: 443 - protein, 17 aa, Artificial Sequence
  Ab28_VH CDRH2
CIYNGDGETYYANWAKG

SEQ ID NO: 444 - DNA, 33 bp, Artificial Sequence; identical to SEQ ID NO: 60
  Ab28_VH CDRH3

SEQ ID NO: 445 - protein, 11 aa, Artificial Sequence
  Ab28_VH CDRH3
GSGVGSYGYNF

SEQ ID NO: 446 - DNA, 333 bp, Artificial Sequence; identical to SEQ ID NO: 222
  Ab28_VL VL

SEQ ID NO: 447 - protein, 111 aa, Artificial Sequence; identical to SEQ ID NO: 223
  Ab28_VL VL

SEQ ID NO: 448 - DNA, 33 bp, Artificial Sequence; identical to SEQ ID NO: 64
  Ab28_VL CDRL1

SEQ ID NO: 449 - protein, 11 aa, Artificial Sequence
  Ab28_VL CDRL1
QASESIYSNLA

SEQ ID NO: 450 - DNA, 21 bp, Artificial Sequence
  Ab28_VL CDRL2
agggcatccaatctggcatct

SEQ ID NO: 451 - protein, 7 aa, Artificial Sequence
  Ab28_VL CDRL2
RASNLAS

SEQ ID NO: 452 - DNA, 33 bp, Artificial Sequence; identical to SEQ ID NO: 228
  Ab28_VL CDRL3

SEQ ID NO: 453 - protein, 11 aa, Artificial Sequence
  Ab28_VL CDRL3
QGYYASTVTNA

SEQ ID NO: 454 - DNA, 360 bp, Artificial Sequence
  Ab29_VH VH
cagcagcagctggaggagtccgggggaggcctggtcaagcctggaggaaccctgacactcacctgcaaagcctctggaatcgacttcagtagttaccactacatgtgctgggtccgccaggctccagggaaggggctggagtggatcgcgtgcatttataatggtgatggtgacacttactacgcgagctgggcgaaaggccgattcaccatctccaaaacctcgtcgaccacggtgactctgcaaatgaccagtctgacagccgcggacacggccacctatttctgtgcgaggggtagtggtgttggtagttatggttacaatttgtggggcccaggcaccctggtcaccgtctcgagc

SEQ ID NO: 455 - protein, 120 aa, Artificial Sequence
  Ab29_VH VH
QQQLEESGGGLVKPGGTLTLTCKASGIDFSSYHYMCWVRQAPGKGLEWIACIYNGDGDTYYASWAKGRFTISKTSSTTVTLQMTSLTAADTATYFCARGSGVGSYGYNLWGPGTLVTVSS

SEQ ID NO: 456 - DNA, 18 bp, Artificial Sequence
  Ab29_VH CDRH1
agttaccactacatgtgc

SEQ ID NO: 457 - protein, 6 aa, Artificial Sequence
  Ab29_VH CDRH1
SYHYMC

SEQ ID NO: 458 - DNA, 51 bp, Artificial Sequence; identical to SEQ ID NO: 426
  Ab29_VH CDRH2

SEQ ID NO: 459 - protein, 17 aa, Artificial Sequence
  Ab29_VH CDRH2
CIYNGDGDTYYASWAKG

SEQ ID NO: 460 - DNA, 33 bp, Artificial Sequence; identical to SEQ ID NO: 428
  Ab29_VH CDRH3

SEQ ID NO: 461 - protein, 11 aa, Artificial Sequence
  Ab29_VH CDRH3
GSGVGSYGYNL

SEQ ID NO: 462 - DNA, 333 bp, Artificial Sequence; identical to SEQ ID NO: 414
  Ab29_VL VL

SEQ ID NO: 463 - protein, 111 aa, Artificial Sequence; identical to SEQ ID NO: 415
  Ab29_VL VL

SEQ ID NO: 464 - DNA, 33 bp, Artificial Sequence; identical to SEQ ID NO: 240
  Ab29_VL CDRL1

SEQ ID NO: 465 - protein, 11 aa, Artificial Sequence
  Ab29_VL CDRL1
QASENIYSNLA

SEQ ID NO: 466 - DNA, 21 bp, Artificial Sequence
  Ab29_VL CDRL2
agggcatccaatctggaatct

SEQ ID NO: 467 - protein, 7 aa, Artificial Sequence
  Ab29_VL CDRL2
RASNLES

SEQ ID NO: 468 - DNA, 33 bp, Artificial Sequence; identical to SEQ ID NO: 420
  Ab29_VL CDRL3

SEQ ID NO: 469 - protein, 11 aa, Artificial Sequence
  Ab29_VL CDRL3
QGYYPSAVTNA